(19) United States
(12) Patent Application Publication
(10) Pub. No.: US 2024/0197902 A1
(43) Pub. Date: Jun. 20, 2024

(54) ACTRII-ALK4 ANTAGONISTS AND METHODS OF TREATING HEART FAILURE

(71) Applicant: Acceleron Pharma Inc., Rahway, NJ (US)

(72) Inventors: Rajasekhar Naga Venkata Sai Suragani, Wrentham, MA (US); Jia Li, Danvers, MA (US); Patrick Andre, Short Hills, NJ (US); Ravindra Kumar, Concord, MA (US)

(73) Assignee: Acceleron Pharma Inc., Rahway, NJ (US)

(21) Appl. No.: 18/549,461

(22) PCT Filed: Mar. 9, 2022
(86) PCT No.: PCT/US2022/019557

§ 371 Date: Sep. 7, 2023

Related U.S. Application Data
(60) Provisional application No. 63/159,059, filed on Mar. 10, 2021.

(51) Int. Cl. A61K 47/68; A61P 9/04

(52) U.S. Cl. CPC A61K 47/6811; A61P 9/04

(57) ABSTRACT

In some aspects, the disclosure relates to ActRII-ALK4 antagonists and methods of using ActRII-ALK4 antagonists to treat, prevent, or reduce the progression rate and/or severity of heart failure (HF), particularly treating, preventing or reducing the progression rate and/or severity of one or more HF-associated comorbidities. The disclosure also provides methods of using an ActRII-ALK4 antagonist to treat, prevent, or reduce the progression rate and/or severity of heart failure associated with a variety of conditions including, but not limited to, heart failure associated with aging.

### CROSS-REFERENCE TO RELATED APPLICATIONS

This application claims the benefit of priority from U.S. Provisional Application No. 63/159,059, filed Mar. 10, 2021. The specification of the foregoing application is incorporated herein by reference in its entirety.

### BACKGROUND

The prevalence of heart failure (HF) depends on the definition applied, but it affects approximately 1-2% of the adult population in developed countries, rising to ≥10% among people 70 years of age. Among people 65 years of age presenting to primary care with breathlessness on exertion, one in six will have unrecognized HF (mainly HFpEF). The lifetime risk of HF at age 55 years is 33% for men and 28% for women. The proportion of patients with HFpEF ranges from 22 to 73%, depending on the definition applied, the clinical setting (primary care, hospital clinic, and hospital admission), age and sex of the studied population, previous myocardial infarction and the year of publication.

With 50% of all heart failure diagnoses and 90% of all heart failure deaths occurring in adults over the age of 70, heart failure is undeniably tied to aging. The Framingham Heart Study found a prevalence of HF in men of 8 per 1000 at age 50 to 59 years, increasing to 66 per 1000 at ages 80 to 89 years; and similar values (8 and 79 per 1000) were noted in women. The prevalence in African-American populations is reported to be 25 percent higher than in white populations. While aging in and of itself is not a cause of heart failure, age does lower the threshold for manifestation of the disease. With the success of treatment options for ischemic and valvular diseases, there is an increasing number of older individuals with some degree of cardiac damage, which are increasingly imperiled by the diminished cardiac reserve associated with normal aging. Commonly, heart failure in aging patients falls under the umbrella of heart failure with preserved ejection fraction (HFpEF). Currently, there is no approved therapy specifically for HFpEF.

Therefore, there is a high, unmet need for effective therapies for treating heart failure associated with aging. Accordingly, it is an object of the present disclosure to provide methods for treating, preventing, or reducing the progression rate and/or severity of heart failure, particularly treating, preventing or reducing the progression rate and/or severity of one or more heart failure-associated comorbidities.

### SUMMARY

As demonstrated herein, an ActRII-ALK4 antagonist is effective in treating heart failure. In particular, an ActRIIB-ALK4 heterodimer protein demonstrated cardio-protective effects in a murine model of physiological cardiac aging using aged C57BL6 mice, displaying characteristics of heart failure associated with preserved ejection fraction (HFpEF). For example, data presented herein shows that treatment with an ActRIIB-ALK4 heterodimer has positive effects on various complications associated with this heart failure model including, but not limited to, LV contractility, hypertrophy, LV wall thickness, heart weight, systolic function, and serum biomarkers of cardiac injury (e.g., cTnI serum levels). While not wishing to be bound to any particular mechanism, it is expected that the effects of the ActRIIB-ALK4 heterodimer on heart failure is caused primarily by antagonizing ligand-signaling as mediated by one or more ligands that bind to the ActRIIB-ALK4 heterodimer protein including, but not limited to, activin A, activin B, GDF8, GDF11, BMP6, and/or BMP10 (referred to herein as “ActRII-ALK4 ligands” or “ActRII-ALK4 ligand”). Regardless of the mechanism, it is apparent from the data presented herein that ActRIIB-ALK4 heterodimers have significant positive effects in ameliorating various complications associated with heart failure and further suggests that other ActRII-ALK4 antagonists may also be useful in treating heart failure associated with aging.

As disclosed herein, the term “ActRII-ALK4 antagonist” refers a variety of agents that may be used to inhibit signaling by one or more ActRII-ALK4 ligands including, for example, antagonists that inhibit one or more ActRII-ALK4 ligands (e.g., activin A, activin B, GDF8, GDF11, BMP6, and/or BMP10); antagonists that inhibit one or more ActRII-ALK4 ligand associated receptors (e.g., ActRIIA, ActRIIB, ALK4, and ALK7); and antagonists that inhibit one or more downstream signaling components (e.g., Smad proteins such as Smads 2 and 3). ActRII-ALK4 antagonists to be used in accordance with the methods and uses of the disclosure include a variety of forms, for example, ActRII-ALK4 ligand traps (e.g., soluble ActRIIA polypeptides or ActRIIB polypeptides including variants as well as heteromultimers and homomultimers thereof), ActRII-ALK4 antibody antagonists (e.g., antibodies that inhibit one or more of activin A, activin B, GDF8, GDF11, BMP6, BMP10, ActRIIB, ActRIIA, ALK4 and/or ALK7), small molecule antagonists (e.g., small molecules that inhibit one or more of activin A, activin B, GDF8, GDF11, BMP6, BMP10, ActRIIB, ActRIIA, ALK4 and/or ALK7) and nucleotide antagonists (e.g., nucleotide sequences that inhibit one or more of activin A, activin B, GDF8, GDF11, BMP6, BMP10, ActRIIB, ActRIIA, ALK4 and/or ALK7).

In certain aspects, the disclosure provides ActRII-ALK4 antagonists comprising soluble ActRIIB, ActRIIA, ALK4, ALK7, or follistatin polypeptides to antagonize the signaling of ActRII-ALK4 ligands generally, in any process associated with heart failure associated with aging. ActRII-ALK4 antagonists of the disclosure may antagonize one or more ligands of ActRII-ALK4, such as activin A, activin B, GDF8, GDF11, BMP6, or BMP10, and may therefore be useful in treating, preventing, or reducing the progression rate and/or severity of heart failure associated with aging, or one or more comorbidities of heart failure (e.g. anemia, angina, arterial hypertension, arthritis, atrial fibrillation, cachexia, cancer, cognitive dysfunction, coronary artery disease (CAD), diabetes, erectile dysfunction, gout, hypercholesterolemia, hyperkalemia, hyperkalemia, hyperlipidemia, hypertension, iron deficiency, kidney dysfunction, metabolic syndrome, obesity, physical deconditioning, potassium disorders, pulmonary disease (e.g., asthma, COPD), sarcopenia, sleep apnea, sleep disturbance, and valvular heart disease (e.g., aortic stenosis, aortic regurgitation, mitral regurgitation, tricuspid regurgitation)).

In certain aspects, an ActRII-ALK4 antagonist to be used in accordance with the methods and uses disclosed herein (e.g., treating, preventing, or reducing the progression rate and/or severity of heart failure associated with aging, or one or more complications of heart failure) is an ActRII-ALK4 ligand trap polypeptide antagonist including variants thereof as well as heterodimers and heteromultimers thereof, an ActRII-ALK4 antibody antagonist, an ActRII-ALK4 polynucleotide antagonist, and/or an ActRII-ALK4 small molecule antagonist. ActRII-ALK4 ligand trap polypeptides include TGF-β superfamily-related proteins, including variants thereof, that are capable of binding to one or more ActRII-ALK4 ligands (e.g., activin A, activin B, GDF8, GDF11, BMP6, BMP10). Therefore, an ActRII-ALK4 ligand trap generally includes polypeptides that are capable of antagonizing one or more ActRII-ALK4 ligands (e.g., activin A, activin B, GDF8, GDF11, BMP6, BMP10). As used herein, the term “ActRII” refers to the family of type II activin receptors. This family includes activin receptor type IIA (ActRIIA) and activin receptor type IIB (ActRIIB). In some embodiments, an ActRII-ALK4 antagonist comprises an ActRII-ALK4 ligand trap. In some embodiments, an ActRII-ALK4 ligand trap comprises an ActRIIB polypeptide, including variants thereof, as well as homomultimers (e.g., ActRIIB homodimers) and heteromultimers (e.g., ActRIIB-ALK4 or ActRIIB-ALK7 heterodimers). In some embodiments, an ActRII-ALK4 ligand trap comprises an ActRIIA polypeptide, including variants thereof, as well as homomultimers (e.g., ActRIIA homodimers) and heteromultimers (e.g., ActRIIA-ALK4 or ActRIIA-ALK7 heterodimers). In other embodiments, an ActRII-ALK ligand trap comprises a soluble ligand trap protein including, but not limited to, or a follistatin polypeptide as well as variants thereof. In some embodiments, an ActRII-ALK4 antagonist comprises an ActRII-ALK4 antibody antagonist. In some embodiments, an ActRII-ALK4 antagonist comprises an ActRII-ALK4 small molecule antagonist. In some embodiments, an ActRII-ALK4 antagonist comprises an ActRII-ALK4 polynucleotide antagonist.

In part, the disclosure provides methods of treating heart failure associated with aging, comprising administering to a patient in need thereof an effective amount of an ActRII-ALK4 antagonist. The disclosure also provides methods of treating, preventing, or reducing the progression rate and/or severity of one or more comorbidities of heart failure associated with aging, comprising administering to a patient in need thereof an effective amount of an ActRII-ALK4 antagonist.

In some embodiments, the patient is at least 40 years old. In some embodiments, the patient is between about 40 and about 100 years old.

In some embodiments of the present disclosure, the heart failure is heart failure with preserved ejection fraction (HFpEF). In some embodiments, a patient has a left ventricular ejection fraction (LVEF) of ≥50%. In some embodiments, the patient has normal systolic function.

In some embodiments of the present disclosure, the patient has dyspnea. In some embodiments, methods of the present disclosure decrease dyspnea.

In some embodiments of the present disclosure, the patient has cardiovascular structural remodeling selected from the group consisting of an increase in vascular intimal thickness, an increase in vascular stiffness, an increase in left ventricular (LV) hypertrophy, and an increase in left atrial enlargement. In some embodiments, methods of the present disclosure improve cardiovascular structural remodeling in the patient selected from the group consisting of an increase in vascular intimal thickness, an increase in vascular stiffness, an increase in LV hypertrophy, and an increase in left atrial enlargement.

In some embodiments of the present disclosure, the patient has LV hypertrophy. In some embodiments, methods of the present disclosure decrease LV hypertrophy in the patient. In some embodiments, the method decreases left ventricular hypertrophy in the patient, wherein the patient's LV hypertrophy is decreased by at least 1% (e.g., 1%, 5%, 10%, 15%, 20%, 25%, 30%, 35%, 40%, 45%, or at least 50%).

In some embodiments, methods of the present disclosure decrease cardiac filling pressure in the patient. In some embodiments, the method improves early diastolic cardiac filling in the patient.

In some embodiments of the present disclosure, the patient has left atrial enlargement. In some embodiments, methods of the present disclosure decrease atrial enlargement in the patient. In some embodiments, the method decreases left atrial enlargement in the patient, wherein the patient's left atrial enlargement is decreased by at least 1% (e.g., 1%, 5%, 10%, 15%, 20%, 25%, 30%, 35%, 40%, 45%, or at least 50%).

In some embodiments, methods of the present disclosure decrease vascular intimal thickness in the patient.

In some embodiments, methods of the present disclosure decrease vascular stiffness in the patient.

In some embodiments of the present disclosure, the patient has a change in ventricular structure in the heart, selected from the group consisting of LV hypertrophy, an increase in cardiomyocyte size, a loss of cardiomyocytes, and a decrease in LV end-diastolic volume.

In some embodiments, methods of the present disclosure improve changes in ventricular structure in the patient's heart, selected from the group consisting of LV hypertrophy, an increase in cardiomyocyte size, a loss of cardiomyocytes, and a decrease in LV end-diastolic volume. In some embodiments, the method decreases cardiomyocyte size in the patient. In some embodiments, the method prevents the loss of cardiomyocytes from worsening in the patient. In some embodiments, the method increases LV end-diastolic volume in the patient.

In some embodiments of the present disclosure, the patient has a change in atrial structure in the heart selected from the group consisting of left atrial hypertrophy, arrhythmia, atrial dilation, aortic root dilation, and atrial fibrillation. In some embodiments, methods of the present disclosure improve changes in atrial structure in the patient's heart selected from the group consisting of left atrial hypertrophy, arrhythmia, atrial dilation, aortic root dilation, and atrial fibrillation.

In some embodiments of the present disclosure, the patient has a functional change in the heart selected from the group consisting of change in diastolic heart function, change in systolic heart function, and change in electrical heart function. In some embodiments, methods of the present disclosure improve a functional change in the patient's heart selected from the group consisting of change in diastolic heart function, change in systolic heart function, and change in electrical heart function.

In some embodiments, the patient has a change in diastolic function. In some embodiments, the patient has diastolic dysfunction. In some embodiments, methods of the present disclosure improve diastolic dysfunction in the patient. In some embodiments, the patient has decreased ventricular relaxation and increased filling pressures. In some embodiments, the method increases ventricular relaxation and decreases filling pressures in the patient. In some embodiments, diastolic dysfunction in the patient is measured by a ratio of early diastolic transmitral flow to early diastolic mitral annular tissue velocity (E/e′). In some embodiments, the patient's E/e′ ratio is increased in comparison to healthy people of similar age and sex. In some embodiments, the patient's E/e′ ratio is less than 8. In some embodiments, the patient's E/e′ ratio is between 8 and 15. In some embodiments, the patient's E/e′ ratio is greater than 15. In some embodiments, methods of the present disclosure decrease a patient's E/e′ ratio, wherein the patient's E/e′ ratio is decreased by at least 5% (e.g., by at least 5, 10, 15, 20, 25, 30, 35, 40, 45, 50, 55, 60, 65, 70, 75, 80, 85, 90, 95, or 100%). In some embodiments, a patient's E/e′ ratio is decreased by at least 1 (e.g., by at least 1, 2, 3, 4, 5, 6, 7, 8, 9, 10, 11, 12, 13, 14, 15, 16, 17, 18, 19, 20, 25, 35, 40, 45, or 50). In some embodiments, the method decreases a patient's E/e′ ratio to below 8.

In some embodiments, the patient has a diastolic dysfunction grade of normal. In some embodiments, the normal grade of diastolic dysfunction of the patient comprises a ratio of early diastolic transmitral flow velocity to late diastolic transmitral flow velocity (E/A) of between 1 and 2, an E/e′ of <8, a normal left atrium volume index (LAVI), and a deceleration time (DT) of <1 60 ms relative to a healthy person of similar age and sex. In some embodiments, the patient has a diastolic dysfunction grade of 1. In some embodiments, Grade 1 diastolic dysfunction of the patient comprises an E/A ratio of <1 due to impaired relaxation, an E/e′ of <8, a normal or increased LAVI, and an increased deceleration time relative to a healthy person of similar age and sex. In some embodiments, the patient has a diastolic dysfunction grade of 2. In some embodiments, Grade 2 diastolic dysfunction of the patient comprises an E/A between 1 and 2, an E/e′ of between 8 and 15, an increased LAVI, and a decreased deceleration time relative to a healthy person of similar age and sex. In some embodiments, the patient has a diastolic dysfunction grade of 3. In some embodiments, Grade 3 diastolic dysfunction of the patient comprises an E/A>2, an E/e′ of greater than 15, an increased LAVI, and a very short E deceleration time (<140 ms) due to severely reduced LV compliance and high LV filling pressure relative to a healthy person of similar age and sex.

In some embodiments, methods of the present disclosure improve the patient's diastolic dysfunction grade. In some embodiments, the method improves the patient's diastolic dysfunction grade from Grade 3 to Grade 2. In some embodiments, the method improves the patient's diastolic dysfunction grade from Grade 3 to Grade 1. In some embodiments, the method improves the patient's diastolic dysfunction grade from Grade 3 to normal. In some embodiments, the method improves the patient's diastolic dysfunction grade from Grade 2 to Grade 1. In some embodiments, the method improves the patient's diastolic dysfunction grade from Grade 2 to normal. In some embodiments, the method improves the patient's diastolic dysfunction grade from Grade 1 to normal.

In some embodiments, methods of the present disclosure increase the patient's LV diastolic function (e.g., at least 5, 10, 15, 20, 25, 30, 35, 40, 45, 50, 55, 60, 65, 70, 75, 80, 85, 90, 95, or 100%).

In some embodiments of the present disclosure, the patient has an ejection fraction of at least 50% (e.g., 50, 55, 60, 65, 70, 75, 80, 85, 90, 95, or 100%).

In some embodiments of the present disclosure, the patient is assessed for electric functional changes using electrocardiography. In some embodiments, the patient's changes in electrocardiogram measurements are selected from the group consisting of an increase in P-wave duration, P-R interval and Q-T interval, and T-wave voltage and a leftward shift of the QRS axis. In some embodiments, methods of the present disclosure improve a patient's electrocardiogram measurements selected from the group consisting of a decrease in P-wave duration, a decrease in P-R interval, a decrease in Q-T interval, an increase in T-wave voltage, and a shift of the QRS axis to a normal position.

In some embodiments of the present disclosure, the patient is assessed for diastolic dysfunction using stress diastolic testing. In some embodiments, the diastolic stress test is performed on a bicycle fixed to a catheterization table. In some embodiments, the diastolic stress test is performed using echocardiography. In some embodiments, the patient has an abnormal diastolic stress test with parameters selected from the group consisting of a septal e′ velocity <7 cm/s or lateral e′ velocity <10 cm/s at rest, an average E/e′>14 or septal E/e′ ratio >15 with exercise a peak tricuspid regurgitation (TR) velocity >2.8 m/s with exercise, and an left atrium volume index (LAVI) of >34 mL/m². In some embodiments, methods of the present disclosure increase the patient's septal e′ velocity to >7 cm/s or lateral e′ velocity to >10 cm/s at rest, decreases average E/e′ to below 14 or septal E/e′ ratio to below 15 with exercise, decreases peak tricuspid regurgitation (TR) velocity to <2.8 m/s with exercise, and decreases left atrium volume index (LAVI) to <34 mL/m².

In some embodiments, methods of the present disclosure decrease a patient's H₂FPEF score (e.g., by 1, 2, 3, 4, 5, 6, 7, 8, 9 points). In some embodiments, the patient is assessed for heart failure using right heart catheterization. In some embodiments, the patient has a pulmonary capillary wedge pressure (PCWP) of ≥15 mmHg at rest and/or a PCWP of ≥25 mmHg during exercise. In some embodiments, methods of the present disclosure decrease the patient's PCWP at rest to at least below 15 mm Hg, and/or decreases PCWP during exercise to at least below 25 mm Hg.

In some embodiments of the present disclosure, the patient has a European Heart Failure Association (EHFA) score of ≥5 points. In some embodiments, an EHFA score of ≥5 points indicates HFpEF. In some embodiments, the patient has an EHFA score of between 2 and 4 points. In some embodiments, an EHFA score of between 2 and 4 points indicates that the patient has HFpEF. In some embodiments, the patient has an EHFA score of 1 point or less. In some embodiments, an EHFA score of 1 or less indicates that the patient does not have HFpEF.

In some embodiments of the present disclosure, the patient has one or more major functional EHFA criteria for HFpEF. In some embodiments, the major functional criterion is selected from the group consisting of a septal e′ velocity <7 cm/s, a lateral e′ velocity <10 cm/s at rest, an average E/e′>14 or septal E/e′ ratio >15 with exercise and a TR velocity >2.8 m/s with exercise. In some embodiments, methods of the present disclosure improve one or more major functional criterion selected from the group consisting of increasing septal e′ velocity to >7 cm/s, increasing lateral e′ velocity to >10 cm/s at rest, decreasing E/e′ to <14 or septal E/e′ ratio to <15 with exercise and decreasing TR velocity to <2.8 m/s with exercise.

In some embodiments of the present disclosure, the patient has one or more major morphological EHFA criteria for HFpEF. In some embodiments, the major morphological criterion is selected from the group consisting of a LAVI≥34 mL/m² and an LVMI≥149 g/m² for men and ≥122 g/m² for women and RWT≥0.42. In some embodiments, methods of the present disclosure improve one or more major morphological criterion selected from the group consisting of decreasing LAVI to <34 mL/m² and decreasing LVMI to <149 g/m² for men and <122 g/m² for women, and decreasing RWT to <0.42.

In some embodiments of the present disclosure, the patient has one or more major biomarker EHFA criteria for HFpEF. In some embodiments, the major biomarker criterion is sinus rhythm, with NT-proBNP>220 pg/mL and/or BNP>80 pg/mL. In some embodiments, the major biomarker criterion is atrial fibrillation, with NT-proBNP>660 pg/mL and/or BNP>240 pg/mL. In some embodiments, the method improves sinus rhythm, comprising decreasing NT-proBNP to <220 pg/mL and/or decreasing BNP to <80 pg/mL. In some embodiments, methods of the present disclosure improve atrial fibrillation, comprising decreasing NT-proBNP to <660 pg/mL and/or decreasing BNP to <240 pg/mL.

In some embodiments of the present disclosure, the patient has one or more minor EHFA criteria for HFpEF. In some embodiments, the patient has one or more minor functional EHFA criteria for HFpEF. In some embodiments, the minor functional criterion is selected from the group consisting of an average E/e′ 9-14 and a GLS<16%. In some embodiments, methods of the present disclosure improve minor functional criteria, comprising decreasing E/e′ to 8 or below and increasing GLS to >16%.

In some embodiments of the present disclosure, the patient has one or more minor morphological EHFA criteria for HFpEF. In some embodiments, the minor morphological criterion is selected from the group consisting of a LAVI 29-34 mL/m², an LVMI>115 g/m² for men, an LVMI of 95 g/m² for women, a RWT>0.42, and an LV wall thickness >12 mm. In some embodiments, methods of the present disclosure improve one or more minor morphological criterion selected from the group consisting of decreasing LAVI to <34 mL/m², decreasing LVMI to <115 g/m² for men, decreasing LVMI to below 95 g/m² for women, decreasing RWT to <0.42, and decreasing LV wall thickness to <12 mm.

In some embodiments of the present disclosure, the patient has one or more minor biomarker EHFA criteria for HFpEF. In some embodiments, the minor biomarker criterion is sinus rhythm, with 5-NT-proBNP 125-220 pg/mL and/or BNP 35-80 pg/mL. In some embodiments, the minor biomarker criterion is atrial fibrillation, with NT-proBNP 365-660 pg/mL and/or BNP 105-240 pg/mL. In some embodiments, methods of the present disclosure improve sinus rhythm, comprising decreasing 5-NT-proBNP to <220 pg/mL and/or decreasing BNP to <80 pg/mL. In some embodiments, methods of the present disclosure improve atrial fibrillation, comprising decreasing NT-proBNP to <660 pg/mL and/or decreasing BNP to <240 pg/mL.

In some embodiments, methods of the present disclosure decrease the patient's EHFA score (e.g., by 1, 2, 3, 4, 5, 6, 7, or 8 points).

In some embodiments of the present disclosure, a patient has New York Heart Association (NYHA) Class I HF. In some embodiments, a patient has NYHA Class II HF., or. In some embodiments, a patient has NYHA Class III HF. In some embodiments, a patient has NYHA Class IV HF.

In some embodiments, methods of the present disclosure reduce a patient's NYHA Class. In some embodiments, the method reduces a patient's NYHA Class from Class IV to Class III. In some embodiments, the method reduces a patient's NYHA Class from Class IV to Class II. In some embodiments, the method reduces a patient's NYHA Class from Class IV to Class I. In some embodiments, the method reduces a patient's NYHA Class from Class III to Class II. In some embodiments, the method reduces a patient's NYHA Class from Class III to Class I. In some embodiments, the method reduces a patient's NYHA Class from Class II to Class I.

In some embodiments of the present disclosure, a patient has American College of Cardiology Foundation/American Heart Association (ACCF/AHA) stage A heart failure. In some embodiments, a patient has ACCF/AHA Stage B heart failure. In some embodiments, a patient has ACCF/AHA Stage C heart failure. In some embodiments, a patient has ACCF/AHA Stage D heart failure.

In some embodiments, methods of the present disclosure reduce a patient's ACCF/AHA stage. In some embodiments, the method reduces a patient's ACCF/AHA stage from Stage D to Stage C. In some embodiments, the method reduces a patient's ACCF/AHA stage from Stage D to Stage B. In some embodiments, the method reduces a patient's ACCF/AHA stage from Stage D to Stage A. In some embodiments, the method reduces a patient's ACCF/AHA stage from Stage C to Stage B. In some embodiments, the method reduces a patient's ACCF/AHA stage from Stage C to Stage A. In some embodiments, the method reduces a patient's ACCF/AHA stage or from Stage B to Stage A.

In some embodiments of the present disclosure, a patient has Killip Classification of HF complicating AMI Class I heart failure. In some embodiments, a patient has Killip Classification of HF complicating AMI Class II heart failure. In some embodiments, a patient has Killip Classification of HF complicating AMI Class III heart failure. In some embodiments, a patient has or Killip Classification of HF complicating AMI Class IV heart failure.

In some embodiments, methods of the present disclosure reduce a patient's Killip Classification of HF complicating AMI class. In some embodiments, the method reduces a patient's Killip Class from Class IV to Class III. In some embodiments, the method reduces a patient's Killip Class from Class IV to Class II. In some embodiments, the method reduces a patient's Killip Class from Class IV to Class I. In some embodiments, the method reduces a patient's Killip Class from Class III to Class II. In some embodiments, the method reduces a patient's Killip Class from Class III to Class I. In some embodiments, the method reduces a patient's Killip Class or from Class II to Class I.

In some embodiments of the present disclosure, a patient has one or more major Framingham criteria for diagnosis of HF. In some embodiments, a patient has one or more conditions selected from the group consisting of paroxysmal nocturnal dyspnea or orthopnea, jugular vein distension, rales, radiographic cardiomegaly, acute pulmonary edema, S3 gallop, increased venous pressure greater than 16 cm of water, circulation time greater than or equal to 25 seconds, hepatojugular reflex, and weight loss greater than or equal to 4.5 kg in 5 days in response to treatment.

In some embodiments of the present disclosure, a patient has one or more minor Framingham criteria for diagnosis of HF. In some embodiments, a patient has one or more conditions selected from the group consisting of bilateral ankle edema, nocturnal cough, dyspnea on ordinary exertion, hepatomegaly, pleural effusion, decrease in vital capacity by ⅓ from maximum recorded, and tachycardia (heart rate greater than 120/min).

In some embodiments of the present disclosure, a patient has at least two Framingham major criteria. In some embodiments, a patient has at least one major Framingham criteria and at least two minor Framingham criteria.

In some embodiments, methods of the present disclosure reduce the number of Framingham criteria for heart failure that a patient has. In some embodiments, the method decreases the number of major Framingham criteria for heart failure that a patient has. In some embodiments, the method decreases the number of minor Framingham criteria for heart failure that a patient has.

In some embodiments of the present disclosure, a patient has one or more conditions selected from the group consisting of typical symptoms, less typical symptoms, specific signs, and less specific signs of HF. In some embodiments, a patient has one or more symptoms selected from the group consisting of breathlessness, orthopnea, paroxysmal nocturnal dyspnea, reduced exercise tolerance, fatigue, tiredness, increased time to recover after exercise, and ankle swelling. In some embodiments, a patient has one or more less typical symptoms selected from the group consisting of nocturnal cough, wheezing, bloated feeling, loss of appetite, confusion (especially in the elderly), depression, palpitations, dizziness, syncope, and bendopnea.

In some embodiments of the present disclosure, a patient has one or more signs of HF. In some embodiments, a patient has one or more signs of HF selected from the group consisting of elevated jugular venous pressure, hepatojugular reflux, third heart sound (gallop rhythm), and laterally displaced apical impulse. In some embodiments, a patient has one or more less specific signs of HF. In some embodiments, a patient has one or more less specific signs of HF. In some embodiments, a patient has one or more less specific signs of HF selected from the group consisting of weight gain (>2 kg/week), weight loss (in advanced HF), tissue wasting (cachexia), cardiac murmur, peripheral edema (ankle, sacral, scrotal), pulmonary crepitations, reduced air entry and dullness to percussion at lung bases (pleural effusion), tachycardia, irregular pulse, tachypnoea, Cheyne Stokes respiration, hepatomegaly, ascites, cold extremities, oliguria, and narrow pulse pressure.

In some embodiments, methods of the present disclosure reduce the number of signs and/or symptoms of heart failure that a patient has. In some embodiments, the method decreases the number of signs of heart failure that a patient has. In some embodiments, the method decreases the number of symptoms of heart failure that a patient has.

In some embodiments of the present disclosure, a patient has elevated brain natriuretic peptide (BNP) levels as compared to a healthy patient. In some embodiments, a patient has a BNP level of at least 35 pg/mL (e.g., 35, 40, 50, 60, 70, 80, 90, 100, 150, 200, 300, 400, 500, 1000, 3000, 5000, 10,000, 15,000, or 20,000 pg/mL). In some embodiments, methods of the present disclosure decrease BNP levels in a patient by at least 5% (e.g., 5%, 10%, 15%, 20%, 25%, 30%, 35%, 40%, 45% 50%, 55%, 60%, 65%, 70%, 75%, or at least 80%). In some embodiments, methods of the present disclosure decrease BNP levels in a patient by at least 5 pg/mL (e.g., 5, 10, 50, 100, 200, 500, 1000, or 5000 pg/mL). In some embodiments, methods of the present disclosure decrease BNP levels to normal levels (i.e., <100 μg/ml).

In some embodiments of the present disclosure, a patient has elevated N-terminal pro-BNP (NT-proBNP) levels as compared to a healthy patient. In some embodiments, a patient has an NT-proBNP level of at least 10 pg/mL (e.g., 10, 25, 50, 100, 150, 200, 300, 400, 500, 1000, 3000, 5000, 10,000, 15,000, or 20,000 pg/mL). In some embodiments, methods of the present disclosure decrease NT-proBNP levels in a patient by at least 5% (e.g., 5%, 10%, 15%, 20%, 25%, 30%, 35%, 40%, 45%, 50%, 55%, 60%, 65%, 70%, 75%, or at least 80%). In some embodiments, methods of the present disclosure decrease NT-proBNP levels in a patient by at least 10 pg/mL (e.g., 10, 25, 50, 100, 200, 500, 1000, 5000, 10,000, 15,000, 20,000, or 25,000 pg/mL). In some embodiments, methods of the present disclosure decrease NT-proBNP levels to normal levels (i.e., <100 μg/ml).

In some embodiments of the present disclosure, a patient has elevated troponin levels as compared to a healthy patient. In some embodiments, methods of the present disclosure decrease troponin levels in a patient by at least 1% (e.g., 1%, 5%, 10%, 15%, 20%, 25%, 30%, 35%, 40%, 45%, 50%, 55%, 60%, 65%, 70%, 75%, or at least 80%).

In some embodiments, methods of the present disclosure reduce a patient's hospitalization rate by at least 1% (e.g., 1%, 2%, 3%, 4%, 5%, 10%, 15%, 20%, 25%, 30%, 35% 40%, 45%, 50%, 55%, 60%, 65%, 70%, 75%, 80%, 85%, 90%, 95%, or 100%). In some embodiments, methods of the present disclosure reduce a patient's rate of worsening of heart failure by at least 1% (e.g., 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, methods of the present disclosure reduce the need to for the patient to stay at the hospital. In some embodiments, methods of the present disclosure reduce the number of total patient hospital visits. In some embodiments, methods of the present disclosure increase the time to initial hospitalization of the patient. In some embodiments, methods of the present disclosure increase the length of life of the patient. In some embodiments, methods of the present disclosure increase the time between hospital visits. In some embodiments, methods of the present disclosure decrease the number of recurrent hospital visits.

In some embodiments, methods of the present disclosure increase a patient's cardiac output by at least 5% (e.g., 5%, 10%, 15%, 20%, 25%, 30%, 35%, 40%, 45%, 50%, 55%, 60%, 65%, 70%, 75%, or at least 80%).

In some embodiments, methods of the present disclosure increase exercise capacity of a patient. In some embodiments, a patient has a 6-minute walk distance from 150 to 400 meters. In some embodiments, methods of the present disclosure increase a patient's 6-minute walk distance. In some embodiments, methods of the present disclosure increase a patient's 6-minute walk distance by at least 10 meters (e.g., at least 10, 20, 30, 40, 50, 60, 70, 80, 90, 100, 125, 150, 175, 200, 250, 300, or more than 400 meters).

In some embodiments, methods of the present disclosure reduce a patient's Borg dyspnea index (BDI). In some embodiments, methods of the present disclosure reduce a patient's BDI by at least 0.5 index points (e.g., at least 0.5, 1, 1.5, 2, 2.5, 3, 3.5, 4, 4.5, 5, 5.5, 6, 6.5, 7, 7.5, 8, 8.5, 9, 9.5, or 10 index points).

In some embodiments of the disclosure, a patient is assessed for heart failure using echocardiography. In some embodiments, a patient is assessed for heart failure using cardiac magnetic resonance imaging (CMR). In some embodiments, a patient is assessed for heart failure using CMR with late gadolinium enhancement (LGE). In some embodiments, a patient is assessed for one or more of conditions selected from the group consisting of LV structure and systolic function (e.g., measured by M-mode in a parasternal short axis view at the papillary muscle level), including, but not limited to LV wall thickness (LVWT), LV mass (LVM), LV end diastolic diameter (LVEDD), LV end systolic diameter (LVESD), fractional shortening (FS) (calculated using the equation FS=100%×[(EDD−ESD)/EDD]), LV end diastolic volume (LVEDV), LV end systolic volume (LVESV), ejection fraction (calculated using the equation EF=100%×[(EDV−ESV)/EDV]), Hypertrophy index (calculated as the ratio of LVM to LVESV), and relative wall thickness (calculated as the ratio of LVWT to LVESD). In some embodiments, heart failure in a patient is assessed using cardiac imaging selected from the group consisting of multigated acquisition (MUGA), Chest X-Ray, single-photon emission computed tomography (SPECT) and radionucleotide ventriculography, positron emission tomography (PET), coronary angiography, and cardiac computing tomography (CT).

In some embodiments, methods of the present disclosure further comprise administering to a patient an additional supportive therapy or active agent. In some embodiments, the additional supportive therapy or active agent is selected from the group consisting of: angiotensin-converting enzyme (ACE) inhibitors, beta blockers, angiotensin II receptor blockers (ARB), mineralocorticoid/aldosterone receptor antagonists (MRAs), glucocorticoids, statins, Sodium-glucose co-transporter 2 (SGLT2) inhibitors, an implantable cardioverter defibrillator (ICD), angiotensin receptor neprilysin inhibitors (ARNI), and diuretics. In some embodiments, the additional active agent and/or supportive therapy is selected from the group consisting of: benazepril, captopril, enalapril, lisinopril, perindopril, ramipril, trandolapril, zofenopril, acebutolol, atenolol, betaxolol, bisoprolol, carteolol, carvedilol, labetalol, metoprolol, nadolol, nebivolol, penbutolol, pindolol, propranolol, sotalol, timolol; losartan, irbesartan, olmesartan, candesartan, valsartan, fimasartan, azilsartan, salprisartan, telmisartan, progesterone, eplerenone and spironolactone, beclomethasone, betamethasone, budesonide, cortisone, deflazacort, dexamethasone, hydrocortisone, methylprednisolone, prednisolone, methylprednisone, prednisone, triamcinolone, finerenone, atorvastatin, fluvastatin, lovastatin, pravastatin, pitavastatin, simvastatin, rosuvastatin, canagliflozin, dapagliflozin, empagliflozin, ertugliflozin, valsartan and sacubitril (a neprilysin inhibitor), furosemide, bumetanide, torasemide, bendroflumethiazide, hydrochlorothiazide, metolazone, indapamidec, spironolactone/eplerenone, amiloride triamterene, hydralazine and isosorbide dinitrate, digoxin, digitalis, N-3 polyunsaturated fatty acids (PUFA), and I_{f}-channel inhibitor.

In some embodiments of the present disclosure, a patient has a comorbidity selected from the group consisting of anemia, angina, arterial hypertension, arthritis, atrial fibrillation, cachexia, cancer, cognitive dysfunction, coronary artery disease (CAD), diabetes, erectile dysfunction, gout, hypercholesterolemia, hyperkalemia, hyperkalemia, hyperlipidemia, hypertension, iron deficiency, kidney dysfunction, metabolic syndrome, obesity, physical deconditioning, potassium disorders, pulmonary disease (e.g., asthma, COPD), sarcopenia, sleep apnea, sleep disturbance, and valvular heart disease (e.g., aortic stenosis, aortic regurgitation, mitral regurgitation, tricuspid regurgitation). In some embodiments, one or more comorbidities to consider in HF are selected from the group consisting of anemia, atrial fibrillation, coronary artery disease (CAD), and sleep apnea.

In some embodiments of the present disclosure, an ActRII-ALK4 antagonist comprises an ActRIIA polypeptide. In some embodiments, an ActRII-ALK4 antagonist is a heteromultimer.

In some embodiments of the present disclosure, an ActRIIA polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 366 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 366.

In some embodiments of the present disclosure, an ActRIIA polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 367.

In some embodiments of the present disclosure, an ActRIIA polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 368.

In some embodiments of the present disclosure, an ActRIIA polypeptide is a fusion polypeptide comprising an ActRIIA polypeptide domain and one or more heterologous domains. In some embodiments, the fusion polypeptide is an ActRIIA-Fc fusion polypeptide. In some embodiments, the fusion polypeptide further comprises a linker domain positioned between the ActRIIA polypeptide domain and i) the one or more heterologous domains or ii) Fc domain. In some embodiments, a linker domain is selected from: TGGG (SEQ ID NO: 265), TGGGG (SEQ ID NO: 263), SGGGG (SEQ ID NO: 264), GGGGS (SEQ ID NO: 267), GGG (SEQ ID NO: 261), GGGG (SEQ ID NO: 262), and SGGG (SEQ ID NO: 266).

In some embodiments of the present disclosure, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 380.

In some embodiments of the present disclosure, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 378.

In some embodiments of the present disclosure, an ActRII-ALK4 antagonist is a homodimer polypeptide. In some embodiments, an ActRII-ALK4 antagonist is a heteromultimer polypeptide. In some embodiments, a heteromultimer polypeptide comprises an ActRIIA polypeptide and an ALK4 polypeptide. In some embodiments, the heteromultimer polypeptide comprises an ActRIIA polypeptide and an ALK7 polypeptide.

In some embodiments of the present disclosure, an ALK4 polypeptide comprises an amino acid sequence that is at least 75%, 80%, 85%, 90%, 91%, 92%, 93%, 94%, 95%, 96%, 97%, 98%, 99%, or 100% identical to an amino acid sequence selected from the group consisting of SEQ ID NOs:84, 85, 86, 87, 88, 89, 92, 93, 247, 249, 421, and 422.

In some embodiments of the present disclosure, an ALK7 polypeptide comprises an amino acid sequence that is at least 75%, 80%, 85%, 90%, 91%, 92%, 93%, 94%, 95%, 96%, 97%, 98%, 99%, or 100% identical to an amino acid sequence selected from the group consisting of SEQ ID NOs: 120, 121, 122, 123, 124, 125, 126, 127, 128, 129, 130, 133, and 134.

In some embodiments of the present disclosure, an ALK4 polypeptide is a fusion polypeptide comprising an ALK4 polypeptide domain and one or more heterologous domains. In some embodiments, an ALK7 polypeptide is a fusion polypeptide comprising an ALK7 polypeptide domain and one or more heterologous domains. In some embodiments, the fusion polypeptide is an ALK4-Fc fusion polypeptide. In some embodiments, the fusion polypeptide is an ALK7-Fc fusion polypeptide. In some embodiments, the ALK4-Fc fusion polypeptide further comprises a linker domain positioned between the ALK4 polypeptide domain and i) the one or more heterologous domains or ii) Fc domain. In some embodiments, the ALK7-Fc fusion polypeptide further comprises a linker domain positioned between the ALK7 polypeptide domain and i) the one or more heterologous domains or ii) Fc domain. In some embodiments, the linker domain is selected from: TGGG (SEQ ID NO: 265), TGGGG (SEQ ID NO: 263), SGGGG (SEQ ID NO: 264), GGGGS (SEQ ID NO: 267), GGG (SEQ ID NO: 261), GGGG (SEQ ID NO: 262), and SGGG (SEQ ID NO: 266).

In some embodiments of the present disclosure, a heteromultimer comprises an Fc domain selected from: a) the ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 13, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13; b) the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14; c.) the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15; d.) the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16; and e.) the ActRIIA-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 17, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17.

In some embodiments of the present disclosure, a heteromultimer comprises an Fc domain selected from: a.) the ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 13, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13; b.) the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14, and the ALK7-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 14; c.) the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15; d.) the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16; and e.) the ActRIIA-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 17, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17.

In some embodiments of the present disclosure, a heteromultimer comprises an Fc domain selected from: a.) the ActRIIA-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 18, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19; and b.) the ActRIIA-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 19, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18.

In some embodiments of the present disclosure, a heteromultimer comprises an Fc domain selected from: a.) The ActRIIA-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 18, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19; and b.) The ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18.

In some embodiments of the present disclosure, a heteromultimer comprises an Fc domain selected from: a.) The ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 20, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21; and b.) The ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 21, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20.

In some embodiments of the present disclosure, a heteromultimer comprises an Fc domain selected from: a.) The ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 20, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21; and b.) The ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 21, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20.

In some embodiments of the present disclosure, a heteromultimer comprises an Fc domain selected from: a.) The ActRIIA-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 22, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23; and b.) The ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 23, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22.

In some embodiments of the present disclosure, a heteromultimer comprises an Fc domain selected from: a.) The ActRIIA-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 22, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23; and b.) The ActRIIA-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 23, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22.

In some embodiments of the present disclosure, a heteromultimer comprises an Fc domain selected from: a.) The ActRIIA-Fc fusion polypeptide comprises an Fc domain that is at least 75%, 80%, 85%, 90%, 91%, 92%, 93%, 94%, 95% 96%0, 97% 98%, 99%, or 100% identical to the amino acid sequence of SEQ ID NO: 24, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25; and b.) The ActRIIA-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 25, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24.

In some embodiments of the present disclosure, a heteromultimer comprises an Fc domain selected from: a.) The ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 24, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25; and b.) The ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 25, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24.

In some embodiments of the present disclosure, a heteromultimer comprises an Fc domain selected from: a.) The ActRIIA-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 26, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27; and b.) The ActRIIA-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 27, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26.

In some embodiments of the present disclosure, a heteromultimer comprises an Fc domain selected from: a.) The ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27; and b.) The ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26.

In some embodiments of the present disclosure, an ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments of the present disclosure, an ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments of the present disclosure, an ActRIIA-Fc fusion polypeptide Fc domain comprises a cysteine at amino acid position 132, glutamic acid at amino acid position 138, a tryptophan at amino acid position 144, and a aspartic acid at amino acid position 217, and wherein the ALK4-Fc fusion polypeptide Fc domain comprises a cysteine at amino acid position 127, a serine at amino acid position 144, an alanine at position 146 an arginine at amino acid position 162, an arginine at amino acid position 179, and a valine at amino acid position 185.

In some embodiments of the present disclosure, an ActRIIA-Fc fusion polypeptide Fc domain comprises a cysteine at amino acid position 132, glutamic acid at amino acid position 138, a tryptophan at amino acid position 144, and a aspartic acid at amino acid position 217, and wherein the ALK7-Fc fusion polypeptide Fc domain comprises a cysteine at amino acid position 127, a serine at amino acid position 144, an alanine at position 146 an arginine at amino acid position 162, an arginine at amino acid position 179, and a valine at amino acid position 185.

In some embodiments of the present disclosure, an ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments of the present disclosure, an ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments of the present disclosure, an ALK4-Fc fusion polypeptide Fc domain comprises a cysteine at amino acid position 132, glutamic acid at amino acid position 138, a tryptophan at amino acid position 144, and a aspartic acid at amino acid position 217, and wherein the ActRIIA-Fc fusion polypeptide Fc domain comprises a cysteine at amino acid position 127, a serine at amino acid position 144, an alanine at position 146 an arginine at amino acid position 162, an arginine at amino acid position 179, and a valine at amino acid position 185.

In some embodiments of the present disclosure, an ALK7-Fc fusion polypeptide Fc domain comprises a cysteine at amino acid position 132, glutamic acid at amino acid position 138, a tryptophan at amino acid position 144, and a aspartic acid at amino acid position 217, and wherein the ActRIIA-Fc fusion polypeptide Fc domain comprises a cysteine at amino acid position 127, a serine at amino acid position 144, an alanine at position 146 an arginine at amino acid position 162, an arginine at amino acid position 179, and a valine at amino acid position 185.

In some embodiments of the present disclosure, an ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments of the present disclosure, an ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments of the present disclosure, an ActRIIA-Fc fusion polypeptide Fc domain comprises a cysteine at amino acid position 132, a tryptophan at amino acid position 144, and a arginine at amino acid position 435, and wherein the ALK4-Fc fusion polypeptide Fc domain comprises cysteine at amino acid position 127, a serine at amino acid position 144, an alanine at amino acid position 146, and a valine at amino acid position 185.

In some embodiments of the present disclosure, an ActRIIA-Fc fusion polypeptide Fc domain comprises a cysteine at amino acid position 132, a tryptophan at amino acid position 144, and a arginine at amino acid position 435, and wherein the ALK7-Fc fusion polypeptide Fc domain comprises cysteine at amino acid position 127, a serine at amino acid position 144, an alanine at amino acid position 146, and a valine at amino acid position 185.

In some embodiments of the present disclosure, an ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments of the present disclosure, an ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments of the present disclosure, an ALK4-Fc fusion polypeptide Fc domain comprises a cysteine at amino acid position 132, a tryptophan at amino acid position 144, and a arginine at amino acid position 435, and wherein the ActRIIA-Fc fusion polypeptide Fc domain comprises cysteine at amino acid position 127, a serine at amino acid position 144, an alanine at amino acid position 146, and a valine at amino acid position 185.

In some embodiments of the present disclosure, an ALK7-Fc fusion polypeptide Fc domain comprises a cysteine at amino acid position 132, a tryptophan at amino acid position 144, and a arginine at amino acid position 435, and wherein the ActRIIA-Fc fusion polypeptide Fc domain comprises cysteine at amino acid position 127, a serine at amino acid position 144, an alanine at amino acid position 146, and a valine at amino acid position 185.

In some embodiments of the present disclosure, an ActRII-ALK4 antagonist comprises an ActRIIB polypeptide.

In some embodiments of the present disclosure, an ActRII-ALK4 antagonist is a heteromultimer.

In some embodiments of the present disclosure, an ActRIIB polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2.

In some embodiments of the present disclosure, an ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 29-109 of SEQ ID NO: 2.

In some embodiments of the present disclosure, an ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 25-131 of SEQ ID NO: 2.

In some embodiments of the present disclosure, an ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 20-134 of SEQ ID NO: 2.

In some embodiments of the present disclosure, an ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 53.

In some embodiments of the present disclosure, an ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 388.

In some embodiments of the present disclosure, an ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 389.

In some embodiments of the present disclosure, an ActRIIB polypeptide is a fusion polypeptide comprising an ActRIIB polypeptide domain and one or more heterologous domains. In some embodiments, the fusion polypeptide is an ActRIIB-Fc fusion polypeptide. In some embodiments, the fusion polypeptide further comprises a linker domain positioned between the ActRIIB polypeptide domain and the one or more heterologous domains or Fc domain. In some embodiments, the linker domain is selected from: TGGG (SEQ ID NO: 265), TGGGG (SEQ ID NO: 263), SGGGG (SEQ ID NO: 264), GGGGS (SEQ ID NO: 267), GGG (SEQ ID NO: 261), GGGG (SEQ ID NO: 262), and SGGG (SEQ ID NO: 266). In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 5. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 12.

In some embodiments of the present disclosure, an ActRIIB polypeptide comprises one or more amino acid substitution with respect to the amino acid sequence of SEQ ID NO: 2 selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N. In some embodiments, an ActRIIB polypeptide comprises one or more amino acid substitution with respect to the amino acid sequence of SEQ ID NO: 2 selected from the group consisting of: L38N, E50L, E52D, E52N, E52Y, L57E, L57I, L57R, L57T, L57V, Y60D, G68R, K74E, W78Y, L79E, L79F, L79H, L79R, L79S, L79T, L79W, F82D, F82E, F82I, F82K, F82L, F82S, F82T, F82Y, N83R, E94K, and V99G.

In some embodiments of the present disclosure, an ActRIIB polypeptide comprises an L substitution at the position corresponding to E50 of SEQ ID NO: 2. In some embodiments, an ActRIIB polypeptide comprises an N substitution at the position corresponding to L38 of SEQ ID NO: 2. In some embodiments, an ActRIIB polypeptide comprises a G substitution at the position corresponding to V99 of SEQ ID NO: 2. In some embodiments, an ActRIIB polypeptide comprises a R substitution at the position corresponding to N83 of SEQ ID NO: 2. In some embodiments, an ActRIIB polypeptide comprises an T substitution at the position corresponding to F82 of SEQ ID NO: 2. In some embodiments, an ActRIIB polypeptide comprises an H substitution at the position corresponding to L79 of SEQ ID NO: 2.

In some embodiments of the present disclosure, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 276. In some embodiments, the polypeptide comprises an amino acid sequence that is at least 75%, 80%, 85%, 90%, 91%, 92%, 93%, 94%, 95% 96%, 97%, 98%, 99%, or 100% identical to the amino acid sequence of SEQ ID NO: 278. In some embodiments, the polypeptide comprises an I substitution at the position corresponding to F82 of SEQ ID NO: 2 and an R substitution at the position corresponding to N83.

In some embodiments of the present disclosure, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 279. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 332. In some embodiments, the polypeptide comprises a K substitution at the position corresponding to F82 of SEQ ID NO: 2 and an R substitution at the position corresponding to N83.

In some embodiments of the present disclosure, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 333. In some embodiments, the polypeptide comprises an amino acid sequence that is at least 75%, 80%, 85%, 90%, 91%, 92%, 93%, 94%, 95% 96%, 97%, 98%, 99%, or 100% identical to the amino acid sequence of SEQ ID NO: 335. In some embodiments, the polypeptide comprises a T substitution at the position corresponding to F82 of SEQ ID NO: 2 and an R substitution at the position corresponding to N83.

In some embodiments of the present disclosure, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 336. In some embodiments, the polypeptide comprises an amino acid sequence that is at least 75%, 80%, 85%, 90%, 91%, 92%, 93%, 94%, 95% 96%, 97%, 98%, 99%, or 100% identical to the amino acid sequence of SEQ ID NO: 338. In some embodiments, the polypeptide comprises a T substitution at the position corresponding to F82 of SEQ ID NO: 2.

In some embodiments of the present disclosure, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 339. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 341. In some embodiments, the polypeptide comprises an H substitution at the position corresponding to L79 of SEQ ID NO: 2 and an I substitution at the position corresponding to F82.

In some embodiments of the present disclosure, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 342. In some embodiments, the polypeptide comprises an amino acid sequence that is at least 75%, 80%, 85%, 90%, 91%, 92%, 93%, 94%, 95% 96%, 97% 98%, 99%, or 100% identical to the amino acid sequence of SEQ ID NO: 344. In some embodiments, the polypeptide comprises an H substitution at the position corresponding to L79 of SEQ ID NO: 2.

In some embodiments of the present disclosure, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 345. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 347. In some embodiments, the polypeptide comprises an H substitution at the position corresponding to L79 of SEQ ID NO: 2 and a K substitution at the position corresponding to F82.

In some embodiments of the present disclosure, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 348. In some embodiments, the polypeptide comprises an amino acid sequence that is at least 75%, 80%, 85%, 90%, 91%, 92%, 93%, 94%, 95% 96%, 97%, 98%, 99%, or 100% identical to the amino acid sequence of SEQ ID NO: 350. In some embodiments, the polypeptide comprises an L substitution at the position corresponding to E50 of SEQ ID NO: 2.

In some embodiments of the present disclosure, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 351. In some embodiments, the polypeptide comprises an amino acid sequence that is at least 75%, 80%, 85%, 90%, 91%, 92%, 93%, 94%, 95% 96%, 97% 98%, 99%, or 100% identical to the amino acid sequence of SEQ ID NO: 353. In some embodiments, the polypeptide comprises an N substitution at the position corresponding to L38 of SEQ ID NO: 2 and an R substitution at the position corresponding to L79.

In some embodiments of the present disclosure, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 354. In some embodiments, the polypeptide comprises an amino acid sequence that is at least 75%, 80%, 85%, 90%, 91%, 92%, 93%, 94%, 95% 96%, 97% 98%, 99%, or 100% identical to the amino acid sequence of SEQ ID NO: 356. In some embodiments, the polypeptide comprises an G substitution at the position corresponding to V99 of SEQ ID NO: 2.

In some embodiments of the present disclosure, an ActRIIB polypeptide is a homodimer polypeptide. In some embodiments, an ActRIIB polypeptide is a heterodimer polypeptide.

In some embodiments of the present disclosure, an ActRIIB polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2 and one or more amino acid substitutions at a position of SEQ ID NO: 2 selected from the group consisting of: L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G.

In some embodiments of the present disclosure, an ActRIIB polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2 and one or more amino acid substitutions at a position of SEQ ID NO: 2 selected from the group consisting of: L38N, E50L, E52D, E52N, E52Y, L57E, L57I, L57R, L57T, L57V, Y60D, G68R, K74E, W78Y, L79E, L79F, L79H, L79R, L79S, L79T, L79W, F82D, F82E, F82I, F82K, F82L, F82S, F82T, F82Y, N83R, E94K, and V99G.

In some embodiments of the present disclosure, an ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 29-109 of SEQ ID NO: 2. In some embodiments, an ActRIIB polypeptide comprises an amino acid sequence that is at least 75%, 80%, 85%, 90%, 91%, 92%, 93%, 94%, 95% 96%, 97% 98%, 99%, or 100% identical to amino acids 25-131 of SEQ ID NO: 2. In some embodiments, an ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 20-134 of SEQ ID NO: 2. In some embodiments, an ActRIIB polypeptide comprises an amino acid sequence that is at least 75%, 80%, 85%, 90%, 91%, 92%, 93%, 94%, 95% 96%, 97% 98%, 99%, or 100% identical to the amino acid sequence of SEQ ID NO: 53. In some embodiments, an ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 388. In some embodiments, an ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 389. In some embodiments, an ActRIIB polypeptide comprises one or more amino acid substitution with respect to the amino acid sequence of SEQ ID NO: 2 selected from the group consisting of: L38N, E50L, E52D, E52N, E52Y, L57E, L57I, L57R, L57T, L57V, Y60D, G68R, K74E, W78Y, L79E, L79F, L79H, L79R, L79S, L79T, L79W, F82D, F82E, F82I, F82K, F82L, F82S, F82T, F82Y, N83R, E94K, and V99G.

In some embodiments of the present disclosure, a heteromultimer polypeptide comprises an ActRIIA polypeptide and an ALK4 polypeptide. In some embodiments, a heteromultimer polypeptide comprises an ActRIIA polypeptide and an ALK7 polypeptide. In some embodiments, an ALK4 polypeptide comprises an amino acid sequence that is at least 75%, 80%, 85%, 90%, 91%, 92%, 93%, 94%, 95%, 96%, 97%, 98%, 99%, or 100% identical to an amino acid sequence selected from the group consisting of SEQ ID NOs: 84, 85, 86, 87, 88, 89, 92, 93, 247, 249, 421, and 422. In some embodiments, an ALK7 polypeptide comprises an amino acid sequence that is at least 75%, 80%, 85%, 90%, 91%, 92%, 93%, 94%, 95%, 96%, 97%, 98%, 99%, or 100% identical to an amino acid sequence selected from the group consisting of SEQ ID NOs: 120, 121, 122, 123, 124, 125, 126, 127, 128, 129, 130, 133, and 134.

In some embodiments of the present disclosure, an ActRIIB polypeptide is a fusion polypeptide comprising an ActRIIB polypeptide domain and one or more heterologous domains. In some embodiments, an ALK4 polypeptide is a fusion polypeptide comprising an ALK4 polypeptide domain and one or more heterologous domains. In some embodiments, an ALK7 polypeptide is a fusion polypeptide comprising an ALK7 polypeptide domain and one or more heterologous domains. In some embodiments, an ActRIIB polypeptide is an ActRIIB-Fc fusion polypeptide. In some embodiments, an ALK4 polypeptide is an ALK4-Fc fusion polypeptide. In some embodiments, an ALK7 polypeptide is an ALK7-Fc fusion polypeptide. In some embodiments, an ActRIIB-Fc fusion polypeptide further comprises a linker domain positioned between the ActRIIB polypeptide domain and the one or more heterologous domains or Fc domain. In some embodiments, the ALK4-Fc fusion polypeptide further comprises a linker domain positioned between the ALK4 polypeptide domain and the one or more heterologous domains or Fc domain. In some embodiments, the ALK7-Fc fusion polypeptide further comprises a linker domain positioned between the ALK7 polypeptide domain and the one or more heterologous domains or Fc domain. In some embodiments, the linker domain is selected from: TGGG (SEQ ID NO: 265), TGGGG (SEQ ID NO: 263), SGGGG (SEQ ID NO: 264), GGGGS (SEQ ID NO: 267), GGG (SEQ ID NO: 261), GGGG (SEQ ID NO: 262), and SGGG (SEQ ID NO: 266).

In some embodiments of the present disclosure, a heteromultimer comprises an Fc domain selected from: a.) the ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 13, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13; b.) the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14; c.) the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15; d.) the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16; and e.) the ActRIIB-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 17, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17.

In some embodiments of the present disclosure, a heteromultimer comprises an Fc domain selected from: a.) the ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 13, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13; b.) the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14; c.) the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15; d.) the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16; and e.) the ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 17, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17.

In some embodiments of the present disclosure, a heteromultimer comprises an Fc domain selected from: a.) the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19; and b) the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18.

In some embodiments of the present disclosure, a heteromultimer comprises an Fc domain selected from: a.) The ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 18, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19; and b.) The ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 19, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18.

In some embodiments of the present disclosure, a heteromultimer comprises an Fc domain selected from: a.) The ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 20, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21; and b.) The ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 21, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20.

In some embodiments of the present disclosure, a heteromultimer comprises an Fc domain selected from: a.) The ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21; and b.) The ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20.

In some embodiments of the present disclosure, a heteromultimer comprises an Fc domain selected from: a.) The ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 22, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23; and b.) The ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 23, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22.

In some embodiments of the present disclosure, a heteromultimer comprises an Fc domain selected from: a.) The ActRIIB-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 22, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23; and b.) The ActRIIB-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 23, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22.

In some embodiments of the present disclosure, a heteromultimer comprises an Fc domain selected from: a.) The ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25; and b.) The ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24.

In some embodiments of the present disclosure, a heteromultimer comprises an Fc domain selected from: a.) The ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 24, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25; and b.) The ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 25, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24.

In some embodiments of the present disclosure, a heteromultimer comprises an Fc domain selected from: a.) The ActRIIB-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 26, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27; and b.) The ActRIIB-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 27, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26.

In some embodiments of the present disclosure, a heteromultimer comprises an Fc domain selected from: a.) The ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27; and b.) The ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26.

In some embodiments of the present disclosure, an ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments of the present disclosure, an ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments of the present disclosure, an ActRIIB-Fc fusion polypeptide Fc domain comprises a cysteine at amino acid position 132, glutamic acid at amino acid position 138, a tryptophan at amino acid position 144, and a aspartic acid at amino acid position 217, and wherein the ALK4-Fc fusion polypeptide Fc domain comprises a cysteine at amino acid position 127, a serine at amino acid position 144, an alanine at position 146 an arginine at amino acid position 162, an arginine at amino acid position 179, and a valine at amino acid position 185.

In some embodiments of the present disclosure, an ActRIIB-Fc fusion polypeptide Fc domain comprises a cysteine at amino acid position 132, glutamic acid at amino acid position 138, a tryptophan at amino acid position 144, and a aspartic acid at amino acid position 217, and wherein the ALK7-Fc fusion polypeptide Fc domain comprises a cysteine at amino acid position 127, a serine at amino acid position 144, an alanine at position 146 an arginine at amino acid position 162, an arginine at amino acid position 179, and a valine at amino acid position 185.

In some embodiments of the present disclosure, an ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments of the present disclosure, an ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments of the present disclosure, an ALK4-Fc fusion polypeptide Fc domain comprises a cysteine at amino acid position 132, glutamic acid at amino acid position 138, a tryptophan at amino acid position 144, and a aspartic acid at amino acid position 217, and wherein the ActRIIB-Fc fusion polypeptide Fc domain comprises a cysteine at amino acid position 127, a serine at amino acid position 144, an alanine at position 146 an arginine at amino acid position 162, an arginine at amino acid position 179, and a valine at amino acid position 185.

In some embodiments of the present disclosure, an ALK7-Fc fusion polypeptide Fc domain comprises a cysteine at amino acid position 132, glutamic acid at amino acid position 138, a tryptophan at amino acid position 144, and a aspartic acid at amino acid position 217, and wherein the ActRIIB-Fc fusion polypeptide Fc domain comprises a cysteine at amino acid position 127, a serine at amino acid position 144, an alanine at position 146 an arginine at amino acid position 162, an arginine at amino acid position 179, and a valine at amino acid position 185.

In some embodiments of the present disclosure, an ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments of the present disclosure, an ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments of the present disclosure, an ActRIIB-Fc fusion polypeptide Fc domain comprises a cysteine at amino acid position 132, a tryptophan at amino acid position 144, and a arginine at amino acid position 435, and wherein the ALK4-Fc fusion polypeptide Fc domain comprises cysteine at amino acid position 127, a serine at amino acid position 144, an alanine at amino acid position 146, and a valine at amino acid position 185.

In some embodiments of the present disclosure, an ActRIIB-Fc fusion polypeptide Fc domain comprises a cysteine at amino acid position 132, a tryptophan at amino acid position 144, and a arginine at amino acid position 435, and wherein the ALK7-Fc fusion polypeptide Fc domain comprises cysteine at amino acid position 127, a serine at amino acid position 144, an alanine at amino acid position 146, and a valine at amino acid position 185.

In some embodiments of the present disclosure, an ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments of the present disclosure, an ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments of the present disclosure, an ALK4-Fc fusion polypeptide Fc domain comprises a cysteine at amino acid position 132, a tryptophan at amino acid position 144, and a arginine at amino acid position 435, and wherein the ActRIIB-Fc fusion polypeptide Fc domain comprises cysteine at amino acid position 127, a serine at amino acid position 144, an alanine at amino acid position 146, and a valine at amino acid position 185.

In some embodiments of the present disclosure, an ALK7-Fc fusion polypeptide Fc domain comprises a cysteine at amino acid position 132, a tryptophan at amino acid position 144, and a arginine at amino acid position 435, and wherein the ActRIIB-Fc fusion polypeptide Fc domain comprises cysteine at amino acid position 127, a serine at amino acid position 144, an alanine at amino acid position 146, and a valine at amino acid position 185.

In some embodiments of the present disclosure, an ActRII-ALK4 antagonist is a follistatin polypeptide. In some embodiments, the follistatin polypeptide an amino acid sequence that is at least 75%, 80%, 85%, 90%, 91%, 92%, 93%, 94%, 95%, 96%, 97%, 98%, 99%, or 100% identical to an amino acid sequence selected from the group consisting of SEQ ID NOs: 390, 391, 392, 393, and 394.

In some embodiments of the present disclosure, an ActRII-ALK4 antagonist inhibits one or more ligands selected from the group consisting of activin A, activin B, GDF8, GDF11, BMP6, BMP10, ALK4, ActRIIA, and ActRIIB.

In some embodiments of the present disclosure, an ActRII-ALK4 antagonist is an antibody or combination of antibodies. In some embodiments, the antibody or combination of antibodies binds to one or more ligands selected from the group consisting of activin A, activin B, GDF8, GDF11, BMP6, BMP10, ALK4, ActRIIA, and ActRIIB. In some embodiments, the antibody is a multispecific antibody. In some embodiments, the antibody is a bi-specific antibody. In some embodiments, the antibody is selected from the group consisting of garetosmab, trevogumab, stamulumab, domagrozumab, landogrozumab, and bimagrumab.

In some embodiments of the present disclosure, an ActRII-ALK4 antagonist is a small molecule or combination of small molecules. In some embodiments, the small molecule or combination of small molecules inhibits one or more ligands selected from the group consisting of activin A, activin B, GDF8, GDF11, BMP6, BMP10, ALK4, ActRIIA, and ActRIIB.

In some embodiments of the present disclosure, an ActRII-ALK4 antagonist is a polynucleotide or combination of polynucleotides. In some embodiments, the polynucleotide or combination of polynucleotides inhibits one or more ligands selected from the group consisting of activin A, activin B, GDF8, GDF11, BMP6, BMP10, ALK4, ActRIIA, and ActRIIB.

### BRIEF DESCRIPTION OF THE DRAWING

FIG. 1 shows an alignment of extracellular domains of human ActRIIB (SEQ ID NO: 1) and human ActRIIA (SEQ ID NO: 367) with the residues that are deduced herein, based on composite analysis of multiple ActRIIB and ActRIIA crystal structures, to directly contact ligand indicated with boxes.

FIG. 2 shows the amino acid sequence of human ActRIIB precursor polypeptide (SEQ ID NO: 2); NCBI Reference Sequence NP_001097.2). The signal peptide is underlined, the extracellular domain is in bold (also referred to as SEQ ID NO: 1), and the potential N-linked glycosylation sites are boxed. SEQ ID NO: 2 is used as the wild-type reference sequence for human ActRIIB in this disclosure, and the numbering for the variants described herein are based on the numbering in SEQ ID NO: 2 FIG. 3 shows the amino acid sequence of a human ActRIIB extracellular domain polypeptide (SEQ ID NO: 1).

FIG. 4 shows a nucleic acid sequence encoding human ActRIIB precursor polypeptide. SEQ ID NO: 4 consists of nucleotides 434-1972 of NCBI Reference Sequence NM_001106.4.

FIG. 5 shows a nucleic acid sequence (SEQ ID NO: 3) encoding a human ActRIIB(20-134) extracellular domain polypeptide.

FIG. 6 shows a multiple sequence alignment of various vertebrate ActRIIB precursor polypeptides without their intracellular domains (SEQ ID NOs: 358-363), human ActRIIA precursor polypeptide without its intracellular domain (SEQ ID NO: 364), and a consensus ActRII precursor polypeptide (SEQ ID NO: 365). Upper case letters in the consensus sequence indicate positions that are conserved. Lower case letters in the consensus sequence indicate an amino acid residue that is the predominant form, but not universal at that position.

FIG. 7 shows multiple sequence alignment of Fc domains from human IgG isotypes using Clustal 2.1. Hinge regions are indicated by dotted underline. Double underline indicates examples of positions engineered in IgG1 (SEQ ID NO: 13) Fc to promote asymmetric chain pairing and the corresponding positions with respect to other isotypes IgG4 (SEQ ID NO: 17), IgG2 (SEQ ID NO: 14), and IgG3 (SEQ ID NO: 15).

FIG. 8A and FIG. 8B show schematic examples of heteromeric polypeptide complexes comprising a variant ActRIIB polypeptide (indicated as “X”) and either an ALK4 polypeptide (indicated as “Y”) or an ALK7 polypeptide (indicated as “Y”). In the illustrated embodiments, the variant ActRIIB polypeptide is part of a fusion polypeptide that comprises a first member of an interaction pair (“C₁”), and either an ALK4 polypeptide or an ALK7 polypeptide is part of a fusion polypeptide that comprises a second member of an interaction pair (“C₂”). Suitable interaction pairs include, for example, heavy chain and/or light chain immunoglobulin interaction pairs, truncations, and variants thereof such as those described herein [e.g., Spiess et al (2015) Molecular Immunology 67(2A): 95-106]. In each fusion polypeptide, a linker may be positioned between the variant ActRIIB polypeptide, ALK4 polypeptide, or ALK7 polypeptide and the corresponding member of the interaction pair. The first and second members of the interaction pair may be unguided, meaning that the members of the pair may associate with each other or self-associate without substantial preference, and they may have the same or different amino acid sequences. See FIG. 8A. Alternatively, the interaction pair may be a guided (asymmetric) pair, meaning that the members of the pair associate preferentially with each other rather than self-associate. See FIG. 8B.

FIG. 9 shows a multiple sequence alignment of various vertebrate ALK4 proteins and human ALK4 (SEQ ID NOs: 414-420).

FIG. 10 shows a multiple sequence alignment of various vertebrate ActRIIA proteins and human ActRIIA (SEQ ID NOs: 367, 371-377).

FIGS. 11A and 11B show two schematic examples of heteromeric protein complexes comprising type I receptor and type II receptor polypeptides. FIG. 11A depicts a heterodimeric protein complex comprising one type I receptor fusion polypeptide and one type II receptor fusion polypeptide, which can be assembled covalently or noncovalently via a multimerization domain contained within each polypeptide chain. Two assembled multimerization domains constitute an interaction pair, which can be either guided or unguided. FIG. 11B depicts a heterotetrameric protein complex comprising two heterodimeric complexes as depicted in FIG. 11A. Complexes of higher order can be envisioned.

FIG. 12 show a schematic example of a heteromeric protein complex comprising a type I receptor polypeptide (indicated as “I”) (e.g. a polypeptide that is at least 70%, 75%, 80%, 85%, 90%, 91%, 92%, 93%, 94%, 95%, 97%, 98%, 99% or 100% identical to an extracellular domain of an ALK4 protein from humans or other species such as those described herein) and a type II receptor polypeptide (indicated as “II”) (e.g. a polypeptide that is at least 70%, 75%, 80%, 85%, 90%, 91%, 92%, 93%, 94%, 95%, 97%, 98%, 99% or 100% identical to an extracellular domain of an ActRIIB protein from humans or other species as such as those described herein). In the illustrated embodiments, the type I receptor polypeptide is part of a fusion polypeptide that comprises a first member of an interaction pair (“C₁”), and the type II receptor polypeptide is part of a fusion polypeptide that comprises a second member of an interaction pair (“C₂”). In each fusion polypeptide, a linker may be positioned between the type I or type II receptor polypeptide and the corresponding member of the interaction pair. The first and second members of the interaction pair may be a guided (asymmetric) pair, meaning that the members of the pair associate preferentially with each other rather than self-associate, or the interaction pair may be unguided, meaning that the members of the pair may associate with each other or self-associate without substantial preference and may have the same or different amino acid sequences. Traditional Fc fusion proteins and antibodies are examples of unguided interaction pairs, whereas a variety of engineered Fc domains have been designed as guided (asymmetric) interaction pairs [e.g., Spiess et al (2015) Molecular Immunology 67(2A): 95-106].

FIGS. 13A-13D show schematic examples of heteromeric protein complexes comprising an ALK4 polypeptide (e.g. a polypeptide that is at least 70%, 75%, 80%, 85%, 90%, 91%, 92%, 93%, 94%, 95%, 97%, 98%, 99% or 100% identical to an extracellular domain of an ALK4 protein from humans or other species such as those described herein) and an ActRIIB polypeptide (e.g. a polypeptide that is at least 70%, 75%, 80%, 85%, 90%, 91%, 92%, 93%, 94%, 95%, 97%, 98%, 99% or 100% identical to an extracellular domain of an ActRIIB protein from humans or other species such as those described herein). In the illustrated embodiments, the ALK4 polypeptide is part of a fusion polypeptide that comprises a first member of an interaction pair (“C₁”), and the ActRIIB polypeptide is part of a fusion polypeptide that comprises a second member of an interaction pair (“C₂”). Suitable interaction pairs included, for example, heavy chain and/or light chain immunoglobulin interaction pairs, truncations, and variants thereof such as those described herein [e.g., Spiess et al (2015) Molecular Immunology 67(2A): 95-106]. In each fusion polypeptide, a linker may be positioned between the ALK4 or ActRIIB polypeptide and the corresponding member of the interaction pair. The first and second members of the interaction pair may be unguided, meaning that the members of the pair may associate with each other or self-associate without substantial preference, and they may have the same or different amino acid sequences. See FIG. 13A. Alternatively, the interaction pair may be a guided (asymmetric) pair, meaning that the members of the pair associate preferentially with each other rather than self-associate. See FIG. 13B. Complexes of higher order can be envisioned. See FIGS. 13C and 13D.

FIG. 14 shows the purification of ActRIIA-hFc expressed in CHO cells. The protein purifies as a single, well-defined peak as visualized by sizing column (top panel) and Coomassie stained SDS-PAGE (bottom panel) (left lane: molecular weight standards; right lane: ActRIIA-hFc).

FIG. 15 shows the binding of ActRIIA-hFc to activin (top panel) and GDF-11 (bottom panel), as measured by Biacore™ assay.

FIG. 16A and FIG. 16B show values for ligand binding kinetics of homodimeric Fc-fusion polypeptides comprising variant or unmodified ActRIIB domains, as determined by surface plasmon resonance at 37° C. Amino acid numbering is based on SEQ ID NO: 2. ND #indicates that the value is not detectable over concentration range tested. Transient* indicates that the value is indeterminate due to transient nature of interaction. Control sample is ActRIIB-G1Fc (SEQ ID NO: 5).

FIG. 17 shows values for ligand binding kinetics of homodimeric Fc-fusion polypeptides comprising variant or unmodified ActRIIB domains, as determined by surface plasmon resonance at 37° C. Amino acid numbering is based on SEQ ID NO: 2. ND #indicates that the value is not detectable over concentration range tested. Transient binding* indicates that the value is indeterminate due to transient nature of interaction. Control sample is ActRIIB-G1Fc (SEQ ID NO: 5).

FIG. 18 shows values for ligand binding kinetics of homodimeric Fc-fusion polypeptides comprising variant or unmodified ActRIIB domains, as determined by surface plasmon resonance at 25° C. ND #indicates that the value is not detectable over concentration range tested. Amino acid numbering is based on SEQ ID NO: 2.

FIG. 19 shows comparative ligand binding data for an ALK4-Fc:ActRIIB-Fc heterodimeric protein complex compared to ActRIIB-Fc homodimer and ALK4-Fc homodimer. For each protein complex, ligands are ranked by k_{off}, a kinetic constant that correlates well with ligand signaling inhibition, and listed in descending order of binding affinity (ligands bound most tightly are listed at the top). At left, yellow, red, green, and blue lines indicate magnitude of the off-rate constant. Solid black lines indicate ligands whose binding to heterodimer is enhanced or unchanged compared with homodimer, whereas dashed red lines indicate substantially reduced binding compared with homodimer. As shown, the ActRIIB-Fc:ALK4-Fc heterodimer displays enhanced binding to activin B compared with either homodimer, retains strong binding to activin A, GDF8, and GDF11 as observed with ActRIIB-Fc homodimer, and exhibits substantially reduced binding to BMP9, BMP10, and GDF3. Like ActRIIB-Fc homodimer, the heterodimer retains intermediate-level binding to BMP6.

FIG. 20 shows comparative ActRIIB-Fc:ALK4-Fc heterodimer/ActRIIB-Fc:ActRIIB-Fc homodimer IC₅₀ data as determined by an A-204 Reporter Gene Assay as described herein. ActRIIB-Fc:ALK4-Fc heterodimer inhibits activin A, activin B, GDF8, and GDF11 signaling pathways similarly to the ActRIIB-Fc:ActRIIB-Fc homodimer. However, ActRIIB-Fc:ALK4-Fc heterodimer inhibition of BMP9 and BMP10 signaling pathways is significantly reduced compared to the ActRIIB-Fc:ActRIIB-Fc homodimer. These data demonstrate that ActRIIB:ALK4 heterodimers are more selective antagonists of activin A, activin B, GDF8, and GDF11 compared to corresponding ActRIIB:ActRIIB homodimers.

FIG. 21 shows comparative ligand binding data for an ActRIIB-Fc:ALK7-Fc heterodimeric protein complex compared to ActRIIB-Fc homodimer and ALK7-Fc homodimer. For each protein complex, ligands are ranked by k_{off}, a kinetic constant that correlates well with ligand signaling inhibition, and listed in descending order of binding affinity (ligands bound most tightly are listed at the top). At left, yellow, red, green, and blue lines indicate magnitude of the off-rate constant. Solid black lines indicate ligands whose binding to heterodimer is enhanced or unchanged compared with homodimer, whereas dashed red lines indicate substantially reduced binding compared with homodimer. As shown, four of the five ligands with strong binding to ActRIIB-Fc homodimer (activin A, BMP10, GDF8, and GDF11) exhibit reduced binding to the ActRIIB-Fc:ALK7-Fc heterodimer, the exception being activin B which retains tight binding to the heterodimer. Similarly, three of four ligands with intermediate binding to ActRIIB-Fc homodimer (GDF3, BMP6, and particularly BMP9) exhibit reduced binding to the ActRIIB-Fc:ALK7-Fc heterodimer, whereas binding to activin AC is increased to become the second strongest ligand interaction with the heterodimer overall. Finally, activin C and BMP5 unexpectedly bind the ActRIIB-Fc:ALK7 heterodimer with intermediate strength despite no binding (activin C) or weak binding (BMP5) to ActRIIB-Fc homodimer. No ligands tested bind to ALK7-Fc homodimer.

FIG. 22 shows a multiple sequence alignment of ALK7 extracellular domains derived from various vertebrate species (SEQ ID NOs: 425-430).

FIG. 23 ActRIIB-Fc:ALK4-Fc rescued cardiac diastolic dysfunction during LV remodeling in the aged heart. Thirteen male mice at 24-months of age (“Old”) and 10 mice at 4-months of age (“Young”) were studied. Groups of “Old” and “Young” mice received phosphate-buffered saline (PBS) twice per week subcutaneously for 8 weeks (“Young-Vehicle” or “Old-Vehicle”). Another group of “Old” mice received ActRIIB-Fc:ALK4-Fc (10 mg/kg) twice per week subcutaneously for 8 weeks (“Old-ActRIIB-Fc:ALK4-Fc”). The volume of vehicle and volume of ActRIIB-Fc:ALK4-Fc administered was the same. E/e′, a measurement of diastolic dysfunction, was increased in “Old-Vehicle” (n=7) mice compared “Young-Vehicle” mice (n=10, p<0.01). ActRIIB-Fc:ALK4-Fc treatment in “Old-ActRIIB-Fc:ALK4-Fc” mice significantly reduced E/e′ (n=6, p<0.05).

### DETAILED DESCRIPTION

### 1. Overview

In certain aspects, the disclosure relates to methods of using TGF-β superfamily ligand antagonists, in particular ActRII-ALK4 antagonists, to treat heart failure. For example, ActRII-ALK4 antagonists as described herein may be used to treat, prevent, or reduce the progression rate and/or severity of heart failure associated with aging, or one or more complications of heart failure associated with aging.

Heart Failure (HF) is a clinical syndrome characterized by symptoms that include breathlessness, ankle swelling and fatigue, that may be accompanied by signs that include elevated jugular venous pressure, pulmonary crackles and peripheral edema caused by a structural and/or functional cardiac abnormality. HF typically results in a reduced cardiac output and/or elevated intracardiac pressure at rest or during stress.

Before clinical symptoms become apparent, patients may present with asymptomatic structural or functional cardiac abnormalities (e.g., systolic or diastolic left ventricular (LV) dysfunction), which are precursors of HF. Recognition of these precursors is important because they are related to poor outcomes, and starting treatment at the precursor stage may reduce mortality in patients with asymptomatic systolic LV dysfunction.

Demonstration of an underlying cardiac cause is central to the diagnosis of HF. This usually includes a myocardial abnormality causing systolic and/or diastolic ventricular dysfunction. However, abnormalities of the valves, pericardium, endocardium, heart rhythm and conduction can also cause HF (and more than one abnormality is often present). Identification of the underlying cardiac problem is crucial for therapeutic reasons, as the precise pathology determines the specific treatment used (e.g., valve repair or replacement for valvular disease, specific pharmacological therapy for HF with reduced EF, reduction of heart rate in tachycardiomyopathy, etc.).

TGF-β superfamily ligand signals are mediated by heteromeric complexes of type I and type II serine/threonine kinase receptors, which phosphorylate and activate downstream Smad proteins upon ligand stimulation (Massagué, 2000, Nat. Rev. Mol. Cell Biol. 1:169-178). These type I and type II receptors are all transmembrane polypeptides, composed of a ligand-binding extracellular domain with cysteine-rich region, a transmembrane domain, and a cytoplasmic domain with predicted serine/threonine specificity. Type I receptors are essential for signaling, and type II receptors are required for binding ligands. Type I and type II activin receptors form a stable complex after ligand binding, resulting in phosphorylation of type I receptors by type II receptors.

Two related type II receptors, ActRIIA and ActRIIB, have been identified as the type II receptors for activins (Mathews and Vale, 1991, Cell 65:973-982; Attisano et al., 1992, Cell 68: 97-108). Besides activins, ActRIIA and ActRIIB can biochemically interact with several other TGF-β family proteins, including BMP7, Nodal, GDF8, and GDF11 (Yamashita et al., 1995, J. Cell Biol. 130:217-226; Lee and McPherron, 2001, Proc. Natl. Acad. Sci. 98:9306-9311; Yeo and Whitman, 2001, Mol. Cell 7: 949-957; Oh et al., 2002, Genes Dev. 16:2749-54). Applicants have found that soluble ActRIIA-Fc fusion polypeptides and ActRIIB-Fc fusion polypeptides have substantially different effects in vivo, with ActRIIA-Fc having primary effects on bone and ActRIIB-Fc having primary effects on skeletal muscle.

Ligands of the TGF-beta superfamily share the same dimeric structure in which the central 3½ turn helix of one monomer packs against the concave surface formed by the beta-strands of the other monomer. The majority of TGF-beta family members are further stabilized by an intermolecular disulfide bond. This disulfide bonds traverses through a ring formed by two other disulfide bonds generating what has been termed a ‘cysteine knot’ motif [Lin et al. (2006) Reproduction 132: 179-190; and Hinck et al. (2012) FEBS Letters 586: 1860-1870].

Activins are members of the TGF-beta superfamily and were initially discovered as regulators of secretion of follicle-stimulating hormone, but subsequently various reproductive and non-reproductive roles have been characterized. There are three principal activin forms (A, B, and AB) that are homo/heterodimers of two closely related β subunits (β_{A}β_{A}, β_{B}β_{B}, and β_{A}β_{B}, respectively). The human genome also encodes an activin C and an activin E, which are primarily expressed in the liver, and heterodimeric forms containing β_{C} or β_{E} are also known. In the TGF-beta superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos [DePaolo et al. (1991) Proc Soc Ep Biol Med. 198:500-512; Dyson et al. (1997) Curr Biol. 7:81-84; and Woodruff (1998) Biochem Pharmacol. 55:953-963]. In several tissues, activin signaling is antagonized by its related heterodimer, inhibin. For example, in the regulation of follicle-stimulating hormone (FSH) secretion from the pituitary, activin promotes FSH synthesis and secretion, while inhibin reduces FSH synthesis and secretion. Other proteins that may regulate activin bioactivity and/or bind to activin include follistatin (FS) and α₂-macroglobulin.

As described herein, agents that bind to “activin A” are agents that specifically bind to the β_{A} subunit, whether in the context of an isolated β_{A} subunit or as a dimeric complex (e.g., a β_{A}β_{A} homodimer or a β_{A}β_{B} heterodimer). In the case of a heterodimer complex (e.g., a β_{A}β_{B} heterodimer), agents that bind to “activin A” are specific for epitopes present within the β_{A} subunit, but do not bind to epitopes present within the non-β_{A} subunit of the complex (e.g., the β_{B} subunit of the complex). Similarly, agents disclosed herein that antagonize (inhibit) “activin A” are agents that inhibit one or more activities as mediated by a βA subunit, whether in the context of an isolated β_{A} subunit or as a dimeric complex (e.g., a β_{A}β_{A} homodimer or a β_{A}β_{B} heterodimer). In the case of β_{A}β_{B} heterodimers, agents that inhibit “activin A” are agents that specifically inhibit one or more activities of the β_{A} subunit, but do not inhibit the activity of the non-β_{A} subunit of the complex (e.g., the β_{B} subunit of the complex). This principle applies also to agents that bind to and/or inhibit “activin B”, “activin C”, and “activin E”. Agents disclosed herein that antagonize “activin AB” are agents that inhibit one or more activities as mediated by the β_{A} subunit and one or more activities as mediated by the β_{B} subunit.

The BMPs and GDFs together form a family of cysteine-knot cytokines sharing the characteristic fold of the TGF-beta superfamily [Rider et al. (2010) Biochem J., 429(1):1-12]. This family includes, for example, BMP2, BMP4, BMP6, BMP7, BMP2a, BMP3, BMP3b (also known as GDF10), BMP4, BMP5, BMP6, BMP7, BMP8, BMP8a, BMP8b, BMP9 (also known as GDF2), BMP10, BMP11 (also known as GDF11), BMP12 (also known as GDF7), BMP13 (also known as GDF6), BMP14 (also known as GDF5), BMP15, GDF1, GDF3 (also known as VGR2), GDF8 (also known as myostatin), GDF9, GDF15, and decapentaplegic. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines. Curiously, a number of these antagonists resemble TGF-beta superfamily ligands.

Growth and differentiation factor-8 (GDF8) is also known as myostatin. GDF8 is a negative regulator of skeletal muscle mass. GDF8 is highly expressed in the developing and adult skeletal muscle. The GDF8 null mutation in transgenic mice is characterized by a marked hypertrophy and hyperplasia of the skeletal muscle (McPherron et al., Nature, 1997, 387:83-90). Similar increases in skeletal muscle mass are evident in naturally occurring mutations of GDF8 in cattle (Ashmore et al., 1974, Growth, 38:501-507; Swatland and Kieffer, J. Anim. Sci., 1994, 38:752-757; McPherron and Lee, Proc. Natl. Acad. Sci. USA, 1997, 94:12457-12461; and Kambadur et al., Genome Res., 1997, 7:910-915) and, strikingly, in humans (Schuelke et al., N Engl J Med 2004; 350:2682-8). Studies have also shown that muscle wasting associated with HIV-infection in humans is accompanied by increases in GDF8 polypeptide expression (Gonzalez-Cadavid et al., Proc Natl Acad Sci USA, 1998, 95:14938-43). In addition, GDF8 can modulate the production of muscle-specific enzymes (e.g., creatine kinase) and modulate myoblast cell proliferation (WO 00/43781). The GDF8 propeptide can noncovalently bind to the mature GDF8 domain dimer, inactivating its biological activity (Miyazono et al. (1988) J. Biol. Chem., 263: 6407-6415; Wakefield et al. (1988) J. Biol. Chem., 263; 7646-7654; and Brown et al. (1990) Growth Factors, 3: 35-43). Other polypeptides which bind to GDF8 or structurally related polypeptides and inhibit their biological activity include follistatin, and potentially, follistatin-related polypeptides (Gamer et al. (1999) Dev. Biol., 208: 222-232).

Growth and differentiation factor-11 (GDF11), also known as BMP11, is a secreted protein (McPherron et al., 1999, Nat. Genet. 22: 260-264). GDF11 is expressed in the tail bud, limb bud, maxillary and mandibular arches, and dorsal root ganglia during mouse development (Nakashima et al., 1999, Mech. Dev. 80: 185-189). GDF11 plays a unique role in patterning both mesodermal and neural tissues (Gamer et al., 1999, Dev Biol., 208:222-32). GDF11 was shown to be a negative regulator of chondrogenesis and myogenesis in developing chick limb (Gamer et al., 2001, Dev Biol. 229:407-20). The expression of GDF11 in muscle also suggests its role in regulating muscle growth in a similar way to GDF8. In addition, the expression of GDF11 in brain suggests that GDF11 may also possess activities that relate to the function of the nervous system. Interestingly, GDF11 was found to inhibit neurogenesis in the olfactory epithelium (Wu et al., 2003, Neuron. 37:197-207).

In part, the examples of the disclosure demonstrate that an ActRIIB:ALK4 heterodimer is effective to improve diastolic dysfunction as measured by E/e′. Ejection fraction was not reduced in aged mice compared to young mice, while BNP levels increased, indicative of HFpEF. The data further suggest that, in addition to ActRIIB:ALK4 heteromultimers, other ActRII-ALK4 antagonists may be useful in treating heart failure associated with aging.

In certain aspects, an ActRII-ALK4 antagonist to be used in accordance with the methods and uses disclosed herein (e.g., treating, preventing, or reducing the progression rate and/or severity of heart failure associated with aging, or one or more complications of heart failure) is an ActRII-ALK4 ligand trap polypeptide antagonist including variants thereof as well as heterodimers and heteromultimers thereof, an ActRII-ALK4 antibody antagonist, an ActRII-ALK4 polynucleotide antagonist, and/or an ActRII-ALK4 small molecule antagonist. ActRII-ALK4 ligand trap polypeptides include TGF-β superfamily-related proteins, including variants thereof, that are capable of binding to one or more ActRII-ALK4 ligands (e.g., activin A, activin B, GDF8, GDF11, BMP6, and/or BMP10). Therefore, an ActRII-ALK4 ligand trap generally includes polypeptides that are capable of antagonizing one or more ActRII-ALK4 ligands (e.g., activin A, activin B, GDF8, GDF11, BMP6, and/or BMP10). In some embodiments, an ActRII-ALK4 antagonist comprises an ActRII-ALK4 ligand trap. In some embodiments, an ActRII-ALK4 ligand trap comprises an ActRIIB polypeptide, including variants thereof, as well as homomultimers (e.g., ActRIIB homodimers) and heteromultimers (e.g., ActRIIB-ALK4 or ActRIIB-ALK7 heterodimers). In some embodiments, an ActRII-ALK4 ligand trap comprises an ActRIIA polypeptide, including variants thereof, as well as homomultimers (e.g., ActRIIA homodimers) and heteromultimers (e.g., ActRIIA-ALK4 or ActRIIA-ALK7 heterodimers). In other embodiments, an ActRII-ALK ligand trap comprises a soluble ligand trap protein including, but not limited to, or a follistatin polypeptide as well as variants thereof. In some embodiments, an ActRII-ALK4 antagonist comprises an ActRII-ALK4 antibody antagonist (antibodies that inhibit one or more of activin A, activin B, GDF8, GDF11, BMP6, BMP10, ActRIIB, ActRIIA, ALK4 and/or ALK7). In some embodiments, an ActRII-ALK4 antagonist comprises an ActRII-ALK4 small molecule antagonist (e.g., small molecules that inhibit one or more of activin A, activin B, GDF8, GDF11, BMP6, BMP10, ActRIIB, ActRIIA, ALK4 and/or ALK7). In some embodiments, an ActRII-ALK4 antagonist comprises an ActRII-ALK4 polynucleotide antagonist (e.g., nucleotide sequences that inhibit one or more of activin A, activin B, GDF8, GDF11, BMP6, BMP10, ActRIIB, ActRIIA, ALK4 and/or ALK7).

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is ±10%. Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. ActRII-ALK4 Ligand Trap Antagonists and Variants Thereof

In certain aspects, an ActRII-ALK4 antagonist to be used in accordance with the methods and uses disclosed herein (e.g., treating, preventing, or reducing the progression rate and/or severity of heart failure associated with aging, or one or more complications of heart failure) is an ActRII-ALK4 ligand trap polypeptide including variants thereof as well as heterodimers and heteromultimers thereof. ActRII-ALK4 ligand trap polypeptides include TGF-β superfamily-related proteins, including variants thereof, that are capable of binding to one or more ActRII-ALK4 ligands (e.g., activin A, activin B, GDF8, GDF11, BMP6, and BMP10). Therefore, ActRII-ALK4 ligand trap generally include polypeptides that are capable of antagonizing one or more ActRII-ALK4 ligands (e.g., activin A, activin B, GDF8, GDF11, BMP6, and BMP10). For example, in some embodiments, an ActRII-ALK4 ligand trap comprises an ActRII polypeptide, including variants thereof, as well as homo- and hetero-multimers thereof (e.g., homodimer and heterodimers, respectively). As used herein, the term “ActRII” refers to the family of type II activin receptors. This family includes activin receptor type IIA (ActRIIA) and activin receptor type IIB (ActRIIB). In some embodiments, an ActRII-ALK4 ligand trap comprises an ActRIIB polypeptide, including variants thereof, as well as homomultimers (e.g., ActRIIB homodimers) and heteromultimers (e.g., ActRIIB-ALK4 or ActRIIB-ALK7 heterodimers). In some embodiments, an ActRII-ALK4 ligand trap comprises an ActRIIA polypeptide, including variants thereof, as well as homomultimers (e.g., ActRIIA homodimers) and heteromultimers (e.g., ActRIIA-ALK4 or ActRIIA-ALK7 heterodimers). In other embodiments, an ActRII-ALK ligand trap comprises a soluble ligand trap protein including, but not limited to, or a follistatin polypeptide as well as variants thereof.

A) ActRIIB Polypeptides

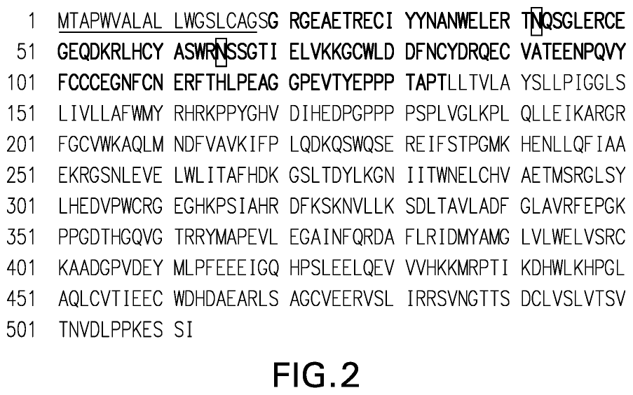

In certain aspects, the disclosure relates to ActRII-ALK4 antagonists comprising an ActRIIB polypeptide, which includes fragments, functional variants, and modified forms thereof as well as uses thereof (e.g., of treating, preventing, or reducing the progression rate and/or severity of heart failure (HF) or one or more complications of HF). As used herein, the term “ActRIIB” refers to a family of activin receptor type IIB (ActRIIB) proteins from any species and variant polypeptides derived from such ActRIIB proteins by mutagenesis or other modifications (including, e.g., mutants, fragments, fusions, and peptidomimetic forms) that retain a useful activity. Examples of such variant ActRIIB polypeptides are provided throughout the present disclosure as well as in International Patent Application Publication Nos. WO 2006/012627, WO 2008/097541, WO 2010/151426, WO 2011/020045, WO 2018/009624, and WO 2018/067874 which are incorporated herein by reference in their entirety. Reference to ActRIIB herein is understood to be a reference to any one of the currently identified forms. Members of the ActRIIB family are generally all transmembrane polypeptides, composed of a ligand-binding extracellular domain with cysteine-rich region, a transmembrane domain, and a cytoplasmic domain with predicted serine/threonine kinase specificity. The amino acid sequence of human ActRIIB precursor polypeptide is shown in FIG. 2 (SEQ ID NO: 2) and below. Preferably, ActRIIB polypeptides to be used in accordance with the methods of the disclosure are soluble. The term “soluble ActRIIB polypeptide,” as used herein, includes any naturally occurring extracellular domain of an ActRIIB polypeptide as well as any variants thereof (including mutants, fragments and peptidomimetic forms) that retain a useful activity. For example, the extracellular domain of an ActRIIB polypeptide binds to a ligand and is generally soluble. Examples of soluble ActRIIB polypeptides include an ActRIIB extracellular domain (SEQ ID NO: 1) shown in FIG. 3 as well as SEQ ID NO: 53. This truncated ActRIIB extracellular domain (SEQ ID NO: 53) is denoted ActRIIB(25-131) based on numbering in SEQ ID NO: 2. Other examples of soluble ActRIIB polypeptides comprise a signal sequence in addition to the extracellular domain of an ActRIIB polypeptide (see Example 4). The signal sequence can be a native signal sequence of an ActRIIB, or a signal sequence from another polypeptide, such as a tissue plasminogen activator (TPA) signal sequence or a honey bee melittin signal sequence. In some embodiments, ActRIIB polypeptides inhibit activity (e.g., Smad signaling) of one or more ActRII-ALK4 ligands (e.g., activin A, activin B, GDF8, GDF11, BMP6, BMP10). In some embodiments, ActRIIB polypeptides bind to one or more ActRII-ALK4 ligands (e.g., activin A, activin B, GDF8, GDF11, BMP6, BMP10). Various examples of methods and assays for determining the ability of an ActRIIB polypeptide to bind to and/or inhibit activity of one or more ActRII-ALK4 ligands are disclosed herein or otherwise well known in the art, which can be readily used to determine if an ActRIIB polypeptide has the desired binding and/or antagonistic activities. Numbering of amino acids for all ActRIIB-related polypeptides described herein is based on the numbering of the human ActRIIB precursor protein sequence provided below (SEQ ID NO: 2), unless specifically designated otherwise.

The human ActRIIB precursor protein sequence is as follows:

| (SEQ ID NO: 2, FIG. 2) | |
|---|---|
| 1 | MTAPWVALAL LWGSLCAGSG RGEAETRECI YYNANWELER T [symbol] QSGLERCE |
| 51 | GEQDKRLHCY ASWR [symbol] SSGTI ELVKKGCWLD DFNCYDRQEC VATEENPQVY |
| 101 | FCCCEGNFCN ERFTHLPEAG GPEVTYEPPP TAPTLLTVLA YSLLPIGGLS |
| 151 | LIVLLAFWMY RHRKPPYGHV DIHEDPGPPP PSPLVGLKPL QLLEIKARGR |
| 201 | FGCVWKAQLM NDFVAVKIFP LQDKQSWQSE REIFSTPGMK HENLLQFIAA |
| 251 | EKRGSNLEVE LWLITAFHDK GSLTDYLKGN IITWNELCHV AETMSRGLSY |
| 301 | LHEDVPWCRG EGHKPSIAHR DFKSKNVLLK SDLTAVLADF GLAVRFEPGK |
| 351 | PPGDTHGQVG TRRYMAPEVL EGAINFQRDA FLRIDMYAMG LVLWELVSRC |
| 401 | KAADGPVDEY MLPFEEEIGQ HPSLEELQEV VVHKKMRPTI KDHWLKHPGL |
| 451 | AQLCVTIEEC WDHDAEARLS AGCVEERVSL IRRSVNGTTS DCLVSLVTSV |
| 501 | TNVDLPPKES SI |

The signal peptide is indicated with a single underline; the extracellular domain is indicated in bold font; and the potential, endogenous N-linked glycosylation sites are indicated with a double underline.

A processed (mature) extracellular ActRIIB polypeptide sequence is as follows:

(SEQ ID NO: 1, FIG. 3)
GRGEAETRECIYYNANWELERTNQSGLERCEGEQDKRLHCYASWRNSSG
TIELVKKGCWLDDFNCYDRQECVATEENPQVYFCCCEGNFCNERFTHLP
EAGGPEVTYEPPPTAPT.

In some embodiments, the protein may be produced with an “SGR . . . ” sequence at the N-terminus. The C-terminal “tail” of the extracellular domain is indicated by a single underline. The sequence with the “tail” deleted (a Δ15 sequence) is as follows:

(SEQ ID NO: 386)
GRGEAETRECIYYNANWELERTNQSGLERCEGEQDKRLHCYASWRN
SSGTIELVKKGCWLDDFNCYDRQECVATEENPQVYFCCCEGNFCNE
RFTHLPEA.

A form of ActRIIB with an alanine at position 64 of SEQ ID NO: 2 (A64) is also reported in the literature. See, e.g., Hilden et al. (1994) Blood, 83(8): 2163-2170. Applicants have ascertained that an ActRIIB-Fc fusion protein comprising an extracellular domain of ActRIIB with the A64 substitution has a relatively low affinity for activin and GDF11. By contrast, the same ActRIIB-Fc fusion protein with an arginine at position 64 (R64) has an affinity for activin and GDF11 in the low nanomolar to high picomolar range. Therefore, sequences with an R64 are used as the “wild-type” reference sequence for human ActRIIB in this disclosure.

The form of ActRIIB precursor protein sequence with an alanine at position 64 is as follows:

```
(SEQ ID NO: 387)
    1 MTAPWVALAL LWGSLCAGSG RGEAETRECI
        YYNANWELER TNQSGLERCE
  51 GEQDKRLHCY ASWANSSGTI ELVKKGCWLD
        DENCYDRQEC VATEENPQVY
101 FCCCEGNFCN ERFTHLPEAG GPEVTYEPPP
        TAPTLLTVLA YSLLPIGGLS
151 LIVLLAFWMY RHRKPPYGHV DIHEDPGPPP
        PSPLVGLKPL QLLEIKARGR
201 FGCVWKAQLM NDEVAVKIFP LQDKQSWQSE
        REIFSTPGMK HENLLQFIAA
251 EKRGSNLEVE LWLITAFHDK GSLTDYLKGN
        IITWNELCHV AETMSRGLSY
301 LHEDVPWCRG EGHKPSIAHR DEKSKNVLLK
        SDLTAVLADF GLAVRFEPGK
351 PPGDTHGQVG TRRYMAPEVL EGAINFQRDA
        FLRIDMYAMG LVLWELVSRC
401 KAADGPVDEY MLPFEEEIGQ HPSLEELQEV
        VVHKKMRPTI KDHWLKHPGL
451 AQLCVTIEEC WDHDAEARLS AGCVEERVSL
        IRRSVNGTTS DCLVSLVTSV
501 TNVDLPPKES SI
```

The signal peptide is indicated by single underline and the extracellular domain is indicated by bold font.

A processed (mature) extracellular ActRIIB polypeptide sequence of the alternative A64 form is as follows:

(SEQ ID NO: 388)
GRGEAETRECIYYNANWELERTNQSGLERCEGEQDKRLHCYASWA
NSSGTIELVKKGCWLDDENCYDRQECVATEENPQVYFCCCEGNFC
NERFTHLPEAGGPEVTYEPPPTAPT

In some embodiments, the protein may be produced with an “SGR . . . ” sequence at the N-terminus. The C-terminal “tail” of the extracellular domain is indicated by single underline. The polypeptide sequence of the alternative A64 form with the “tail” deleted (a Δ15 sequence) is as follows:

(SEQ ID NO: 389)
GRGEAETRECIYYNANWELERTNQSGLERCEGEQDKRLHCYASWA
NSSGTIELVKKGCWLDDFNCYDRQECVATEENPQVYFCCCEGNFC
NERFTHLPEA

A nucleic acid sequence encoding the human ActRIIB precursor protein is shown below (SEQ ID NO: 4), representing nucleotides 25-1560 of GenBank Reference Sequence NM_001106.3, which encodes amino acids 1-513 of the ActRIIB precursor. The nucleotide sequence as shown encodes a polypeptide with an arginine at position 64 and may be modified to encode a polypeptide with an alanine instead. The signal sequence is underlined.

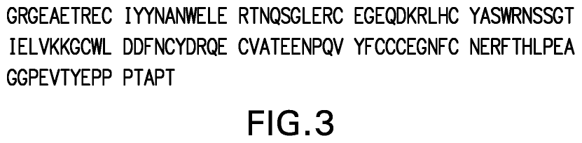

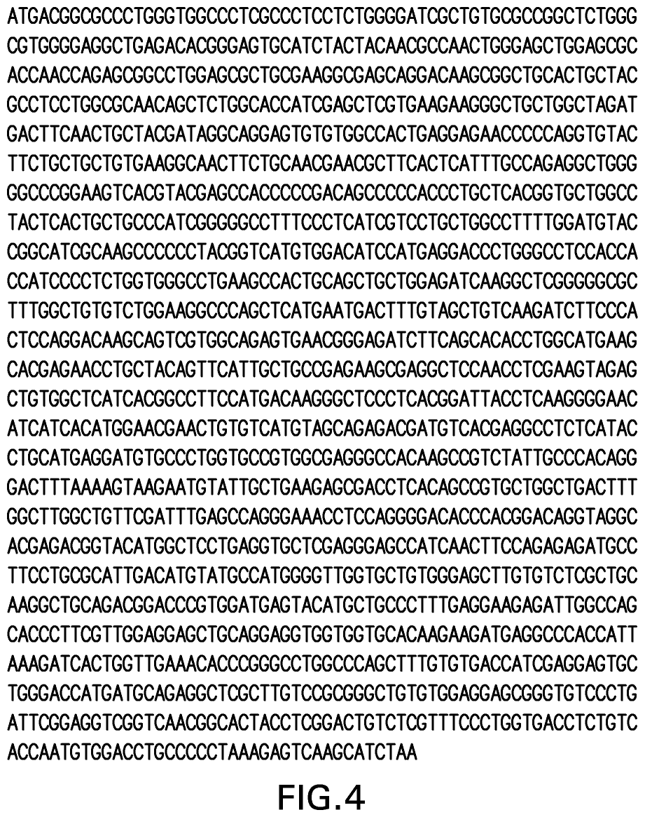

```
(SEQ ID NO: 4, FIG. 4)
      1 ATGACGGCGC CCTGGGTGGC CCTCGCCCTC
          CTCTGGGGAT CGCTGTGCGC
    51 CGGCTCTGGG CGTGGGGAGG CTGAGACACG
          GGAGTGCATC TACTACAACG
  101 CCAACTGGGA GCTGGAGCGC ACCAACCAGA
          GCGGCCTGGA GCGCTGCGAA
  151 GGCGAGCAGG ACAAGCGGCT GCACTGCTAC
          GCCTCCTGGC GCAACAGCTC
  201 TGGCACCATC GAGCTCGTGA AGAAGGGCTG
          CTGGCTAGAT GACTTCAACT
  251 GCTACGATAG GCAGGAGTGT GTGGCCACTG
          AGGAGAACCC CCAGGTGTAC
  301 TTCTGCTGCT GTGAAGGCAA CTTCTGCAAC
          GAACGCTTCA CTCATTTGCC
  351 AGAGGCTGGG GGCCCGGAAG TCACGTACGA
          GCCACCCCCG ACAGCCCCCA
  401 CCCTGCTCAC GGTGCTGGCC TACTCACTGC
          TGCCCATCGG GGGCCTTTCC
  451 CTCATCGTCC TGCTGGCCTT TTGGATGTAC
          CGGCATCGCA AGCCCCCCTA
  501 CGGTCATGTG GACATCCATG AGGACCCTGG
          GCCTCCACCA CCATCCCCTC
  551 TGGTGGGCCT GAAGCCACTG CAGCTGCTGG
          AGATCAAGGC TCGGGGGCGC
  601 TTTGGCTGTG TCTGGAAGGC CCAGCTCATG
          AATGACTTTG TAGCTGTCAA
  651 GATCTTCCCA CTCCAGGACA AGCAGTCGTG
          GCAGAGTGAA CGGGAGATCT
  701 TCAGCACACC TGGCATGAAG CACGAGAACC
          TGCTACAGTT CATTGCTGCC
  751 GAGAAGCGAG GCTCCAACCT CGAAGTAGAG
          CTGTGGCTCA TCACGGCCTT
  801 CCATGACAAG GGCTCCCTCA CGGATTACCT
          CAAGGGGAAC ATCATCACAT
  851 GGAACGAACT GTGTCATGTA GCAGAGACGA
          TGTCACGAGG CCTCTCATAC
  901 CTGCATGAGG ATGTGCCCTG GTGCCGTGGC
          GAGGGCCACA AGCCGTCTAT
  951 TGCCCACAGG GACTTTAAAA GTAAGAATGT
          ATTGCTGAAG AGCGACCTCA
1001 CAGCCGTGCT GGCTGACTTT GGCTTGGCTG
          TTCGATTTGA GCCAGGGAAA
1051 CCTCCAGGGG ACACCCACGG ACAGGTAGGC
          ACGAGACGGT ACATGGCTCC
1101 TGAGGTGCTC GAGGGAGCCA TCAACTTCCA
          GAGAGATGCC TTCCTGCGCA
1151 TTGACATGTA TGCCATGGGG TTGGTGCTGT
          GGGAGCTTGT GTCTCGCTGC
1201 AAGGCTGCAG ACGGACCCGT GGATGAGTAC
          ATGCTGCCCT TTGAGGAAGA
1251 GATTGGCCAG CACCCTTCGT TGGAGGAGCT
          GCAGGAGGTG GTGGTGCACA
1301 AGAAGATGAG GCCCACCATT AAAGATCACT
          GGTTGAAACA CCCGGGCCTG
1351 GCCCAGCTTT GTGTGACCAT CGAGGAGTGC
          TGGGACCATG ATGCAGAGGC
1401 TCGCTTGTCC GCGGGCTGTG TGGAGGAGCG
          GGTGTCCCTG ATTCGGAGGT
1451 CGGTCAACGG CACTACCTCG GACTGTCTCG
          TTTCCCTGGT GACCTCTGTC
1501 ACCAATGTGG ACCTGCCCCC TAAAGAGTCA
          AGCATC
```

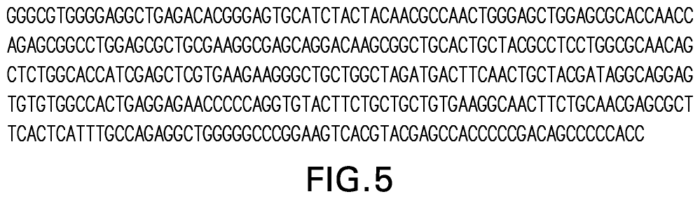

A nucleic acid sequence encoding a processed extracellular human ActRIIB polypeptide is as follows (SEQ ID NO: 3). The nucleotide sequence as shown encodes a polypeptide with an arginine at position 64, and may be modified to encode a polypeptide with an alanine instead (See FIG. 5, SEQ ID NO: 3).

```
(SEQ ID NO: 3)
    1 GGGCGTGGGG AGGCTGAGAC ACGGGAGTGC
        ATCTACTACA ACGCCAACTG
  51 GGAGCTGGAG CGCACCAACC AGAGCGGCCT
        GGAGCGCTGC GAAGGCGAGC
101 AGGACAAGCG GCTGCACTGC TACGCCTCCT
        GGCGCAACAG CTCTGGCACC
151 ATCGAGCTCG TGAAGAAGGG CTGCTGGCTA
        GATGACTTCA ACTGCTACGA
201 TAGGCAGGAG TGTGTGGCCA CTGAGGAGAA
        CCCCCAGGTG TACTTCTGCT
251 GCTGTGAAGG CAACTTCTGC AACGAACGCT
        TCACTCATTT GCCAGAGGCT
301 GGGGGCCCGG AAGTCACGTA CGAGCCACCC
        CCGACAGCCC CCACC
```

B) Variant ActRIIB Polypeptides

In certain specific embodiments, the present disclosure contemplates making mutations in the extracellular domain (also referred to as ligand-binding domain) of an ActRIIB polypeptide such that the variant (or mutant) ActRIIB polypeptide has altered ligand-binding activities (e.g., binding affinity or binding selectivity). In certain cases, such variant ActRIIB polypeptides have altered (elevated or reduced) binding affinity for a specific ligand. In other cases, the variant ActRIIB polypeptides have altered binding selectivity for their ligands. For example, the disclosure provides a number of variant ActRIIB polypeptides that have reduced binding affinity to BMP9, compared to a non-modified ActRIIB polypeptide, but retain binding affinity for one or more of activin A, activin B, GDF8, GDF11, and BMP10. Optionally, the variant ActRIIB polypeptides have similar or the same biological activities of their corresponding wild-type ActRIIB polypeptides. For example, a variant ActRIIB polypeptide of the disclosure may bind to and inhibit function of an ActRIIB ligand (e.g., activin A, activin B, GDF8, GDF11 or BMP10). In some embodiments, a variant ActRIIB polypeptide of the disclosure treats, prevents, or reduces the progression rate and/or severity of heart failure or one or more complications of heart failure. Examples of ActRIIB polypeptides include human ActRIIB precursor polypeptide (SEQ ID NO: 2 and SEQ ID NO:387), and soluble human ActRIIB polypeptides (e.g., SEQ ID NOs: 1, 5, 6, 12, 276, 278, 279, 332, 333, 335, 336, 338, 339, 341, 342, 344, 345, 347, 348, 350, 351, 353, 354, 356, 357, 385, 386, 387, 388, 389, 396, 398, 402, 403, 406, 408, and 409). In some embodiments, the variant ActRIIB polypeptide is a member of a homomultimer (e.g., homodimer). In some embodiments, the variant ActRIIB polypeptide is a member of a heteromultimer (e.g., a heterodimer). In some embodiments, any of the variant ActRIIB polypeptides may be combined (e.g., heteromultimerized with and/or fused to) with any of polypeptides disclosed herein.

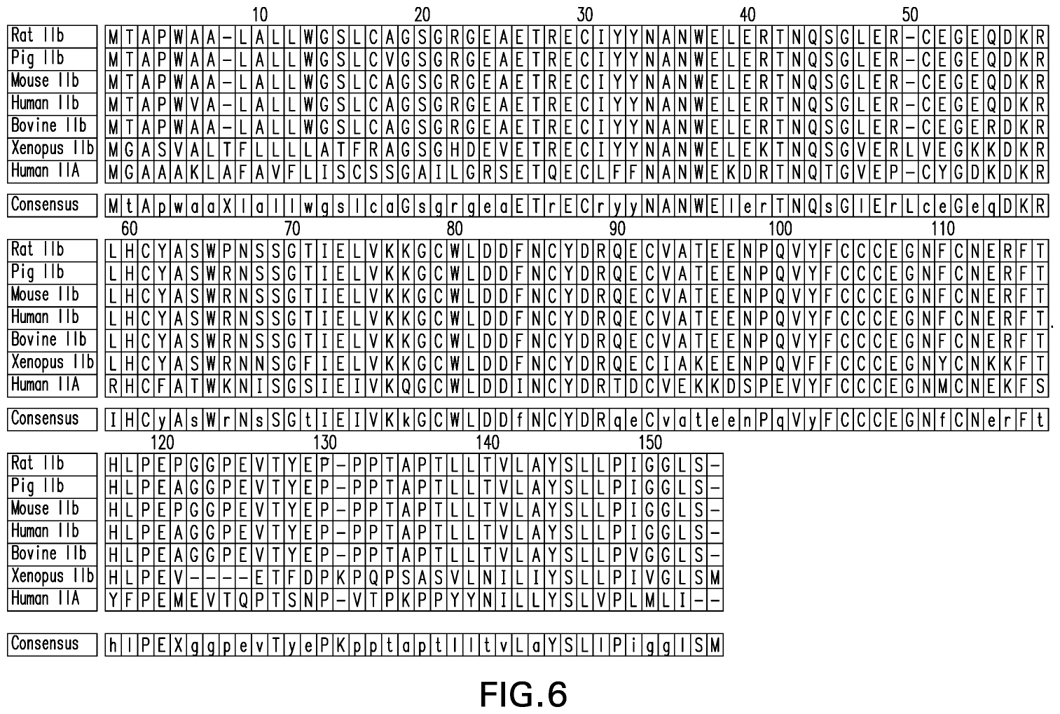

ActRIIB is well-conserved across nearly all vertebrates, with large stretches of the extracellular domain conserved completely. See, e.g., FIG. 6. Many of the ligands that bind to ActRIIB are also highly conserved. Accordingly, comparisons of ActRIIB sequences from various vertebrate organisms provide insights into residues that may be altered. Therefore, an active, human ActRIIB variant may include one or more amino acids at corresponding positions from the sequence of another vertebrate ActRIIB, or may include a residue that is similar to that in the human or other vertebrate sequence.

The disclosure identifies functionally active portions and variants of ActRIIB. Applicant has previously ascertained that an Fc fusion polypeptide having the sequence disclosed by Hilden et al. (Blood. 1994 Apr. 15; 83(8):2163-70), which has an alanine at the position corresponding to amino acid 64 of SEQ ID NO: 2 (A64), has a relatively low affinity for activin and GDF11. By contrast, the same Fc fusion polypeptide with an arginine at position 64 (R64) has an affinity for activin and GDF-11 in the low nanomolar to high picomolar range. Therefore, a sequence with an R64 (SEQ ID NO: 2) is used as the wild-type reference sequence for human ActRIIB in this disclosure, and the numbering for the variants described herein are based on the numbering in SEQ ID NO: 2. Additionally, one of skill in the art can make any of the ActRIIB variants described herein in the A64 background.

A processed extracellular ActRIIB polypeptide sequence is shown in SEQ ID NO: 1 (see, e.g., FIG. 3). In some embodiments, a processed ActRIIB polypeptide may be produced with an “SGR . . . ” sequence at the N-terminus. In some embodiments, a processed ActRIIB polypeptide may be produced with a “GRG . . . ” sequence at the N-terminus. For example, it is expected that some constructs, if expressed with a TPA leader, will lack the N-terminal serine. Accordingly, mature ActRIIB sequences described herein may begin with either an N-terminal serine or an N-terminal glycine (lacking the N-terminal serine).

Attisano et al. (Cell. 1992 Jan. 10; 68(1):97-108) showed that a deletion of the proline knot at the C-terminus of the extracellular domain of ActRIIB reduced the affinity of the receptor for activin. Data disclosed in WO2008097541 show that an ActRIIB-Fc fusion polypeptide containing amino acids 20-119 of SEQ ID NO: 2, “ActRIIB(20-119)-Fc” has reduced binding to GDF11 and activin relative to an ActRIIB(20-134)-Fc, which includes the proline knot region and the complete juxtamembrane domain. However, an ActRIIB(20-129)-Fc polypeptide retains similar but somewhat reduced activity relative to the wild type, even though the proline knot region is disrupted. Thus, ActRIIB extracellular domains that stop at amino acid 134, 133, 132, 131, 130 and 129 are all expected to be active, but constructs stopping at 134 or 133 may be most active. Similarly, mutations at any of residues 129-134 are not expected to alter ligand binding affinity by large margins. In support of this, mutations of P129 and P130 do not substantially decrease ligand binding. Therefore, an ActRIIB-Fc fusion polypeptide may end as early as amino acid 109 (the final cysteine), however, forms ending at or between 109 and 119 are expected to have reduced ligand binding. Amino acid 119 is poorly conserved and so is readily altered or truncated. Forms ending at 128 or later retain ligand binding activity. Forms ending at or between 119 and 127 will have an intermediate binding ability. Any of these forms may be desirable to use, depending on the clinical or experimental setting.

At the N-terminus of ActRIIB, it is expected that a polypeptide beginning at amino acid 29 or before will retain ligand binding activity. Amino acid 29 represents the initial cysteine. An alanine-to-asparagine mutation at position 24 introduces an N-linked glycosylation sequence without substantially affecting ligand binding. This confirms that mutations in the region between the signal cleavage peptide and the cysteine cross-linked region, corresponding to amino acids 20-29, are well tolerated. In particular, constructs beginning at position 20, 21, 22, 23 and 24 will retain activity, and constructs beginning at positions 25, 26, 27, 28 and 29 are also expected to retain activity. Data shown in WO2008097541 demonstrate that, surprisingly, a construct beginning at 22, 23, 24 or 25 will have the most activity.

Taken together, an active portion of ActRIIB comprises amino acids 29-109 of SEQ ID NO: 2, and constructs may, for example, begin at a residue corresponding to amino acids 20-29 and end at a position corresponding to amino acids 109-134. Other examples include constructs that begin at a position from 20-29 or 21-29 and end at a position from 119-134, 119-133 or 129-134, 129-133. Other examples include constructs that begin at a position from 20-24 (or 21-24, or 22-25) and end at a position from 109-134 (or 109-133), 119-134 (or 119-133) or 129-134 (or 129-133). Variants within these ranges are also contemplated, particularly those having at least 80%, 85%, 90%, 91%, 92%, 93%, 94%, 95%, 96%, 97%, 98%, or 99% identity to the corresponding portion of SEQ ID NO: 1.

The variations described herein may be combined in various ways. In some embodiments, ActRIIB variants comprise no more than 1, 2, 5, 6, 7, 8, 9, 10 or 15 conservative amino acid changes in the ligand-binding pocket, optionally zero, one or more non-conservative alterations at positions 40, 53, 55, 74, 79 and/or 82 in the ligand-binding pocket. Sites outside the binding pocket, at which variability may be particularly well tolerated, include the amino and carboxy termini of the extracellular domain (as noted above), and positions 42-46 and 65-73 (with respect to SEQ ID NO: 2). An asparagine-to-alanine alteration at position 65 (N65A) does not appear to decrease ligand binding in the R64 background [U.S. Pat. No. 7,842,663]. This change probably eliminates glycosylation at N65 in the A64 background, thus demonstrating that a significant change in this region is likely to be tolerated. While an R64A change is poorly tolerated, R64K is well-tolerated, and thus another basic residue, such as H may be tolerated at position 64 [U.S. Pat. No. 7,842,663]. Additionally, the results of the mutagenesis program described in the art indicate that there are amino acid positions in ActRIIB that are often beneficial to conserve. With respect to SEQ ID NO: 2, these include position 80 (acidic or hydrophobic amino acid), position 78 (hydrophobic, and particularly tryptophan), position 37 (acidic, and particularly aspartic or glutamic acid), position 56 (basic amino acid), position 60 (hydrophobic amino acid, particularly phenylalanine or tyrosine). Thus, the disclosure provides a framework of amino acids that may be conserved in ActRIIB polypeptides. Other positions that may be desirable to conserve are as follows: position 52 (acidic amino acid), position 55 (basic amino acid), position 81 (acidic), 98 (polar or charged, particularly E, D, R or K), all with respect to SEQ ID NO: 2.

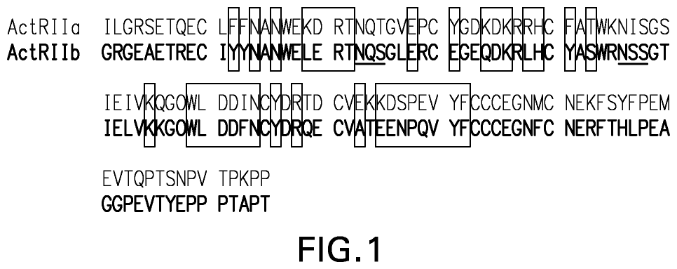

It has been previously demonstrated that the addition of a further N-linked glycosylation site (N-X-S/T) into the ActRIIB extracellular domain is well-tolerated (see, e.g., U.S. Pat. No. 7,842,663). Therefore, N-X-S/T sequences may be generally introduced at positions outside the ligand binding pocket defined in FIG. 1 in ActRIIB polypeptide of the present disclosure. Particularly suitable sites for the introduction of non-endogenous N-X-S/T sequences include amino acids 20-29, 20-24, 22-25, 109-134, 120-134 or 129-134 (with respect to SEQ ID NO: 2). N-X-S/T sequences may also be introduced into the linker between the ActRIIB sequence and an Fc domain or other fusion component as well as optionally into the fusion component itself. Such a site may be introduced with minimal effort by introducing an N in the correct position with respect to a pre-existing S or T, or by introducing an S or T at a position corresponding to a pre-existing N. Thus, desirable alterations that would create an N-linked glycosylation site are: A24N, R64N, S67N (possibly combined with an N65A alteration), E105N, R112N, G120N, E123N, P129N, A132N, R112S and R112T (with respect to SEQ ID NO: 2). Any S that is predicted to be glycosylated may be altered to a T without creating an immunogenic site, because of the protection afforded by the glycosylation. Likewise, any T that is predicted to be glycosylated may be altered to an S. Thus, the alterations S67T and S44T (with respect to SEQ ID NO: 2) are contemplated. Likewise, in an A24N variant, an S26T alteration may be used. Accordingly, an ActRIIB polypeptide of the present disclosure may be a variant having one or more additional, non-endogenous N-linked glycosylation consensus sequences as described above.

In certain embodiments, a variant ActRIIB polypeptide has an amino acid sequence that is at least 75% identical to an amino acid sequence selected from SEQ ID NOs: 1, 2, and 53. In certain cases, the variant ActRIIB polypeptide has an amino acid sequence at least 80%, 85%, 90%, 91%, 92%, 93%, 94%, 95%, 96%, 97%, 98%, 99%, or 100% identical to an amino acid sequence selected from SEQ ID NOs: 1, 2, and 53. In certain cases, the variant ActRIIB polypeptide has an amino acid sequence at least 80%, 85%, 90%, 91%, 92%, 93%, 94%, 95%, 96%, 97%, 98%, 99%, or 100% identical to SEQ ID NO: 1. In certain cases, the variant ActRIIB polypeptide has an amino acid sequence at least 80%, 85%, 90%, 91%, 92%, 93%, 94%, 95%, 96%, 97%, 98%, 99%, or 100% identical to SEQ ID NO: 2. In certain cases, the variant ActRIIB polypeptide has an amino acid sequence at least 80%, 85%, 90%, 91%, 92%, 93%, 94%, 95%, 96%, 97%, 98%, 99%, or 100% identical to SEQ ID NO: 53.

In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of any one of SEQ ID NOs: 1, 2, 5, 6, 12, 31, 33, 34, 36, 37, 39, 40, 42, 43, 45, 46, 48, 49, 50, 51, 52, 53, 276, 278, 279, 332, 333, 335, 336, 338, 339, 341, 342, 344, 345, 347, 348, 350, 351, 353, 354, 356, 357, 385, 386, 387, 388, 389, 396, 398, 402, 403, 406, 408, and 409. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 1. An ActRIIB-Fc fusion protein comprising SEQ ID NO: 1 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97%, 98%, 99%, or 100% identical to the amino acid sequence of SEQ ID NO: 2. An ActRIIB-Fc fusion protein comprising SEQ ID NO: 2 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 5. An ActRIIB-Fc fusion protein comprising SEQ ID NO: 5 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97%, 98%, 99%, or 100% identical to the amino acid sequence of SEQ ID NO: 6. An ActRIIB-Fc fusion protein comprising SEQ ID NO: 6 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 12. An ActRIIB-Fc fusion protein comprising SEQ ID NO: 12 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 31. An ActRIIB-Fc fusion protein comprising SEQ ID NO: 31 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 33. An ActRIIB-Fc fusion protein comprising SEQ ID NO: 33 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97%, 98%, 99%, or 100% identical to the amino acid sequence of SEQ ID NO: 34. An ActRIIB-Fc fusion protein comprising SEQ ID NO: 34 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 36. An ActRIIB-Fc fusion protein comprising SEQ ID NO: 36 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97%, 98%, 99%, or 100% identical to the amino acid sequence of SEQ ID NO: 37. An ActRIIB-Fc fusion protein comprising SEQ ID NO: 37 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 39. An ActRIIB-Fc fusion protein comprising SEQ ID NO: 39 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97% 98%, 99%, or 100% identical to the amino acid sequence of SEQ ID NO: 40. An ActRIIB-Fc fusion protein comprising SEQ ID NO: 40 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 42. An ActRIIB-Fc fusion protein comprising SEQ ID NO: 42 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 43. An ActRIIB-Fc fusion protein comprising SEQ ID NO: 43 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 45. An ActRIIB-Fc fusion protein comprising SEQ ID NO: 45 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97%, 98%, 99%, or 100% identical to the amino acid sequence of SEQ ID NO: 46. An ActRIIB-Fc fusion protein comprising SEQ ID NO: 46 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 48. An ActRIIB-Fc fusion protein comprising SEQ ID NO: 48 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97% 98%, 99%, or 100% identical to the amino acid sequence of SEQ ID NO: 49. An ActRIIB-Fc fusion protein comprising SEQ ID NO: 49 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 50. An ActRIIB-Fc fusion protein comprising SEQ ID NO: 50 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97%, 98%, 99%, or 100% identical to the amino acid sequence of SEQ ID NO: 51. An ActRIIB-Fc fusion protein comprising SEQ ID NO: 51 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 52. An ActRIIB-Fc fusion protein comprising SEQ ID NO: 52 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 53. An ActRIIB-Fc fusion protein comprising SEQ ID NO: 53 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 276. An ActRIIB-Fc fusion protein comprising SEQ ID NO: 276 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97%, 98%, 99%, or 100% identical to the amino acid sequence of SEQ ID NO: 278. An ActRIIB-Fc fusion protein comprising SEQ ID NO: 278 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 279. An ActRIIB-Fc fusion protein comprising SEQ ID NO: 279 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97%, 98%, 99%, or 100% identical to the amino acid sequence of SEQ ID NO: 332. An ActRIIB-Fc fusion protein comprising SEQ ID NO: 332 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 333. An ActRIIB-Fc fusion protein comprising SEQ ID NO: 333 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97% 98%, 99%, or 100% identical to the amino acid sequence of SEQ ID NO: 335. An ActRIIB-Fc fusion protein comprising SEQ ID NO: 335 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 336. An ActRIIB-Fc fusion protein comprising SEQ ID NO: 336 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 338. An ActRIIB-Fc fusion protein comprising SEQ ID NO: 338 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 339. An ActRIIB-Fc fusion protein comprising SEQ ID NO: 339 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97%, 98%, 99%, or 100% identical to the amino acid sequence of SEQ ID NO: 341. An ActRIIB-Fc fusion protein comprising SEQ ID NO: 341 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 342. An ActRIIB-Fc fusion protein comprising SEQ ID NO: 342 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97% 98%, 99%, or 100% identical to the amino acid sequence of SEQ ID NO: 344. An ActRIIB-Fc fusion protein comprising SEQ ID NO: 344 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 345. An ActRIIB-Fc fusion protein comprising SEQ ID NO: 345 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97% 98%, 99%, or 100% identical to the amino acid sequence of SEQ ID NO: 347. An ActRIIB-Fc fusion protein comprising SEQ ID NO: 347 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 348. An ActRIIB-Fc fusion protein comprising SEQ ID NO: 348 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97% 98%, 99%, or 100% identical to the amino acid sequence of SEQ ID NO: 350. An ActRIIB-Fc fusion protein comprising SEQ ID NO: 350 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 351. An ActRIIB-Fc fusion protein comprising SEQ ID NO: 351 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97% 98%, 99%, or 100% identical to the amino acid sequence of SEQ ID NO: 353. An ActRIIB-Fc fusion protein comprising SEQ ID NO: 353 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 354. An ActRIIB-Fc fusion protein comprising SEQ ID NO: 354 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97% 98%, 99%, or 100% identical to the amino acid sequence of SEQ ID NO: 356. An ActRIIB-Fc fusion protein comprising SEQ ID NO: 356 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 357. An ActRIIB-Fc fusion protein comprising SEQ ID NO: 357 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97% 98%, 99%, or 100% identical to the amino acid sequence of SEQ ID NO: 385. An ActRIIB-Fc fusion protein comprising SEQ ID NO: 385 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 386. An ActRIIB-Fc fusion protein comprising SEQ ID NO: 386 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97%, 98%, 99%, or 100% identical to the amino acid sequence of SEQ ID NO: 387. An ActRIIB-Fc fusion protein comprising SEQ ID NO: 387 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 388. An ActRIIB-Fc fusion protein comprising SEQ ID NO: 388 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97% 98%, 99%, or 100% identical to the amino acid sequence of SEQ ID NO: 389. An ActRIIB-Fc fusion protein comprising SEQ ID NO: 389 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 396. An ActRIIB-Fc fusion protein comprising SEQ ID NO: 396 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97% 98%, 99%, or 100% identical to the amino acid sequence of SEQ ID NO: 398. An ActRIIB-Fc fusion protein comprising SEQ ID NO: 398 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 402. An ActRIIB-Fc fusion protein comprising SEQ ID NO: 402 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 403. An ActRIIB-Fc fusion protein comprising SEQ ID NO: 403 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 406. An ActRIIB-Fc fusion protein comprising SEQ ID NO: 406 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95% 96%, 97% 98%, 99%, or 100% identical to the amino acid sequence of SEQ ID NO: 408. An ActRIIB-Fc fusion protein comprising SEQ ID NO: 408 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 409. An ActRIIB-Fc fusion protein comprising SEQ ID NO: 409 may optionally be provided with the lysine removed from the C-terminus.

In certain aspects, the disclosure relates to variant ActRIIB polypeptides comprising an amino acid sequence that is at least 70%, 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2, and wherein the polypeptide comprises one or more amino acid substitutions at a position of SEQ ID NO: 2 selected from the group consisting of: K55, F82, L79, A24, K74, R64, P129, P130, E37, R40, D54, R56, W78, D80, and F82 as well as heteromultimer complexes comprising one or more such variant ActRIIB polypeptides. In certain aspects, the disclosure relates to variant ActRIIB polypeptides comprising an amino acid sequence that is at least 70%, 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2, and wherein the polypeptide comprises one or more amino acid substitutions at a position of SEQ ID NO: 2, but wherein the amino acid at position corresponding to 79 of SEQ ID NO:2 is leucine as well as heteromultimer complexes comprising one or more such variant ActRIIB polypeptides. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 29-109 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 25-131 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 20-134 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 53. In some embodiments, the variant ActRIIB polypeptide comprises an amino acid sequence that is at least 75%, 80%, 85%, 90%, 91%, 92%, 93%, 94%, 95% 96%, 97% 98%, 99%, or 100% identical to the amino acid sequence of SEQ ID NO: 12. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 5. In some embodiments, the polypeptide comprises an amino acid substitution at the amino acid position corresponding to A24 of SEQ ID NO: 2. For example, in some embodiments, the substitution is A24N. In some embodiments, the polypeptide comprises an amino acid substitution at the amino acid position corresponding to S26 of SEQ ID NO: 2. For example, in some embodiments, the substitution is S26T. In some embodiments, the polypeptide comprises an amino acid substitution at the amino acid position corresponding to N35 of SEQ ID NO: 2. For example, in some embodiments, the substitution is N35E. In some embodiments, the polypeptide comprises an amino acid substitution at the amino acid position corresponding to E37 of SEQ ID NO: 2. For example, in some embodiments, the substitution is E37A. In some embodiments, the substitution is E37D. In some embodiments, the polypeptide comprises an amino acid substitution at the amino acid position corresponding to L38 of SEQ ID NO: 2. For example, in some embodiments, the substitution is L38N. In some embodiments, the polypeptide comprises an amino acid substitution at the amino acid position corresponding to R40 of SEQ ID NO: 2. For example, in some embodiments, the substitution is R40A. In some embodiments, the substitution is R40K. In some embodiments, the polypeptide comprises an amino acid substitution at the amino acid position corresponding to S44 of SEQ ID NO: 2. For example, in some embodiments, the substitution is S44T. In some embodiments, the polypeptide comprises an amino acid substitution at the amino acid position corresponding to L46 of SEQ ID NO: 2. For example, in some embodiments, the substitution is L46A. For example, in some embodiments, the substitution is L46I. For example, in some embodiments, the substitution is L46F. For example, in some embodiments, the substitution is L46V. In some embodiments, the polypeptide comprises an amino acid substitution at the amino acid position corresponding to E50 of SEQ ID NO: 2. For example, in some embodiments, the substitution is E50K. In some embodiments, the substitution is E50L. In some embodiments, the substitution is E50P. In some embodiments, the polypeptide comprises an amino acid substitution at the amino acid position corresponding to E52 of SEQ ID NO: 2. For example, in some embodiments, the substitution is E52A. In some embodiments, the substitution is E52D. In some embodiments, the substitution is E52G. In some embodiments, the substitution is E52H. In some embodiments, the substitution is E52K. In some embodiments, the substitution is E52N. In some embodiments, the substitution is E52P. In some embodiments, the substitution is E52R. In some embodiments, the substitution is E52S. In some embodiments, the substitution is E52T. In some embodiments, the substitution is E52Y. In some embodiments, the polypeptide comprises an amino acid substitution at the amino acid position corresponding to Q53 of SEQ ID NO: 2. For example, in some embodiments, the substitution is Q53R. For example, in some embodiments, the substitution is Q53K. For example, in some embodiments, the substitution is Q53N. For example, in some embodiments, the substitution is Q53H. In some embodiments, the polypeptide comprises an amino acid substitution at the amino acid position corresponding to D54 of SEQ ID NO: 2. For example, in some embodiments, the substitution is D54A. In some embodiments, the polypeptide comprises an amino acid substitution at the amino acid position corresponding to K55 of SEQ ID NO: 2. For example, in some embodiments, the substitution is K55A. In some embodiments, the substitution is K55E. In some embodiments, the substitution is K55D. In some embodiments, the substitution is K55R. In some embodiments, the polypeptide comprises an amino acid substitution at the amino acid position corresponding to R56 of SEQ ID NO: 2. For example, in some embodiments, the substitution is R56A. In some embodiments, the polypeptide comprises an amino acid substitution at the amino acid position corresponding to L57 of SEQ ID NO: 2. For example, in some embodiments, the substitution is L57R. In some embodiments, the substitution is L57E. In some embodiments, the substitution is L57I. In some embodiments, the substitution is L57T. In some embodiments, the substitution is L57V. In some embodiments, the polypeptide comprises an amino acid substitution at the amino acid position corresponding to Y60 of SEQ ID NO: 2. For example, in some embodiments, the substitution is Y60F. In some embodiments, the substitution is Y60D. In some embodiments, the substitution is Y60K. In some embodiments, the substitution is Y60P. In some embodiments, the polypeptide comprises an amino acid substitution at the amino acid position corresponding to R64 of SEQ ID NO: 2. For example, in some embodiments, the substitution is R64K. In some embodiments, the substitution is R64N. In some embodiments, the substitution is R64A. In some embodiments, the substitution is R64H. In some embodiments, the polypeptide comprises an amino acid substitution at the amino acid position corresponding to N65 of SEQ ID NO: 2. For example, in some embodiments, the substitution is N65A. In some embodiments, the polypeptide comprises an amino acid substitution at the amino acid position corresponding to S67 of SEQ ID NO: 2. For example, in some embodiments, the substitution is S67N. In some embodiments, the substitution is S67T. In some embodiments, the polypeptide comprises an amino acid substitution at the amino acid position corresponding to G68 of SEQ ID NO: 2. For example, in some embodiments, the substitution is G68R. In some embodiments, the polypeptide comprises an amino acid substitution at the amino acid position corresponding to K74 of SEQ ID NO: 2. For example, in some embodiments, the substitution is K74A. In some embodiments, the substitution is K74E. In some embodiments, the substitution is K74F. In some embodiments, the substitution is K74I. In some embodiments, the substitution is K74Y. In some embodiments, the substitution is K74R. In some embodiments, the polypeptide comprises an amino acid substitution at the amino acid position corresponding to W78 of SEQ ID NO: 2. For example, in some embodiments, the substitution is W78A. In some embodiments, the substitution is W78Y. In some embodiments, the polypeptide comprises an amino acid substitution at the amino acid position corresponding to L79 of SEQ ID NO: 2. For example, in some embodiments, the substitution is L79D. In some embodiments, the substitution does not comprise an acidic amino acid at the position corresponding to L79 of SEQ ID NO: 2. In some embodiments, the substitution is not at position L79 of SEQ ID NO: 2. In some embodiments, position L79 of SEQ ID NO: 2 is not substituted. In some embodiments, the substitution does not comprise an aspartic acid (D) at the position corresponding to L79 of SEQ ID NO: 2. In some embodiments, the substitution is L79A. In some embodiments, the substitution is L79E. In some embodiments, the substitution is L79F. In some embodiments, the substitution is L79H. In some embodiments, the substitution is L79K. In some embodiments, the substitution is L79P. In some embodiments, the substitution is L79R. In some embodiments, the substitution is L79S. In some embodiments, the substitution is L79T. In some embodiments, the substitution is L79W. In some embodiments, the polypeptide comprises an amino acid substitution at the amino acid position corresponding to D80 of SEQ ID NO: 2. For example, in some embodiments, the substitution is D80A. In some embodiments, the substitution is D80F. In some embodiments, the substitution is D80K. In some embodiments, the substitution is D80G. In some embodiments, the substitution is D80M. In some embodiments, the substitution is D80I. In some embodiments, the substitution is D80N. In some embodiments, the substitution is D80R. In some embodiments, the polypeptide comprises an amino acid substitution at the amino acid position corresponding to F82 of SEQ ID NO: 2. For example, in some embodiments, the substitution is F82I. In some embodiments, the substitution is F82K. In some embodiments, the substitution is F82A. In some embodiments, the substitution is F82W. In some embodiments, the substitution is F82D. In some embodiments, the substitution is F82Y. In some embodiments, the substitution is F82E. In some embodiments, the substitution is F82L. In some embodiments, the substitution is F82T. In some embodiments, the substitution is F82S. In some embodiments, the polypeptide comprises an amino acid substitution at the amino acid position corresponding to N83 of SEQ ID NO: 2. For example, in some embodiments, the substitution is N83A. In some embodiments, the substitution is N83R. In some embodiments, the polypeptide comprises an amino acid substitution at the amino acid position corresponding to T93 of SEQ ID NO: 2. For example, in some embodiments, the substitution is T93D. In some embodiments, the substitution is T93E. In some embodiments, the substitution is T93H. In some embodiments, the substitution is T93G. In some embodiments, the substitution is T93K. In some embodiments, the substitution is T93P. In some embodiments, the substitution is T93R. In some embodiments, the substitution is T93S. In some embodiments, the substitution is T93Y. In some embodiments, the polypeptide comprises an amino acid substitution at the amino acid position corresponding to E94 of SEQ ID NO: 2. For example, in some embodiments, the substitution is E94K. In some embodiments, the polypeptide comprises an amino acid substitution at the amino acid position corresponding to Q98 of SEQ ID NO: 2. For example, in some embodiments, the substitution is Q98D. In some embodiments, the substitution is Q98E. In some embodiments, the substitution is Q98K. In some embodiments, the substitution is Q98R. In some embodiments, the polypeptide comprises an amino acid substitution at the amino acid position corresponding to V99 of SEQ ID NO: 2. For example, in some embodiments, the substitution is V99E. In some embodiments, the substitution is V99G. In some embodiments, the substitution is V99K. In some embodiments, the polypeptide comprises an amino acid substitution at the amino acid position corresponding to E105 of SEQ ID NO: 2. For example, in some embodiments, the substitution is E105N. In some embodiments, the polypeptide comprises an amino acid substitution at the amino acid position corresponding to E106 of SEQ ID NO: 2. For example, in some embodiments, the substitution is E106N. In some embodiments, the polypeptide comprises an amino acid substitution at the amino acid position corresponding to F108 of SEQ ID NO: 2. For example, in some embodiments, the substitution is F108I. In some embodiments, the substitution is F108L. In some embodiments, the substitution is F108V. In some embodiments, the substitution is F108Y. In some embodiments, the polypeptide comprises an amino acid substitution at the amino acid position corresponding to E111 of SEQ ID NO: 2. For example, in some embodiments, the substitution is E111K. In some embodiments, the substitution is E111D. In some embodiments, the substitution is E111R. In some embodiments, the substitution is E111H. In some embodiments, the substitution is E111Q. In some embodiments, the substitution is E111N. In some embodiments, the polypeptide comprises an amino acid substitution at the amino acid position corresponding to R112 of SEQ ID NO: 2. For example, in some embodiments, the substitution is R112H. In some embodiments, the substitution is R112K. In some embodiments, the substitution is R112N. In some embodiments, the substitution is R112S. In some embodiments, the substitution is R112T. In some embodiments, the polypeptide comprises an amino acid substitution at the amino acid position corresponding to A119 of SEQ ID NO: 2. For example, in some embodiments, the substitution is A119P. In some embodiments, the substitution is A119V. In some embodiments, the polypeptide comprises an amino acid substitution at the amino acid position corresponding to G120 of SEQ ID NO: 2. For example, in some embodiments, the substitution is G120N. In some embodiments, the polypeptide comprises an amino acid substitution at the amino acid position corresponding to E123 of SEQ ID NO: 2. For example, in some embodiments, the substitution is E123N. In some embodiments, the polypeptide comprises an amino acid substitution at the amino acid position corresponding to P129 of SEQ ID NO: 2. For example, in some embodiments, the substitution is P129S. In some embodiments, the substitution is P129N. In some embodiments, the polypeptide comprises an amino acid substitution at the amino acid position corresponding to P130 of SEQ ID NO: 2. For example, in some embodiments, the substitution is P130A. In some embodiments, the substitution is P130R. In some embodiments, the polypeptide comprises an amino acid substitution at the amino acid position corresponding to A132 of SEQ ID NO: 2. For example, in some embodiments, the substitution is A132N.

In some embodiments, any of the variant ActRIIB polypeptides disclosed herein comprises a substitution at a position of SEQ ID NO: 2 selected from the group consisting of: A24, E37, R40, D54, K55, R56, R64, K74, W78, L79, D80, F82, P129, and P130. In some embodiments, the variant ActRIIB polypeptide comprises a substitution at position A24 with respect to SEQ ID NO: 2. In some embodiments, the variant ActRIIB polypeptide comprises a substitution at position E37 with respect to SEQ ID NO: 2. In some embodiments, the variant ActRIIB polypeptide comprises a substitution at position R40 with respect to SEQ ID NO: 2. In some embodiments, the variant ActRIIB polypeptide comprises a substitution at position D54 with respect to SEQ ID NO: 2. In some embodiments, the variant ActRIIB polypeptide comprises a substitution at position K55 with respect to SEQ ID NO: 2. In some embodiments, the variant ActRIIB polypeptide comprises a substitution at position R56 with respect to SEQ ID NO: 2. In some embodiments, the variant ActRIIB polypeptide comprises a substitution at position R64 with respect to SEQ ID NO: 2. In some embodiments, the variant ActRIIB polypeptide comprises a substitution at position K74 with respect to SEQ ID NO: 2. In some embodiments, the variant ActRIIB polypeptide comprises a substitution at position W78 with respect to SEQ ID NO: 2. In some embodiments, the variant ActRIIB polypeptide comprises a substitution at position L79 with respect to SEQ ID NO: 2. In some embodiments, the variant ActRIIB polypeptide comprises a substitution at position D80 with respect to SEQ ID NO: 2. In some embodiments, the variant ActRIIB polypeptide comprises a substitution at position F82 with respect to SEQ ID NO: 2. In some embodiments, the variant ActRIIB polypeptide comprises a substitution at position P129 with respect to SEQ ID NO: 2. In some embodiments, the variant ActRIIB polypeptide comprises a substitution at position P130 with respect to SEQ ID NO: 2.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 31. In some embodiments, the variant ActRIIB polypeptide comprises an alanine at the position corresponding to K55 of SEQ ID NO: 2. In some embodiments, the amino acid sequence of SEQ ID NO: 31 may optionally be provided with the lysine removed from the C-terminus. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 33. In some embodiments, the variant ActRIIB polypeptide comprises an alanine at the position corresponding to K55 of SEQ ID NO: 2. In some embodiments, the amino acid sequence of SEQ ID NO: 33 may optionally be provided with the lysine removed from the C-terminus. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 34. In some embodiments, the variant ActRIIB polypeptide comprises a glutamic acid at the position corresponding to K55 of SEQ ID NO: 2. In some embodiments, the amino acid sequence of SEQ ID NO: 34 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 36. In some embodiments, the variant ActRIIB polypeptide comprises a glutamic acid at the position corresponding to K55 of SEQ ID NO: 2. In some embodiments, the amino acid sequence of SEQ ID NO: 36 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 37. In some embodiments, the variant ActRIIB polypeptide comprises an isoleucine at the position corresponding to F82 of SEQ ID NO: 2. In some embodiments, the amino acid sequence of SEQ ID NO: 37 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 39. In some embodiments, the variant ActRIIB polypeptide comprises an isoleucine at the position corresponding to F82 of SEQ ID NO: 2. In some embodiments, the amino acid sequence of SEQ ID NO: 39 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 40. In some embodiments, the variant ActRIIB polypeptide comprises a lysine at the position corresponding to F82 of SEQ ID NO: 2. In some embodiments, the amino acid sequence of SEQ ID NO: 40 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 42. In some embodiments, the variant ActRIIB polypeptide comprises a lysine at the position corresponding to F82 of SEQ ID NO: 2. In some embodiments, the amino acid sequence of SEQ ID NO: 42 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 43. In some embodiments, the variant ActRIIB polypeptide comprises a glutamic acid at the position corresponding to L79 of SEQ ID NO: 2. In some embodiments, the amino acid sequence of SEQ ID NO: 43 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 45. In some embodiments, the variant ActRIIB polypeptide comprises a glutamic acid at the position corresponding to L79 of SEQ ID NO: 2. In some embodiments, the amino acid sequence of SEQ ID NO: 45 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 336. In some embodiments, the variant ActRIIB polypeptide comprises a threonine at the position corresponding to F82 of SEQ ID NO: 2. In some embodiments, the amino acid sequence of SEQ ID NO: 336 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 338. In some embodiments, the variant ActRIIB polypeptide comprises a threonine at the position corresponding to F82 of SEQ ID NO: 2. In some embodiments, the amino acid sequence of SEQ ID NO: 338 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 342. In some embodiments, the variant ActRIIB polypeptide comprises a histidine at the position corresponding to L79 of SEQ ID NO: 2. In some embodiments, the amino acid sequence of SEQ ID NO: 342 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 344. In some embodiments, the variant ActRIIB polypeptide comprises a histidine at the position corresponding to L79 of SEQ ID NO: 2. In some embodiments, the amino acid sequence of SEQ ID NO: 344 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 348. In some embodiments, the variant ActRIIB polypeptide comprises a leucine at the position corresponding to E50 of SEQ ID NO: 2. In some embodiments, the amino acid sequence of SEQ ID NO: 348 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 350. In some embodiments, the variant ActRIIB polypeptide comprises a leucine at the position corresponding to E50 of SEQ ID NO: 2. In some embodiments, the amino acid sequence of SEQ ID NO: 350 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 354. In some embodiments, the variant ActRIIB polypeptide comprises a glycine at the position corresponding to V99 of SEQ ID NO: 2. In some embodiments, the amino acid sequence of SEQ ID NO: 354 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 356. In some embodiments, the variant ActRIIB polypeptide comprises a glycine at the position corresponding to V99 of SEQ ID NO: 2. In some embodiments, the amino acid sequence of SEQ ID NO: 356 may optionally be provided with the lysine removed from the C-terminus.

In some embodiments, any of the variant ActRIIB polypeptides disclosed herein comprises at least 2, 3, 4, 5, 6, 7, 8, 9, or 10 of any of the amino acid substitutions disclosed herein. In some embodiments, any of the variant ActRIIB polypeptides disclosed herein comprises 2 of any of the amino acid substitutions disclosed herein. In some embodiments, any of the variant ActRIIB polypeptides disclosed herein comprises 3 of any of the amino acid substitutions disclosed herein. In some embodiments, any of the variant ActRIIB polypeptides disclosed herein comprises 4 of any of the amino acid substitutions disclosed herein. In some embodiments, any of the variant ActRIIB polypeptides disclosed herein comprises 5 of any of the amino acid substitutions disclosed herein. In some embodiments, any of the variant ActRIIB polypeptides disclosed herein comprises 6 of any of the amino acid substitutions disclosed herein. In some embodiments, any of the variant ActRIIB polypeptides disclosed herein comprises 7 of any of the amino acid substitutions disclosed herein. In some embodiments, any of the variant ActRIIB polypeptides disclosed herein comprises 8 of any of the amino acid substitutions disclosed herein. In some embodiments, any of the variant ActRIIB polypeptides disclosed herein comprises 9 of any of the amino acid substitutions disclosed herein. In some embodiments, any of the variant ActRIIB polypeptides disclosed herein comprises 10 of any of the amino acid substitutions disclosed herein.

In certain aspects, the disclosure relates to a variant ActRIIB polypeptide comprising two or more amino acid substitutions as compared to the reference amino acid sequence of SEQ ID NO: 2. For example, in some embodiments, the variant ActRIIB polypeptide comprises an A24N substitution and a K74A substitution. In some embodiments, the variant ActRIIB polypeptide comprises a L79P substitution and a K74A substitution. In some embodiments, the variant ActRIIB polypeptide comprises a P129S substitution and a P130A substitution. In some embodiments, the variant ActRIIB polypeptide comprises a L38N substitution and a L79R substitution. In some embodiments, the variant ActRIIB polypeptide comprises a F82I substitution and a N83R substitution. In some embodiments, the variant ActRIIB polypeptide comprises a F82K substitution and a N83R substitution. In some embodiments, the variant ActRIIB polypeptide comprises a F82T substitution and a N83R substitution. In some embodiments, the variant ActRIIB polypeptide comprises a L79H substitution and a F82K substitution. In some embodiments, the variant ActRIIB polypeptide comprises a L79H substitution and a F82I substitution. In some embodiments, the variant ActRIIB polypeptide comprises a F82D substitution and a N83R substitution. In some embodiments, the variant ActRIIB polypeptide comprises a F82E substitution and a N83R substitution. In some embodiments, the variant ActRIIB polypeptide comprises a L79F substitution and a F82D substitution. In some embodiments, the variant ActRIIB polypeptide comprises a L79F substitution and a F82T substitution. In some embodiments, the variant ActRIIB polypeptide comprises a E52D substitution and a F82D substitution. In some embodiments, the variant ActRIIB polypeptide comprises an E52D substitution and a F82T substitution. In some embodiments, the variant ActRIIB polypeptide comprises a L57R substitution and a F82D substitution. In some embodiments, the variant ActRIIB polypeptide comprises a L57R substitution and a F82T substitution. In some embodiments, the variant ActRIIB polypeptide comprises a F82I substitution and an E94K substitution. In some embodiments, the variant ActRIIB polypeptide comprises a F82S substitution and a N83R substitution. In some embodiments, the variant ActRIIB polypeptide comprises a L57R substitution and a F82S substitution. In some embodiments, the variant ActRIIB polypeptide comprises a K74A substitution and a L79P substitution. In some embodiments, the variant ActRIIB polypeptide comprises a K55A substitution and a F82I substitution. In some embodiments, the variant ActRIIB polypeptide comprises a L79K substitution and a F82K substitution. In some embodiments, the variant ActRIIB polypeptide comprises a F82W substitution and a N83A substitution.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 276. In some embodiments, the variant ActRIIB polypeptide comprises an isoleucine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an isoleucine at the position corresponding to F82 of SEQ ID NO: 2 and an arginine at the position corresponding to N83 of SEQ ID NO: 2. In some embodiments, the amino acid sequence of SEQ ID NO: 276 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 278. In some embodiments, the variant ActRIIB polypeptide comprises an isoleucine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an isoleucine at the position corresponding to F82 of SEQ ID NO: 2 and an arginine at the position corresponding to N83 of SEQ ID NO: 2. In some embodiments, the amino acid sequence of SEQ ID NO: 278 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 279. In some embodiments, the variant ActRIIB polypeptide comprises an lysine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a lysine at the position corresponding to F82 of SEQ ID NO: 2 and an arginine at the position corresponding to N83 of SEQ ID NO: 2. In some embodiments, the amino acid sequence of SEQ ID NO: 279 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 332. In some embodiments, the variant ActRIIB polypeptide comprises an lysine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a lysine at the position corresponding to F82 of SEQ ID NO: 2 and an arginine at the position corresponding to N83 of SEQ ID NO: 2. In some embodiments, the amino acid sequence of SEQ ID NO: 332 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 333. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a threonine at the position corresponding to F82 of SEQ ID NO: 2 and an arginine at the position corresponding to N83 of SEQ ID NO: 2. In some embodiments, the amino acid sequence of SEQ ID NO: 333 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 335. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a threonine at the position corresponding to F82 of SEQ ID NO: 2 and an arginine at the position corresponding to N83 of SEQ ID NO: 2. In some embodiments, the amino acid sequence of SEQ ID NO: 335 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 339. In some embodiments, the variant ActRIIB polypeptide comprises a histidine at the position corresponding to L79 of SEQ ID NO: 2. In some embodiments, the variant ActRIIB polypeptide comprises an isoleucine at the position corresponding to F82 of SEQ ID NO: 2. In some embodiments, the variant ActRIIB polypeptide comprises a histidine at the position corresponding to L79 of SEQ ID NO: 2 and an isoleucine at the position corresponding to F82 of SEQ ID NO: 2. In some embodiments, the amino acid sequence of SEQ ID NO: 339 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 341. In some embodiments, the variant ActRIIB polypeptide comprises a histidine at the position corresponding to L79 of SEQ ID NO: 2. In some embodiments, the variant ActRIIB polypeptide comprises an isoleucine at the position corresponding to F82 of SEQ ID NO: 2. In some embodiments, the variant ActRIIB polypeptide comprises a histidine at the position corresponding to L79 of SEQ ID NO: 2 and an isoleucine at the position corresponding to F82 of SEQ ID NO: 2. In some embodiments, the amino acid sequence of SEQ ID NO: 341 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 345. In some embodiments, the variant ActRIIB polypeptide comprises a histidine at the position corresponding to L79 of SEQ ID NO: 2. In some embodiments, the variant ActRIIB polypeptide comprises a lysine at the position corresponding to F82 of SEQ ID NO: 2. In some embodiments, the variant ActRIIB polypeptide comprises a histidine at the position corresponding to L79 of SEQ ID NO: 2, and a lysine at the position corresponding to F82 of SEQ ID NO: 2. In some embodiments, the amino acid sequence of SEQ ID NO: 345 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 347. In some embodiments, the variant ActRIIB polypeptide comprises a histidine at the position corresponding to L79 of SEQ ID NO: 2. In some embodiments, the variant ActRIIB polypeptide comprises a lysine at the position corresponding to F82 of SEQ ID NO: 2. In some embodiments, the variant ActRIIB polypeptide comprises a histidine at the position corresponding to L79 of SEQ ID NO: 2, and a lysine at the position corresponding to F82 of SEQ ID NO: 2. In some embodiments, the amino acid sequence of SEQ ID NO: 347 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 351. In some embodiments, the variant ActRIIB polypeptide comprises an asparagine at the position corresponding to L38 of SEQ ID NO: 2. In some embodiments, the variant ActRIIB polypeptide comprises an arginine at the position corresponding to L79 of SEQ ID NO: 2. In some embodiments, the variant ActRIIB polypeptide comprises an asparagine at the position corresponding to L38 of SEQ ID NO: 2, and an arginine at the position corresponding to L79 of SEQ ID NO: 2. In some embodiments, the amino acid sequence of SEQ ID NO: 351 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 353. In some embodiments, the variant ActRIIB polypeptide comprises an asparagine at the position corresponding to L38 of SEQ ID NO: 2. In some embodiments, the variant ActRIIB polypeptide comprises an arginine at the position corresponding to L79 of SEQ ID NO: 2. In some embodiments, the variant ActRIIB polypeptide comprises an asparagine at the position corresponding to L38 of SEQ ID NO: 2, and an arginine at the position corresponding to L79 of SEQ ID NO: 2. In some embodiments, the amino acid sequence of SEQ ID NO: 353 may optionally be provided with the lysine removed from the C-terminus.

In certain aspects, the disclosure relates to a variant ActRIIB polypeptide comprising three or more amino acid substitutions as compared to the reference amino acid sequence of SEQ ID NO: 2. In some embodiments, the variant ActRIIB polypeptide comprises a G68R substitution, a F82S substitution, and a N83R substitution. In some embodiments, the variant ActRIIB polypeptide comprises a G68R substitution, a W78Y substitution, and a F82Y substitution. In some embodiments, the variant ActRIIB polypeptide comprises a E52D substitution, a F82D substitution, and a N83R substitution. In some embodiments, the variant ActRIIB polypeptide comprises an E52Y substitution, a F82D substitution, and a N83R substitution. In some embodiments, the variant ActRIIB polypeptide comprises an E52D substitution, a F82E substitution, and a N83R substitution. In some embodiments, the variant ActRIIB polypeptide comprises an E52D substitution, a F82T substitution, and a N83R substitution. In some embodiments, the variant ActRIIB polypeptide comprises an E52N substitution, a F82I substitution, and a N83R substitution. In some embodiments, the variant ActRIIB polypeptide comprises an E52N substitution, a F82Y substitution, and a N83R substitution. In some embodiments, the variant ActRIIB polypeptide comprises an E50L substitution, a F82D substitution, and a N83R substitution. In some embodiments, the variant ActRIIB polypeptide comprises a L57I substitution, a F82D substitution, and a N83R substitution. In some embodiments, the variant ActRIIB polypeptide comprises a L57V substitution, a F82D substitution, and a N83R substitution. In some embodiments, the variant ActRIIB polypeptide comprises a L57R substitution, a F82D substitution, and a N83R substitution. In some embodiments, the variant ActRIIB polypeptide comprises a L57E substitution, a F82E substitution, and a N83R substitution. In some embodiments, the variant ActRIIB polypeptide comprises a L57R substitution, a F82E substitution, and a N83R substitution. In some embodiments, the variant ActRIIB polypeptide comprises a L57I substitution, a F82E substitution, and a N83R substitution. In some embodiments, the variant ActRIIB polypeptide comprises a L57R substitution, a F82L substitution, and a N83R substitution. In some embodiments, the variant ActRIIB polypeptide comprises a L57T substitution, a F82Y substitution, and a N83R substitution. In some embodiments, the variant ActRIIB polypeptide comprises a L57V substitution, a F82Y substitution, and a N83R substitution. In some embodiments, the variant ActRIIB polypeptide may comprise at least two of the amino acid substitutions described in any of the variant ActRIIB polypeptides above.

In certain aspects, the disclosure relates to a variant ActRIIB polypeptide comprising four or more amino acid substitutions as compared to the reference amino acid sequence of SEQ ID NO: 2. For example, in some embodiments, the variant ActRIIB polypeptide comprises a G68R substitution, a L79E substitution, a F82Y substitution, and a N83R substitution. In some embodiments, the variant ActRIIB polypeptide comprises a G68R substitution, a L79E substitution, a F82T substitution, and a N83R substitution. In some embodiments, the variant ActRIIB polypeptide comprises a G68R substitution, a L79T substitution, a F82T substitution, and a N83R substitution. In some embodiments, the variant ActRIIB polypeptide comprises an E52N substitution, a G68R substitution, a F82Y substitution, and a N83R substitution. In some embodiments, the variant ActRIIB polypeptide may comprise at least two of the amino acid substitutions described in any of the variant ActRIIB polypeptides above. In some embodiments, the variant ActRIIB polypeptide may comprise at least three of the amino acid substitutions described in any of the variant ActRIIB polypeptides above.

C) ActRIIA Polypeptides

In certain embodiments, the disclosure relates to ActRII-ALK4 antagonists that comprise an ActRIIA polypeptide, which includes fragments, functional variants, and modified forms thereof as well as uses thereof (e.g., of treating, preventing, or reducing the progression rate and/or severity of heart failure (HF) or one or more complications of HF). As used herein, the term “ActRIIA” refers to a family of activin receptor type IIA (ActRIIA) proteins from any species and variant polypeptides derived from such ActRIIA proteins by mutagenesis or other modification (including, e.g., mutants, fragments, fusions, and peptidomimetic forms) that retain a useful activity. Examples of such variant ActRIIA polypeptides are provided throughout the present disclosure as well as in International Patent Application Publication Nos. WO 2006/012627 and WO 2007/062188, which are incorporated herein by reference in their entirety. Reference to ActRIIA herein is understood to be a reference to any one of the currently identified forms. Members of the ActRIIA family are generally transmembrane proteins, composed of a ligand-binding extracellular domain comprising a cysteine-rich region, a transmembrane domain, and a cytoplasmic domain with predicted serine/threonine kinase activity. Preferably, ActRIIA polypeptides to be used in accordance with the methods of the disclosure are soluble (e.g., an extracellular domain of ActRIIA). In some embodiments, ActRIIA polypeptides inhibit activity (e.g., Smad signaling) of one or more ActRII-ALK4 ligands (e.g., activin A, activin B, GDF8, GDF11, BMP6, BMP10). In some embodiments, ActRIIA polypeptides bind to one or more ActRII-ALK4 ligands (e.g., activin A, activin B, GDF8, GDF11, BMP6, BMP10). Various examples of methods and assays for determining the ability of an ActRIIA polypeptide to bind to and/or inhibit activity of one or more ActRII-ALK4 ligands are disclosed herein or otherwise well known in the art, which can be readily used to determine if an ActRIIA polypeptide has the desired binding and/or antagonistic activities. Numbering of amino acids for all ActRIIA-related polypeptides described herein is based on the numbering of the human ActRIIA precursor protein sequence provided below (SEQ ID NO: 366), unless specifically designated otherwise.

The canonical human ActRIIA precursor protein sequence is as follows:

```
(SEQ ID NO: 366)
    1 MGAAAKLAFA VFLISCSSGA ILGRSETQEC
        LFFNANWEKD RT [symbol] QTGVEPC
  51 YGDKDKRRHC FATWK [symbol] ISGS IEIVKQGCWL
        DDINCYDRTD CVEKKDSPEV
101 YFCCCEGNMC NEKFSYFPEM EVTQPTSNPV
        TPKPPYYNIL LYSLVPLMLI
151 AGIVICAFWV YRHHKMAYPP VLVPTQDPGP
        PPPSPLLGLK PLQLLEVKAR
201 GREGCVWKAQ LLNEYVAVKI FPIQDKQSWQ
        NEYEVYSLPG MKHENILQFI
251 GAEKRGTSVD VDLWLITAFH EKGSLSDELK
        ANVVSWNELC HIAETMARGL
301 AYLHEDIPGL KDGHKPAISH RDIKSKNVLL
        KNNLTACIAD FGLALKFEAG
351 KSAGDTHGQV GTRRYMAPEV LEGAINFQRD
        AFLRIDMYAM GLVLWELASR
401 CTAADGPVDE YMLPFEEEIG QHPSLEDMQE
        VVVHKKKRPV LRDYWQKHAG
451 MAMLCETIEE CWDHDAEARL SAGCVGERIT
        QMQRLTNIIT TEDIVTVVTM
501 VTNVDFPPKE SSL
```

The signal peptide is indicated by a single underline; the extracellular domain is indicated in bold font; and the potential, endogenous N-linked glycosylation sites are indicated by a double underline.

A processed (mature) extracellular human ActRIIA polypeptide sequence is as follows:

(SEQ ID NO: 367)
ILGRSETQECLFFNANWEKDRTNQTGVEPCYGDKDKRRHCFATWKN
ISGSIEIVKQGCWLDDINCYDRTDCVEKKDSPEVYFCCCEGNMCNE
KFSYFPEMEVTQPTSNPVTPKPP

The C-terminal “tail” of the extracellular domain is indicated by single underline. The sequence with the “tail” deleted (a Δ15 sequence) is as follows:

(SEQ ID NO: 368)
ILGRSETQECLFFNANWEKDRTNQTGVEPCYGDKDKRRHCFATWKN
ISGSIEIVKQGCWLDDINCYDRTDCVEKKDSPEVYFCCCEGNMCNE
KFSYFPEM

A nucleic acid sequence encoding human ActRIIA precursor protein is shown below (SEQ ID NO: 369), corresponding to nucleotides 159-1700 of GenBank Reference Sequence NM_001616.4. The signal sequence is underlined.

```
(SEQ ID NO: 369)
    1 ATGGGAGCTG CTGCAAAGTT GGCGTTTGCC GTCTTTCTTA TCTCCTGTTC
  51 TTCAGGTGCT ATACTTGGTA GATCAGAAAC TCAGGAGTGT CTTTTCTTTA
101 ATGCTAATTG GGAAAAAGAC AGAACCAATC AAACTGGTGT TGAACCGTGT
151 TATGGTGACA AAGATAAACG GCGGCATTGT TTTGCTACCT GGAAGAATAT
201 TTCTGGTTCC ATTGAAATAG TGAAACAAGG TTGTTGGCTG GATGATATCA
251 ACTGCTATGA CAGGACTGAT TGTGTAGAAA AAAAAGACAG CCCTGAAGTA
301 TATTTTTGTT GCTGTGAGGG CAATATGTGT AATGAAAAGT TTTCTTATTT
351 TCCGGAGATG GAAGTCACAC AGCCCACTTC AAATCCAGTT ACACCTAAGC
401 CACCCTATTA CAACATCCTG CTCTATTCCT TGGTGCCACT TATGTTAATT
451 GCGGGGATTG TCATTTGTGC ATTTTGGGTG TACAGGCATC ACAAGATGGC
501 CTACCCTCCT GTACTTGTTC CAACTCAAGA CCCAGGACCA CCCCCACCTT
551 CTCCATTACT AGGTTTGAAA CCACTGCAGT TATTAGAAGT GAAAGCAAGG
601 GGAAGATTTG GTTGTGTCTG GAAAGCCCAG TTGCTTAACG AATATGTGGC
651 TGTCAAAATA TTTCCAATAC AGGACAAACA GTCATGGCAA AATGAATACG
701 AAGTCTACAG TTTGCCTGGA ATGAAGCATG AGAACATATT ACAGTTCATT
751 GGTGCAGAAA AACGAGGCAC CAGTGTTGAT GTGGATCTTT GGCTGATCAC
801 AGCATTTCAT GAAAAGGGTT CACTATCAGA CTTTCTTAAG GCTAATGTGG
851 TCTCTTGGAA TGAACTGTGT CATATTGCAG AAACCATGGC TAGAGGATTG
901 GCATATTTAC ATGAGGATAT ACCTGGCCTA AAAGATGGCC ACAAACCTGC
951 CATATCTCAC AGGGACATCA AAAGTAAAAA TGTGCTGTTG AAAAACAACC
1001 TGACAGCTTG CATTGCTGAC TTTGGGTTGG CCTTAAAATT TGAGGCTGGC
1051 AAGTCTGCAG GCGATACCCA TGGACAGGTT GGTACCCGGA GGTACATGGC
1101 TCCAGAGGTA TTAGAGGGTG CTATAAACTT CCAAAGGGAT GCATTTTTGA
1151 GGATAGATAT GTATGCCATG GGATTAGTCC TATGGGAACT GGCTTCTCGC
1201 TGTACTGCTG CAGATGGACC TGTAGATGAA TACATGTTGC CATTTGAGGA
1251 GGAAATTGGC CAGCATCCAT CTCTTGAAGA CATGCAGGAA GTTGTTGTGC
1301 ATAAAAAAAA GAGGCCTGTT TTAAGAGATT ATTGGCAGAA ACATGCTGGA
1351 ATGGCAATGC TCTGTGAAAC CATTGAAGAA TGTTGGGATC ACGACGCAGA
1401 AGCCAGGTTA TCAGCTGGAT GTGTAGGTGA AAGAATTACC CAGATGCAGA
1451 GACTAACAAA TATTATTACC ACAGAGGACA TTGTAACAGT GGTCACAATG
1501 GTGACAAATG TTGACTTTCC TCCCAAAGAA TCTAGTCTA
```

A nucleic acid sequence encoding processed soluble (extracellular) human ActRIIA polypeptide is as follows:

```
(SEQ ID NO: 370)
    1 ATACTTGGTA GATCAGAAAC TCAGGAGTGT CTTTTCTTTA ATGCTAATTG
  51 GGAAAAAGAC AGAACCAATC AAACTGGTGT TGAACCGTGT TATGGTGACA
101 AAGATAAACG GCGGCATTGT TTTGCTACCT GGAAGAATAT TTCTGGTTCC
151 ATTGAAATAG TGAAACAAGG TTGTTGGCTG GATGATATCA ACTGCTATGA
201 CAGGACTGAT TGTGTAGAAA AAAAAGACAG CCCTGAAGTA TATTTTTGTT
251 GCTGTGAGGG CAATATGTGT AATGAAAAGT TTTCTTATTT TCCGGAGATG
301 GAAGTCACAC AGCCCACTTC AAATCCAGTT ACACCTAAGC CACCC
```

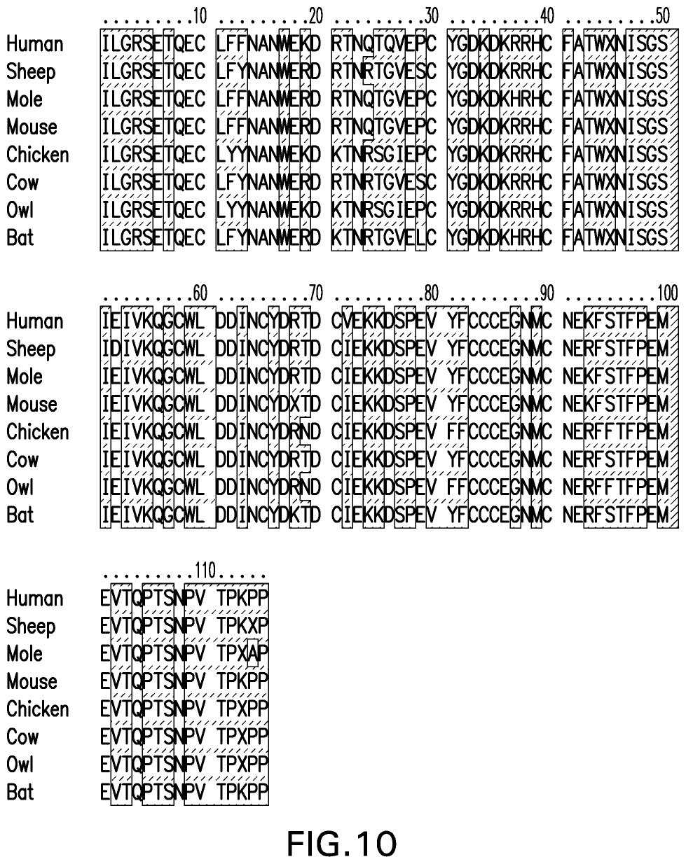

ActRIIA is well-conserved among vertebrates, with large stretches of the extracellular domain completely conserved. For example, FIG. 10 depicts a multi-sequence alignment of a human ActRIIA extracellular domain (SEQ ID NO: 367) compared to various ActRIIA orthologs (SEQ ID NOs: 371-377). Many of the ligands that bind to ActRIIA are also highly conserved. Accordingly, from these alignments, it is possible to predict key amino acid positions within the ligand-binding domain that are important for normal ActRIIA-ligand binding activities as well as to predict amino acid positions that are likely to be tolerant to substitution without significantly altering normal ActRIIA-ligand binding activities. Therefore, an active, human ActRIIA variant polypeptide useful in accordance with the presently disclosed methods may include one or more amino acids at corresponding positions from the sequence of another vertebrate ActRIIA, or may include a residue that is similar to that in the human or other vertebrate sequences.

Without meaning to be limiting, the following examples illustrate this approach to defining an active ActRIIA variant. As illustrated in FIG. 10, F13 in the human extracellular domain is Y in Ovis aries (SEQ ID NO: 371), Gallus gallus (SEQ ID NO: 374), Bos Taurus (SEQ ID NO: 375), Tyto alba (SEQ ID NO: 376), and Myotis davidii (SEQ ID NO: 377) ActRIIA, indicating that aromatic residues are tolerated at this position, including F, W, and Y. Q24 in the human extracellular domain is R in Bos Taurus ActRIIA, indicating that charged residues will be tolerated at this position, including D, R, K, H, and E. S95 in the human extracellular domain is F in Gallus gallus and Tyto alba ActRIIA, indicating that this site may be tolerant of a wide variety of changes, including polar residues, such as E, D, K, R, H, S, T, P, G, Y, and probably hydrophobic residue such as L, I, or F. E52 in the human extracellular domain is D in Ovis aries ActRIIA, indicating that acidic residues are tolerated at this position, including D and E. P29 in the human extracellular domain is relatively poorly conserved, appearing as S in Ovis aries ActRIIA and L in Myotis davidii ActRIIA, thus essentially any amino acid should be tolerated at this position.

Moreover, as discussed above, ActRII proteins have been characterized in the art in terms of structural/functional characteristics, particularly with respect to ligand binding [Attisano et al. (1992) Cell 68(1):97-108; Greenwald et al. (1999) Nature Structural Biology 6(1): 18-22; Allendorph et al. (2006) Proc Natl Acad Sci USA 103(20: 7643-7648; Thompson et al. (2003) The EMBO Journal 22(7): 1555-1566; as well as U.S. Pat. Nos. 7,709,605, 7,612,041, and 7,842,663]. In addition to the teachings herein, these references provide ample guidance for how to generate ActRII variants that retain one or more desired activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIA, as demarcated by the outermost of these conserved cysteines, corresponds to positions 30-110 of SEQ ID NO: 366 (ActRIIA precursor). Therefore, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRIIA extracellular domains truncations include SEQ ID NOs: 367 and 368.

Accordingly, a general formula for an active portion (e.g., ligand binding) of ActRIIA is a polypeptide that comprises, consists essentially of, or consists of amino acids 30-110 of SEQ ID NO: 366. Therefore ActRIIA polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to any one of amino acids 21-30 (e.g., beginning at any one of amino acids 21, 22, 23, 24, 25, 26, 27, 28, 29, or 30) of SEQ ID NO: 366 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 366. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 366, and end at a position selected from 111-135 (e.g., ending at any one of amino acids 111, 112, 113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113, 114, 115, 116, 117, 118, 119, 120, 121, 122, 123, 124, 125, 126, 127, 128, 129, 130, 131, or 132), or 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 366. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 366. Thus, in some embodiments, an ActRIIA polypeptide may comprise, consists essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 366. Optionally, ActRIIA polypeptides comprise a polypeptide that is at least 70%, 75%, 80%, 85%, 86%, 87%, 88%, 89%, 90%, 91%, 92%, 93%, 94%, 95% 96%, 97% 98%, 99%, or 100% identical to amino acids 30-110 of SEQ ID NO: 366, and comprising no more than 1, 2, 5, 10 or 15 conservative amino acid changes in the ligand-binding pocket. In some embodiments, ActRIIA polypeptide of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to amino acids 21-30 (e.g., beginning at any one of amino acids 21, 22, 23, 24, 25, 26, 27, 28, 29, or 30) of SEQ ID NO: 366 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134 or 135) of SEQ ID NO: 366. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 366. In certain embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 366. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 366. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 367. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 368. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 380. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 381. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 384. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 364 In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 378.

D) ALK4 Polypeptides

In certain aspects, the disclosure relates to ActRII-ALK4 antagonists comprising an ALK4 polypeptide, which includes fragments, functional variants, and modified forms thereof as well as uses thereof (e.g., of treating, preventing, or reducing the progression rate and/or severity of heart failure (HF) or one or more complications of HF). As used herein, the term “ALK4” refers to a family of activin receptor-like kinase-4 (ALK4) proteins from any species and variant polypeptides derived from such ALK4 proteins by mutagenesis or other modifications (including, e.g., mutants, fragments, fusions, and peptidomimetic forms) that retain a useful activity. Examples of such variant ALK4 polypeptides are provided throughout the present disclosure as well as in International Patent Application Publication Nos. WO/2016/164089, WO/2016/164497, and WO/2018/067879, which are incorporated herein by reference in their entirety. Reference to ALK4 herein is understood to be a reference to any one of the currently identified forms. Members of the ALK4 family are generally transmembrane proteins, composed of a ligand-binding extracellular domain with a cysteine-rich region, a transmembrane domain, and a cytoplasmic domain with predicted serine/threonine kinase activity. Preferably, ALK4 polypeptides to be used in accordance with the methods of the disclosure are soluble. The term “soluble ALK4 polypeptide,” as used herein, includes any naturally occurring extracellular domain of an ALK4 polypeptide as well as any variants thereof (including mutants, fragments and peptidomimetic forms) that retain a useful activity. For example, the extracellular domain of an ALK4 polypeptide binds to a ligand and is generally soluble. Examples of soluble ALK4 polypeptides include an ALK4 extracellular domain (SEQ ID NO: 86) shown below, Other examples of soluble ALK4 polypeptides comprise a signal sequence in addition to the extracellular domain of an ALK4 polypeptide. The signal sequence can be a native signal sequence of an ALK4 polypeptide, or a signal sequence from another polypeptide, such as a tissue plasminogen activator (TPA) signal sequence or a honey bee melittin signal sequence. In some embodiments, ALK4 polypeptides inhibit (e.g., Smad signaling) activity of one or more ActRII-ALK4 ligands (e.g., activin A, activin B, GDF8, GDF11, BMP6, BMP10). In some embodiments, ALK4 polypeptides bind to one or more ActRII-ALK4 ligands (e.g., activin A, activin B, GDF8, GDF11, BMP6, BMP10). Various examples of methods and assays for determining the ability of an ALK4 polypeptide to bind to and/or inhibit activity of one or more ActRII-ALK4 ligands are disclosed herein or otherwise well known in the art, which can be readily used to determine if an ActRIIB polypeptide has the desired binding and/or antagonistic activities. Numbering of amino acids for all ALK4-related polypeptides described herein is based on the numbering of the human ALK4 precursor protein sequence provided below (SEQ ID NO: 84), unless specifically designated otherwise.

A human ALK4 precursor polypeptide sequence (NCBI Ref Seq NP_004293) is as follows:

```
(SEQ ID NO: 84)
    1 MAESAGASSF FPLVVLLLAG SGGSGPRGVQ ALLCACTSCL QANYTCETDG ACMVSIFNLD
  61 GMEHHVRTCI PKVELVPAGK PFYCLSSEDL RNTHCCYTDY CNRIDLRVPS GHLKEPEHPS
121 MWGPVELVGI IAGPVFLLFL IIIIVELVIN YHQRVYHNRQ RLDMEDPSCE MCLSKDKTLQ
181 DLVYDLSTSG SGSGLPLFVQ RTVARTIVLQ EIIGKGREGE VWRGRWRGGD VAVKIFSSRE
241 ERSWFREAEI YQTVMLRHEN ILGFIAADNK DNGTWTQLWL VSDYHEHGSL FDYLNRYTVT
301 IEGMIKLALS AASGLAHLHM EIVGTQGKPG IAHRDLKSKN ILVKKNGMCA IADLGLAVRH
361 DAVIDTIDIA PNQRVGTKRY MAPEVLDETI NMKHEDSFKC ADIYALGLVY WEIARRCNSG
421 GVHEEYQLPY YDLVPSDPSI EEMRKVVCDQ KLRPNIPNWW QSYEALRVMG KMMRECWYAN
481 GAARLTALRI KKTLSQLSVQ EDVKI
```

The signal peptide is indicated by a single underline and the extracellular domain is indicated in bold font.

A processed extracellular human ALK4 polypeptide sequence is as follows:

(SEQ ID NO: 86)
SGPRGVQALLCACTSCLQANYTCETDGACMVSIFNLDGMEHHVR
TCIPKVELVPAGKPFYCLSSEDLRNTHCCYTDYCNRIDLRVPSG
HLKEPEHPSMWGPVE

A nucleic acid sequence encoding an ALK4 precursor polypeptide is shown in SEQ ID NO: 221), corresponding to nucleotides 78-1592 of GenBank Reference Sequence NM_004302.4.

The signal sequence is underlined and the extracellular domain is indicated in bold font.

(SEQ ID NO: 221)
ATGGCGGAGTCGGCCGGAGCCTCCTCCTTCTTCCCCCTTGTTGTC
CTCCTGCTCGCCGGCAGCGGCGGGTCCGGGCCCCGGGGGGTCCAG
GCTCTGCTGTGTGCGTGCACCAGCTGCCTCCAGGCCAACTACACG
TGTGAGACAGATGGGGCCTGCATGGTTTCCATTTTCAATCTGGAT
GGGATGGAGCACCATGTGCGCACCTGCATCCCCAAAGTGGAGCTG
GTCCCTGCCGGGAAGCCCTTCTACTGCCTGAGCTCGGAGGACCTG
CGCAACACCCACTGCTGCTACACTGACTACTGCAACAGGATCGAC
TTGAGGGTGCCCAGTGGTCACCTCAAGGAGCCTGAGCACCCGTCC
ATGTGGGGCCCGGTGGAGCTGGTAGGCATCATCGCCGGCCCGGTG
TTCCTCCTGTTCCTCATCATCATCATTGTTTTCCTTGTCATTAAC
TATCATCAGCGTGTCTATCACAACCGCCAGAGACTGGACATGGAA
GATCCCTCATGTGAGATGTGTCTCTCCAAAGACAAGACGCTCCAG
GATCTTGTCTACGATCTCTCCACCTCAGGGTCTGGCTCAGGGTTA
CCCCTCTTTGTCCAGCGCACAGTGGCCCGAACCATCGTTTTACAA
GAGATTATTGGCAAGGGTCGGTTTGGGGAAGTATGGCGGGGCCGC
TGGAGGGGTGGTGATGTGGCTGTGAAAATATTCTCTTCTCGTGAA
GAACGGTCTTGGTTCAGGGAAGCAGAGATATACCAGACGGTCATG
CTGCGCCATGAAAACATCCTTGGATTTATTGCTGCTGACAATAAA
GATAATGGCACCTGGACACAGCTGTGGCTTGTTTCTGACTATCAT
GAGCACGGGTCCCTGTTTGATTATCTGAACCGGTACACAGTGACA
ATTGAGGGGATGATTAAGCTGGCCTTGTCTGCTGCTAGTGGGCTG
GCACACCTGCACATGGAGATCGTGGGCACCCAAGGGAAGCCTGGA
ATTGCTCATCGAGACTTAAAGTCAAAGAACATTCTGGTGAAGAAA
AATGGCATGTGTGCCATAGCAGACCTGGGCCTGGCTGTCCGTCAT
GATGCAGTCACTGACACCATTGACATTGCCCCGAATCAGAGGGTG
GGGACCAAACGATACATGGCCCCTGAAGTACTTGATGAAACCATT
AATATGAAACACTTTGACTCCTTTAAATGTGCTGATATTTATGCC
CTCGGGCTTGTATATTGGGAGATTGCTCGAAGATGCAATTCTGGA
GGAGTCCATGAAGAATATCAGCTGCCATATTACGACTTAGTGCCC
TCTGACCCTTCCATTGAGGAAATGCGAAAGGTTGTATGTGATCAG
AAGCTGCGTCCCAACATCCCCAACTGGTGGCAGAGTTATGAGGCA
CTGCGGGTGATGGGGAAGATGATGCGAGAGTGTTGGTATGCCAAC
GGCGCAGCCCGCCTGACGGCCCTGCGCATCAAGAAGACCCTCTCC
CAGCTCAGCGTGCAGGAAGACGTGAAGATC

A nucleic acid sequence encoding an extracellular ALK4 polypeptide is shown in SEQ ID NO: 222.

(SEQ ID NO: 222)
TCCGGGCCCCGGGGGGTCCAGGCTCTGCTGTGTGCGTGCACCAGC
TGCCTCCAGGCCAACTACACGTGTGAGACAGATGGGGCCTGCATG
GTTTCCATTTTCAATCTGGATGGGATGGAGCACCATGTGCGCACC
TGCATCCCCAAAGTGGAGCTGGTCCCTGCCGGGAAGCCCTTCTAC
TGCCTGAGCTCGGAGGACCTGCGCAACACCCACTGCTGCTACACT
GACTACTGCAACAGGATCGACTTGAGGGTGCCCAGTGGTCACCTC
AAGGAGCCTGAGCACCCGTCCATGTGGGGCCCGGTGGAG

An alternative isoform of human ALK4 precursor protein sequence, isoform B (NCBI Ref Seq NP_064732.3), is as follows:

| (SEQ ID NO: 421) | |
|---|---|
| 1 | MVSIFNLDGM EHHVRTCIPK VELVPAGKPF YCLSSEDLRN THCCYTDYCN RIDLRVPSGH |
| 61 | LKEPEHPSMW GPVELVGIIA GPVFLLFLII IIVFLVINYH QRVYHNRQRL DMEDPSCEMC |
| 121 | LSKDKTLQDL VYDLSTSGSG SGLPLFVQRT VARTIVLQEI IGKGRFGEVW RGRWRGGDVA |
| 181 | VKIFSSREER SWFREAEIYQ TVMLRHENIL GFIAADNKDN GTWTQLWLVS DYHEHGSLFD |
| 241 | YLNRYTVTIE GMIKLALSAA SGLAHLHMEI VGTQGKPGIA HRDLKSKNIL VKKNGMCAIA |
| 301 | DLGLAVRHDA VTDTIDIAPN QRVGTKRYMA PEVLDETINM KHFDSFKCAD IYALGLVYWE |
| 361 | IARRCNSGGV HEEYQLPYYD LVPSDPSIEE MRKVVCDQKL RPNIPNWWQS YEALRVMGKM |
| 421 | MRECWYANGA ARLTALRIKK TLSQLSVQED VKI |

The extracellular domain is indicated in bold font.

A processed extracellular ALK4 polypeptide sequence corresponding to isoform B above is as follows:
1 MVSIFNLDGM EHHVRTCIPK VELVPAGKPF YCLSSEDLRN THCCYTDYCN RIDLRVPSGH 61 LKEPEHPSMW GPVE (SEQ ID NO: 422)

A nucleic acid sequence encoding the ALK4 precursor protein (isoform B) is shown below (SEQ ID NO: 423), corresponding to nucleotides 186-1547 of GenBank Reference Sequence NM_020327.3. The nucleotides encoding the extracellular domain are indicated in bold font.

| (SEQ ID NO: 423) | |
|---|---|
| 1 | ATGGTTTCCA TTTTCAATCT GGATGGGATG GAGCACCATG TGCGCACCTG |
| 51 | CATCCCCAAA GTGGAGCTGG TCCCTGCCGG GAAGCCCTTC TACTGCCTGA |
| 101 | GCTCGGAGGA CCTGCGCAAC ACCCACTGCT GCTACACTGA CTACTGCAAC |
| 151 | AGGATCGACT TGAGGGTGCC CAGTGGTCAC CTCAAGGAGC CTGAGCACCC |
| 201 | GTCCATGTGG GGCCCGGTGG AGCTGGTAGG CATCATCGCC GGCCCGGTGT |
| 251 | TCCTCCTGTT CCTCATCATC ATCATTGTTT TCCTTGTCAT TAACTATCAT |
| 301 | CAGCGTGTCT ATCACAACCG CCAGAGACTG GACATGGAAG ATCCCTCATG |
| 351 | TGAGATGTGT CTCTCCAAAG ACAAGACGCT CCAGGATCTT GTCTACGATC |
| 401 | TCTCCACCTC AGGGTCTGGC TCAGGGTTAC CCCTCTTTGT CCAGCGCACA |
| 451 | GTGGCCCGAA CCATCGTTTT ACAAGAGATT ATTGGCAAGG GTCGGTTTGG |
| 501 | GGAAGTATGG CGGGGCCGCT GGAGGGGIGG TGATGTGGCT GTGAAAATAT |
| 551 | TCTCTTCTCG TGAAGAACGG TCTTGGTTCA GGGAAGCAGA GATATACCAG |
| 601 | ACGGTCATGC TGCGCCATGA AAACATCCTT GGATTTATTG CTGCTGACAA |
| 651 | TAAAGATAAT GGCACCTGGA CACAGCTGTG GCTTGTTTCT GACTATCATG |
| 701 | AGCACGGGTC CCTGITTGAT TATCTGAACC GGTACACAGT GACAATTGAG |
| 751 | GGGATGATTA AGCTGGCCTT GTCTGCTGCT AGTGGGCTGG CACACCTGCA |
| 801 | CATGGAGATC GTGGGCACCC AAGGGAAGCC TGGAATTGCT CATCGAGACT |
| 851 | TAAAGTCAAA GAACATTCTG GTGAAGAAAA ATGGCATGTG TGCCATAGCA |
| 901 | GACCTGGGCC TGGCTGTCCG TCATGATGCA GTCACTGACA CCATTGACAT |
| 951 | TGCCCCGAAT CAGAGGGTGG GGACCAAACG ATACATGGCC CCTGAAGTAC |
| 1001 | TTGATGAAAC CATTAATATG AAACACTTTG ACTCCTTTAA ATGTGCTGAT |
| 1051 | ATTTATGCCC TCGGGCTTGT ATATTGGGAG ATTGCTCGAA GATGCAATTC |
| 1101 | TGGAGGAGTC CATGAAGAAT ATCAGCTGCC ATATTACGAC TTAGTGCCCT |
| 1151 | CTGACCCTTC CATTGAGGAA ATGCGAAAGG TIGTATGTGA TCAGAAGCTG |
| 1201 | CGTCCCAACA TCCCCAACTG GTGGCAGAGT TATGAGGCAC TGCGGGTGAT |
| 1251 | GGGGAAGATG ATGCGAGAGT GTTGGTATGC CAACGGCGCA GCCCGCCTGA |
| 1301 | CGGCCCTGCG CATCAAGAAG ACCCTCTCCC AGCTCAGCGT GCAGGAAGAC |
| 1351 | GTGAAGATCT AA |

A nucleic acid sequence encoding the extracellular ALK4 polypeptide (isoform B) is as follows:

| (SEQ ID NO: 424) | |
|---|---|
| 1 | ATGGTTTCCA TTTTCAATCT GGATGGGATG GAGCACCATG TGCGCACCTG |
| 51 | CATCCCCAAA GTGGAGCTGG TCCCTGCCGG GAAGCCCTTC TACTGCCTGA |
| 101 | GCTCGGAGGA CCTGCGCAAC ACCCACTGCT GCTACACTGA CTACTGCAAC |
| 151 | AGGATCGACT TGAGGGTGCC CAGTGGTCAC CTCAAGGAGC CTGAGCACCC |
| 201 | GTCCATGTGG GGCCCGGTGG AGCTGGTAGG |

An alternative isoform of human ALK4 precursor polypeptide sequence, isoform C (NCBI Ref Seq NP_064733.3), is as follows:

| (SEQ ID NO: 85) | |
|---|---|
| 1 | MAESAGASSF FPLVVLLLAG SGGSGPRGVQ ALLCACTSCL QANYTCETDG ACMVSIFNLD |
| 61 | GMEHHVRTCI PKVELVPAGK PFYCLSSEDL RNTHCCYTDY CNRIDLRVPS GHLKEPEHPS |
| 121 | MWGPVELVGI IAGPVFLLFL IIIIVELVIN YHQRVYHNRQ RLDMEDPSCE MCLSKDKTLQ |
| 181 | DLVYDLSTSG SGSGLPLFVQ RTVARTIVLQ EIIGKGRFGE VWRGRWRGGD VAVKIFSSRE |
| 241 | ERSWFREAEI YQTVMLRHEN ILGFIAADNK ADCSFLTLPW EVVMVSAAPK LRSLRLQYKG |
| 301 | GRGRARFLFP LNNGTWTQLW LVSDYHEHGS LFDYLNRYTV TIEGMIKLAL SAASGLAHLH |
| 361 | MEIVGTQGKP GIAHRDLKSK NILVKKNGMC AIADLGLAVR HDAVIDTIDI APNQRVGTKR |
| 421 | YMAPEVLDET INMKHFDSFK CADIYALGLV YWEIARRCNS GGVHEEYQLP YYDLVPSDPS |
| 481 | IEEMRKVVCD QKLRPNIPNW WQSYEALRVM GKMMRECWYA NGAARLTALR IKKTLSQLSV |
| 541 | QEDVKI |

The signal peptide is indicated by a single underline and the extracellular domain is indicated in bold font.

A processed extracellular ALK4 polypeptide sequence (isoform C) is as follows:

(SEQ ID NO: 87)
SGPRGVQALLCACTSCLQANYTCETDGACMVSIFNLDGMEHHVRTCIPK
VELVPAGKPFYCLSSEDLRNTHCCYTDYCNRIDLRVPSGHLKEPEHPSM
WGPVE

A nucleic acid sequence encoding an ALK4 precursor polypeptide (isoform C) is shown in SEQ ID NO: 223, corresponding to nucleotides 78-1715 of GenBank Reference Sequence NM_020328.3.

(SEQ ID NO: 223)
ATGGCGGAGTCGGCCGGAGCCTCCTCCTTCTTCCCCCTTGTTGTCCTCC
TGCTCGCCGGCAGCGGCGGGTCCGGGCCCCGGGGGGTCCAGGCTCTGCT
GTGTGCGTGCACCAGCTGCCTCCAGGCCAACTACACGTGTGAGACAGAT
GGGGCCTGCATGGTTTCCATTTTCAATCTGGATGGGATGGAGCACCATG
TGCGCACCTGCATCCCCAAAGTGGAGCTGGTCCCTGCCGGGAAGCCCTT
CTACTGCCTGAGCTCGGAGGACCTGCGCAACACCCACTGCTGCTACACT
GACTACTGCAACAGGATCGACTTGAGGGTGCCCAGTGGTCACCTCAAGG
AGCCTGAGCACCCGTCCATGTGGGGCCCGGTGGAGCTGGTAGGCATCAT
CGCCGGCCCGGTGTTCCTCCTGTTCCTCATCATCATCATTGTTTTCCTT
GTCATTAACTATCATCAGCGTGTCTATCACAACCGCCAGAGACTGGACA
TGGAAGATCCCTCATGTGAGATGTGTCTCTCCAAAGACAAGACGCTCCA
GGATCTTGTCTACGATCTCTCCACCTCAGGGTCTGGCTCAGGGTTACCC
CTCTTTGTCCAGCGCACAGTGGCCCGAACCATCGTTTTACAAGAGATTA
TTGGCAAGGGTCGGTTTGGGGAAGTATGGCGGGGCCGCTGGAGGGGTGG
TGATGTGGCTGTGAAAATATTCTCTTCTCGTGAAGAACGGTCTTGGTTC
AGGGAAGCAGAGATATACCAGACGGTCATGCTGCGCCATGAAAACATCC
TTGGATTTATTGCTGCTGACAATAAAGCAGACTGCTCATTCCTCACATT
GCCATGGGAAGTTGTAATGGTCTCTGCTGCCCCCAAGCTGAGGAGCCTT
AGACTCCAATACAAGGGAGGAAGGGGAAGAGCAAGATTTTTATTCCCAC
TGAATAATGGCACCTGGACACAGCTGTGGCTTGTTTCTGACTATCATGA
GCACGGGTCCCTGTTTGATTATCTGAACCGGTACACAGTGACAATTGAG
GGGATGATTAAGCTGGCCTTGTCTGCTGCTAGTGGGCTGGCACACCTGC
ACATGGAGATCGTGGGCACCCAAGGGAAGCCTGGAATTGCTCATCGAGA
CTTAAAGTCAAAGAACATTCTGGTGAAGAAAAATGGCATGTGTGCCATA
GCAGACCTGGGCCTGGCTGTCCGTCATGATGCAGTCACTGACACCATTG
ACATTGCCCCGAATCAGAGGGTGGGGACCAAACGATACATGGCCCCTGA
AGTACTTGATGAAACCATTAATATGAAACACTTTGACTCCTTTAAATGT
GCTGATATTTATGCCCTCGGGCTTGTATATTGGGAGATTGCTCGAAGAT
GCAATTCTGGAGGAGTCCATGAAGAATATCAGCTGCCATATTACGACTT
AGTGCCCTCTGACCCTTCCATTGAGGAAATGCGAAAGGTTGTATGTGAT
CAGAAGCTGCGTCCCAACATCCCCAACTGGTGGCAGAGTTATGAGGCAC
TGCGGGTGATGGGGAAGATGATGCGAGAGTGTTGGTATGCCAACGGCGC
AGCCCGCCTGACGGCCCTGCGCATCAAGAAGACCCTCTCCCAGCTCAGC
GTGCAGGAAGACGTGAAGATC

A nucleic acid sequence encoding the extracellular ALK4 polypeptide (isoform C) is shown in SEQ ID NO: 224.

(SEQ ID NO: 224)
TCCGGGCCCCGGGGGGTCCAGGCTCTGCTGTGTGCGTGCACCAGCTGCC
TCCAGGCCAACTACACGTGTGAGACAGATGGGGCCTGCATGGTTTCCAT
TTTCAATCTGGATGGGATGGAGCACCATGTGCGCACCTGCATCCCCAAA
GTGGAGCTGGTCCCTGCCGGGAAGCCCTTCTACTGCCTGAGCTCGGAGG
ACCTGCGCAACACCCACTGCTGCTACACTGACTACTGCAACAGGATCGA
CTTGAGGGTGCCCAGTGGTCACCTCAAGGAGCCTGAGCACCCGTCCATG
TGGGGCCCGGTGGAG

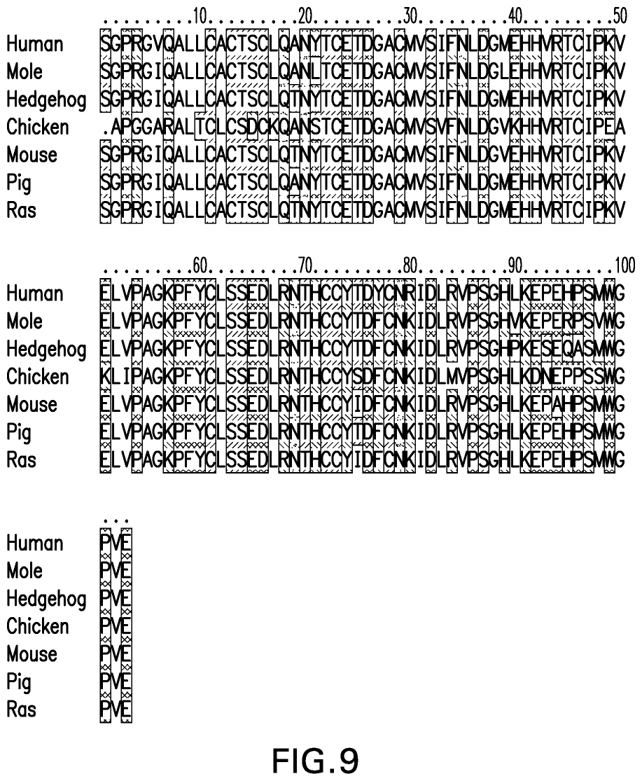

ALK4 is well-conserved among vertebrates, with large stretches of the extracellular domain completely conserved. For example, FIG. 9 depicts a multi-sequence alignment of a human ALK4 extracellular domain compared to various ALK4 orthologs. Many of the ligands that bind to ALK4 are also highly conserved. Accordingly, from these alignments, it is possible to predict key amino acid positions within the ligand-binding domain that are important for normal ALK4-ligand binding activities as well as to predict amino acid positions that are likely to be tolerant to substitution without significantly altering normal ALK4-ligand binding activities. Therefore, an active, human ALK4 variant polypeptide useful in accordance with the presently disclosed methods may include one or more amino acids at corresponding positions from the sequence of another vertebrate ALK4, or may include a residue that is similar to that in the human or other vertebrate sequences.

Without meaning to be limiting, the following examples illustrate this approach to defining an active ALK4 variant. As illustrated in FIG. 9, V6 in the human ALK4 extracellular domain (SEQ ID NO: 414) is isoleucine in Mus muculus ALK4 (SEQ ID NO: 418), and so the position may be altered, and optionally may be altered to another hydrophobic residue such as L, I, or F, or a non-polar residue such as A, as is observed in Gallus gallus ALK4 (SEQ ID NO: 417). E40 in the human extracellular domain is K in Gallus gallus ALK4, indicating that this site may be tolerant of a wide variety of changes, including polar residues, such as E, D, K, R, H, S, T, P, G, Y, and probably a non-polar residue such as A. S15 in the human extracellular domain is D in Gallus gallus ALK4, indicating that a wide structural variation is tolerated at this position, with polar residues favored, such as S, T, R, E, K, H, G, P, G and Y. E40 in the human extracellular domain is K in Gallus gallus ALK4, indicating that charged residues will be tolerated at this position, including D, R, K, H, as well as Q and N. R80 in the human extracellular domain is K in Condylura cristata ALK4 (SEQ ID NO: 415), indicating that basic residues are tolerated at this position, including R, K, and H. Y77 in the human extracellular domain is F in Sus scrofa ALK4 (SEQ ID NO: 419), indicating that aromatic residues are tolerated at this position, including F, W, and Y. P93 in the human extracellular domain is relatively poorly conserved, appearing as S in Erinaceus europaeus ALK4 (SEQ ID NO: 416) and N in Gallus gallus ALK4, thus essentially any amino acid should be tolerated at this position.

Moreover, ALK4 proteins have been characterized in the art in terms of structural and functional characteristics, particularly with respect to ligand binding [e.g., Harrison et al. (2003) J Biol Chem 278(23):21129-21135; Romano et al. (2012) J Mol Model 18(8):3617-3625; and Calvanese et al. (2009) 15(3):175-183]. In addition to the teachings herein, these references provide ample guidance for how to generate ALK4 variants that retain one or more normal activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ALK4, as demarcated by the outermost of these conserved cysteines, corresponds to positions 34-101 of SEQ ID NO: 84 (ALK4 precursor). The structurally less-ordered amino acids flanking these cysteine-demarcated core sequences can be truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 residues at the N-terminus and/or by 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ALK4 extracellular domains for N-terminal and/or C-terminal truncation include SEQ ID NOs: 86, 87, and 422.

In certain embodiments, the disclosure relates to heteromultimers that comprise at least one ALK4 polypeptide, which includes fragments, functional variants, and modified forms thereof. Preferably, ALK4 polypeptides for use as disclosed herein (e.g., heteromultimers comprising an ALK4 polypeptide and uses thereof) are soluble (e.g., an extracellular domain of ALK4). In other embodiments, ALK4 polypeptides for use as disclosed herein bind to and/or inhibit (antagonize) activity (e.g., induction of Smad signaling) of one or more TGF-beta superfamily ligands. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to the amino acid sequence of SEQ ID NO: 84, 85, 86, 87, 88, 89, 92, 93, 421, and 422. In some embodiments, heteromultimers of the disclosure consist or consist essentially of at least one ALK4 polypeptide that is at least 70%, 75%, 80%, 85%, 90%, 91%, 92%, 93%, 94%, 95%, 96%, 97%, 98%, 99%, or 100% identical to the amino acid sequence of SEQ ID NO: 84, 85, 86, 87, 88, 89, 92, 93, 422.

In certain aspects, the disclosure relates to a heteromultimer that comprises an ALK4-Fc fusion polypeptide. In some embodiments, the ALK4-Fc fusion polypeptide comprises an ALK4 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23-34 (e.g., amino acid residues 23, 24, 25, 26, 27, 28, 29, 30, 31, 32, 33, 34) SEQ ID NO: 84, 85, or 421 and ends at any one of amino acids 101-126 (e.g., amino acid residues 101, 102, 103, 104, 105, 106, 107, 108, 109, 110, 111, 112, 113, 114, 115, 116, 117, 118, 119, 120, 121, 122, 123, 124, 125, and 126) of SEQ ID NO: 84, 85, or 421. In some embodiments, the ALK4-Fc fusion polypeptide comprises an ALK4 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 34-101 of SEQ ID NOs: 84, 85, or 421. In some embodiments, the ALK4-Fc fusion polypeptide comprises an ALK4 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 23-126 of SEQ ID Nos: 84, 85, or 421. In some embodiments, the ALK4-Fc fusion polypeptide comprises an ALK4 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of any one of SEQ ID Nos: 84, 85, 86, 87, 88, 89, 92, 93, 247, 249, 421, 422.

E) ALK7 Polypeptides

In certain aspects, the disclosure relates to ActRII-ALK4 antagonists comprising an ALK7 polypeptide, which includes fragments, functional variants, and modified forms thereof as well as uses thereof (e.g., of treating, preventing, or reducing the progression rate and/or severity of heart failure (HF) or one or more complications of HF). As used herein, the term “ALK7” refers to a family of activin receptor-like kinase-7 (ALK7) proteins from any species and variant polypeptides derived from such ALK7 proteins by mutagenesis or other modifications (including, e.g., mutants, fragments, fusions, and peptidomimetic forms) that retain a useful activity. Examples of such variant ALK7 polypeptides are provided throughout the present disclosure as well as in International Patent Application Publication Nos. WO/2016/164089 and WO/2016/164503, which are incorporated herein by reference in their entirety. Reference to ALK7 herein is understood to be a reference to any one of the currently identified forms. Members of the ALK7 family are generally all transmembrane polypeptides, composed of a ligand-binding extracellular domain with cysteine-rich region, a transmembrane domain, and a cytoplasmic domain with predicted serine/threonine kinase specificity. The amino acid sequence of a human ALK7 precursor polypeptide is shown in (SEQ ID NO: 120) below. Preferably, ALK7 polypeptides to be used in accordance with the methods of the disclosure are soluble. The term “soluble ALK7 polypeptide,” as used herein, includes any naturally occurring extracellular domain of an ALK7 polypeptide as well as any variants thereof (including mutants, fragments and peptidomimetic forms) that retain a useful activity. For example, the extracellular domain of an ALK7 polypeptide binds to a ligand and is generally soluble. Examples of soluble ALK7 polypeptides include an ALK7 extracellular domain (SEQ ID NO: 123) below. Other examples of soluble ALK7 polypeptides comprise a signal sequence in addition to the extracellular domain of an ALK7 polypeptide. The signal sequence can be a native signal sequence of an ALK7, or a signal sequence from another polypeptide, such as a tissue plasminogen activator (TPA) signal sequence or a honey bee melittin signal sequence. In some embodiments, ALK7 polypeptides inhibit activity (e.g., Smad signaling) of one or more ActRII-ALK4 ligands (e.g., activin A, activin B, GDF8, GDF11, BMP6, BMP10). In some embodiments, ALK7 polypeptides bind to one or more ActRII-ALK4 ligands (e.g., activin A, activin B, GDF8, GDF11, BMP6, BMP10). Various examples of methods and assays for determining the ability of an ALK7 polypeptide to bind to and/or inhibit activity of one or more ActRII-ALK4 ligands are disclosed herein or otherwise well known in the art, which can be readily used to determine if an ALK7 polypeptide has the desired binding and/or antagonistic activities. Numbering of amino acids for all ALK7-related polypeptides described herein is based on the numbering of the human ALK7 precursor protein sequence provided below (SEQ ID NO: 120), unless specifically designated otherwise.

Four naturally occurring isoforms of human ALK7 have been described. The sequence of human ALK7 isoform 1 precursor polypeptide (NCBI Ref Seq NP_660302.2) is as follows:

| (SEQ ID NO: 120) | |
|---|---|
| 1 | MTRALCSALR QALLLLAAAA ELSPGLKCVC LLCDSSNFTC QTEGACWASV MLTNGKEQVI |
| 61 | KSCVSLPELN AQVFCHSSNN VTKTECCFTD FCNNITLHLP TASPNAPKLG PMELAIIITV |
| 121 | PVCLLSIAAM LTVWACQGRQ CSYRKKKRPN VEEPLSECNL VNAGKTLKDL IYDVTASGSG |
| 181 | SGLPLLVQRT IARTIVLQEI VGKGRFGEVW HGRWCGEDVA VKIFSSRDER SWFREAEIYQ |
| 241 | TVMLRHENIL GFIAADNKDN GTWTQLWLVS EYHEQGSLYD YLNRNIVTVA GMIKLALSIA |
| 301 | SGLAHLHMEI VGTQGKPAIA HRDIKSKNIL VKKCETCAIA DLGLAVKHDS ILNTIDIPQN |
| 361 | PKVGTKRYMA PEMLDDTMNV NIFESFKRAD IYSVGLVYWE IARRCSVGGI VEEYQLPYYD |
| 421 | MVPSDPSIEE MRKVVCDQKF RPSIPNQWQS CEALRVMGRI MRECWYANGA ARLTALRIKK |
| 481 | TISQLCVKED CKA |

The signal peptide is indicated by a single underline and the extracellular domain is indicated in bold font.

A processed extracellular ALK7 isoform 1 polypeptide sequence is as follows:

(SEQ ID NO: 123)
ELSPGLKCVCLLCDSSNFTCQTEGACWASVMLTNGKEQVIKSCVSLPEL
NAQVFCHSSNNVTKTECCFTDFCNNITLHLPTASPNAPKLGPME

A nucleic acid sequence encoding human ALK7 isoform 1 precursor polypeptide is shown below in SEQ ID NO: 233, corresponding to nucleotides 244-1722 of GenBank Reference Sequence NM_145259.2.

(SEQ ID NO: 233)
ATGACCCGGGCGCTCTGCTCAGCGCTCCGCCAGGCTCTCCTGCTGCTCG
CAGCGGCCGCCGAGCTCTCGCCAGGACTGAAGTGTGTATGTCTTTTGTG
TGATTCTTCAAACTTTACCTGCCAAACAGAAGGAGCATGTTGGGCATCA
GTCATGCTAACCAATGGAAAAGAGCAGGTGATCAAATCCTGTGTCTCCC
TTCCAGAACTGAATGCTCAAGTCTTCTGTCATAGTTCCAACAATGTTAC
CAAAACCGAATGCTGCTTCACAGATTTTTGCAACAACATAACACTGCAC
CTTCCAACAGCATCACCAAATGCCCCAAAACTTGGACCCATGGAGCTGG
CCATCATTATTACTGTGCCTGTTTGCCTCCTGTCCATAGCTGCGATGCT
GACAGTATGGGCATGCCAGGGTCGACAGTGCTCCTACAGGAAGAAAAAG
AGACCAAATGTGGAGGAACCACTCTCTGAGTGCAATCTGGTAAATGCTG
GAAAAACTCTGAAAGATCTGATTTATGATGTGACCGCCTCTGGATCTGG
CTCTGGTCTACCTCTGTTGGTTCAAAGGACAATTGCAAGGACGATTGTG
CTTCAGGAAATAGTAGGAAAAGGTAGATTTGGTGAGGTGTGGCATGGAA
GATGGTGTGGGGAAGATGTGGCTGTGAAAATATTCTCCTCCAGAGATGA
AAGATCTTGGTTTCGTGAGGCAGAAATTTACCAGACGGTCATGCTGCGA
CATGAAAACATCCTTGGTTTCATTGCTGCTGACAACAAAGATAATGGAA
CTTGGACTCAACTTTGGCTGGTATCTGAATATCATGAACAGGGCTCCTT
ATATGACTATTTGAATAGAAATATAGTGACCGTGGCTGGAATGATCAAG
CTGGCGCTCTCAATTGCTAGTGGTCTGGCACACCTTCATATGGAGATTG
TTGGTACACAAGGTAAACCTGCTATTGCTCATCGAGACATAAAATCAAA
GAATATCTTAGTGAAAAAGTGTGAAACTTGTGCCATAGCGGACTTAGGG
TTGGCTGTGAAGCATGATTCAATACTGAACACTATCGACATACCTCAGA
ATCCTAAAGTGGGAACCAAGAGGTATATGGCTCCTGAAATGCTTGATGA
TACAATGAATGTGAATATCTTTGAGTCCTTCAAACGAGCTGACATCTAT
TCTGTTGGTCTGGTTTACTGGGAAATAGCCCGGAGGTGTTCAGTCGGAG
GAATTGTTGAGGAGTACCAATTGCCTTATTATGACATGGTGCCTTCAGA
TCCCTCGATAGAGGAAATGAGAAAGGTTGTTTGTGACCAGAAGTTTCGA
CCAAGTATCCCAAACCAGTGGCAAAGTTGTGAAGCACTCCGAGTCATGG
GGAGAATAATGCGTGAGTGTTGGTATGCCAACGGAGCGGCCCGCCTAAC
TGCTCTTCGTATTAAGAAGACTATATCTCAACTTTGTGTCAAAGAAGAC
TGCAAAGCC

A nucleic acid sequence encoding the processed extracellular ALK7 polypeptide (isoform 1) is show in SEQ ID NO: 234.

(SEQ ID NO: 234)
GAGCTCTCGCCAGGACTGAAGTGTGTATGTCTTTTGTGTGATTCTTCAA
ACTTTACCTGCCAAACAGAAGGAGCATGTTGGGCATCAGTCATGCTAAC
CAATGGAAAAGAGCAGGTGATCAAATCCTGTGTCTCCCTTCCAGAACTG
AATGCTCAAGTCTTCTGTCATAGTTCCAACAATGTTACCAAAACCGAAT
GCTGCTTCACAGATTTTTGCAACAACATAACACTGCACCTTCCAACAGC
ATCACCAAATGCCCCAAAACTTGGACCCATGGAG

An amino acid sequence of an alternative isoform of human ALK7, isoform 2 (NCBI Ref Seq NP_001104501.1), is shown in its processed form as follows (SEQ ID NO: 124), where the extracellular domain is indicated in bold font.

| (SEQ ID NO: 124) | |
|---|---|
| 1 | MLTNGKEQVI KSCVSLPELN AQVFCHSSNN VTKTECCFTD FCNNITLHLP TASPNAPKLG |
| 61 | PMELAIIITV PVCLLSIAAM LTVWACQGRQ CSYRKKKRPN VEEPLSECNL VNAGKTLKDL |
| 121 | IYDVTASGSG SGLPLLVQRT IARTIVLOEI VGKGRFGEVW HGRWCGEDVA VKIFSSRDER |
| 181 | SWFREAEIYQ TVMLRHENIL GFIAADNKDN GTWTOLWLVS EYHEQGSLYD YLNRNIVTVA |
| 241 | GMIKLALSIA SGLAHLHMEI VGTQGKPAIA HRDIKSKNIL VKKCETCAIA DLGLAVKHDS |
| 301 | ILNTIDIPQN PKVGTKRYMA PEMLDDTMNV NIFESFKRAD IYSVGLVYWE IARRCSVGGI |
| 361 | VEEYQLPYYD MVPSDPSIEE MRKVVCDQKF RPSIPNQWQS CEALRVMGRI MRECWYANGA |
| 421 | ARLTALRIKK TISQLCVKED CKA |

An amino acid sequence of the extracellular ALK7 polypeptide (isoform 2) is as follows:

(SEQ ID NO: 125)
MLTNGKEQVIKSCVSLPELNAQVFCHSSNNVTKTECCFTDFCNNITLHL
PTASPNAPKLGPME.

A nucleic acid sequence encoding the processed ALK7 polypeptide (isoform 2) is shown below in SEQ ID NO: 235, corresponding to nucleotides 279-1607 of NCBI Reference Sequence NM_001111031.1.

(SEQ ID NO: 235)
ATGCTAACCAATGGAAAAGAGCAGGTGATCAAATCCTGTGTCTCCCTTC
CAGAACTGAATGCTCAAGTCTTCTGTCATAGTTCCAACAATGTTACCAA
AACCGAATGCTGCTTCACAGATTTTTGCAACAACATAACACTGCACCTT
CCAACAGCATCACCAAATGCCCCAAAACTTGGACCCATGGAGCTGGCCA
TCATTATTACTGTGCCTGTTTGCCTCCTGTCCATAGCTGCGATGCTGAC
AGTATGGGCATGCCAGGGTCGACAGTGCTCCTACAGGAAGAAAAAGAGA
CCAAATGTGGAGGAACCACTCTCTGAGTGCAATCTGGTAAATGCTGGAA
AAACTCTGAAAGATCTGATTTATGATGTGACCGCCTCTGGATCTGGCTC
TGGTCTACCTCTGTTGGTTCAAAGGACAATTGCAAGGACGATTGTGCTT
CAGGAAATAGTAGGAAAAGGTAGATTTGGTGAGGTGTGGCATGGAAGAT
GGTGTGGGGAAGATGTGGCTGTGAAAATATTCTCCTCCAGAGATGAAAG
ATCTTGGTTTCGTGAGGCAGAAATTTACCAGACGGTCATGCTGCGACAT
GAAAACATCCTTGGTTTCATTGCTGCTGACAACAAAGATAATGGAACTT
GGACTCAACTTTGGCTGGTATCTGAATATCATGAACAGGGCTCCTTATA
TGACTATTTGAATAGAAATATAGTGACCGTGGCTGGAATGATCAAGCTG
GCGCTCTCAATTGCTAGTGGTCTGGCACACCTTCATATGGAGATTGTTG
GTACACAAGGTAAACCTGCTATTGCTCATCGAGACATAAAATCAAAGAA
TATCTTAGTGAAAAAGTGTGAAACTTGTGCCATAGCGGACTTAGGGTTG
GCTGTGAAGCATGATTCAATACTGAACACTATCGACATACCTCAGAATC
CTAAAGTGGGAACCAAGAGGTATATGGCTCCTGAAATGCTTGATGATAC
AATGAATGTGAATATCTTTGAGTCCTTCAAACGAGCTGACATCTATTCT
GTTGGTCTGGTTTACTGGGAAATAGCCCGGAGGTGTTCAGTCGGAGGAA
TTGTTGAGGAGTACCAATTGCCTTATTATGACATGGTGCCTTCAGATCC
CTCGATAGAGGAAATGAGAAAGGTTGTTTGTGACCAGAAGTTTCGACCA
AGTATCCCAAACCAGTGGCAAAGTTGTGAAGCACTCCGAGTCATGGGGA
GAATAATGCGTGAGTGTTGGTATGCCAACGGAGCGGCCCGCCTAACTGC
TCTTCGTATTAAGAAGACTATATCTCAACTTTGTGTCAAAGAAGACTGC
AAAGCC

A nucleic acid sequence encoding an extracellular ALK7 polypeptide (isoform 2) is shown in SEQ ID NO: 236.

(SEQ ID NO: 236)
ATGCTAACCAATGGAAAAGAGCAGGTGATCAAATCCTGTGTCTCCCTTC
CAGAACTGAATGCTCAAGTCTTCTGTCATAGTTCCAACAATGTTACCAA
AACCGAATGCTGCTTCACAGATTTTTGCAACAACATAACACTGCACCTT
CCAACAGCATCACCAAATGCCCCAAAACTTGGACCCATGGAG

An amino acid sequence of an alternative human ALK7 precursor polypeptide, isoform 3 (NCBI Ref Seq NP_001104502.1), is shown as follows (SEQ ID NO: 121), where the signal peptide is indicated by a single underline.

```
(SEQ ID NO: 121)
    1 MTRALCSALR QALLLLAAAA ELSPGLKCVC
        LLCDSSNFTC QTEGACWASV MLTNGKEQVI
  61 KSCVSLPELN AQVFCHSSNN VIKTECCFTD
        FCNNITLHLP TGLPLLVQRT IARTIVLQEI
121 VGKGRFGEVW HGRWCGEDVA VKIFSSRDER
        SWFREAEIYQ TVMLRHENIL GFIAADNKDN
181 GTWTQLWLVS EYHEQGSLYD YLNRNIVIVA
        GMIKLALSIA SGLAHLHMEI VGTQGKPAIA
241 HRDIKSKNIL VKKCETCAIA DLGLAVKHDS
        ILNTIDIPQN PKVGTKRYMA PEMLDDTMNV
301 NIFESFKRAD IYSVGLVYWE IARRCSVGGI
        VEEYQLPYYD MVPSDPSIEE MRKVVCDQKF
361 RPSIPNQWQS CEALRVMGRI MRECWYANGA
        ARLTALRIKK TISQLCVKED CKA
```

The amino acid sequence of a processed ALK7 polypeptide (isoform 3) is as follows (SEQ ID NO: 126). This isoform lacks a transmembrane domain and is therefore proposed to be soluble in its entirety (Roberts et al., 2003, Biol Reprod 68:1719-1726). N-terminal variants of SEQ ID NO: 126 are predicted as described below.

```
(SEQ ID NO: 126)
    1 ELSPGLKCVC LLCDSSNFTC QTEGACWASV
        MLTNGKEQVI KSCVSLPELN AQVFCHSSNN
  61 VIKTECCFTD FCNNITLHLP TGLPLLVQRT
        IARTIVLQEI VGKGREGEVW HGRWCGEDVA
121 VKIFSSRDER SWFREAEIYQ TVMLRHENIL
        GFIAADNKDN GTWTQLWLVS EYHEQGSLYD
181 YLNRNIVIVA GMIKLALSIA SGLAHLHMEI
        VGTQGKPAIA HRDIKSKNIL VKKCETCAIA
241 DLGLAVKHDS ILNTIDIPQN PKVGTKRYMA
        PEMLDDTMNV NIFESFKRAD IYSVGLVYWE
301 IARRCSVGGI VEEYQLPYYD MVPSDPSIEE
        MRKVVCDQKF RPSIPNQWQS CEALRVMGRI
361 MRECWYANGA ARLTALRIKK TISQLCVKED
        CKA
```

A nucleic acid sequence encoding an unprocessed ALK7 polypeptide precursor polypeptide (isoform 3) is shown in SEQ ID NO: 237, corresponding to nucleotides 244-1482 of NCBI Reference Sequence NM_001111032.1.

(SEQ ID NO: 237)
ATGACCCGGGCGCTCTGCTCAGCGCTCCGCCAGGCTCTCCTGCTG
CTCGCAGCGGCCGCCGAGCTCTCGCCAGGACTGAAGTGTGTATGT
CTTTTGTGTGATTCTTCAAACTTTACCTGCCAAACAGAAGGAGCA
TGTTGGGCATCAGTCATGCTAACCAATGGAAAAGAGCAGGTGATC
AAATCCTGTGTCTCCCTTCCAGAACTGAATGCTCAAGTCTTCTGT
CATAGTTCCAACAATGTTACCAAAACCGAATGCTGCTTCACAGAT
TTTTGCAACAACATAACACTGCACCTTCCAACAGGTCTACCTCTG
TTGGTTCAAAGGACAATTGCAAGGACGATTGTGCTTCAGGAAATA
GTAGGAAAAGGTAGATTTGGTGAGGTGTGGCATGGAAGATGGTGT
GGGGAAGATGTGGCTGTGAAAATATTCTCCTCCAGAGATGAAAGA
TCTTGGTTTCGTGAGGCAGAAATTTACCAGACGGTCATGCTGCGA
CATGAAAACATCCTTGGTTTCATTGCTGCTGACAACAAAGATAAT
GGAACTTGGACTCAACTTTGGCTGGTATCTGAATATCATGAACAG
GGCTCCTTATATGACTATTTGAATAGAAATATAGTGACCGTGGCT
GGAATGATCAAGCTGGCGCTCTCAATTGCTAGTGGTCTGGCACAC
CTTCATATGGAGATTGTTGGTACACAAGGTAAACCTGCTATTGCT
CATCGAGACATAAAATCAAAGAATATCTTAGTGAAAAAGTGTGAA
ACTTGTGCCATAGCGGACTTAGGGTTGGCTGTGAAGCATGATTCA
ATACTGAACACTATCGACATACCTCAGAATCCTAAAGTGGGAACC
AAGAGGTATATGGCTCCTGAAATGCTTGATGATACAATGAATGTG
AATATCTTTGAGTCCTTCAAACGAGCTGACATCTATTCTGTTGGT
CTGGTTTACTGGGAAATAGCCCGGAGGTGTTCAGTCGGAGGAATT
GTTGAGGAGTACCAATTGCCTTATTATGACATGGTGCCTTCAGAT
CCCTCGATAGAGGAAATGAGAAAGGTTGTTTGTGACCAGAAGTTT
CGACCAAGTATCCCAAACCAGTGGCAAAGTTGTGAAGCACTCCGA
GTCATGGGGAGAATAATGCGTGAGTGTTGGTATGCCAACGGAGCG
GCCCGCCTAACTGCTCTTCGTATTAAGAAGACTATATCTCAACTT
TGTGTCAAAGAAGACTGCAAAGCC

A nucleic acid sequence encoding a processed ALK7 polypeptide (isoform 3) is shown in SEQ ID NO: 238.

(SEQ ID NO: 238)
GAGCTCTCGCCAGGACTGAAGTGTGTATGTCTTTTGTGTGATTCT
TCAAACTTTACCTGCCAAACAGAAGGAGCATGTTGGGCATCAGTC
ATGCTAACCAATGGAAAAGAGCAGGTGATCAAATCCTGTGTCTCC
CTTCCAGAACTGAATGCTCAAGTCTTCTGTCATAGTTCCAACAAT
GTTACCAAAACCGAATGCTGCTTCACAGATTTTTGCAACAACATA
ACACTGCACCTTCCAACAGGTCTACCTCTGTTGGTTCAAAGGACA
ATTGCAAGGACGATTGTGCTTCAGGAAATAGTAGGAAAAGGTAGA
TTTGGTGAGGTGTGGCATGGAAGATGGTGTGGGGAAGATGTGGCT
GTGAAAATATTCTCCTCCAGAGATGAAAGATCTTGGTTTCGTGAG
GCAGAAATTTACCAGACGGTCATGCTGCGACATGAAAACATCCTT
GGTTTCATTGCTGCTGACAACAAAGATAATGGAACTTGGACTCAA
CTTTGGCTGGTATCTGAATATCATGAACAGGGCTCCTTATATGAC
TATTTGAATAGAAATATAGTGACCGTGGCTGGAATGATCAAGCTG
GCGCTCTCAATTGCTAGTGGTCTGGCACACCTTCATATGGAGATT
GTTGGTACACAAGGTAAACCTGCTATTGCTCATCGAGACATAAAA
TCAAAGAATATCTTAGTGAAAAAGTGTGAAACTTGTGCCATAGCG
GACTTAGGGTTGGCTGTGAAGCATGATTCAATACTGAACACTATC
GACATACCTCAGAATCCTAAAGTGGGAACCAAGAGGTATATGGCT
CCTGAAATGCTTGATGATACAATGAATGTGAATATCTTTGAGTCC
TTCAAACGAGCTGACATCTATTCTGTTGGTCTGGTTTACTGGGAA
ATAGCCCGGAGGTGTTCAGTCGGAGGAATTGTTGAGGAGTACCAA
TTGCCTTATTATGACATGGTGCCTTCAGATCCCTCGATAGAGGAA
ATGAGAAAGGTTGTTTGTGACCAGAAGTTTCGACCAAGTATCCCA
AACCAGTGGCAAAGTTGTGAAGCACTCCGAGTCATGGGGAGAATA
ATGCGTGAGTGTTGGTATGCCAACGGAGCGGCCCGCCTAACTGCT
CTTCGTATTAAGAAGACTATATCTCAACTTTGTGTCAAAGAAGAC
TGCAAAGCC

An amino acid sequence of an alternative human ALK7 precursor polypeptide, isoform 4 (NCBI Ref Seq NP_001104503.1), is shown as follows (SEQ ID NO: 122), where the signal peptide is indicated by a single underline.

```
(SEQ ID NO: 122)
    1 MTRALCSALR QALLLLAAAA ELSPGLKCVC
        LLCDSSNFTC QTEGACWASV MLTNGKEQVI
  61 KSCVSLPELN AQVFCHSSNN VIKTECCFTD
        FCNNITLHLP TDNGTWTQLW LVSEYHEQGS
121 LYDYLNRNIV TVAGMIKLAL SIASGLAHLH
        MEIVGTQGKP AIAHRDIKSK NILVKKCETC
181 AIADLGLAVK HDSILNTIDI PQNPKVGTKR
        YMAPEMLDDT MNVNIFESFK RADIYSVGLV
241 YWEIARRCSV GGIVEEYQLP YYDMVPSDPS
        IEEMRKVVCD QKFRPSIPNQ WQSCEALRVM
301 GRIMRECWYA NGAARLTALR IKKTISQLCV
        KEDCKA
```

An amino acid sequence of a processed ALK7 polypeptide (isoform 4) is as follows (SEQ ID NO: 127). Like ALK7 isoform 3, isoform 4 lacks a transmembrane domain and is therefore proposed to be soluble in its entirety (Roberts et al., 2003, Biol Reprod 68:1719-1726). N-terminal variants of SEQ ID NO: 127 are predicted as described below.

```
(SEQ ID NO: 127)
    1 ELSPGLKCVC LLCDSSNFTC QTEGACWASV
        MLTNGKEQVI KSCVSLPELN AQVFCHSSNN
  61 VIKTECCFTD FCNNITLHLP TDNGTWTQLW
        LVSEYHEQGS LYDYLNRNIV TVAGMIKLAL
121 SIASGLAHLH MEIVGTQGKP AIAHRDIKSK
        NILVKKCETC AIADLGLAVK HDSILNTIDI
181 PQNPKVGTKR YMAPEMLDDT MNVNIFESFK
        RADIYSVGLV YWEIARRCSV GGIVEEYQLP
240 YYDMVPSDPS IEEMRKVVCD QKFRPSIPNQ
        WQSCEALRVM GRIMRECWYA NGAARLTALR
301 IKKTISQLCV KEDCKA
```

A nucleic acid sequence encoding the unprocessed ALK7 polypeptide precursor polypeptide (isoform 4) is shown in SEQ ID NO: 239, corresponding to nucleotides 244-1244 of NCBI Reference Sequence NM_001111033.1.

(SEQ ID NO: 239)
ATGACCCGGGCGCTCTGCTCAGCGCTCCGCCAGGCTCTCCTGCTG
CTCGCAGCGGCCGCCGAGCTCTCGCCAGGACTGAAGTGTGTATGT
CTTTTGTGTGATTCTTCAAACTTTACCTGCCAAACAGAAGGAGCA
TGTTGGGCATCAGTCATGCTAACCAATGGAAAAGAGCAGGTGATC
AAATCCTGTGTCTCCCTTCCAGAACTGAATGCTCAAGTCTTCTGT
CATAGTTCCAACAATGTTACCAAAACCGAATGCTGCTTCACAGAT
TTTTGCAACAACATAACACTGCACCTTCCAACAGATAATGGAACT
TGGACTCAACTTTGGCTGGTATCTGAATATCATGAACAGGGCTCC
TTATATGACTATTTGAATAGAAATATAGTGACCGTGGCTGGAATG
ATCAAGCTGGCGCTCTCAATTGCTAGTGGTCTGGCACACCTTCAT
ATGGAGATTGTTGGTACACAAGGTAAACCTGCTATTGCTCATCGA
GACATAAAATCAAAGAATATCTTAGTGAAAAAGTGTGAAACTTGT
GCCATAGCGGACTTAGGGTTGGCTGTGAAGCATGATTCAATACTG
AACACTATCGACATACCTCAGAATCCTAAAGTGGGAACCAAGAGG
TATATGGCTCCTGAAATGCTTGATGATACAATGAATGTGAATATC
TTTGAGTCCTTCAAACGAGCTGACATCTATTCTGTTGGTCTGGTT
TACTGGGAAATAGCCCGGAGGTGTTCAGTCGGAGGAATTGTTGAG
GAGTACCAATTGCCTTATTATGACATGGTGCCTTCAGATCCCTCG
ATAGAGGAAATGAGAAAGGTTGTTTGTGACCAGAAGTTTCGACCA
AGTATCCCAAACCAGTGGCAAAGTTGTGAAGCACTCCGAGTCATG
GGGAGAATAATGCGTGAGTGTTGGTATGCCAACGGAGCGGCCCGC
CTAACTGCTCTTCGTATTAAGAAGACTATATCTCAACTTTGTGTC
AAAGAAGACTGCAAAGCCTAA

A nucleic acid sequence encoding the processed ALK7 polypeptide (isoform 4) is shown in SEQ ID NO: 240.

(SEQ ID NO: 240)
GAGCTCTCGCCAGGACTGAAGTGTGTATGTCTTTTGTGTGATTCT
TCAAACTTTACCTGCCAAACAGAAGGAGCATGTTGGGCATCAGTC
ATGCTAACCAATGGAAAAGAGCAGGTGATCAAATCCTGTGTCTCC
CTTCCAGAACTGAATGCTCAAGTCTTCTGTCATAGTTCCAACAAT
GTTACCAAAACCGAATGCTGCTTCACAGATTTTTGCAACAACATA
ACACTGCACCTTCCAACAGATAATGGAACTTGGACTCAACTTTGG
CTGGTATCTGAATATCATGAACAGGGCTCCTTATATGACTATTTG
AATAGAAATATAGTGACCGTGGCTGGAATGATCAAGCTGGCGCTC
TCAATTGCTAGTGGTCTGGCACACCTTCATATGGAGATTGTTGGT
ACACAAGGTAAACCTGCTATTGCTCATCGAGACATAAAATCAAAG
AATATCTTAGTGAAAAAGTGTGAAACTTGTGCCATAGCGGACTTA
GGGTTGGCTGTGAAGCATGATTCAATACTGAACACTATCGACATA
CCTCAGAATCCTAAAGTGGGAACCAAGAGGTATATGGCTCCTGAA
ATGCTTGATGATACAATGAATGTGAATATCTTTGAGTCCTTCAAA
CGAGCTGACATCTATTCTGTTGGTCTGGTTTACTGGGAAATAGCC
CGGAGGTGTTCAGTCGGAGGAATTGTTGAGGAGTACCAATTGCCT
TATTATGACATGGTGCCTTCAGATCCCTCGATAGAGGAAATGAGA
AAGGTTGTTTGTGACCAGAAGTTTCGACCAAGTATCCCAAACCAG
TGGCAAAGTTGTGAAGCACTCCGAGTCATGGGGAGAATAATGCGT
GAGTGTTGGTATGCCAACGGAGCGGCCCGCCTAACTGCTCTTCGT
ATTAAGAAGACTATATCTCAACTTTGTGTCAAAGAAGACTGCAAA
GCCTAA

Based on the signal sequence of full-length ALK7 (isoform 1) in the rat (see NCBI Reference Sequence NP_620790.1) and on the high degree of sequence identity between human and rat ALK7, it is predicted that a processed form of human ALK7 isoform 1 is as follows (SEQ ID NO: 128).

```
(SEQ ID NO: 128)
  1 LKCVCLLCDS SNFTCQTEGA CWASVMLING
      KEQVIKSCVS LPELNAQVFC HSSNNVTKTE
61 CCFTDFCNNI TLHLPTASPN APKLGPME
```

Active variants of processed ALK7 isoform 1 are predicted in which SEQ ID NO: 123 is truncated by 1, 2, 3, 4, 5, 6, or 7 amino acids at the N-terminus and SEQ ID NO: 128 is truncated by 1 or 2 amino acids at the N-terminus. Consistent with SEQ ID NO: 128, it is further expected that leucine is the N-terminal amino acid in the processed forms of human ALK7 isoform 3 (SEQ ID NO: 126) and human ALK7 isoform 4 (SEQ ID NO: 127).

In certain embodiments, the disclosure relates to heteromultimers that comprise at least one ALK7 polypeptide, which includes fragments, functional variants, and modified forms thereof. Preferably, ALK7 polypeptides for use in accordance with inventions of the disclosure (e.g., heteromultimers comprising an ALK7 polypeptide and uses thereof) are soluble (e.g., an extracellular domain of ALK7). In other embodiments, ALK7 polypeptides for use in accordance with the disclosure bind to one or more ActRII-ALK4 ligand. Therefore, in some embodiments, ALK7 polypeptides for use in accordance with the disclosure inhibit (antagonize) activity (e.g., induction of Smad signaling) of one or more ActRII-ALK4 ligands. In some embodiments, heteromultimers of the disclosure comprise at least one ALK7 polypeptide that is at least 70%, 75%, 80%, 85%, 86%, 87%, 88%, 89%, 90%, 91%, 92%, 93%, 94%, 95%, 97%, 98%, or 99% identical to the amino acid sequence of SEQ ID NO:120, 123, 124, 125, 121, 126, 122, 127, 128, 129, 255, 133, and 134. In some embodiments, heteromultimers of the disclosure consist or consist essentially of at least one ALK7 polypeptide that is at least 70%, 75%, 80%, 85%, 86%, 87%, 88%, 89%, 90%, 91%, 92%, 93%, 94%, 95%, 97%, 98%, or 99% identical to the amino acid sequence of SEQ ID NO:120, 123, 124, 125, 121, 126, 122, 127, 128, 129, 255, 133, and 134.

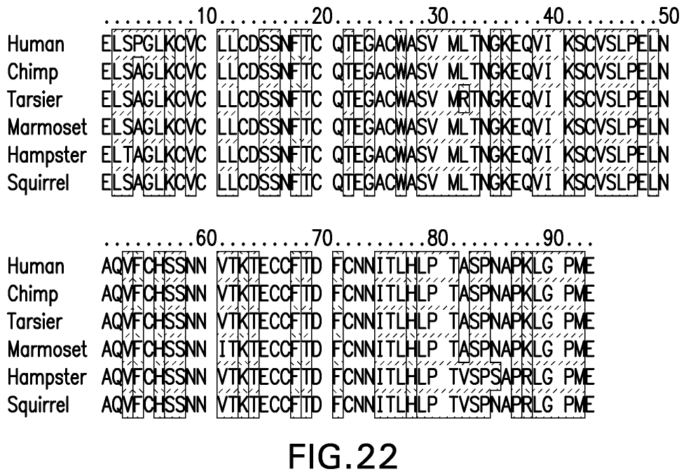

ALK7 is well-conserved among vertebrates, with large stretches of the extracellular domain completely conserved. For example, FIG. 22 depicts a multi-sequence alignment of a human ALK7 extracellular domain compared to various ALK7 orthologs. Accordingly, from these alignments, it is possible to predict key amino acid positions within the ligand-binding domain that are important for normal ALK7-ligand binding activities as well as to predict amino acid positions that are likely to be tolerant to substitution without significantly altering normal ALK7-ligand binding activities. Therefore, an active, human ALK7 variant polypeptide useful in accordance with the presently disclosed methods may include one or more amino acids at corresponding positions from the sequence of another vertebrate ALK7, or may include a residue that is similar to that in the human or other vertebrate sequences. Without meaning to be limiting, the following examples illustrate this approach to defining an active ALK7 variant. V61 in the human ALK7 extracellular domain (SEQ ID NO: 425) is isoleucine in Callithrix jacchus ALK7 (SEQ ID NO: 428), and so the position may be altered, and optionally may be altered to another hydrophobic residue such as L, I, or F, or a non-polar residue such as A. L32 in the human extracellular domain is R in Tarsius syrichta (SEQ ID NO: 429) ALK7, indicating that this site may be tolerant of a wide variety of changes, including polar residues, such as E, D, K, R, H, S, T, P, G, Y, and probably a non-polar residue such as A. K37 in the human extracellular domain is R in Pan troglodytes ALK7 (SEQ ID NO: 426), indicating that basic residues are tolerated at this position, including R, K, and H. P4 in the human extracellular domain is relatively poorly conserved, appearing as A in Pan troglodytes ALK7 thus indicating that a wide variety of amino acid should be tolerated at this position.

Moreover, ALK7 proteins have been characterized in the art in terms of structural and functional characteristics [e.g., Romano et al (2012) Journal of Molecular Modeling 18(8): 3617-3625]. For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ALK7, as demarcated by the outermost of these conserved cysteines, corresponds to positions 28-92 of SEQ ID NO: 120. The structurally less-ordered amino acids flanking these cysteine-demarcated core sequences can be truncated by 1, 2, 3, 4, 5, 6, 7, 8, 9, 10, 11, 12, 13, 14, 15, 16, 17, 18, 19, 20, 21, 22, 23, 24, 25, 26, or 27 residues at the N-terminus and by 1, 2, 3, 4, 5, 6, 7, 8, 9, 10, 11, 12, 13, 14, 15, 16, 17, 18, 19, 20, or 21 residues at the C-terminus without necessarily altering ligand binding. Exemplary ALK7 extracellular domains for N-terminal and/or C-terminal truncation include SEQ ID NOs: 123, 125, 126, and 127.

Accordingly, a general formula for an active portion (e.g., a ligand-binding portion) of ALK7 comprises amino acids 28-92 of SEQ ID NO: 120. Therefore ALK7 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ALK7 beginning at a residue corresponding to any one of amino acids 20-28 (e.g., beginning at any one of amino acids 20, 21, 22, 23, 24, 25, 26, 27, or 28) of SEQ ID NO: 120 and ending at a position corresponding to any one amino acids 92-113 (e.g., ending at any one of amino acids 92, 93, 94, 95, 96, 97, 98, 99, 100, 101, 102, 103, 104, 105, 106, 107, 108, 109, 110, 111, 112, or 113) of SEQ ID NO: 120.

Other examples include constructs that begin at a position from 21-28 (e.g., any one of positions 21, 22, 23, 24, 25, 26, 27, or 28), 24-28 (e.g., any one of positions 24, 25, 26, 27, or 28), or 25-28 (e.g., any one of positions 25, 26, 27, or 28) of SEQ ID NO: 120 and end at a position from 93-112 (e.g., any one of positions 93, 94, 95, 96, 97, 98, 99, 100, 101, 102, 103, 104, 105, 106, 107, 108, 109, 110, 111, or 112), 93-110 (e.g., any one of positions 93, 94, 95, 96, 97, 98, 99, 100, 101, 102, 103, 104, 105, 106, 107, 108, 109, or 110), 93-100 (e.g., any one of positions 93, 94, 95, 96, 97, 98, 99, or 100), or 93-95 (e.g., any one of positions 93, 94, or 95) of SEQ ID NO: 120. Variants within these ranges are also contemplated, particularly those having at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 120.

The variations described herein may be combined in various ways. In some embodiments, ALK7 variants comprise no more than 1, 2, 5, 6, 7, 8, 9, 10 or 15 conservative amino acid changes in the ligand-binding pocket. Sites outside the binding pocket, at which variability may be particularly well tolerated, include the amino and carboxy termini of the extracellular domain (as noted above).

F) Follistatin Polypeptides

In other aspects, an ActRII-ALK4 antagonist is a follistatin polypeptide. As described herein, follistatin polypeptides may be used to treat, prevent, or reduce the progression rate and/or severity of heart failure associated with aging, particularly treating, preventing or reducing the progression rate and/or severity of one or more heart failure-associated complications.

The term “follistatin polypeptide” includes polypeptides comprising any naturally occurring polypeptide of follistatin as well as any variants thereof (including mutants, fragments, fusions, and peptidomimetic forms) that retain a useful activity, and further includes any functional monomer or multimer of follistatin. In certain embodiments, follistatin polypeptides of the disclosure bind to and/or inhibit activin activity, particularly activin A. Variants of follistatin polypeptides that retain activin binding properties can be identified based on previous studies involving follistatin and activin interactions. For example, WO2008/030367 discloses specific follistatin domains (“FSDs”) that are shown to be important for activin binding. As shown below in SEQ ID NOs: 392-394, the follistatin N-terminal domain (“FSND” SEQ ID NO: 392), FSD2 (SEQ ID NO: 394), and to a lesser extent FSD1 (SEQ ID NO: 393) represent exemplary domains within follistatin that are important for activin binding. In addition, methods for making and testing libraries of polypeptides are described above in the context of ActRII polypeptides, and such methods also pertain to making and testing variants of follistatin. Follistatin polypeptides include polypeptides derived from the sequence of any known follistatin having a sequence at least about 80% identical to the sequence of a follistatin polypeptide, and optionally at least 70%, 75%, 80%, 85%, 90%, 91%, 92%, 93%, 94%, 95%, 96%, 97%, 98%, 99% or greater identity. Examples of follistatin polypeptides include the mature follistatin polypeptide or shorter isoforms or other variants of the human follistatin precursor polypeptide (SEQ ID NO: 390) as described, for example, in WO2005/025601.

The human follistatin precursor polypeptide isoform FST344 is as follows:

```
(SEQ ID NO: 390; NCBI Reference No. NP_037541.1)
    1 MVRARHQPGG LCLLLLLLCQ FMEDRSAQAG
        NCWLRQAKNG RCQVLYKTEL
  51 SKEECCSTGR LSTSWTEEDV NDNTLFKWMI
        ENGGAPNCIP CKETCENVDC
101 GPGKKCRMNK KNKPRCVCAP DCSNITWKGP
        VCGLDGKTYR NECALLKARC
151 KEQPELEVQY QGRCKKTCRD VFCPGSSTCV
        VDQTNNAYCV TCNRICPEPA
201 SSEQYLCGND GVTYSSACHL RKATCLLGRS
        IGLAYEGKCI KAKSCEDIQC
251 TGGKKCLWDF KVGRGRCSLC DELCPDSKSD
        EPVCASDNAT YASECAMKEA
301 ACSSGVLLEV KHSGSCNSIS EDTEEEEEDE
        DQDYSFPISS ILEW
```

The signal peptide is underlined; also underlined above are the last 27 residues which represent the C-terminal extension distinguishing this follistatin isoform from the shorter follistatin isoform FST317 shown below.

The human follistatin precursor polypeptide isoform FST317 is as follows:

```
(SEQ ID NO: 391; NCBI Reference No. NP_006341.1)
    1 MVRARHQPGG LCLLLLLLCQ FMEDRSAQAG
        NCWLRQAKNG RCQVLYKTEL
  51 SKEECCSTGR LSTSWTEEDV NDNTLEKWMI
        ENGGAPNCIP CKETCENVDC
101 GPGKKCRMNK KNKPRCVCAP DCSNITWKGP
        VCGLDGKTYR NECALLKARC
151 KEQPELEVQY QGRCKKTCRD VFCPGSSTCV
        VDQTNNAYCV TCNRICPEPA
201 SSEQYLCGND GVTYSSACHL RKATCLLGRS
        IGLAYEGKCI KAKSCEDIQC
251 TGGKKCLWDF KVGRGRCSLC DELCPDSKSD
        EPVCASDNAT YASECAMKEA
301 ACSSGVLLEV KHSGSCN
```

The signal peptide is underlined.

The follistatin N-terminal domain (FSND) sequence is as follows:

(SEQ ID NO: 392; FSND)
GNCWLRQAKNGRCQVLYKTELSKEECCSTGRLSTSWTEED
VNDNTLFKWMIFNGGAPNCIPCK

The FSD1 and FSD2 sequences are as follows:

(SEQ ID NO: 393; FSD1)
ETCENVDCGPGKKCRMNKKNKPRCV
(SEQ ID NO: 394; FSD2)
KTCRDVFCPGSSTCVVDQTNNAYCVT

G) Fusion Polypeptides

In certain aspects, the disclosure provides for ActRII-ALK4 antagonists that are fusion polypeptides. The fusion polypeptides may be prepared according to any of the methods disclosed herein or that are known in the art.

In some embodiments, any of the fusion polypeptides disclosed herein comprises the following components: a) any of the polypeptides disclosed herein (“A”) (e.g., an ActRIIA, ActRIIB, ALK4, ALK7, or follistatin polypeptide), b) any of the linkers disclosed herein (“B”), c) any of the heterologous portions disclosed herein (“C”) (e.g., an Fc immunoglobulin domain), and optionally a leader sequence (“X”) (e.g., a tissue plasminogen activator leader sequence). In such embodiments, the fusion polypeptide may be arranged in a manner as follows (N-terminus to C-terminus): A-B-C or C-B-A. In such embodiments, the fusion polypeptide may be arranged in a manner as follows (N-terminus to C-terminus): X-A-B-C or X-C-B-A. In some embodiments, the fusion polypeptide comprises each of A, B and C (and optionally a leader sequence), and comprises no more than 100, 90, 80, 70, 60, 50, 40, 30, 20, 10, 5, 4, 3, 2 or 1 additional amino acids (but which may include further post-translational modifications, such as glycosylation).

In some embodiments, the fusion polypeptide comprises a leader sequence positioned in a manner as follows (N-terminus to C-terminus): X-A-B-C, and the fusion polypeptide comprises 1, 2, 3, 4, or 5 amino acids between X and A. In some embodiments, the fusion polypeptide comprises a leader sequence positioned in a manner as follows (N-terminus to C-terminus): X-C-B-A, and the fusion polypeptide comprises 1, 2, 3, 4, or 5 amino acids between X and C. In some embodiments, the fusion polypeptide comprises a leader sequence positioned in a manner as follows (N-terminus to C-terminus): X-A-B-C, and the fusion polypeptide comprises an alanine between X and A. In some embodiments, the fusion polypeptide comprises a leader sequence positioned in a manner as follows (N-terminus to C-terminus): X-C-B-A, and the fusion polypeptide comprises an alanine between X and C. In some embodiments, the fusion polypeptide comprises a leader sequence positioned in a manner as follows (N-terminus to C-terminus): X-A-B-C, and the fusion polypeptide comprises a glycine and an alanine between X and A. In some embodiments, the fusion polypeptide comprises a leader sequence positioned in a manner as follows (N-terminus to C-terminus): X-C-B-A, and the fusion polypeptide comprises a glycine and an alanine between X and C. In some embodiments, the fusion polypeptide comprises a leader sequence positioned in a manner as follows (N-terminus to C-terminus): X-A-B-C, and the fusion polypeptide comprises a threonine between X and A. In some embodiments, the fusion polypeptide comprises a leader sequence positioned in a manner as follows (N-terminus to C-terminus): X-C-B-A, and the fusion polypeptide comprises a threonine between X and C. In some embodiments, the fusion polypeptide comprises a leader sequence positioned in a manner as follows (N-terminus to C-terminus): X-A-B-C, and the fusion polypeptide comprises a threonine between A and B. In some embodiments, the fusion polypeptide comprises a leader sequence positioned in a manner as follows (N-terminus to C-terminus): X-C-B-A, and the fusion polypeptide comprises a threonine between C and B.

In certain aspects, fusion proteins of the disclosure comprise at least a portion of an ActRII-ALK4 ligand trap (e.g., an ActRIIA, ActRIIB, ALK4, ALK7, or follistatin polypeptide) and one or more heterologous portions (e.g., an immunoglobulin Fc domain), optionally with one or more linker domain sequence positioned between the ActRII-ALK4 ligand trap domain and the one or more heterologous portions. Well-known examples of such heterologous portions include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin.

A heterologous portion may be selected so as to confer a desired property. For example, some heterologous portions are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS6) fusion partners. As another example, a heterologous portion may be selected so as to facilitate detection of the fusion polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the heterologous portions have a protease cleavage site, such as for Factor Xa or Thrombin, which allows the relevant protease to partially digest the fusion proteins and thereby liberate the recombinant proteins therefrom. The liberated proteins can then be isolated from the heterologous portion by subsequent chromatographic separation.

In certain embodiments, an ActRII-ALK4 ligand trap domain (e.g., an ActRIIA, ActRIIB, ALK4, ALK7, or follistatin polypeptide) is fused, optionally with an intervening linker domain, to a heterologous domain that stabilizes the ActRII-ALK4 ligand trap domain in vivo (a “stabilizer” domain). In general, the term “stabilizing” means anything that increases serum half-life, regardless of whether this is because of decreased destruction, decreased clearance by the kidney, or other pharmacokinetic effect of the agent. Fusion polypeptides with the Fc portion of an immunoglobulin are known to confer desirable pharmacokinetic properties on a wide range of proteins. Likewise, fusions to human serum albumin can confer desirable properties. Other types of heterologous portions that may be selected include multimerizing (e.g., dimerizing, tetramerizing) domains and functional domains. In some embodiments, a stabilizing domain may also function as a multimerization domain. Such multifunctional domains include, for example, Fc immunoglobulin domains. Various examples of Fc immunoglobulin domains and Fc-fusion proteins comprising one or more ActRII-ALK4 ligand trap domains are described throughout the disclosure.

In some embodiments, fusion proteins of the disclosure may additionally include any of various leader sequences at the N-terminus. Such a sequence would allow the peptides to be expressed and targeted to the secretion pathway in a eukaryotic system. See, e.g., Ernst et al., U.S. Pat. No. 5,082,783 (1992). Alternatively, a native signal sequence may be used to effect extrusion from the cell. Possible leader sequences include native leaders, tissue plasminogen activator (TPA) and honeybee melittin. Examples of fusion proteins incorporating a TPA leader sequence include SEQ ID NOs: 6, 31, 34, 37, 40, 43, 46, 49, 51, 88, 92, 129, 133, 247, 276, 279, 333, 336, 339, 342, 345, 348, 351, 354, 381, 396, 402, and 406. Processing of signal peptides may vary depending on the leader sequence chosen, the cell type used and culture conditions, among other variables, and therefore actual N-terminal start sites for mature (e.g., an ActRIIA, ActRIIB, ALK4, ALK7, or follistatin polypeptide) polypeptides may shift by 1, 2, 3, 4 or 5 amino acids in either the N-terminal or C-terminal direction.

Preferred fusion proteins comprise the amino acid sequence set forth in any one of SEQ ID NOs: 5, 6, 12, 31, 33, 34, 36, 37, 39, 40, 42, 43, 45, 46, 48, 49, 50, 51, 52, 54, 55, 88, 89, 92, 93, 129, 130, 133, 134, 247, 249, 276, 278, 279, 332, 333, 335, 336, 338, 339, 341, 342, 344, 345, 347, 348, 350, 351, 353, 354, 356, 378, 380, 381, 385, 396, 398, 401, 402, 403, 406, 408, and 409.

I Multimerization Domains

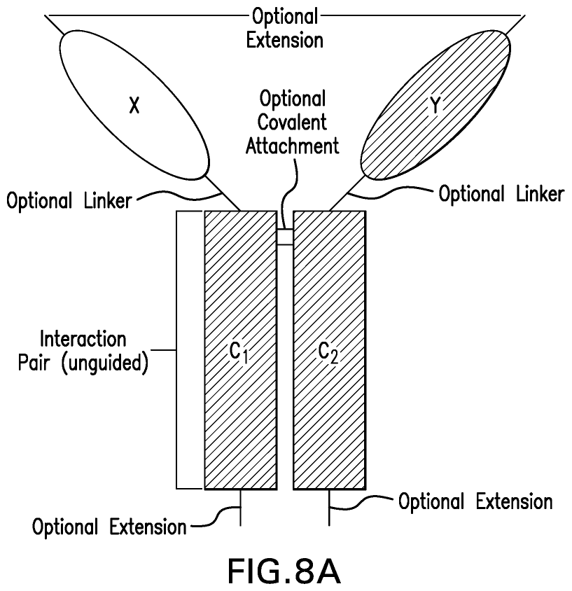

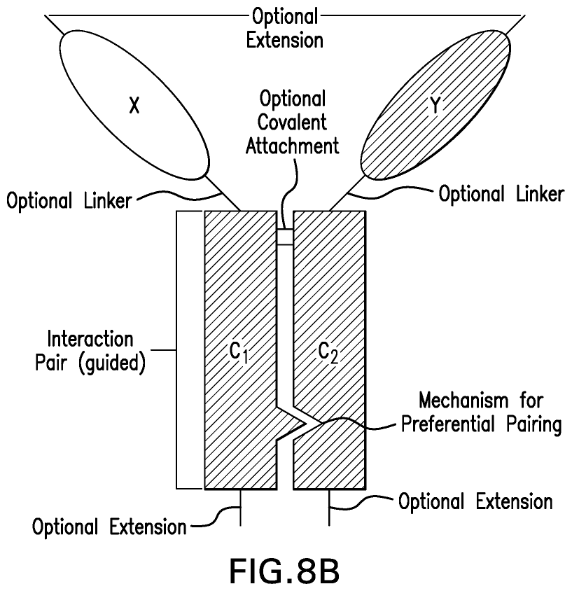

In certain embodiments, polypeptides (e.g., ActRIIA, ActRIIB, ALK4, ALK7, and follistatin polypeptides) of the present disclosure comprise at least one multimerization domain. As disclosed herein, the term “multimerization domain” refers to an amino acid or sequence of amino acids that promote covalent or non-covalent interaction between at least a first polypeptide and at least a second polypeptide. Polypeptides (e.g., ActRIIA, ActRIIB, ALK4, ALK7, and follistatin polypeptides) may be joined covalently or non-covalently to a multimerization domain. In some embodiments, a multimerization domain promotes interaction between a first polypeptide (e.g., ActRIIB or ActRIIA polypeptide) and a second polypeptide (e.g., an ALK4 polypeptide or an ALK7 polypeptide) to promote heteromultimer formation (e.g., heterodimer formation), and optionally hinders or otherwise disfavors homomultimer formation (e.g., homodimer formation), thereby increasing the yield of desired heteromultimer (see, e.g., FIG. 8B). In some embodiments, polypeptides (e.g., ActRIIA, ActRIIB, ALK4, ALK7, and follistatin polypeptides) may form heterodimers through covalent interactions. In some embodiments, polypeptides (e.g., ActRIIA, ActRIIB, ALK4, ALK7, and follistatin polypeptides) may form heterodimers through non-covalent interactions. In some embodiments, polypeptides (e.g., ActRIIA, ActRIIB, ALK4, ALK7, and follistatin polypeptides) may form heterodimers through both covalent and non-covalent interactions. In some embodiments, a multimerization domain promotes interaction between a first polypeptide and a second polypeptide to promote homomultimer formation, and optionally hinders or otherwise disfavors heteromultimer formation, thereby increasing the yield of desired homomultimer. In some embodiments, polypeptides (e.g., ActRIIA, ActRIIB, ALK4, ALK7, and follistatin polypeptides) form homodimers. In some embodiments, polypeptides (e.g., ActRIIA, ActRIIB, ALK4, ALK7, and follistatin polypeptides) may form homodimers through covalent interactions. In some embodiments, polypeptides (e.g., ActRIIA, ActRIIB, ALK4, ALK7, and follistatin polypeptides) may form homodimers through non-covalent interactions. In some embodiments, polypeptides (e.g., ActRIIA, ActRIIB, ALK4, ALK7, and follistatin polypeptides) may form homodimers through both covalent and non-covalent interactions.

In certain aspects, a multimerization domain may comprise one component of an interaction pair. In some embodiments, the polypeptides disclosed herein may form polypeptide complexes comprising a first polypeptide covalently or non-covalently associated with a second polypeptide, wherein the first polypeptide comprises the amino acid sequence of a first ActRII-ALK4 ligand trap polypeptide (e.g., a ActRIIA, ActRIIB, ALK4, ALK7, and follistatin polypeptide) and the amino acid sequence of a first member of an interaction pair (e.g., a first immunoglobulin Fc domain); and the second polypeptide comprises the amino acid sequence of a second ActRII-ALK4 ligand trap polypeptide (e.g., a ActRIIA, ActRIIB, ALK4, ALK7, and follistatin polypeptide), and the amino acid sequence of a second member of an interaction pair (e.g., a second immunoglobulin Fc domain). In some embodiments, the polypeptides disclosed herein may form polypeptide complexes comprising a first polypeptide covalently or non-covalently associated with a second polypeptide, wherein the first polypeptide comprises the amino acid sequence of an ActRIIA polypeptide and the amino acid sequence of a first member of an interaction pair; and the second polypeptide comprises the amino acid sequence of an ALK4 polypeptide or an ALK7 polypeptide, and the amino acid sequence of a second member of an interaction pair. In some embodiments, the polypeptides disclosed herein may form polypeptide complexes comprising a first polypeptide covalently or non-covalently associated with a second polypeptide, wherein the first polypeptide comprises the amino acid sequence of an ActRIIB polypeptide and the amino acid sequence of a first member of an interaction pair; and the second polypeptide comprises the amino acid sequence of an ALK4 polypeptide or an ALK7 polypeptide, and the amino acid sequence of a second member of an interaction pair. In some embodiments, the interaction pair may be any two polypeptide sequences that interact to form a dimeric complex, either a heterodimeric or homodimeric complex. An interaction pair may be selected to confer an improved property/activity such as increased serum half-life, or to act as an adaptor onto which another moiety is attached to provide an improved property/activity. For example, a polyethylene glycol or glycosylation moiety may be attached to one or both components of an interaction pair to provide an improved property/activity such as improved serum half-life.

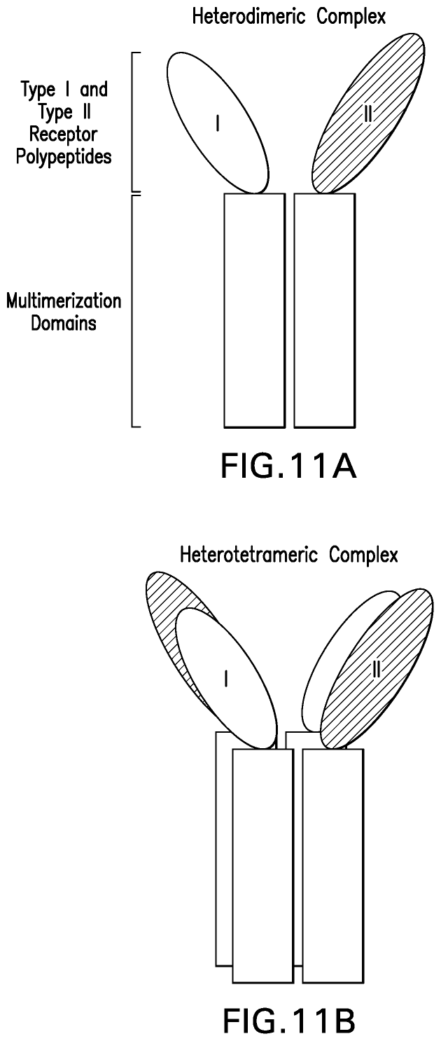

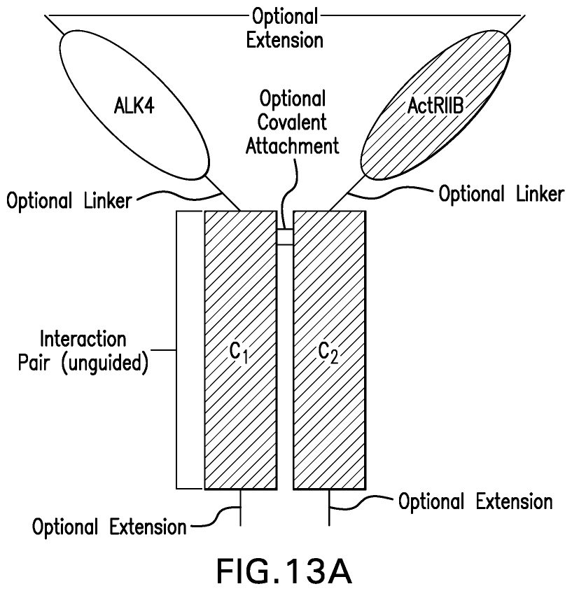

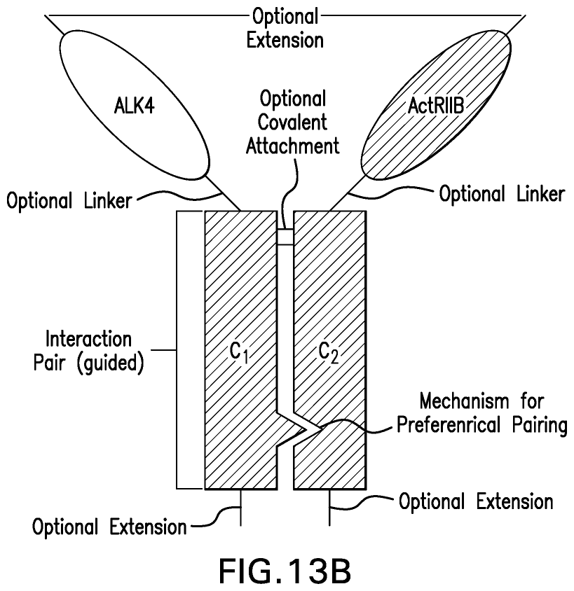

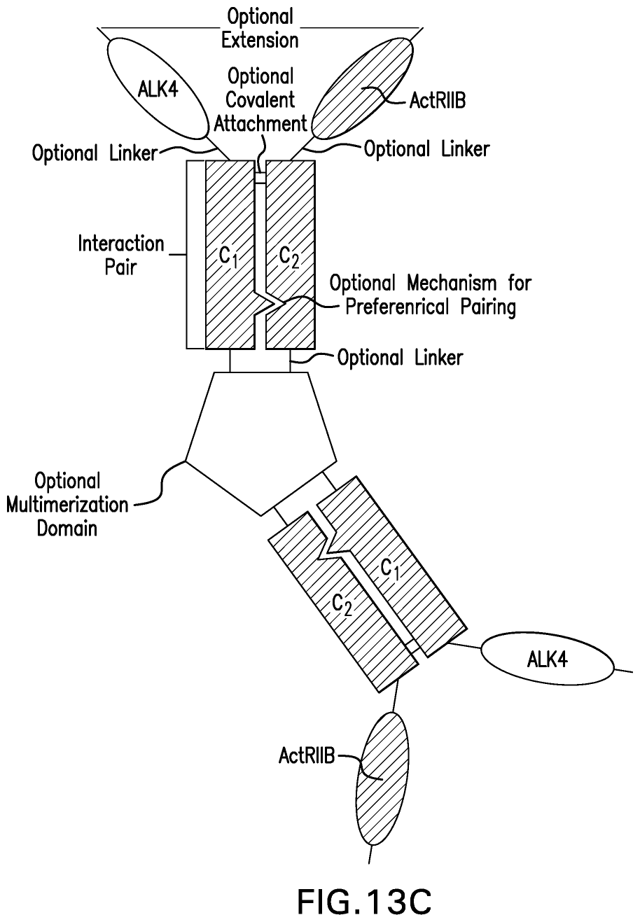

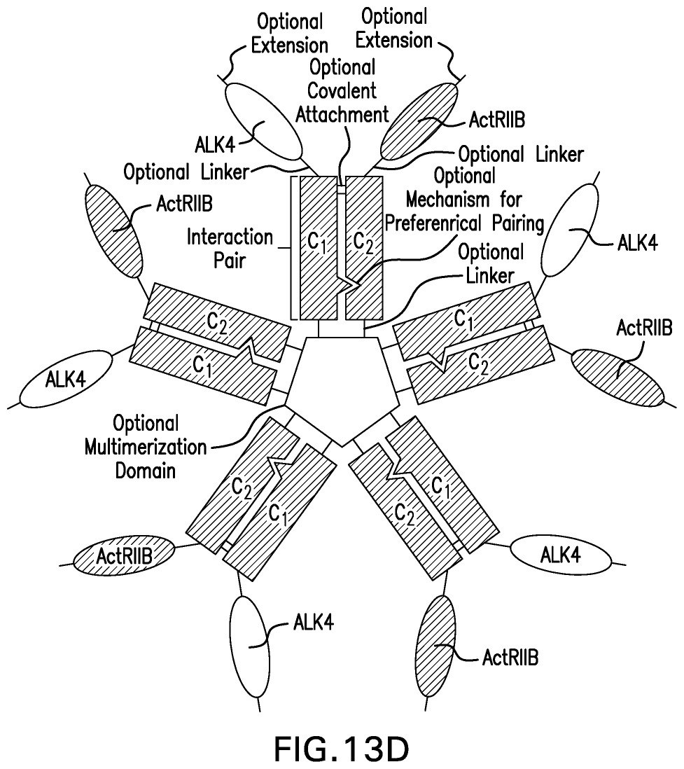

The first and second members of the interaction pair may be an asymmetric pair, meaning that the members of the pair preferentially associate with each other rather than self-associate. Accordingly, first and second members of an asymmetric interaction pair may associate to form a heterodimeric complex (see, e.g., FIG. 8B). Alternatively, the interaction pair may be unguided, meaning that the members of the pair may associate with each other or self-associate without substantial preference and thus may have the same or different amino acid sequences (see, e.g., FIG. 8A). Accordingly, first and second members of an unguided interaction pair may associate to form a homodimer complex or a heterodimeric complex. Optionally, the first member of the interaction pair (e.g., an asymmetric pair or an unguided interaction pair) associates covalently with the second member of the interaction pair. Optionally, the first member of the interaction pair (e.g., an asymmetric pair or an unguided interaction pair) associates non-covalently with the second member of the interaction pair. In certain embodiments, polypeptides disclosed herein form heterodimeric or homodimeric complexes, although higher order heteromultimeric and homomultimeric complexes are also included such as, but not limited to, heterotrimers, homotrimers, heterotetramers, homotetramers, and further oligomeric structures (see, e.g., FIG. 11-13, which may also be applied to both ActRII-ALK4 and ActRII-ALK7 oligomeric structures).

Ia Fc-Fusion Proteins

As specific examples of fusion polypeptides comprising a multimerization domain, the disclosure provides fusion polypeptides comprising an ActRII-ALK4 ligand trap polypeptide (e.g., an ActRIIA, ActRIIB, ALK4, ALK7, and follistatin polypeptide) fused to a polypeptide comprising a constant domain of an immunoglobulin, such as a CH1, CH2, or CH3 domain of an immunoglobulin or an immunoglobulin Fc domain. As used herein, the term “immunoglobulin Fc domain” or simply “Fc” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In one embodiment the immunoglobulin Fc region comprises at least an immunoglobulin hinge region a CH2 domain and a CH3 domain, and preferably lacks the CH1 domain. In some embodiments, the immunoglobulin Fc region is a human immunoglobulin Fc region. In some embodiments, the class of immunoglobulin from which the heavy chain constant region is derived is IgG (Igγ) (γ subclasses 1, 2, 3, or 4). In certain embodiments, the constant region is derived from IgG1. Other classes of immunoglobulin, IgA (Igα), IgD (Igδ), IgE (Igε) and IgM (Igμ), may be used. The choice of appropriate immunoglobulin heavy chain constant region is discussed in detail in U.S. Pat. Nos. 5,541,087 and 5,726,044, which is incorporated herein in its entirety. The choice of particular immunoglobulin heavy chain constant region sequences from certain immunoglobulin classes and subclasses to achieve a particular result is considered to be within the level of skill in the art. In some embodiments, a portion of the DNA construct encoding the immunoglobulin Fc region preferably comprises at least a portion of a hinge domain, and preferably at least a portion of a CH3 domain of Fc gamma or the homologous domains in any of IgA, IgD, Igε, or IgM. Furthermore, it is contemplated that substitution or deletion of amino acids within the immunoglobulin heavy chain constant regions may be useful in the practice of the methods and compositions disclosed herein. One example is to introduce amino acid substitutions in the upper CH2 region to create an Fc variant with reduced affinity for Fc receptors (Cole et al. (1997) J. Immunol. 159:3613). Fc domains derived from human IgG1, IgG2, IgG3, and IgG4 are provided herein.

An example of a native amino acid sequence that may be used for the Fc portion of human IgG1 (G1Fc) is shown below (SEQ ID NO: 13). Dotted underline indicates the hinge region, and solid underline indicates positions with naturally occurring variants. In part, the disclosure provides polypeptides (e.g., ActRIIA, ActRIIB, ALK4, ALK7, and follistatin polypeptides) comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 13.

Naturally occurring variants in G1Fc include E134D and M136L according to the numbering system used in SEQ ID NO: 13 (see Uniprot P01857).

| (SEQ ID NO: 13) | |
|---|---|
| 1 | 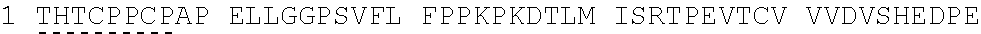 |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |

In some embodiments, the disclosure provides Fc fusion polypeptides comprising an ActRII-ALK4 ligand trap polypeptide domain (e.g., an ActRIIA, ActRIIB, ALK4, ALK7, and follistatin polypeptide domain), including variants as well as homomultimers (e.g., homodimers) and heteromultimers (e.g., heterodimers including, for example, ActRIIA:ALK4, ActRIIB:ALK4, ActRIIA:ALK7, and ActRIIB:ALK7 heterodimers) thereof, fused to one or more Fc polypeptide domains that are at least 75%, 80%, 85%, 90%, 91%, 92%, 93%, 94%, 95%, 96%, 97%, 98%, 99%, or 100% identical to the amino acid sequence of SEQ ID NO: 13.

An example of a native amino acid sequence that may be used for the Fc portion of human IgG2 (G2Fc) is shown below (SEQ ID NO: 14). Dotted underline indicates the hinge region and double underline indicates positions where there are database conflicts in the sequence (according to UniProt P01859). In part, the disclosure provides polypeptides (e.g., ActRIIA, ActRIIB, ALK4, ALK7, and follistatin polypeptides) comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 14.

| (SEQ ID NO: 14) | |
|---|---|
| 1 | 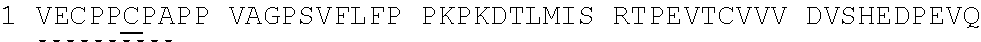 |
| 51 | FNWYVDGVEV HNAKTKPREE QFNSTERVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PGK |

In some embodiments, the disclosure provides Fc fusion polypeptides comprising an ActRII-ALK4 ligand trap polypeptide domain (e.g., an ActRIIA, ActRIIB, ALK4, ALK7, and follistatin polypeptide domain), including variants as well as homomultimers (e.g., homodimers) and heteromultimers (e.g., heterodimers including, for example, ActRIIA:ALK4, ActRIIB:ALK4, ActRIIA:ALK7, and ActRIIB:ALK7 heterodimers) thereof, fused to one or more Fc polypeptide domains that are at least 75%, 80%, 85%, 90%, 91%, 92%, 93%, 94%, 95%, 96%, 97%, 98%, 99%, or 100% identical to the amino acid sequence of SEQ ID NO: 14.

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 15) contains a short hinge region consisting of a single 15-residue segment, whereas the second G3Fc sequence (SEQ ID NO: 16) contains a full-length hinge region. In each case, dotted underline indicates the hinge region, and solid underline indicates positions with naturally occurring variants according to UniProt P01859. In part, the disclosure provides polypeptides (e.g., ActRIIA, ActRIIB, ALK4, ALK7, and follistatin polypeptides) comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NOs: 15. In part, the disclosure provides polypeptides (e.g., ActRIIA, ActRIIB, ALK4, ALK7, and follistatin polypeptides) comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NOs: 16.

| (SEQ ID NO: 15) | |
|---|---|
| 1 | 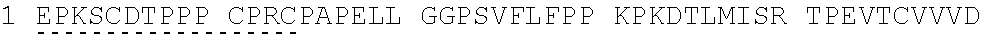 |
| 51 | VSHEDPEVQF KWYVDGVEVH NAKTKPREEQ YNSTERVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP GK |
| (SEQ ID NO: 16) | |
| 1 | 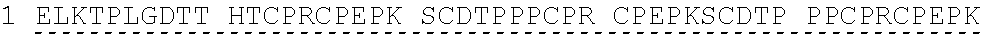 |
| 51 | 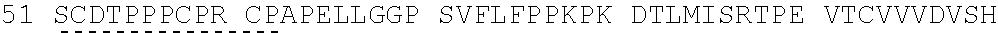 |
| 101 | EDPEVQFKWY VDGVEVHNAK TKPREEQYNS TFRVVSVLTV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 15, and the present disclosure provides fusion polypeptides comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion polypeptides comprising G3Fc domains containing one or more of these variants.

In some embodiments, the disclosure provides Fc fusion polypeptides comprising an ActRII-ALK4 ligand trap polypeptide domain (e.g., an ActRIIA, ActRIIB, ALK4, ALK7, and follistatin polypeptide domain), including variants as well as homomultimers (e.g., homodimers) and heteromultimers (e.g., heterodimers including, for example, ActRIIA:ALK4, ActRIIB:ALK4, ActRIIA:ALK7, and ActRIIB:ALK7 heterodimers) thereof, fused to one or more Fc polypeptide domains that are at least 75%, 80%, 85%, 90%, 91%, 92%, 93%, 94%, 95%, 96%, 97%, 98%, 99%, or 100% identical to the amino acid sequence of SEQ ID NO: 15.

In some embodiments, the disclosure provides Fc fusion polypeptides comprising an ActRII-ALK4 ligand trap polypeptide domain (e.g., an ActRIIA, ActRIIB, ALK4, ALK7, and follistatin polypeptide domain), including variants as well as homomultimers (e.g., homodimers) and heteromultimers (e.g., heterodimers including, for example, ActRIIA:ALK4, ActRIIB:ALK4, ActRIIA:ALK7, and ActRIIB:ALK7 heterodimers) thereof, fused to one or more Fc polypeptide domains that are at least 75%, 80%, 85%, 90%, 91%, 92%, 93%, 94%, 95%, 96%, 97%, 98%, 99%, or 100% identical to the amino acid sequence of SEQ ID NO: 16.

An example of a native amino acid sequence that may be used for the Fc portion of human IgG4 (G4Fc) is shown below (SEQ ID NO: 17). Dotted underline indicates the hinge region. In part, the disclosure provides polypeptides (e.g., ActRIIA, ActRIIB, ALK4, ALK7, and follistatin polypeptides) comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 17.

| (SEQ ID NO: 17) | |
|---|---|
| 1 | 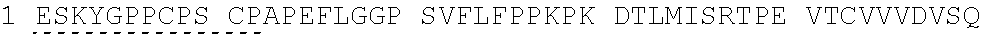 |
| 51 | EDPEVQFNWY VDGVEVHNAK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHYTQ KSLSLSLGK |

In some embodiments, the disclosure provides Fc fusion polypeptides comprising an ActRII-ALK4 ligand trap polypeptide domain (e.g., an ActRIIA, ActRIIB, ALK4, ALK7, and follistatin polypeptide domain), including variants as well as homomultimers (e.g., homodimers) and heteromultimers (e.g., heterodimers including, for example, ActRIIA:ALK4, ActRIIB:ALK4, ActRIIA:ALK7, and ActRIIB:ALK7 heterodimers) thereof, fused to one or more Fc polypeptide domains that are at least 75%, 80%, 85%, 90%, 91%, 92%, 93%, 94%, 95%, 96%, 97%, 98%, 99%, or 100% identical to the amino acid sequence of SEQ ID NO: 17.

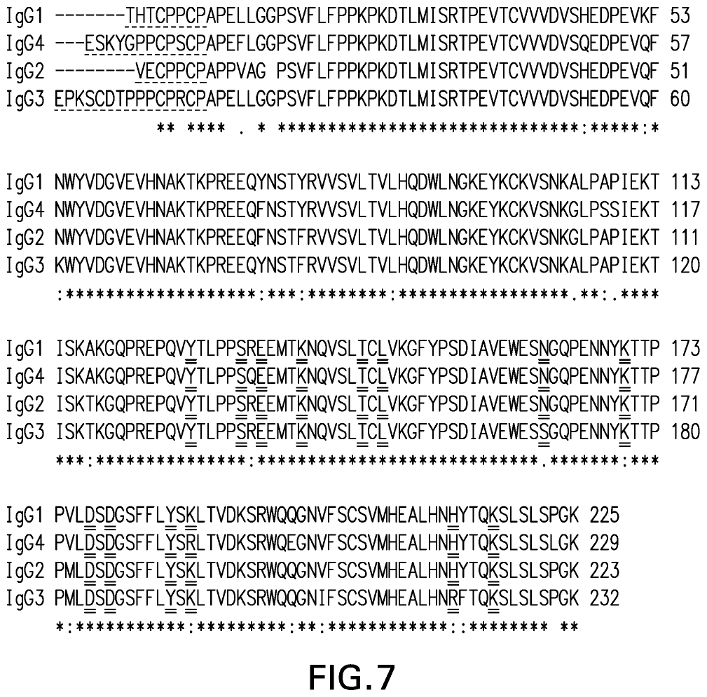

A variety of engineered mutations in the Fc domain are presented herein with respect to the G1Fc sequence (SEQ ID NO: 13). Analogous mutations in G2Fc, G3Fc, and G4Fc can be derived from their alignment with G1Fc in FIG. 7. Due to unequal hinge lengths, analogous Fc positions based on isotype alignment (FIG. 7) possess different amino acid numbers in SEQ ID NOs: 13, 14, 15, and 17. It can also be appreciated that a given amino acid position in an immunoglobulin sequence consisting of hinge, C_{H}2, and C_{H}3 regions (e.g., SEQ ID NOs: 13, 14, 15, 16, or 17) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 13), the human IgG1 heavy chain constant domain (Uniprot P01857), and the human IgG1 heavy chain is as follows.

Correspondence of C_{H}3 Positions in Different Numbering Systems

| G1Fc | IgG1 heavy chain | IgG1 heavy chain |
|---|---|---|
| (Numbering begins | constant domain | (EU numbering |
| at first threonine | (Numbering begins | scheme of Kabat |
| in hinge region) | at C_{H}1) | et al., 1991*) |
| Y127 | Y232 | Y349 |
| S132 | S237 | S354 |
| E134 | E239 | E356 |
| K138 | K243 | K360 |
| T144 | T249 | T366 |
| L146 | L251 | L368 |
| N162 | N267 | N384 |
| K170 | K275 | K392 |
| D177 | D282 | D399 |
| D179 | D284 | D401 |
| Y185 | Y290 | Y407 |
| K187 | K292 | K409 |
| H213 | H318 | H435 |
| K217 | K322 | K439 |

*Kabat et al. (eds) 1991; pp. 688-696 in Sequences of Proteins of Immunological Interest, 5^{th} ed., Vol. 1, NIH, Bethesda, MD.

In some embodiments, the disclosure provides antibodies and Fc fusion proteins with engineered or variant Fc regions. Such antibodies and Fc fusion proteins may be useful, for example, in modulating effector functions, such as, antigen-dependent cytotoxicity (ADCC) and complement-dependent cytotoxicity (CDC). Additionally, the modifications may improve the stability of the antibodies and Fc fusion proteins. Amino acid sequence variants of the antibodies and Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the antibodies and Fc fusion proteins, such as changing the number or position of glycosylation sites.

Antibodies and Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26). Thus, in certain embodiments, Fc fusion proteins of the disclosure with mutations within the constant region including the Ala-Ala mutation may be used to reduce or abolish effector function. According to these embodiments, antibodies and Fc fusion proteins may comprise a mutation to an alanine at position 234 or a mutation to an alanine at position 235, or a combination thereof. In one embodiment, the antibody or Fc fusion protein comprises an IgG4 framework, wherein the Ala-Ala mutation describes a mutation(s) from phenylalanine to alanine at position 234 and/or a mutation from leucine to alanine at position 235. In another embodiment, the antibody or Fc fusion protein comprises an IgG1 framework, wherein the Ala-Ala mutation describes a mutation(s) from leucine to alanine at position 234 and/or a mutation from leucine to alanine at position 235. While alanine substitutions at these sites are effective in reducing ADCC in both human and murine antibodies, these substitutions are less effective at reducing CDC activity. Another single variant P329A, identified by a random mutagenesis approach to map the Clq binding site of the Fc, is highly effective at reducing CDC activity while retaining ADCC activity. A combination of L234A, L235A, and P329A (LALA-PG, Kabat positions) substitutions have been shown to effectively silence the effector function of human IgG1 antibodies. For a detailed discussion of LALA, LALA-PG, and other mutations, see Lo et al. (2017) 1 Biol. Chem. 292:3900-3908, the contents of which are hereby incorporated herein by reference in their entirety. In some embodiments, Fc fusion proteins of the disclosure comprise L234A, L235A, and P329G mutations (LALA-PG; Kabat positions) in the Fc region of the heavy chain. The antibody or Fc fusion protein may alternatively or additionally carry other mutations, including the point mutation K322A in the CH2 domain (Hezareh et al. 2001 J Virol. 75: 12161-8).

In particular embodiments, the antibody or Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively, or additionally, cysteine residue(s) may be introduced in the Fc region, thereby allowing interchain disulfide bond formation in this region. The homodimeric antibody or Fc fusion protein thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. Nos. 5,648,260; 5,624,821; and WO94/29351.

Ib Heteromultimers

Many methods known in the art can be used to generate ActRIIB:ALK4 heteromultimers, ActRIIB:ALK7 heteromultimers, ActRIIA:ALK4 heteromultimers, and ActRIIA:ALK7 heteromultimers as disclosed herein. For example, non-naturally occurring disulfide bonds may be constructed by replacing on a first polypeptide (e.g., an ActRIIB or ActRIIA polypeptide) a naturally occurring amino acid with a free thiol-containing residue, such as cysteine, such that the free thiol interacts with another free thiol-containing residue on a second polypeptide (e.g., an ALK4 or ALK7 polypeptide) such that a disulfide bond is formed between the first and second polypeptides. Additional examples of interactions to promote heteromultimer formation include, but are not limited to, ionic interactions such as described in Kjaergaard et al., WO2007147901; electrostatic steering effects such as described in Kannan et al., U.S. Pat. No. 8,592,562; coiled-coil interactions such as described in Christensen et al., U.S. 20120302737; leucine zippers such as described in Pack & Plueckthun, (1992) Biochemistry 31: 1579-1584; and helix-turn-helix motifs such as described in Pack et al., (1993) Bio/Technology 11: 1271-1277. Linkage of the various segments may be obtained via, e.g., covalent binding such as by chemical cross-linking, peptide linkers, disulfide bridges, etc., or affinity interactions such as by avidin-biotin or leucine zipper technology.

As specific examples, the present disclosure provides fusion proteins comprising ActRIIB, ActRIIA, ALK4, or ALK7 fused to a polypeptide comprising a constant domain of an immunoglobulin, such as a CH1, CH2, or CH3 domain derived from human IgG1, IgG2, IgG3, and/or IgG4 that has been modified to promote heteromultimer formation. A problem that arises in large-scale production of asymmetric immunoglobulin-based proteins from a single cell line is known as the “chain association issue”. As confronted prominently in the production of bispecific antibodies, the chain-association issue concerns the challenge of efficiently producing a desired multichain protein from among the multiple combinations that inherently result when different heavy chains and/or light chains are produced in a single cell line [see, for example, Klein et al (2012) mAbs 4:653-663]. This problem is most acute when two different heavy chains and two different light chains are produced in the same cell, in which case there are a total of 16 possible chain combinations (although some of these are identical) when only one is typically desired. Nevertheless, the same principle accounts for diminished yield of a desired multichain fusion protein that incorporates only two different (asymmetric) heavy chains.

Various methods are known in the art that increase desired pairing of Fc-containing fusion polypeptide chains in a single cell line to produce a preferred asymmetric fusion protein at acceptable yields [see, for example, Klein et al (2012) mAbs 4:653-663; and Spiess et al (2015) Molecular Immunology 67(2A): 95-106]. Methods to obtain desired pairing of Fc-containing chains include, but are not limited to, charge-based pairing (electrostatic steering), “knobs-into-holes” steric pairing, SEEDbody pairing, and leucine zipper-based pairing. See, for example, Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605. As described herein, these methods may be used to generate heterodimers comprising an ActRIIB polypeptide and another, optionally different, ActRIIB polypeptide, an ActRIIA polypeptide and another, optionally different, ActRIIA polypeptide, an ActRIIB polypeptide and an ActRIIA polypeptide, an ActRIIB polypeptide and an ALK4 polypeptide, an ActRIIB polypeptide and an ALK7 polypeptide, an ActRIIA polypeptide and an ALK4 polypeptide, or an ActRIIA polypeptide and an ALK7 polypeptide.

For example, one means by which interaction between specific polypeptides may be promoted is by engineering protuberance-into-cavity (knob-into-holes) complementary regions such as described in Arathoon et al., U.S. Pat. No. 7,183,076 and Carter et al., U.S. Pat. No. 5,731,168. “Protuberances” are constructed by replacing small amino acid side chains from the interface of the first polypeptide (e.g., a first interaction pair) with larger side chains (e.g., tyrosine or tryptophan). Complementary “cavities” of identical or similar size to the protuberances are optionally created on the interface of the second polypeptide (e.g., a second interaction pair) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface.

At neutral pH (7.0), aspartic acid and glutamic acid are negatively charged, and lysine, arginine, and histidine are positively charged. These charged residues can be used to promote heterodimer formation and at the same time hinder homodimer formation. Attractive interactions take place between opposite charges and repulsive interactions occur between like charges. In part, polypeptide complexes disclosed herein make use of the attractive interactions for promoting heteromultimer formation (e.g., heterodimer formation), and optionally repulsive interactions for hindering homodimer formation (e.g., homodimer formation) by carrying out site directed mutagenesis of charged interface residues.

For example, the IgG1 CH3 domain interface comprises four unique charge residue pairs involved in domain-domain interactions: Asp356-Lys439′, Glu357-Lys370′, Lys392-Asp399′, and Asp399-Lys409′ [residue numbering in the second chain is indicated by (′)]. It should be noted that the numbering scheme used here to designate residues in the IgG1 CH3 domain conforms to the EU numbering scheme of Kabat. Due to the 2-fold symmetry present in the CH3-CH3 domain interactions, each unique interaction will be represented twice in the structure (e.g., Asp-399-Lys409′ and Lys409-Asp399′). In the wild-type sequence, K409-D399′ favors both heterodimer and homodimer formation. A single mutation switching the charge polarity (e.g., K409E; positive to negative charge) in the first chain leads to unfavorable interactions for the formation of the first chain homodimer. The unfavorable interactions arise due to the repulsive interactions occurring between the same charges (negative-negative; K409E-D399′ and D399-K409E′). A similar mutation switching the charge polarity (D399K′; negative to positive) in the second chain leads to unfavorable interactions (K409′-D399K′ and D399K-K409′) for the second chain homodimer formation. But, at the same time, these two mutations (K409E and D399K′) lead to favorable interactions (K409E-D399K′ and D399-K409′) for the heterodimer formation.

The electrostatic steering effect on heterodimer formation and homodimer discouragement can be further enhanced by mutation of additional charge residues which may or may not be paired with an oppositely charged residue in the second chain including, for example, Arg355 and Lys360. The table below lists possible charge change mutations that can be used, alone or in combination, to enhance heteromultimer formation of the heteromultimers disclosed herein.

Examples of Pair-Wise Charged Residue Mutations
to Enhance Heterodimer Formation

| | | Interacting | Corresponding |
|---|---|---|---|
| Position in | Mutation in | position in | mutation in |
| first chain | first chain | second chain | second chain |
| Lys409 | Asp or Glu | Asp399′ | Lys, Arg, or His |
| Lys392 | Asp or Glu | Asp399′ | Lys, Arg, or His |
| Lys439 | Asp or Glu | Asp356′ | Lys, Arg, or His |
| Lys370 | Asp or Glu | Glu357′ | Lys, Arg, or His |
| Asp399 | Lys, Arg, or His | Lys409′ | Asp or Glu |
| Asp399 | Lys, Arg, or His | Lys392′ | Asp or Glu |
| Asp356 | Lys, Arg, or His | Lys439′ | Asp or Glu |
| Glu357 | Lys, Arg, or His | Lys370′ | Asp or Glu |

In some embodiments, one or more residues that make up the CH3-CH3 interface in a fusion polypeptide of the instant application are replaced with a charged amino acid such that the interaction becomes electrostatically unfavorable. For example, a positive-charged amino acid in the interface (e.g., a lysine, arginine, or histidine) is replaced with a negatively charged amino acid (e.g., aspartic acid or glutamic acid). Alternatively, or in combination with the forgoing substitution, a negative-charged amino acid in the interface is replaced with a positive-charged amino acid. In certain embodiments, the amino acid is replaced with a non-naturally occurring amino acid having the desired charge characteristic. It should be noted that mutating negatively charged residues (Asp or Glu) to His will lead to increase in side chain volume, which may cause steric issues. Furthermore, His proton donor- and acceptor-form depends on the localized environment. These issues should be taken into consideration with the design strategy. Because the interface residues are highly conserved in human and mouse IgG subclasses, electrostatic steering effects disclosed herein can be applied to human and mouse IgG1, IgG2, IgG3, and IgG4. This strategy can also be extended to modifying uncharged residues to charged residues at the CH3 domain interface.

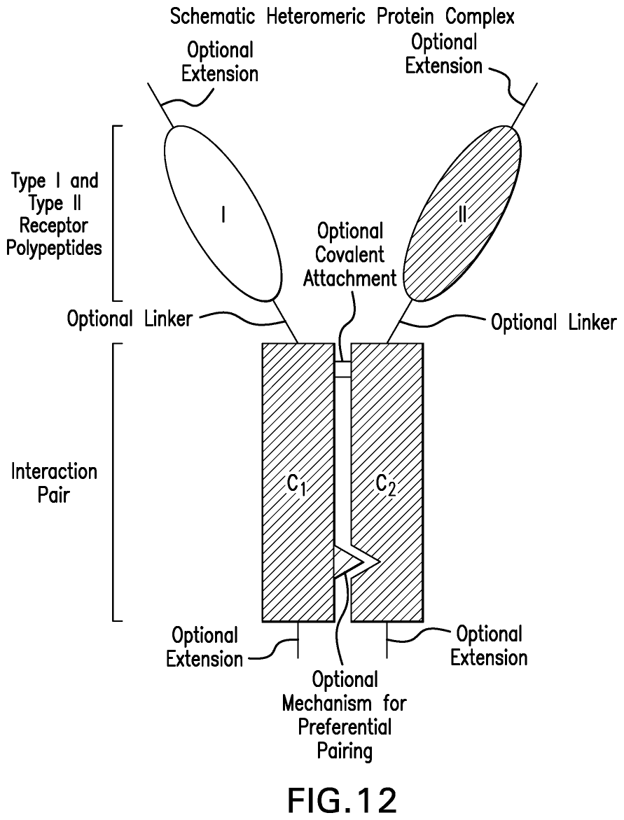

In certain aspects, the ActRII-ALK4 ligand trap to be used in accordance with the methods disclosed herein is a heteromultimer complex comprising at least one ALK polypeptide (e.g., an ALK4 or ALK7 polypeptide) associated, covalently or non-covalently, with at least one ActRII polypeptide (e.g., an ActRIIA or ActRIIB polypeptide). Preferably, polypeptides disclosed herein form heterodimeric complexes, although higher order heteromultimeric complexes (heteromultimers) are also included such as, but not limited to, heterotrimers, heterotetramers, and further oligomeric structures (see, e.g., FIGS. 11-13, which may also be applied to both ActRII-ALK4 and ActRII-ALK7 oligomeric structures). In some embodiments, ALK and/or ActRII polypeptides comprise at least one multimerization domain. Polypeptides disclosed herein may be joined covalently or non-covalently to a multimerization domain. Preferably, a multimerization domain promotes interaction between a first polypeptide (e.g., an ActRIIB or ActRIIA polypeptide) and a second polypeptide (e.g., an ALK4 or ALK7 polypeptide) to promote heteromultimer formation (e.g., heterodimer formation), and optionally hinders or otherwise disfavors homomultimer formation (e.g., homodimer formation), thereby increasing the yield of desired heteromultimer (see, e.g., FIG. 12).

In part, the disclosure provides desired pairing of asymmetric Fc-containing polypeptide chains using Fc sequences engineered to be complementary on the basis of charge pairing (electrostatic steering). One of a pair of Fc sequences with electrostatic complementarity can be arbitrarily fused to an ActRIIB polypeptide, ActRIIA polypeptide, ALK4 polypeptide, or an ALK7 polypeptide of the construct, with or without an optional linker, to generate an ActRIIB-Fc, ActRIIA-Fc, ALK4-Fc, or ALK7-Fc fusion polypeptide. This single chain can be coexpressed in a cell of choice along with the Fc sequence complementary to the first Fc sequence to favor generation of the desired multichain construct (e.g., an ActRIIB-Fc-ALK4-Fc heteromultimer). In this example based on electrostatic steering, SEQ ID NO: 18 [human G1Fc(E134K/D177K)] and SEQ ID NO: 19 [human G1Fc(K170D/K187D)] are examples of complementary Fc sequences in which the engineered amino acid substitutions are double underlined, and an ActRIIB polypeptide, ActRIIA polypeptide, ALK4 polypeptide, or an ALK7 polypeptide of the construct can be fused to either SEQ ID NO: 18 or SEQ ID NO: 19, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 7) will generate complementary Fc pairs which may be used instead of the complementary hG1Fc pair below (SEQ ID NOs: 18 and 19).

```
(SEQ ID NO: 18)
    1 THTCPPCPAP ELLGGPSVEL FPPKPKDTLM
        ISRTPEVTCV VVDVSHEDPE
  51 VKFNWYVDGV EVHNAKTKPR EEQYNSTYRV
        VSVLTVLHQD WLNGKEYKCK
101 VSNKALPAPI EKTISKAKGQ PREPQVYTLP
        PSRKEMTKNQ VSLTCLVKGE
151 YPSDIAVEWE SNGQPENNYK TTPPVLKSDG
        SFFLYSKLTV DKSRWQQGNV
201 FSCSVMHEAL FSCSVMHEAL HNHYTQ
(SEQ ID NO: 19)
    1 THTCPPCPAP ELLGGPSVEL FPPKPKDTLM
        ISRTPEVTCV VVDVSHEDPE
  51 VKFNWYVDGV EVHNAKTKPR EEQYNSTYRV
        VSVLIVLHQD WINGKEYKCK
101 VSNKALPAPI EKTISKAKGQ PREPQVYTLP
        PSREEMTKNQ VSLTCLVKGE
151 YPSDIAVEWE SNGQPENNYD TTPPVLDSDG
        SFFLYSDLTV DKSRWQQGNV
201 FSCSVMHEAL HNHYTQKSLS LSPGK
```

In some embodiments, the disclosure relates to ActRIIB:ALK4 heteromultimer polypeptides comprising an ActRIIB-Fc fusion polypeptide and an ALK4-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18. In some embodiments, the disclosure relates to ActRIIB heteromultimer polypeptides comprising an ActRIIB-Fc fusion polypeptide and an ALK4-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19.

In some embodiments, the disclosure relates to ActRIIB:ALK7 heteromultimer polypeptides comprising an ActRIIB-Fc fusion polypeptide and an ALK7-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18. In some embodiments, the disclosure relates to ActRIIB:ALK7 heteromultimer polypeptides comprising an ActRIIB-Fc fusion polypeptide and an ALK7-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18, and the ALK7-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 19.

In some embodiments, the disclosure relates to ActRIIA-ALK4 heteromultimer polypeptides comprising an ActRIIA-Fc fusion polypeptide and an ALK4-Fc fusion polypeptide wherein the ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 19, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18. In some embodiments, the disclosure relates to ActRIIA heteromultimer polypeptides comprising an ActRIIA-Fc fusion polypeptide and an ALK4-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19.

In some embodiments, the disclosure relates to ActRIIA:ALK7 heteromultimer polypeptides comprising an ActRIIA-Fc fusion polypeptide and an ALK7-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18. In some embodiments, the disclosure relates to ActRIIA:ALK7 heteromultimer polypeptides comprising an ActRIIA-Fc fusion polypeptide and an ALK7-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18, and the ALK7-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 19.

In part, the disclosure provides desired pairing of asymmetric Fc-containing polypeptide chains using Fc sequences engineered for steric complementarity. In part, the disclosure provides knobs-into-holes pairing as an example of steric complementarity. One of a pair of Fc sequences with steric complementarity can be arbitrarily fused to an ActRIIB polypeptide, an ActRIIA polypeptide, an ALK4 polypeptide, or an ALK7 polypeptide of the construct, with or without an optional linker, to generate an ActRIIB-Fc, ActRIIA-Fc, ALK4-Fc, or ALK7-Fc fusion polypeptide. This single chain can be coexpressed in a cell of choice along with the Fc sequence complementary to the first Fc sequence to favor generation of the desired multichain construct. In this example based on knobs-into-holes pairing, SEQ ID NO: 20 [human G1Fc(T144Y)] and SEQ ID NO: 21 [human G1Fc(Y185T)] are examples of complementary Fc sequences in which the engineered amino acid substitutions are double underlined, and an ActRIIB polypeptide, ActRIIA polypeptide, ALK4 polypeptide, or ALK7 polypeptide of the construct can be fused to either SEQ ID NO: 20 or SEQ ID NO: 21, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 7) will generate complementary Fc pairs which may be used instead of the complementary hG1Fc pair below (SEQ ID NOs: 20 and 21).

```
(SEQ ID NO: 20)
    1 THTCPPCPAP ELLGGPSVEL FPPKPKDTLM
        ISRTPEVTCV VVDVSHEDPE
  51 VKFNWYVDGV EVHNAKTKPR EEQYNSTYRV
        VSVLTVLHQD WLNGKEYKCK
101 VSNKALPAPI EKTISKAKGQ PREPQVYTLP
        PSREEMTKNQ VSLYCLVKGE
151 YPSDIAVEWE SNGQPENNYK TTPPVLDSDG
        SFFLYSKLTV DKSRWQQGNV
201 FSCSVMHEAL HNHYTQKSLS LSPGK
(SEQ ID NO: 21)
    1 THTCPPCPAP ELLGGPSVEL FPPKPKDTLM
        ISRTPEVTCV VVDVSHEDPE
  51 VKFNWYVDGV EVHNAKTKPR EEQYNSTYRV
        VSVLTVLHQD WLNGKEYKCK
101 VSNKALPAPI EKTISKAKGQ PREPQVYTLP
        PSREEMTKNQ VSLTCLVKGE
151 YPSDIAVEWE SNGQPENNYK TTPPVLDSDG
        SFFLTSKLTV DKSRWQQGNV
201 FSCSVMHEAL HNHYTQKSLS LSPGK
```

In some embodiments, the disclosure relates to ActRIIB:ALK4 heteromultimer polypeptides comprising an ActRIIB-Fc fusion polypeptide and an ALK4-Fc fusion polypeptide wherein the ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 21, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20. In some embodiments, the disclosure relates to ActRIIB:ALK4 heteromultimer polypeptides comprising an ActRIIB-Fc fusion polypeptide and an ALK4-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20, and the ALK4-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 21.

In some embodiments, the disclosure relates to ActRIIB:ALK7 heteromultimer polypeptides comprising an ActRIIB-Fc fusion polypeptide and an ALK7-Fc fusion polypeptide wherein the ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 21, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20. In some embodiments, the disclosure relates to ActRIIB:ALK7 heteromultimer polypeptides comprising an ActRIIB-Fc fusion polypeptide and an ALK7-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20, and the ALK7-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 21.

In some embodiments, the disclosure relates to ActRIIA:ALK4 heteromultimer polypeptides comprising an ActRIIA-Fc fusion polypeptide and an ALK4-Fc fusion polypeptide wherein the ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 21, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20. In some embodiments, the disclosure relates to ActRIIA:ALK4 heteromultimer polypeptides comprising an ActRIIA-Fc fusion polypeptide and an ALK4-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20, and the ALK4-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 21.

In some embodiments, the disclosure relates to ActRIIA:ALK7 heteromultimer polypeptides comprising an ActRIIA-Fc fusion polypeptide and an ALK7-Fc fusion polypeptide wherein the ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 21, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20. In some embodiments, the disclosure relates to ActRIIA:ALK7 heteromultimer polypeptides comprising an ActRIIA-Fc fusion polypeptide and an ALK7-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20, and the ALK7-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 21.

An example of Fc complementarity based on knobs-into-holes pairing combined with an engineered disulfide bond is disclosed in SEQ ID NO: 22 [hG1Fc(S132C/T144W)] and SEQ ID NO: 23 [hG1Fc(Y127C/T144S/L146A/Y185V)]. The engineered amino acid substitutions in these sequences are double underlined, and an ActRIIB polypeptide, ActRIIA polypeptide, ALK4 polypeptide, or ALK7 polypeptide of the construct can be fused to either SEQ ID NO: 22 or SEQ ID NO: 23, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 7) will generate complementary Fc pairs which may be used instead of the complementary hG1Fc pair below (SEQ ID NOs: 22 and 23).

| (SEQ ID NO: 22) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WINGKEYKCK |
| 101 | VSNKALPAPI EKTISKAKGQ PREPQVYTLP PCREEMTKNQ VSLWCLVKGE |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| (SEQ ID NO: 23) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGF |
| 151 | YPSDIAVEWE SNGQPENNYK TTPPVLDSDG SFFLVSKLTV DKSRWQQGNV |
| 201 | FSCSVMHEAL HNHYTQKSLS LSPGK |

In some embodiments, the disclosure relates to ActRIIB:ALK4 heteromultimer polypeptides comprising an ActRIIB-Fc fusion polypeptide and an ALK4-Fc fusion polypeptide wherein the ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 23, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22. In some embodiments, the disclosure relates to ActRIIB:ALK4 heteromultimer polypeptides comprising an ActRIIB-Fc fusion polypeptide and an ALK4-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22, and the ALK4-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 23.

In some embodiments, the disclosure relates to ActRIIB:ALK7 heteromultimer polypeptides comprising an ActRIIB-Fc fusion polypeptide and an ALK7-Fc fusion polypeptide wherein the ActRIIB-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 23, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22. In some embodiments, the disclosure relates to ActRIIB:ALK7 heteromultimer polypeptides comprising an ActRIIB-Fc fusion polypeptide and an ALK7-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22, and the ALK7-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 23.

In some embodiments, the disclosure relates to ActRIIA:ALK4 heteromultimer polypeptides comprising an ActRIIA-Fc fusion polypeptide and an ALK4-Fc fusion polypeptide wherein the ActRIIA-Fc fusion polypeptide comprises an Fc domain that is at least 75%, 80%, 85%, 90%, 91%, 92%, 93%, 94%, 95% 96%0, 97% 98%, 99%, or 100% identical to the amino acid sequence of SEQ ID NO: 23, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22. In some embodiments, the disclosure relates to ActRIIA:ALK4 heteromultimer polypeptides comprising an ActRIIA-Fc fusion polypeptide and an ALK4-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22, and the ALK4-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 23.

In some embodiments, the disclosure relates to ActRIIA:ALK7 heteromultimer polypeptides comprising an ActRIIA-Fc fusion polypeptide and an ALK7-Fc fusion polypeptide wherein the ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 23, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22. In some embodiments, the disclosure relates to ActRIIA:ALK7 heteromultimer polypeptides comprising an ActRIIA-Fc fusion polypeptide and an ALK7-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22, and the ALK7-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 23.

In part, the disclosure provides desired pairing of asymmetric Fc-containing polypeptide chains using Fc sequences engineered to generate interdigitating β-strand segments of human IgG and IgA C_{H}3 domains. Such methods include the use of strand-exchange engineered domain (SEED) C_{H}3 heterodimers allowing the formation of SEEDbody fusion polypeptides [see, for example, Davis et al (2010) Protein Eng Design Sel 23:195-202]. One of a pair of Fc sequences with SEEDbody complementarity can be arbitrarily fused to a first ActRIIB polypeptide or second ActRIIB polypeptide of the construct, with or without an optional linker, to generate an ActRIIB-Fc fusion polypeptide. This single chain can be coexpressed in a cell of choice along with the Fc sequence complementary to the first Fc sequence to favor generation of the desired multichain construct. In this example based on SEEDbody (Sb) pairing, SEQ ID NO: 24 [hG1Fc(Sb_{AG})] and SEQ ID NO: 25 [hG1Fc(Sb_{GA})] are examples of complementary IgG Fc sequences in which the engineered amino acid substitutions from IgA Fc are double underlined, and a first ActRIIB polypeptide or second variant ActRIIB polypeptide, of the construct can be fused to either SEQ ID NO: 24 or SEQ ID NO: 25, but not both. Given the high degree of amino acid sequence identity between native hG1Fc, native hG2Fc, native hG3Fc, and native hG4Fc, it can be appreciated that amino acid substitutions at corresponding positions in hG1Fc, hG2Fc, hG3Fc, or hG4Fc (see FIG. 7) will generate an Fc monomer which may be used in the complementary IgG-IgA pair below (SEQ ID NOs: 24 and 25).

| (SEQ ID NO: 24) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PFRPEVHLLP PSREEMTKNQ VSLTCLARGE |
| 151 | YPKDIAVEWE SNGQPENNYK TTPSRQEPSQ GTTTFAVTSK LTVDKSRWQQ |
| 201 | GNVFSCSVMH EALHNHYTQK TISLSPGK |
| (SEQ ID NO: 25) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PPSEELALNE LVTLTCLVKG |
| 151 | FYPSDIAVEW ESNGQELPRE KYLTWAPVLD SDGSFFLYSI LRVAAEDWKK |
| 201 | GDTFSCSVMH EALHNHYTQK SLDRSPGK |

In some embodiments, the disclosure relates to ActRIIB:ALK4 heteromultimer polypeptides comprising an ActRIIB-Fc fusion polypeptide and an ALK4-Fc fusion polypeptide wherein the ActRIIB-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 25, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24. In some embodiments, the disclosure relates to ActRIIB:ALK4 heteromultimer polypeptides comprising an ActRIIB-Fc fusion polypeptide and an ALK4-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24, and the ALK4-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 25.

In some embodiments, the disclosure relates to ActRIIB:ALK7 heteromultimer polypeptides comprising an ActRIIB-Fc fusion polypeptide and an ALK7-Fc fusion polypeptide wherein the ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 25, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24. In some embodiments, the disclosure relates to ActRIIB:ALK7 heteromultimer polypeptides comprising an ActRIIB-Fc fusion polypeptide and an ALK7-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24, and the ALK7-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 25.

In some embodiments, the disclosure relates to ActRIIA:ALK4 heteromultimer polypeptides comprising an ActRIIA-Fc fusion polypeptide and an ALK4-Fc fusion polypeptide wherein the ActRIIA-Fc fusion polypeptide comprises an Fc domain that is at least 75%, 80%, 85%, 90%, 91%, 92%, 93%, 94%, 95% 96%0, 97% 98%, 99%, or 100% identical to the amino acid sequence of SEQ ID NO: 25, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24. In some embodiments, the disclosure relates to ActRIIA:ALK4 heteromultimer polypeptides comprising an ActRIIA-Fc fusion polypeptide and an ALK4-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24, and the ALK4-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 25.

In some embodiments, the disclosure relates to ActRIIA:ALK7 heteromultimer polypeptides comprising an ActRIIA-Fc fusion polypeptide and an ALK7-Fc fusion polypeptide wherein the ActRIIA-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 25, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24. In some embodiments, the disclosure relates to ActRIIA:ALK7 heteromultimer polypeptides comprising an ActRIIA-Fc fusion polypeptide and an ALK7-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24, and the ALK7-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 25.

In part, the disclosure provides desired pairing of asymmetric Fc-containing polypeptide chains with a cleavable leucine zipper domain attached at the C-terminus of the Fc C_{H}3 domains. Attachment of a leucine zipper is sufficient to cause preferential assembly of heterodimeric antibody heavy chains. See, e.g., Wranik et al (2012) J Biol Chem 287:43331-43339. As disclosed herein, one of a pair of Fc sequences attached to a leucine zipper-forming strand can be arbitrarily fused to a first ActRIIB polypeptide or second ActRIIB polypeptide, of the construct, with or without an optional linker, to generate an ActRIIB-Fc fusion polypeptide. This single chain can be coexpressed in a cell of choice along with the Fc sequence attached to a complementary leucine zipper-forming strand to favor generation of the desired multichain construct. Proteolytic digestion of the construct with the bacterial endoproteinase Lys-C post purification can release the leucine zipper domain, resulting in an Fc construct whose structure is identical to that of native Fc. In this example based on leucine zipper pairing, SEQ ID NO: 26 [hG1Fc-Ap1 (acidic)] and SEQ ID NO: 27 [hG1Fc-Bp1 (basic)] are examples of complementary IgG Fc sequences in which the engineered complimentary leucine zipper sequences are underlined, and a ActRIIB polypeptide or second variant ActRIIB polypeptide of the construct can be fused to either SEQ ID NO: 26 or SEQ ID NO: 27, but not both. Given the high degree of amino acid sequence identity between native hG1Fc, native hG2Fc, native hG3Fc, and native hG4Fc, it can be appreciated that leucine zipper-forming sequences attached, with or without an optional linker, to hG1Fc, hG2Fc, hG3Fc, or hG4Fc (see FIG. 7) will generate an Fc monomer which may be used in the complementary leucine zipper-forming pair below (SEQ ID NOs: 26 and 27).

| (SEQ ID NO: 26) | |
|---|---|
| 1 | THTCPPCPAP ELLGGPSVEL FPPKPKDTLM ISRTPEVTCV VVDVSHEDPE |
| 51 | VKFNWYVDGV EVHNAKTKPR EEQYNSTYRV VSVLTVLHQD WINGKEYKCK |
| 101 | VSNKALPAPI EKTISKAKGQ PREPQVYTLP PSREEMTKNQ VSLTCLVKGE |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LEKELQALEK ENAQLEWELQ |
| 251 | ALEKELAQGA T |
| (SEQ ID NO: 27) | |
| 1 | THTCPPCPAP ELLGGPSVEL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGE |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LKKKLQALKK KNAQLKWKLQ |
| 251 | ALKKKLAQGA T |

In some embodiments, the disclosure relates to ActRIIB:ALK4 heteromultimer polypeptides comprising an ActRIIB-Fc fusion polypeptide and an ALK4-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26. In some embodiments, the disclosure relates to ActRIIB:ALK4 heteromultimer polypeptides comprising an ActRIIB-Fc fusion polypeptide and an ALK4-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27.

In some embodiments, the disclosure relates to ActRIIB:ALK7 heteromultimer polypeptides comprising an ActRIIB-Fc fusion polypeptide and an ALK7-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26. In some embodiments, the disclosure relates to ActRIIB:ALK7 heteromultimer polypeptides comprising an ActRIIB-Fc fusion polypeptide and an ALK7-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26, and the ALK7-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 27.

In some embodiments, the disclosure relates to ActRIIA:ALK4 heteromultimer polypeptides comprising an ActRIIA-Fc fusion polypeptide and an ALK4-Fc fusion polypeptide wherein the ActRIIA-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 27, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26. In some embodiments, the disclosure relates to ActRIIA:ALK4 heteromultimer polypeptides comprising an ActRIIA-Fc fusion polypeptide and an ALK4-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27.

In some embodiments, the disclosure relates to ActRIIA:ALK7 heteromultimer polypeptides comprising an ActRIIA-Fc fusion polypeptide and an ALK7-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26. In some embodiments, the disclosure relates to ActRIIA:ALK7 heteromultimer polypeptides comprising an ActRIIA-Fc fusion polypeptide and an ALK7-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27.

In part, the disclosure provides desired pairing of asymmetric Fc-containing polypeptide chains by methods described above in combination with additional mutations in the Fc domain which facilitate purification of the desired heteromeric species. An example uses complementarity of Fc domains based on knobs-into-holes pairing combined with an engineered disulfide bond, as disclosed in SEQ ID NOs: 22 and 23, plus additional substitution of two negatively charged amino acids (aspartic acid or glutamic acid) in one Fc-containing polypeptide chain and two positively charged amino acids (e.g., arginine) in the complementary Fc-containing polypeptide chain (SEQ ID NOs: 28-29). These four amino acid substitutions facilitate selective purification of the desired heteromeric fusion polypeptide from a heterogeneous polypeptide mixture based on differences in isoelectric point or net molecular charge. The engineered amino acid substitutions in these sequences are double underlined below, and an ActRIIB polypeptide, an ActRIIA polypeptide, an ALK4 polypeptide, or an ALK7 polypeptide of the construct can be fused to either SEQ ID NO: 28 or SEQ ID NO: 29, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 7) will generate complementary Fc pairs which may be used instead of the complementary hG1Fc pair below (SEQ ID NOs: 28-29).

| (SEQ ID NO: 28) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTENQ VSLWCLVKGE |
| 151 | YPSDIAVEWE SNGQPENNYK TTPPVLDSDG SFFLYSKLTV DKSRWQQGNV |
| 201 | FSCSVMHEAL HNHYTQDSLS LSPGK |
| (SEQ ID NO: 29) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGE |
| 151 | YPSDIAVEWE SRGQPENNYK TTPPVLDSRG SFFLVSKLTV DKSRWQQGNV |
| 201 | FSCSVMHEAL HNHYTQKSLS LSPGK |

In some embodiments, the disclosure relates to ActRIIB:ALK4 heteromultimer polypeptides comprising an ActRIIB-Fc fusion polypeptide and an ALK4-Fc fusion polypeptide wherein the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments, the disclosure relates to ActRIIB:ALK7 heteromultimer polypeptides comprising an ActRIIB-Fc fusion polypeptide and an ALK7-Fc fusion polypeptide wherein the ActRIIB-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 28, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments, the ActRIIB-Fc fusion polypeptide Fc domain comprises a cysteine at amino acid position 132, glutamic acid at amino acid position 138, a tryptophan at amino acid position 144, and an aspartic acid at amino acid position 217. In some embodiments, the ALK4-Fc fusion polypeptide Fc domain comprises a cysteine at amino acid position 127, a serine at amino acid position 144, an alanine at amino acid position 146, an arginine at amino acid position 162, an arginine at amino acid position 179, and a valine at amino acid position 185. In some embodiments, the ALK7-Fc fusion polypeptide Fc domain comprises a cysteine at amino acid position 127, a serine at amino acid position 144, an alanine at amino acid position 146, an arginine at amino acid position 162, an arginine at amino acid position 179, and a valine at amino acid position 185.

In some embodiments, the disclosure relates to ActRIIA:ALK4 heteromultimer polypeptides comprising an ActRIIA-Fc fusion polypeptide and an ALK4-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments, the disclosure relates to ActRIIA:ALK7 heteromultimer polypeptides comprising an ActRIIA-Fc fusion polypeptide and an ALK7-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

Another example involves complementarity of Fc domains based on knobs-into-holes pairing combined with an engineered disulfide bond, as disclosed in SEQ ID NOs: 22-23, plus a histidine-to-arginine substitution at position 213 in one Fc-containing polypeptide chain (SEQ ID NO: 30). This substitution (denoted H435R in the numbering system of Kabat et al.) facilitates separation of desired heterodimer from undesirable homodimer based on differences in affinity for protein A. The engineered amino acid substitution is indicated by double underline, and an ActRIIB polypeptide, ActRIIA polypeptide, ALK4 polypeptide, or ALK7 polypeptide of the construct can be fused to either SEQ ID NO: 30 or SEQ ID NO: 23, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 7) will generate complementary Fc pairs which may be used instead of the complementary hG1Fc pair of SEQ ID NO: 30 (below) and SEQ ID NO: 23.

| (SEQ ID NO: 30) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WINGKEYKCK |
| 101 | VSNKALPAPI EKTISKAKGQ PREPQVYTLP PCREEMTKNQ VSLWCLVKGE |
| 151 | YPSDIAVEWE SNGQPENNYK TTPPVLDSDG SFFLYSKLTV DKSRWQQGNV |
| 201 | FSCSVMHEAL HNRYTQKSLS LSPGK |

In some embodiments, the disclosure relates to ActRIIB:ALK4 heteromultimer polypeptides comprising an ActRIIB-Fc fusion polypeptide and an ALK4-Fc fusion polypeptide wherein the ActRIIB-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 30, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments, the disclosure relates to ActRIIB:ALK7 heteromultimer polypeptides comprising an ActRIIB-Fc fusion polypeptide and an ALK7-Fc fusion polypeptide wherein the ActRIIB-Fc fusion polypeptide comprises an Fc domain that is at least 75%, 80%, 85%, 90%, 91%, 92%, 93%, 94%, 95% 96%0, 97% 98%, 99%, or 100% identical to the amino acid sequence of SEQ ID NO: 30, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments, the ActRIIB-Fc fusion polypeptide Fc domain comprises a cysteine at amino acid position 132, a tryptophan at amino acid position 144, and an arginine at amino acid position 435. In some embodiments, the ALK4-Fc fusion polypeptide Fc domain comprises cysteine at amino acid position 127, a serine at amino acid position 144, an alanine at amino acid position 146, and a valine at amino acid position 185. In some embodiments, the ALK7-Fc fusion polypeptide Fc domain comprises cysteine at amino acid position 127, a serine at amino acid position 144, an alanine at amino acid position 146, and a valine at amino acid position 185.

In some embodiments, the disclosure relates to ActRIIB:ALK4 heteromultimer polypeptides comprising an ActRIIB-Fc fusion polypeptide and an ALK4-Fc fusion polypeptide wherein the ALK4-Fc fusion polypeptide comprises an Fc domain that is at least 75%, 80%, 85%, 90%, 91%, 92%, 93%, 94%, 95%, 96%, 97% 98%, 99%, or 100% identical to the amino acid sequence of SEQ ID NO: 30, and the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments, the disclosure relates to ActRIIB:ALK7 heteromultimer polypeptides comprising an ActRIIB-Fc fusion polypeptide and an ALK7-Fc fusion polypeptide wherein the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23

In some embodiments, the ActRIIA-Fc fusion polypeptide Fc domain comprises a cysteine at amino acid position 132, glutamic acid at amino acid position 138, a tryptophan at amino acid position 144, and an aspartic acid at amino acid position 217. In some embodiments, the ALK4-Fc fusion polypeptide Fc domain comprises a cysteine at amino acid position 127, a serine at amino acid position 144, an alanine at amino acid position 146, an arginine at amino acid position 162, an arginine at amino acid position 179, and a valine at amino acid position 185. In some embodiments, the ALK7-Fc fusion polypeptide Fc domain comprises a cysteine at amino acid position 127, a serine at amino acid position 144, an alanine at amino acid position 146, an arginine at amino acid position 162, an arginine at amino acid position 179, and a valine at amino acid position 185.

In some embodiments, the disclosure relates to ActRIIB:ALK4 heteromultimer polypeptides comprising an ActRIIB-Fc fusion polypeptide and an ALK4-Fc fusion polypeptide wherein the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments, the disclosure relates to ActRIIB:ALK7 heteromultimer polypeptides comprising an ActRIIB-Fc fusion polypeptide and an ALK7-Fc fusion polypeptide wherein the ALK7-Fc fusion polypeptide comprises an Fc domain that is at least 75%, 80%, 85%, 90%, 91%, 92%, 93%, 94%, 95% 96%, 97%, 98%, 99%, or 100% identical to the amino acid sequence of SEQ ID NO: 28, and the ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments, the ALK4-Fc fusion polypeptide Fc domain comprises a cysteine at amino acid position 132, glutamic acid at amino acid position 138, a tryptophan at amino acid position 144, and an aspartic acid at amino acid position 217. In some embodiments, the ActRIIB-Fc fusion polypeptide Fc domain comprises a cysteine at amino acid position 127, a serine at amino acid position 144, an alanine at amino acid position 146, an arginine at amino acid position 162, an arginine at amino acid position 179, and a valine at amino acid position 185. In some embodiments, the ALK7-Fc fusion polypeptide Fc domain comprises a cysteine at amino acid position 132, glutamic acid at amino acid position 138, a tryptophan at amino acid position 144, and an aspartic acid at amino acid position 217. In some embodiments, the ActRIIB-Fc fusion polypeptide Fc domain comprises a cysteine at amino acid position 127, a serine at amino acid position 144, an alanine at amino acid position 146, an arginine at amino acid position 162, an arginine at amino acid position 179, and a valine at amino acid position 185.

In some embodiments, the disclosure relates to ActRIIA:ALK4 heteromultimer polypeptides comprising an ActRIIA-Fc fusion polypeptide and an ALK4-Fc fusion polypeptide wherein the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments, the disclosure relates to ActRIIA:ALK7 heteromultimer polypeptides comprising an ActRIIA-Fc fusion polypeptide and an ALK7-Fc fusion polypeptide wherein the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29.

In some embodiments, the ALK4-Fc fusion polypeptide Fc domain comprises a cysteine at amino acid position 132, glutamic acid at amino acid position 138, a tryptophan at amino acid position 144, and an aspartic acid at amino acid position 217. In some embodiments, the ActRIIA-Fc fusion polypeptide Fc domain comprises a cysteine at amino acid position 127, a serine at amino acid position 144, an alanine at amino acid position 146, an arginine at amino acid position 162, an arginine at amino acid position 179, and a valine at amino acid position 185. In some embodiments, the ALK7-Fc fusion polypeptide Fc domain comprises a cysteine at amino acid position 132, glutamic acid at amino acid position 138, a tryptophan at amino acid position 144, and an aspartic acid at amino acid position 217. In some embodiments, the ActRIIA-Fc fusion polypeptide Fc domain comprises a cysteine at amino acid position 127, a serine at amino acid position 144, an alanine at amino acid position 146, an arginine at amino acid position 162, an arginine at amino acid position 179, and a valine at amino acid position 185.

In some embodiments, the ALK4-Fc fusion polypeptide Fc domain comprises a cysteine at amino acid position 132, a tryptophan at amino acid position 144, and an arginine at amino acid position 435. In some embodiments, the ALK7-Fc fusion polypeptide Fc domain comprises a cysteine at amino acid position 132, a tryptophan at amino acid position 144, and an arginine at amino acid position 435. In some embodiments, the ActRIIB-Fc fusion polypeptide Fc domain comprises cysteine at amino acid position 127, a serine at amino acid position 144, an alanine at amino acid position 146, and a valine at amino acid position 185.

In some embodiments, the disclosure relates to ActRIIA:ALK4 heteromultimer polypeptides comprising an ActRIIA-Fc fusion polypeptide and an ALK4-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments, the disclosure relates to ActRIIA:ALK7 heteromultimer polypeptides comprising an ActRIIA-Fc fusion polypeptide and an ALK7-Fc fusion polypeptide wherein the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ALK7-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments, the ActRIIA-Fc fusion polypeptide Fc domain comprises a cysteine at amino acid position 132, a tryptophan at amino acid position 144, and an arginine at amino acid position 435. In some embodiments, the ALK4-Fc fusion polypeptide Fc domain comprises cysteine at amino acid position 127, a serine at amino acid position 144, an alanine at amino acid position 146, and a valine at amino acid position 185. In some embodiments, the ALK7-Fc fusion polypeptide Fc domain comprises cysteine at amino acid position 127, a serine at amino acid position 144, an alanine at amino acid position 146, and a valine at amino acid position 185.

In some embodiments, the disclosure relates to ActRIIA:ALK4 heteromultimer polypeptides comprising an ActRIIA-Fc fusion polypeptide and an ALK4-Fc fusion polypeptide wherein the ALK4-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In some embodiments, the disclosure relates to ActRIIA:ALK7 heteromultimer polypeptides comprising an ActRIIA-Fc fusion polypeptide and an ALK7-Fc fusion polypeptide wherein the ALK7-Fc fusion polypeptide comprises an Fc domain that is at least 75%, 80%, 85%, 90%, 91%, 92%, 93%, 94%, 95% 96%, 97% 98%, 99%, or 100% identical to the amino acid sequence of SEQ ID NO: 30, and the ActRIIA-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23

In some embodiments, the ALK4-Fc fusion polypeptide Fc domain comprises a cysteine at amino acid position 132, a tryptophan at amino acid position 144, and an arginine at amino acid position 435. In some embodiments, the ALK7-Fc fusion polypeptide Fc domain comprises a cysteine at amino acid position 132, a tryptophan at amino acid position 144, and an arginine at amino acid position 435. In some embodiments, the ActRIIA-Fc fusion polypeptide Fc domain comprises cysteine at amino acid position 127, a serine at amino acid position 144, an alanine at amino acid position 146, and a valine at amino acid position 185.

In certain embodiments, the disclosure relates to a heteromultimer comprising a first variant ActRIIB-Fc fusion polypeptide and a second variant ActRIIB-Fc fusion polypeptide, wherein the first variant ActRIIB polypeptide does not comprise the amino acid sequence of the second variant ActRIIB polypeptide. In some embodiments, an ActRIIB-Fc:ActRIIB-Fc heteromultimer binds to one or more ActRII-ALK4 ligands (e.g., activin A, activin B, GDF8, GDF11, BMP6, BMP10). In some embodiments, an ActRIIB-Fc:ActRIIB-Fc heteromultimer inhibits signaling of one or more ActRII-ALK4 ligands (e.g., activin A, activin B, GDF8, GDF11, BMP6, BMP10). In some embodiments, an ActRIIB-Fc:ActRIIB-Fc heteromultimer is a heterodimer.

In some embodiments, the first ActRIIB polypeptide comprises one or more amino acid substitutions at the amino acid positions corresponding to any one of F82, L79, A24, K74, R64, P129, P130, E37, R40, D54, R56, W78, D80, and F82 of SEQ ID NO: 2. In some embodiments, the first ActRIIB polypeptide comprises one or more amino acid substitutions at the amino acid positions corresponding to any one of L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the one or more amino acid substitutions is selected from the group consisting of: L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G. In some embodiments, the second ActRIIB polypeptide comprises one or more amino acid substitutions at the amino acid positions corresponding to any one of F82, L79, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the second ActRIIB polypeptide comprises one or more amino acid substitutions at the amino acid positions corresponding to any one of L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G of SEQ ID NO: 2. In some embodiments, the one or more amino acid substitutions is selected from the group consisting of: L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G. In some embodiments, the first ActRIIB polypeptide and/or the second ActRIIB polypeptide comprise one or more amino acid modification that promote heteromultimer formation. In some embodiments, the first ActRIIB polypeptide and/or the second ActRIIB polypeptide comprise one or more amino acid modification that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 36, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 5, wherein the first ActRIIB polypeptide does not comprise the amino acid sequence of the second ActRIIB polypeptide. In some embodiments, the first ActRIIB polypeptide comprises a glutamic acid at the amino acid position corresponding to 55 of SEQ ID NO: 2. In some embodiments, the second ActRIIB polypeptide does not comprise a glutamic acid at the amino acid position corresponding to 55 of SEQ ID NO: 2. In some embodiments, the second ActRIIB polypeptide comprises a lysine at the amino acid position corresponding to 55 of SEQ ID NO: 2. In some embodiments, the first ActRIIB polypeptide comprises one or more amino acid substitutions at the amino acid positions corresponding to any one of F82, L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the second ActRIIB polypeptide comprises one or more amino acid substitutions at the amino acid positions corresponding to any one of F82, L79, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the first ActRIIB polypeptide and/or the second ActRIIB polypeptide comprise one or more amino acid modification that promote heteromultimer formation. In some embodiments, the first ActRIIB polypeptide and/or the second ActRIIB polypeptide comprise one or more amino acid modification that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 39, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 5, wherein the first ActRIIB polypeptide does not comprise the amino acid sequence of the second ActRIIB polypeptide. In some embodiments, the first ActRIIB polypeptide comprises an isoleucine at the amino acid position corresponding to 82 of SEQ ID NO: 2. In some embodiments, the second ActRIIB polypeptide does not comprise an isoleucine acid at the amino acid position corresponding to 82 of SEQ ID NO: 2. In some embodiments, the second ActRIIB polypeptide comprises a phenylalanine at the amino acid position corresponding to 82 of SEQ ID NO: 2. In some embodiments, the first ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, and D80R. In some embodiments, the second ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, and D80R. In some embodiments, the first ActRIIB polypeptide and/or the second ActRIIB polypeptide comprise one or more amino acid modifications that promote heteromultimer formation. In some embodiments, the first ActRIIB polypeptide and/or the second ActRIIB polypeptide comprise one or more amino acid modification that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 42, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 5, wherein the first ActRIIB polypeptide does not comprise the amino acid sequence of the second ActRIIB polypeptide. In some embodiments, first ActRIIB polypeptide comprises a lysine at the amino acid position corresponding to 82 of SEQ ID NO: 2. In some embodiments, the second ActRIIB polypeptide does not comprise a lysine acid at the amino acid position corresponding to 82 of SEQ ID NO: 2. In some embodiments, the second ActRIIB polypeptide comprises a phenylalanine at the amino acid position corresponding to 82 of SEQ ID NO: 2. In some embodiments, the first ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, and D80R. In some embodiments, the second ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, and D80R. In some embodiments, the first ActRIIB polypeptide and/or the second ActRIIB polypeptide comprise one or more amino acid modifications that promote heteromultimer formation. In some embodiments, the first ActRIIB polypeptide and/or the second ActRIIB polypeptide comprise one or more amino acid modifications that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 45, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 48, wherein the first ActRIIB polypeptide does not comprise the amino acid sequence of the second ActRIIB polypeptide. In some embodiments, the first ActRIIB polypeptide comprises an acidic amino acid position corresponding to 79 of SEQ ID NO: 2. In some embodiments, the acidic amino acid is an aspartic acid. In some embodiments, the acidic amino acid is a glutamic acid. In some embodiments, the second ActRIIB polypeptide does not comprise an acidic acid (e.g., aspartic acid or glutamic acid) at the amino acid position corresponding to 79 of SEQ ID NO: 2. In some embodiments, the second ActRIIB polypeptide comprises a leucine at the amino acid position corresponding to 79 of SEQ ID NO: 2. In some embodiments, the first ActRIIB polypeptide comprises one or more amino acid substitutions at the amino acid positions corresponding to any one of F82, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the second ActRIIB polypeptide comprises one or more amino acid substitutions at the amino acid positions corresponding to any one of F82, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the first ActRIIB polypeptide and/or the second ActRIIB polypeptide comprise one or more amino acid modifications that promote heteromultimer formation. In some embodiments, the first ActRIIB polypeptide and/or the second ActRIIB polypeptide comprise one or more amino acid modifications that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 50, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 52, wherein the first ActRIIB polypeptide does not comprise the amino acid sequence of the second ActRIIB polypeptide. In some embodiments, the first ActRIIB polypeptide comprises an acidic amino acid position corresponding to 79 of SEQ ID NO: 2. In some embodiments, the acidic amino acid is an aspartic acid. In some embodiments, the acidic amino acid is a glutamic acid. In some embodiments, the second ActRIIB polypeptide does not comprise an acidic acid (e.g., aspartic acid or glutamic acid) at the amino acid position corresponding to 79 of SEQ ID NO: 2. In some embodiments, the second ActRIIB polypeptide comprises a leucine at the amino acid position corresponding to 79 of SEQ ID NO: 2. In some embodiments, the first ActRIIB polypeptide comprises one or more amino acid substitutions at the amino acid positions corresponding to any one of F82, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the second ActRIIB polypeptide comprises one or more amino acid substitutions at the amino acid positions corresponding to any one of F82, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, P129S, P130A, P130R, E37A, R40A, D54A, R56A, K74F, K74I, K74Y, W78A, D80A, D80F, D80G, D80I, D80K, D80M, D80M, D80N, D80R, and F82A.

In certain aspects, the present disclosure relates to heteromultimers comprising one or more ALK4 receptor polypeptides (e.g., SEQ ID Nos: 84, 85, 86, 87, 88, 89, 92, 93, 247, 249, 421, 422 and variants thereof) and one or more ActRIIB receptor polypeptides (e.g., SEQ ID NOs: 1, 2, 5, 6, 12, 31, 33, 34, 36, 37, 39, 40, 42, 43, 45, 46, 48, 49, 50, 51, 52, 53, 276, 278, 279, 332, 333, 335, 336, 338, 339, 341, 342, 344, 345, 347, 348, 350, 351, 353, 354, 356, 357, 385, 386, 387, 388, 389, 396, 398, 402, 403, 406, 408, 409 and variants thereof), including uses thereof (e.g. treating heart failure in a patient in need thereof), which are generally referred to herein as “ActRIIB:ALK4 heteromultimer” or “ActRIIB-ALK4 heteromultimers”, including uses thereof (e.g., treating heart failure in a patient in need thereof). Preferably, ActRIIB:ALK4 heteromultimers are soluble [e.g., a heteromultimer complex comprises a soluble portion (domain) of an ALK4 receptor and a soluble portion (domain) of an ActRIIB receptor]. In general, the extracellular domains of ALK4 and ActRIIB correspond to soluble portions of these receptors. Therefore, in some embodiments, ActRIIB:ALK4 heteromultimers comprise an extracellular domain of an ALK4 receptor and an extracellular domain of an ActRIIB receptor. In some embodiments, ActRIIB:ALK4 heteromultimers inhibit activity (e.g., Smad signaling) of one or more ActRII-ALK4 ligands (e.g., activin A, activin B, GDF8, GDF11, BMP6, BMP10). In some embodiments, ActRIIB:ALK4 heteromultimers bind to one or more ActRII-ALK4 ligands (e.g., activin A, activin B, GDF8, GDF11, BMP6, BMP10). In some embodiments, ActRIIB:ALK4 heteromultimers comprise at least one ALK4 polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 84, 85, 86, 87, 88, 89, 92, 93, 247, 249, 421, and 422. In some embodiments, ActRIIB:ALK4 heteromultimer complexes of the disclosure comprise at least one ALK4 polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to a portion of ALK4 beginning at a residue corresponding to any one of amino acids 24-34, 25-34, or 26-34 of SEQ ID NO: 84 and ending at a position from 101-126, 102-126, 101-125, 101-124, 101-121, 111-126, 111-125, 111-124, 121-126, 121-125, 121-124, or 124-126 of SEQ ID NO: 84. In some embodiments, ActRIIB:ALK4 heteromultimers comprise at least one ALK4 polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to amino acids 34-101 with respect to SEQ ID NO: 84. In some embodiments, ActRIIB-ALK4 heteromultimers comprise at least one ActRIIB polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of any one of SEQ ID NOs: 2, 5, 6, 12, 31, 33, 34, 36, 37, 39, 40, 42, 43, 45, 46, 48, 49, 50, 51, 52, 53, 276, 278, 279, 332, 333, 335, 336, 338, 339, 341, 342, 344, 345, 347, 348, 350, 351, 353, 354, 356, 357, 385, 386, 387, 388, 389, 396, 398, 402, 403, 406, 408, and 409. In some embodiments, ActRIIB:ALK4 heteromultimer complexes of the disclosure comprise at least one ActRIIB polypeptide that comprises, consists essentially of, consists of a sequence that is at least 70%, 5% 80%, 85%, 86%, 87%, 88%, 89%, 90%, 91%, 92%, 93%, 94% 95%, 97%, 98%, 99%, or 100% identical to a portion of ActRIIB beginning at a residue corresponding to any one of amino acids 20-29, 20-24, 21-24, 22-25, or 21-29 and end at a position from 109-134, 119-134, 119-133, 129-134, or 129-133 of SEQ ID NO: 2. In some embodiments, ActRIIB:ALK4 heteromultimers comprise at least one ActRIIB polypeptide that comprises, consists essentially of, consists of a sequence that is at least 70%, 75%, 80%, 85%, 86%, 87%, 88%, 89%, 90%, 91%, 92%, 93%, 94% 95%, 97% 98%, 99%, or 100% identical to amino acids 29-109 of SEQ ID NO: 2. In some embodiments, ActRIIB:ALK4 heteromultimers comprise at least one ActRIIB polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to amino acids 25-131 of SEQ ID NO: 2. In certain embodiments, ActRIIB:ALK4 heteromultimer complexes of the disclosure comprise at least one ActRIIB polypeptide wherein the position corresponding to L79 of SEQ ID NO: 2 is not an acidic amino acid (i.e., not naturally occurring D or E amino acid residues or an artificial acidic amino acid residue). ActRIIB:ALK4 heteromultimers of the disclosure include, e.g., heterodimers, heterotrimers, heterotetramers and further higher order oligomeric structures. See, e.g., FIGS. 11-13, which may also be applied to ActRII:ALK7 oligomeric structures. In certain embodiments, heteromultimer complexes of the disclosure are ActRIIB:ALK7 heterodimers.

In certain embodiments, the disclosure relates to a heteromultimer comprising at least one ALK7-Fc fusion polypeptide and at least one ActRIIB-Fc fusion polypeptide. In some embodiments, an ActRIIB-Fc:ALK7-Fc heteromultimer binds to one or more ActRII-ALK4 ligands (e.g., activin A, activin B, GDF8, GDF11, BMP6, BMP10). In some embodiments, an ActRIIB-Fc:ALK7-Fc heteromultimer inhibits signaling of one or more ActRII-ALK4 (e.g., activin A, activin B, GDF8, GDF11, BMP6, BMP10). In some embodiments, an ActRIIB-Fc:ALK7-Fc heteromultimer is a heterodimer.

In certain embodiments, the disclosure relates to heteromultimers that comprise at least one ALK7 polypeptide, which includes fragments, functional variants, and modified forms thereof. Preferably, ALK7 polypeptides for use as disclosed herein (e.g., heteromultimers comprising an ALK7 polypeptide and uses thereof) are soluble (e.g., an extracellular domain of ALK7). In other embodiments, ALK7 polypeptides for use as disclosed herein bind to and/or inhibit (antagonize) activity (e.g., induction of Smad signaling) of one or more ActRII-ALK4 ligands (e.g., activin A, activin B, GDF8, GDF11, BMP6, BMP10) superfamily ligands. In some embodiments, the ALK7-Fc fusion polypeptide comprises an ALK7 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21-28 (e.g., amino acid residues 21, 22, 23, 24, 25, 26, 27, and 28) SEQ ID NO: 120, 121, or 122, and ends at any one of amino acids 92-113 (e.g., amino acid residues 92, 93, 94, 95, 96, 97, 98, 99, 100, 101, 102, 103, 104, 105, 106, 107, 108, 109, 110, 111, 112, and 113) of SEQ ID NO: 120, 121, or 122. In some embodiments, the ALK7-Fc fusion polypeptide comprises an ALK7 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 28-92 of SEQ ID NOs: 120, 121, or 122. In some embodiments, the ALK7-Fc fusion polypeptide comprises an ALK7 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 21-113 of SEQ ID NOs: 120, 121, or 122. In some embodiments, the ALK7-Fc fusion polypeptide comprises an ALK7 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of any one of SEQ ID Nos: 120, 123, 124, 125, 121, 126, 122, 127, 128, 129, 130, 131, 132, 133, or 134. In some embodiments, heteromultimers of the disclosure consist or consist essentially of at least one ALK7 polypeptide that is at least 70%, 75%, 80%, 85%, 90%, 91%, 92%, 93%, 94%, 95%, 96%, 97%, 98%, 99%, or 100% identical to the amino acid sequence of SEQ ID NO: 120, 121, 122, 123, 124, 125, 126, 127, 128, 129, 130, 133, or 134.

In certain aspects, the present disclosure relates to heteromultimer complexes comprising one or more ALK7 receptor polypeptides (e.g., SEQ ID Nos: 120, 121, 122, 123, 124, 125, 126, 127, 128, 129, 130, 133, 134 and variants thereof) and one or more ActRIIB receptor polypeptides (e.g., SEQ ID NOs: 1, 2, 5, 6, 12, 31, 33, 34, 36, 37, 39, 40, 42, 43, 45, 46, 48, 49, 50, 51, 52, 53, 276, 278, 279, 332, 333, 335, 336, 338, 339, 341, 342, 344, 345, 347, 348, 350, 351, 353, 354, 356, 357, 385, 386, 387, 388, 389, 396, 398, 402, 403, 406, 408, 409 and variants thereof), which are generally referred to herein as “ActRIIB:ALK7 heteromultimer” or “ActRIIB-ALK7 heteromultimers”, including uses thereof (e.g., treating heart failure in a patient in need thereof). Preferably, ActRIIB-ALK7 heteromultimers are soluble [e.g., a heteromultimer complex comprises a soluble portion (domain) of an ALK7 receptor and a soluble portion (domain) of an ActRIIB receptor]. In general, the extracellular domains of ALK7 and ActRIIB correspond to soluble portions of these receptors. Therefore, in some embodiments, ActRIIB-ALK7 heteromultimers comprise an extracellular domain of an ALK7 receptor and an extracellular domain of an ActRIIB receptor. In some embodiments, ActRIIB-ALK7 heteromultimers inhibit activity (e.g., Smad signaling) of one or more ActRII-ALK4 ligands (e.g., activin A, activin B, GDF8, GDF11, BMP6, BMP10). In some embodiments, ActRIIB-ALK7 heteromultimers bind to one or more ActRII-ALK4 ligands (e.g., activin A, activin B, GDF8, GDF11, BMP6, BMP10). In some embodiments, ActRIIB-ALK7 heteromultimers comprise at least one ALK7 polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 120, 121, 122, 123, 124, 125, 126, 127, 128, 129, 130, 133, and 134. In some embodiments, ActRIIB-ALK7 heteromultimers comprise at least one ActRIIB polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of any one of SEQ ID NOs: 2, 5, 6, 12, 31, 33, 34, 36, 37, 39, 40, 42, 43, 45, 46, 48, 49, 50, 51, 52, 53, 276, 278, 279, 332, 333, 335, 336, 338, 339, 341, 342, 344, 345, 347, 348, 350, 351, 353, 354, 356, 357, 385, 386, 387, 388, 389, 396, 398, 402, 403, 406, 408, and 409. In some embodiments, ActRIIB-ALK7 heteromultimer complexes of the disclosure comprise at least one ActRIIB polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to a portion of ActRIIB beginning at a residue corresponding to any one of amino acids 20-29, 20-24, 21-24, 22-25, or 21-29 and end at a position from 109-134, 119-134, 119-133, 129-134, or 129-133 of SEQ ID NO: 2. In some embodiments, ActRIIB-ALK7 heteromultimers comprise at least one ActRIIB polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to amino acids 29-109 of SEQ ID NO: 2. In some embodiments, ActRIIB-ALK7 heteromultimers comprise at least one ActRIIB polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to amino acids 25-131 of SEQ ID NO: 2. In certain embodiments, ActRIIB-ALK7 heteromultimer complexes of the disclosure comprise at least one ActRIIB polypeptide wherein the position corresponding to L79 of SEQ ID NO: 2 is not an acidic amino acid (i.e., not naturally occurring D or E amino acid residues or an artificial acidic amino acid residue). ActRIIB-ALK7 heteromultimers of the disclosure include, e.g., heterodimers, heterotrimers, heterotetramers and further higher order oligomeric structures. See, e.g., FIGS. 11-13, which may also be applied to both ActRII-ALK4 and ActRII-ALK7 oligomeric structures. In certain embodiments, heteromultimer complexes of the disclosure are ActRIIB-ALK7 heterodimers.

In certain aspects, the present disclosure relates to heteromultimer complexes comprising one or more ALK7 receptor polypeptides (e.g., SEQ ID Nos: 120, 121, 122, 123, 124, 125, 126, 127, 128, 129, 130, 133, 134 and variants thereof) and one or more ActRIIA receptor polypeptides (e.g., SEQ ID NOs: 364, 366, 367, 368, 369, 378, 380, 381, 384 and variants thereof), which are generally referred to herein as “ActRIIA:ALK7 heteromultimer” or “ActRIIA-ALK7 heteromultimers”, including uses thereof (e.g., treating heart failure in a patient in need thereof). Preferably, ActRIIA-ALK7 heteromultimers are soluble [e.g., a heteromultimer complex comprises a soluble portion (domain) of an ALK7 receptor and a soluble portion (domain) of an ActRIIA receptor]. In general, the extracellular domains of ALK7 and ActRIIA correspond to soluble portions of these receptors. Therefore, in some embodiments, ActRIIA-ALK7 heteromultimers comprise an extracellular domain of an ALK7 receptor and an extracellular domain of an ActRIIA receptor. In some embodiments, ActRIIA-ALK7 heteromultimers inhibit activity (e.g., Smad signaling) of one or more ActRII-ALK4 ligands (e.g., activin A, activin B, GDF8, GDF11, BMP6, BMP10). In some embodiments, ActRIIA-ALK7 heteromultimers bind to one or more ActRII-ALK4 ligands (e.g., activin A, activin B, GDF8, GDF11, BMP6, BMP10). In some embodiments, ActRIIA-ALK7 heteromultimers comprise at least one ALK7 polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 120, 121, 122, 123, 124, 125, 126, 127, 128, 129, 130, 133, and 134. In some embodiments, ActRIIA-ALK7 heteromultimers comprise at least one ActRIIA polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of any one of SEQ ID NOs: 364, 366, 367, 368, 369, 378, 380, 381, 384. In certain embodiments, heteromultimer complexes of the disclosure are ActRIIA-ALK7 heterodimers.

In certain aspects, the present disclosure relates to heteromultimer complexes comprising one or more ALK4 receptor polypeptides (e.g., SEQ ID Nos: 84, 85, 86, 87, 88, 89, 92, 93, 247, 249, 421, 422 and variants thereof) and one or more ActRIIA receptor polypeptides (e.g., SEQ ID NOs: 364, 366, 367, 368, 369, 378, 380, 381, 384 and variants thereof), which are generally referred to herein as “ActRIIA:ALK4 heteromultimer” or “ActRIIA-ALK4 heteromultimers”, including uses thereof (e.g., treating heart failure in a patient in need thereof). Preferably, ActRIIA-ALK4 heteromultimers are soluble [e.g., a heteromultimer complex comprises a soluble portion (domain) of an ALK4 receptor and a soluble portion (domain) of an ActRIIA receptor]. In general, the extracellular domains of ALK4 and ActRIIA correspond to soluble portions of these receptors. Therefore, in some embodiments, ActRIIA-ALK4 heteromultimers comprise an extracellular domain of an ALK4 receptor and an extracellular domain of an ActRIIA receptor. In some embodiments, ActRIIA-ALK4 heteromultimers inhibit activity (e.g., Smad signaling) of one or more ActRII-ALK4 ligands (e.g., activin A, activin B, GDF8, GDF11, BMP6, BMP10). In some embodiments, ActRIIA-ALK4 heteromultimers bind to one or more ActRII-ALK4 ligands (e.g., activin A, activin B, GDF8, GDF11, BMP6, BMP10). In some embodiments, ActRIIA-ALK4 heteromultimers comprise at least one ALK4 polypeptide that comprises, consists essentially of, or consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of SEQ ID NO: 84, 85, 86, 87, 88, 89, 92, 93, 247, 249, 421, and 422. In some embodiments, ActRIIA-ALK4 heteromultimer complexes of the disclosure comprise at least one ALK4 polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to a portion of ALK4 beginning at a residue corresponding to any one of amino acids 24-34, 25-34, or 26-34 of SEQ ID NO: 84 and ending at a position from 101-126, 102-126, 101-125, 101-124, 101-121, 111-126, 111-125, 111-124, 121-126, 121-125, 121-124, or 124-126 of SEQ ID NO: 84. In some embodiments, ActRIIA-ALK4 heteromultimers comprise at least one ALK4 polypeptide that comprises, consists essentially of, consists of a sequence that is at least 70%, 75%, 80%, 85%, 86%, 87%, 88%, 89%, 90%, 91%, 92%, 93%, 94% 95%, 97% 98%, 99%, or 100% identical to amino acids 34-101 with respect to SEQ ID NO: 84. In some embodiments, ActRIIA-ALK4 heteromultimers comprise at least one ActRIIA polypeptide that comprises, consists essentially of, consists of a sequence that is at least 70%, 75%, 80%, 85%, 86%, 87%, 88%, 89%, 90%, 91%, 92%, 93%, 94% 95%, 97%, 98%, 99%, or 100% identical to the amino acid sequence of any one of SEQ ID NOs: 364, 366, 367, 368, 369, 378, 380, 381, 384. In certain embodiments, heteromultimer complexes of the disclosure are ActRIIA-ALK4 heterodimers.

In certain embodiments, the disclosure relates to a heteromultimer comprising a first ActRIIA-Fc fusion polypeptide and a second ActRIIA-Fc fusion polypeptide, wherein the second variant ActRIIA-Fc fusion polypeptide differs from that present in the first polypeptide. In some embodiments, an ActRIIA-Fc:ActRIIA-Fc heteromultimers binds to one or more ActRII-ALK4 ligands (e.g., activin A, activin B, GDF8, GDF11, BMP6, BMP10). In some embodiments, an ActRIIA-Fc:ActRIIA-Fc heteromultimers inhibit signaling of one or more ActRII-ALK4 ligands (e.g., activin A, activin B, GDF8, GDF11, BMP6, BMP10). In some embodiments, an ActRIIA-Fc:ActRIIA-Fc heteromultimers is a heterodimer.

II. Linkers

The disclosure provides for an ActRII-ALK4 ligand trap polypeptide (e.g., ActRIIB, ActRIIA, ALK4, ALK7, and follistatin polypeptides including variants thereof) that may be fused to an additional polypeptide disclosed herein including, for example, fused to a heterologous portion (e.g., an Fc portion). In these embodiments, the polypeptide portion (e.g., ActRIIB, ActRIIA, ALK4, ALK7, and follistatin polypeptides including variants thereof) is connected to the additional polypeptide (e.g., a heterologous portion such as an Fc domain) by means of a linker. In some embodiments, the linkers are glycine and serine rich linkers. In some embodiments, the linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 261), GGGG (SEQ ID NO: 262), TGGGG (SEQ ID NO: 263), SGGGG (SEQ ID NO: 264), TGGG (SEQ ID NO: 265), or SGGG (SEQ ID NO: 266) singlets, or repeats. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 25, 22 or 20 amino acids. In some embodiments, the linker is 10-50, 10-40, 10-30, 10-25, 10-21, 10-15, 10, 15-25, 17-22, 20, or 21 amino acids in length. In some embodiments, the linker comprises the amino acid sequence GlyGlyGlyGlySer (GGGGS) (SEQ ID NO: 267), or repetitions thereof (GGGGS)n, where n≥2. In particular embodiments n≥3, or n=3-10. In some embodiments, n≥4, or n=4-10. In some embodiments, n is not greater than 4 in a (GGGGS)n linker. In some embodiments, n=4-10, 4-9, 4-8, 4-7, 4-6, 4-5, 5-8, 5-7, or 5-6. In some embodiments, n=3, 4, 5, 6, or 7. In particular embodiments, n=4. In some embodiments, a linker comprising a (GGGGS)n sequence also comprises an N-terminal threonine. In some embodiments, the linker is any one of the following:

(SEQ ID NO: 268)
GGGGGGGGS
(SEQ ID NO: 269)
TGGGGSGGGGS
(SEQ ID NO: 270)
TGGGGSGGGGSGGGGS
(SEQ ID NO: 271)
TGGGGSGGGGSGGGGSGGGGS
(SEQ ID NO: 272)
TGGGGSGGGGSGGGGSGGGGSGGGGS
(SEQ ID NO: 273)
TGGGGSGGGGSGGGGSGGGGSGGGGSGGGGS
or
(SEQ ID NO: 274)
TGGGGSGGGGSGGGGSGGGGSGGGGSGGGGSGGGGS.

In some embodiments, the linker comprises the amino acid sequence of TGGGPKSCDK (SEQ ID NO: 275). In some embodiments, the linker is any one of SEQ ID NOs: 268-275 lacking the N-terminal threonine. In some embodiments, the linker does not comprise the amino acid sequence of SEQ ID NO: 273 or 274.

In some embodiments, a polypeptide described (e.g., ActRIIB, ActRIIA, ALK4, ALK7, and follistatin, polypeptides including variants thereof) herein may include a polypeptide fused to a moiety by way of a linker. In some embodiments, the moiety increases stability of the polypeptide. In some embodiments, the moiety is selected from the group consisting of an Fc domain monomer, a wild-type Fc domain, an Fc domain with amino acid substitutions (e.g., one or more substitutions that reduce dimerization), an albumin-binding peptide, a fibronectin domain, and a human serum albumin. Suitable peptide linkers are known in the art, and include, for example, peptide linkers containing flexible amino acid residues such as glycine, alanine, and serine. In some embodiments, a linker can contain motifs, e.g., multiple or repeating motifs, of GA, GS, GG, GGA, GGS, GGG (SEQ ID NO: 261), GGGA (SEQ ID NO: 280), GGGS (SEQ ID NO: 281), GGGG (SEQ ID NO: 262), GGGGA (SEQ ID NO: 282), GGGGS (SEQ ID NO: 267), GGGGG (SEQ ID NO: 283), GGAG (SEQ ID NO: 284), GGSG (SEQ ID NO: 285), AGGG (SEQ ID NO: 286), or SGGG (SEQ ID NO: 266). In some embodiments, a linker can contain 2 to 12 amino acids including motifs of GA or GS, e.g., GA, GS, GAGA (SEQ ID NO: 287), GSGS (SEQ ID NO: 288), GAGAGA (SEQ ID NO: 289), GSGSGS (SEQ ID NO: 290), GAGAGAGA (SEQ ID NO: 291), GSGSGSGS (SEQ ID NO: 292), GAGAGAGAGA (SEQ ID NO: 293), GSGSGSGSGS (SEQ ID NO: 294), GAGAGAGAGAGA (SEQ ID NO: 295), and GSGSGSGSGSGS (SEQ ID NO: 296). In some embodiments, a linker can contain 3 to 12 amino acids including motifs of GGA or GGS, e.g., GGA, GGS, GGAGGA (SEQ ID NO: 297), GGSGGS (SEQ ID NO: 298), GGAGGAGGA (SEQ ID NO: 299), GGSGGSGGS (SEQ ID NO: 300), GGAGGAGGAGGA (SEQ ID NO: 301), and GGSGGSGGSGGS (SEQ ID NO: 302). In some embodiments, a linker can contain 4 to 12 amino acids including motifs of GGAG (SEQ ID NO: 303), GGSG (SEQ ID NO: 304), GGAGGGAG (SEQ ID NO: 305), GGSGGGSG (SEQ ID NO: 306), GGAGGGAGGGAG (SEQ ID NO: 307), and GGSGGGSGGGSG (SEQ ID NO: 308). In some embodiments, a linker can contain motifs of GGGGA (SEQ ID NO: 309) or GGGGS (SEQ ID NO: 267), e.g., GGGGAGGGGAGGGGA (SEQ ID NO: 310) and GGGGSGGGGSGGGGS (SEQ ID NO: 311). In some embodiments, an amino acid linker between a moiety (e.g., an Fc domain monomer, a wild-type Fc domain, an Fc domain with amino acid substitutions (e.g., one or more substitutions that reduce dimerization), an albumin-binding peptide, a fibronectin domain, or a human serum albumin) and a polypeptide (e.g., ActRIIB, ActRIIA, ALK4, ALK7, and follistatin polypeptides including variants thereof) may be GGG, GGGA (SEQ ID NO: 280), GGGG (SEQ ID NO: 262), GGGAG (SEQ ID NO: 312), GGGAGG (SEQ ID NO: 313), or GGGAGGG (SEQ ID NO: 314).

In some embodiments, a linker can also contain amino acids other than glycine, alanine, and serine, e.g., AAAL (SEQ ID NO: 315), AAAK (SEQ ID NO: 316), AAAR (SEQ ID NO: 317), EGKSSGSGSESKST (SEQ ID NO: 318), GSAGSAAGSGEF (SEQ ID NO: 319), AEAAAKEAAAKA (SEQ ID NO: 320), KESGSVSSEQLAQFRSLD (SEQ ID NO: 321), GENLYFQSGG (SEQ ID NO: 322), SACYCELS (SEQ ID NO: 323), RSIAT (SEQ ID NO: 324), RPACKIPNDLKQKVMNH (SEQ ID NO: 325), GGSAGGSGSGSSGGSSGASGTGTAGGTGSGSGTGSG (SEQ ID NO: 326), AAANSSIDLISVPVDSR (SEQ ID NO: 327), or GGSGGGSEGGGSEGGGSEGGGSEGGGSEGGGSGGGS (SEQ ID NO: 328). In some embodiments, a linker can contain motifs, e.g., multiple or repeating motifs, of EAAAK (SEQ ID NO: 329). In some embodiments, a linker can contain motifs, e.g., multiple or repeating motifs, of praline-rich sequences such as (XP)n, in which X may be any amino acid (e.g., A, K, or E) and n is from 1-5, and PAPAP(SEQ ID NO: 330).

The length of the peptide linker and the amino acids used can be adjusted depending on the two polypeptides involved and the degree of flexibility desired in the final polypeptide fusion polypeptide. The length of the linker can be adjusted to ensure proper polypeptide folding and avoid aggregate formation.

H) Polypeptide Variants and Modifications

In part, the disclosure relates to ActRII-ALK4 antagonists that are variant polypeptides (e.g., a variant ActRIIA, ActRIIB, ALK4, ALK7, or follistatin polypeptide). Variant polypeptides of the disclosure included, for example, variant polypeptides produced by one or more amino acid substitutions, deletions, additions or combinations thereof as well as variants of one or more post-translational modifications (e.g., including, but are not limited to, acetylation, carboxylation, glycosylation, phosphorylation, lipidation, and acylation). Methods for generating variant polypeptides comprising one or more amino acid modifications, particularly methods for generating variant polypeptides that have one or more desired properties, are described herein or otherwise well known in the art. Likewise, various methods for determining if a variant polypeptide has retained or developed one or more desired properties (e.g., alterations in ligand binding and/or antagonistic activities) are described herein or otherwise well known in the art. These methods can be used to generate variant polypeptides (e.g., variant ActRIIA, ActRIIB, ALK4, ALK7, or follistatin polypeptides) as well as validate their activity (or other properties) as described here.

As described above, the disclosure provides polypeptides (e.g., ActRIIA, ActRIIB, ALK4, ALK7, or follistatin polypeptides) sharing a specified degree of sequence identity or similarity to a naturally occurring polypeptide. To determine the percent identity of two amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues at corresponding amino acid positions are then compared. When a position in the first sequence is occupied by the same amino acid residue as the corresponding position in the second sequence, then the molecules are identical at that position (as used herein amino acid “identity” is equivalent to amino acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

In one embodiment, the percent identity between two amino acid sequences is determined using the Needleman and Wunsch (J Mol. Biol. (48):444-453 (1970)) algorithm which has been incorporated into the GAP program in the GCG software package (available at http://www.gcg.com). In a specific embodiment, the following parameters are used in the GAP program: either a Blosum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (Devereux, J., et al., Nucleic Acids Res. 12(1):387 (1984)) (available at http://www.gcg.com). Exemplary parameters include using a NWSgapdna.CMP matrix and a gap weight of 40, 50, 60, 70, or 80 and a length weight of 1, 2, 3, 4, 5, or 6. Unless otherwise specified, percent identity between two amino acid sequences is to be determined using the GAP program using a Blosum 62 matrix, a GAP weight of 10 and a length weight of 3, and if such algorithm cannot compute the desired percent identity, a suitable alternative disclosed herein should be selected.

In another embodiment, the percent identity between two amino acid sequences is determined using the algorithm of E. Myers and W. Miller (CABIOS, 4:11-17 (1989)) which has been incorporated into the ALIGN program (version 2.0), using a PAM120 weight residue table, a gap length penalty of 12 and a gap penalty of 4.

Another embodiment for determining the best overall alignment between two amino acid sequences can be determined using the FASTDB computer program based on the algorithm of Brutlag et al. (Comp. App. Biosci., 6:237-245 (1990)). In a sequence alignment the query and subject sequences are both amino acid sequences. The result of said global sequence alignment is presented in terms of percent identity. In one embodiment, amino acid sequence identity is performed using the FASTDB computer program based on the algorithm of Brutlag et al. (Comp. App. Biosci., 6:237-245 (1990)). In a specific embodiment, parameters employed to calculate percent identity and similarity of an amino acid alignment comprise: Matrix=PAM 150, k-tuple=2, Mismatch Penalty=1, Joining Penalty=20, Randomization Group Length=0, Cutoff Score=1, Gap Penalty=5 and Gap Size Penalty=0.05.

In some embodiments, the disclosure contemplates making functional variant polypeptides by modifying the structure of a polypeptide (e.g., an ActRIIA, ActRIIB, ALK4, ALK7, or follistatin polypeptide) for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, or to bind to one or more ActRII-ALK4 ligands including, for example, activin A, activin B, GDF8, GDF11, BMP6, and BMP10.

In certain embodiments, the disclosure contemplates specific mutations of a polypeptide (e.g., an ActRIIA, ActRIIB, ALK4, ALK7, or follistatin polypeptide) so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, polypeptides of the present disclosure for use in humans may be expressed in a mammalian cell line that provides proper glycosylation, such as HEK293 or CHO cell lines, although other mammalian expression cell lines are expected to be useful as well. In some embodiments, polypeptides of the disclosure (e.g., an ActRIIA, ActRIIB, ALK4, ALK7, or follistatin polypeptides) are glycosylated and have a glycosylation pattern obtainable from of the polypeptide in a CHO cell.

The disclosure further contemplates a method of generating mutants, particularly sets of combinatorial mutants of a polypeptide (e.g., an ActRIIA, ActRIIB, ALK4, ALK7, or follistatin polypeptide) as well as truncation mutants. Pools of combinatorial mutants are especially useful for identifying functionally active (e.g., ActRII-ALK4 ligand binding) sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, polypeptide (e.g., an ActRIIA, ActRIIB, ALK4, ALK7, or follistatin polypeptide) variants, homomultimers, and heteromultimers comprising the same, may be screened for ability to bind to one or more ActRII-ALK4 ligands (e.g., activin A, activin B, GDF8, GDF11, BMP6, BMP10), to prevent binding of an ActRII-ALK4 ligand to an ActRII and/or ALK4 polypeptide, as well as homomultimers of heteromultimers thereof, and/or to interfere with signaling caused by an ActRII-ALK4 ligand.

The activity of a polypeptide (e.g., an ActRIIA, ActRIIB, ALK4, ALK7, or follistatin polypeptide), including homomultimers and heteromultimers thereof, or variant thereof may also be tested in a cell-based or in vivo assay. For example, the effect of a polypeptide, including homomultimers and heteromultimers thereof, or a variant thereof on the expression of genes involved in heart failure pathogenesis assessed. This may, as needed, be performed in the presence of one or more recombinant ligand proteins (e.g., activin A, activin B, GDF8, GDF11, BMP6, BMP10), and cells may be transfected so as to produce polypeptide (e.g., an ActRIIA, ActRIIB, ALK4, ALK7, or follistatin polypeptide) r, and optionally, an ActRII-ALK4 ligand. Likewise, a polypeptide, including homomultimers and heteromultimers thereof, or a variant thereof may be administered to a mouse or other animal and effects on heart failure pathogenesis may be assessed using art-recognized methods. Similarly, the activity of a polypeptide, including homomultimers and heteromultimers thereof, or variant thereof may be tested in blood cell precursor cells for any effect on growth of these cells, for example, by the assays as described herein and those of common knowledge in the art. A SMAD-responsive reporter gene may be used in such cell lines to monitor effects on downstream signaling.

In certain aspects, a polypeptide (e.g., an ActRIIA, ActRIIB, ALK4, ALK7, or follistatin polypeptide), including heteromultimers or homomultimers thereof, of the disclosure bind to one or more ActRII-ALK4 ligands. In some embodiments, a polypeptide, including heteromultimers or homomultimers thereof, of the disclosure bind to one or more ActRII-ALK4 ligands with a K_{D} of at least 1×10⁻⁷ M. In some embodiments, the one or more ActRII-ALK4 ligands is selected from the group consisting of: activin A, activin B, GDF8, GDF11, and BMP10.

In certain aspects, a polypeptide (e.g., an ActRIIA, ActRIIB, ALK4, ALK7, or follistatin polypeptide), including heteromultimers or homomultimers thereof, of the disclosure inhibits one or more ActRII-ALK4 family ligands. In some embodiments, a polypeptide, including heteromultimers or homomultimers thereof, of the disclosure inhibits signaling of one or more ActRII-ALK4 ligands. In some embodiments, a polypeptide, including heteromultimers or homomultimers thereof, of the disclosure inhibits Smad signaling of one or more ActRII-ALK4 ligands. In some embodiments, a polypeptide, including heteromultimers or homomultimers thereof, of the disclosure inhibits signaling of one or more ActRII-ALK4 ligands in a cell-based assay. In some embodiments, a polypeptide, including heteromultimers or homomultimers thereof, of the disclosure inhibits one or more ActRII-ALK4 ligands selected from the group consisting of: activin A, activin B, GDF8, GDF11, and BMP10.

Combinatorial-derived variants can be generated which have increased selectivity or generally increased potency relative to a reference polypeptide (e.g., an ActRIIA, ActRIIB, ALK4, ALK7, or follistatin polypeptide), including homomultimers and heteromultimers thereof. Such variants, when expressed from recombinant DNA constructs, can be used in gene therapy protocols. Likewise, mutagenesis can give rise to variants which have intracellular half-lives dramatically different than the corresponding unmodified a polypeptide, including homomultimers and heteromultimers thereof. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter the half-life of the polypeptide, including homomultimers and heteromultimers thereof.

A combinatorial library may be produced by way of a degenerate library of genes encoding a library of polypeptides which each include at least a portion of a polypeptide (e.g., an ActRIIA, ActRIIB, ALK4, ALK7, or follistatin polypeptide), including homomultimers and heteromultimers thereof. For instance, a mixture of synthetic oligonucleotides can be enzymatically ligated into gene sequences such that the degenerate set of potential ActRIIA, ActRIIB, ALK4, ALK7, or follistatin encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art [Narang, SA (1983) Tetrahedron 39:3; Itakura et al. (1981) Recombinant DNA, Proc. 3rd Cleveland Sympos. Macromolecules, ed. AG Walton, Amsterdam: Elsevier pp 273-289; Itakura et al. (1984) Annu. Rev. Biochem. 53:323; Itakura et al. (1984) Science 198:1056; and Ike et al. (1983) Nucleic Acid Res. 11:477]. Such techniques have been employed in the directed evolution of other proteins [Scott et al., (1990) Science 249:386-390; Roberts et al. (1992) Proc Natl Acad Sci USA 89:2429-2433; Devlin et al. (1990) Science 249: 404-406; Cwirla et al., (1990) Proc Natl Acad Sci USA 87: 6378-6382; as well as U.S. Pat. Nos. 5,223,409, 5,198,346, and 5,096,815].

Alternatively, other forms of mutagenesis can be utilized to generate a combinatorial library. For example, a polypeptide (e.g., an ActRIIA, ActRIIB, ALK4, ALK7, or follistatin polypeptide), including homomultimers and heteromultimers thereof of the disclosure can be generated and isolated from a library by screening using, for example, alanine scanning mutagenesis [Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [Gustin et al. (1993) Virology 193:653-660; and Brown et al. (1992) Mol. Cell Biol. 12:2644-2652; McKnight et al. (1982) Science 232:316], by saturation mutagenesis [Meyers et al., (1986) Science 232:613]; by PCR mutagenesis [Leung et al. (1989) Method Cell Mol Biol 1:11-19]; or by random mutagenesis, including chemical mutagenesis [Miller et al. (1992) A Short Course in Bacterial Genetics, CSHL Press, Cold Spring Harbor, NY; and Greener et al. (1994) Strategies in Mol Biol 7:32-34]. Linker scanning mutagenesis, particularly in a combinatorial setting, is an attractive method for identifying truncated (bioactive) forms of a polypeptide (e.g., an ActRIIA, ActRIIB, ALK4, ALK7, or follistatin polypeptide), including homomultimers and heteromultimers thereof.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of a polypeptide (e.g., an ActRIIA, ActRIIB, ALK4, ALK7, or follistatin polypeptide), including homomultimers and heteromultimers thereof. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include ligand (e.g., activin A, activin B, GDF8, GDF11, BMP6, BMP10) binding assays and/or ligand-mediated cell signaling assays.

As will be recognized by one of skill in the art, most of the described mutations, variants or modifications described herein may be made at the nucleic acid level or, in some cases, by post-translational modification or chemical synthesis. Such techniques are well known in the art and some of which are described herein. In part, the present disclosure identifies functionally active portions (fragments) and variants of a polypeptide (e.g., an ActRIIA, ActRIIB, ALK4, ALK7, or follistatin polypeptide), including homomultimers and heteromultimers thereof that can be used as guidance for generating and using other variant polypeptides within the scope of the methods and uses described herein.

In certain embodiments, functionally active fragments of a polypeptide (e.g., an ActRIIA, ActRIIB, ALK4, ALK7, or follistatin polypeptide), including homomultimers and heteromultimers thereof of the disclosure can be obtained by screening polypeptides recombinantly produced from the corresponding fragment of the nucleic acid encoding polypeptides disclosed herein. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function as antagonists (inhibitors) of ActRII and/or ALK4 receptors and/or one or more ligands (e.g., activin A, activin B, GDF8, GDF11, BMP6, BMP10).

In certain embodiments, a polypeptide (e.g., an ActRIIA, ActRIIB, ALK4, ALK7, or follistatin polypeptide), including homomultimers and heteromultimers thereof or variant thereof of the disclosure may further comprise post-translational modifications in addition to any that are naturally present in the polypeptide. Such modifications include, but are not limited to, acetylation, carboxylation, glycosylation, phosphorylation, lipidation, and acylation. As a result, the polypeptide, including homomultimers and heteromultimers thereof, may contain non-amino acid elements, such as polyethylene glycols, lipids, polysaccharide or monosaccharide, and phosphates. Effects of such non-amino acid elements on the functionality of a polypeptide may be tested as described herein for other polypeptide variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, W138, NIH-3T3 or HEK293) have specific cellular machinery and characteristic mechanisms for such post-translational activities and may be chosen to ensure the correct modification and processing of the polypeptides.

I) Nucleic Acids and Method of Manufacture

In certain aspects, the disclosure provides isolated and/or recombinant nucleic acids encoding any of the polypeptides disclosed herein including, for example, ActRIIB, ActRIIA, ALK4, or ALK7 polypeptides (e.g., soluble ActRIIB, ActRIIA, ALK4, or ALK7 polypeptides), or follistatin polypeptides, as well as any of the variants disclosed herein. For example, SEQ ID NO: 4 encodes a naturally occurring ActRIIB precursor polypeptide, while SEQ ID NO: 3 encodes a soluble ActRIIB polypeptide. The subject nucleic acids may be single-stranded or double stranded. Such nucleic acids may be DNA or RNA molecules. These nucleic acids are may be used, for example, in methods for making ActRIIB, ActRIIA, ALK4, or ALK7 polypeptides or as direct therapeutic agents (e.g., in a gene therapy approach).

In certain aspects, the disclosure relates to isolated and/or recombinant nucleic acids comprising a coding sequence for one or more of the ActRIIB, ActRIIA, ALK4, ALK7, or follistatin polypeptide(s) as described herein. For example, in some embodiments, the disclosure relates to an isolated and/or recombinant nucleic acid that is at least 75%, 80%, 85%, 90%, 91%, 92%, 93%, 94%, 95% 96%, 97%, 98%, 99%, or 100% identical to the nucleic acid sequence corresponding to any one of SEQ ID Nos: 3, 4, 10, 32, 35, 38, 41, 44, 47, 221, 222, 223, 224, 233, 234, 235, 236, 237, 238, 239, 240, 243, 248, 250, 251, 252, 255, 277, 331, 334, 337, 340, 343, 346, 349, 352, 355, 369, 370, 382, 397, 407, 423, and 424. In some embodiments, an isolated and/or recombinant polynucleotide sequence of the disclosure comprises a promoter sequence operably linked to a coding sequence described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 4, 10, 32, 35, 38, 41, 44, 47, 221, 222, 223, 224, 233, 234, 235, 236, 237, 238, 239, 240, 243, 248, 250, 251, 252, 255, 277, 331, 334, 337, 340, 343, 346, 349, 352, 355, 369, 370, 382, 397, 407, 423, and 424). In some embodiments, the disclosure relates to vectors comprising an isolated and/or recombinant nucleic acid described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 4, 10, 32, 35, 38, 41, 44, 47, 221, 222, 223, 224, 233, 234, 235, 236, 237, 238, 239, 240, 243, 248, 250, 251, 252, 255, 277, 331, 334, 337, 340, 343, 346, 349, 352, 355, 369, 370, 382, 397, 407, 423, and 424). In some embodiments, the disclosure relates to a cell comprising an isolated and/or recombinant polynucleotide sequence described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 4, 10, 32, 35, 38, 41, 44, 47, 221, 222, 223, 224, 233, 234, 235, 236, 237, 238, 239, 240, 243, 248, 250, 251, 252, 255, 277, 331, 334, 337, 340, 343, 346, 349, 352, 355, 369, 370, 382, 397, 407, 423, and 424). In some embodiments, the cell is a CHO cell. In some embodiments, the cell is a COS cell.

In certain embodiments, nucleic acids encoding variant ActRIIB (or homomultimers or heteromultimers thereof), ALK4 or ALK7 polypeptides of the disclosure are understood to include nucleic acids that are variants of any one of SEQ ID NOs: 3, 4, 10, 32, 35, 38, 41, 44, 47, 221, 222, 223, 224, 233, 234, 235, 236, 237, 238, 239, 240, 243, 248, 250, 251, 252, 255, 277, 331, 334, 337, 340, 343, 346, 349, 352, 355, 369, 370, 382, 397, 407, 423, and 424. Variant nucleotide sequences include sequences that differ by one or more nucleotide substitutions, additions, or deletions including allelic variants, and therefore, will include coding sequence that differ from the nucleotide sequence designated in any one of SEQ ID NOs: 3, 4, 10, 32, 35, 38, 41, 44, 47, 221, 222, 223, 224, 233, 234, 235, 236, 237, 238, 239, 240, 243, 248, 250, 251, 252, 255, 277, 331, 334, 337, 340, 343, 346, 349, 352, 355, 369, 370, 382, 397, 407, 423, and 424.

In certain embodiments, variant ActRIIB (or homomultimers or heteromultimers thereof), ALK4, or ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to any one of SEQ ID NOs: 3, 4, 10, 32, 35, 38, 41, 44, 47, 221, 222, 223, 224, 233, 234, 235, 236, 237, 238, 239, 240, 243, 248, 250, 251, 252, 255, 277, 331, 334, 337, 340, 343, 346, 349, 352, 355, 369, 370, 382, 397, 407, 423, and 424. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 3. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95% 96%, 97% 98%, 99%, or 100% identical to SEQ ID NO: 4. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 10. In certain embodiments, variant ActRIIB polypeptides (or homomultimers or heteromultimers thereof) of the disclosure are encoded by isolated and/or recombinant nucleic acid sequences that are at least 70%, 75% 80%, 85%, 90%, 91%, 92%, 93%, 94%, 95% 96%, 97% 98%, 99%, or 100% identical to SEQ ID NO: 32. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 35. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95% 96%, 97%, 98%, 99%, or 100% identical to SEQ ID NO: 38. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 41. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95% 96%, 97% 98%, 99%, or 100% identical to SEQ ID NO: 44. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 47.

In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 277. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 331. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 334. In certain embodiments, variant ActRIIB polypeptides (or homomultimers or heteromultimers thereof) of the disclosure are encoded by isolated and/or recombinant nucleic acid sequences that are at least 70%, 7 80% 85%, 90%, 91%, 92%, 93%, 94%, 95%, 96%, 97%, 98%, 99%, or 100% identical to SEQ ID NO: 337. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 340. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98% 99%, or 100% identical to SEQ ID NO: 343. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 346. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 349. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 352. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 355. In certain embodiments, variant ActRIIB polypeptides (or homomultimers or heteromultimers thereof) of the disclosure are encoded by isolated and/or recombinant nucleic acid sequences that are at least 70%, 75% 80%, 85%, 90%, 91%, 92%, 93%, 94%, 95% 96%, 97% 98%, 99%, or 100% identical to SEQ ID NO: 382. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 397. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98% 99%, or 100% identical to SEQ ID NO: 407.

In certain embodiments, variant ActRIIA polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 369. In certain embodiments, variant ActRIIA polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 370.

In certain embodiments, ALK4 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 221. In certain embodiments, ALK4 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 222. In certain embodiments, ALK4 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 223. In certain embodiments, ALK4 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 224. In certain embodiments, ALK4 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 423. In certain embodiments, ALK4 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 424.

In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 233. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 234. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 235. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 236. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 237. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 238. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 239. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 240.

In certain embodiments, ALK4-Fc fusion polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 243. In certain embodiments, ALK4-Fc fusion polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 248. In certain embodiments, ALK4-Fc fusion polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 250. In certain embodiments, ALK4-Fc fusion polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 251. In certain embodiments, ALK4-Fc fusion polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 252. In certain embodiments, ALK7-Fc fusion polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 255.

In certain aspects, the subject nucleic acids encoding variant ActRIIB polypeptides are further understood to include nucleic acids that are variants of SEQ ID NO: 3. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID NO: 4.

In certain embodiments, the disclosure provides isolated or recombinant nucleic acid sequences that are at least 75%, 80%, 85%, 90%, 91%, 92%, 93%, 94%, 95%, 96%, 97%, 98%, 99%, or 100% identical to SEQ ID NO: 3. One of ordinary skill in the art will appreciate that nucleic acid sequences complementary to SEQ ID NO: 3, and variants of SEQ ID NO: 3 are also within the scope of this disclosure. In further embodiments, the nucleic acid sequences of the disclosure can be isolated, recombinant, and/or fused with a heterologous nucleotide sequence, or in a DNA library.

In other embodiments, nucleic acids of the disclosure also include nucleotide sequences that hybridize under highly stringent conditions to nucleic acids encoding ActRIIB or ActRIIA polypeptides in either homomeric or heteromeric forms, ALK4, or ALK7 polypeptides of the disclosure, or follistatin polypeptides of the disclosure, the complement sequence, or fragments thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in the disclosure due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the polypeptide. However, it is expected that DNA sequence polymorphisms that do lead to changes in the amino acid sequences of the subject polypeptides will exist among mammalian cells. One skilled in the art will appreciate that these variations in one or more nucleotides (up to about 3-5% of the nucleotides) of the nucleic acids encoding a particular polypeptide may exist among individuals of a given species due to natural allelic variation. Any and all such nucleotide variations and resulting amino acid polymorphisms are within the scope of this disclosure.

In certain embodiments, the recombinant nucleic acids of the disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In one embodiment, the expression vector contains a selectable marker gene to allow the selection of transformed host cells. Selectable marker genes are well known in the art and will vary with the host cell used.

In certain aspects, the subject nucleic acid is provided in an expression vector comprising a nucleotide sequence encoding polypeptides of the disclosure (e.g., a variant ActRIIA, ActRIIB, ALK4, ALK7, or follistatin polypeptide), operably linked to at least one regulatory sequence. Regulatory sequences are art-recognized and are selected to direct expression of the polypeptides of the disclosure (e.g., a variant ActRIIA, ActRIIB, ALK4, ALK7, or follistatin polypeptide). Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding polypeptides of the disclosure (e.g., a variant ActRIIA, ActRIIB, ALK4, ALK7, or follistatin polypeptide). Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of polypeptide desired to be expressed. Moreover, the vector's copy number, the ability to control that copy number and the expression of any other polypeptide encoded by the vector, such as antibiotic markers, should also be considered.

A recombinant nucleic acid of the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant variant ActRIIB polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of polypeptides in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the ß-gal containing pBlueBac III).

In one embodiment, a vector will be designed for production of the polypeptides of the disclosure (e.g., a variant ActRIIA, ActRIIB, ALK4, ALK7, or follistatin polypeptide) in CHO cells, such as a Pcmv-Script vector (Stratagene, La Jolla, Calif.), pcDNA4 vectors (Invitrogen, Carlsbad, Calif.) and pCI-neo vectors (Promega, Madison, Wisc.). As will be apparent, the subject gene constructs can be used to cause expression of the polypeptides of the disclosure (e.g., a variant ActRIIA, ActRIIB, ALK4, ALK7, or follistatin polypeptide) in cells propagated in culture, e.g., to produce polypeptides, including fusion polypeptides or polypeptides, for purification.

In certain embodiments, the disclosure relates to methods of making polypeptides of the disclosure (e.g., a variant ActRIIA, ActRIIB, ALK4, ALK7, or follistatin polypeptide) as well as homomultimer and heteromultimers comprising the same, as described herein. Such a method may include expressing any of the nucleic acids disclosed herein in a suitable cell (e.g., a CHO cell or COS cell). Such a method may comprise: a) culturing a cell under conditions suitable for expression of the soluble polypeptides of the disclosure (e.g., a variant ActRIIA, ActRIIB, ALK4, ALK7, or follistatin polypeptide), wherein said cell comprise with an expression construct of polypeptides of the disclosure (e.g., a variant ActRIIA, ActRIIB, ALK4, ALK7, or follistatin polypeptide). In some embodiments, the method further comprises recovering the expressed polypeptides of the disclosure (e.g., a variant ActRIIA, ActRIIB, ALK4, ALK7, or follistatin polypeptide). Polypeptides of the disclosure (e.g., a variant ActRIIA, ActRIIB, ALK4, ALK7, or follistatin polypeptide) may be recovered as crude, partially purified or highly purified fractions using any of the well-known techniques for obtaining protein from cell cultures.

This disclosure also pertains to a host cell transfected with a recombinant gene including a coding sequence for one or more polypeptides of the disclosure (e.g., a variant ActRIIA, ActRIIB, ALK4, ALK7, or follistatin polypeptide). The host cell may be any prokaryotic or eukaryotic cell. For example, polypeptides of the disclosure (e.g., a variant ActRIIA, ActRIIB, ALK4, ALK7, or follistatin polypeptide) may be expressed in bacterial cells such as E. coli, insect cells (e.g., using a baculovirus expression system), yeast, or mammalian cells. Other suitable host cells are known to those skilled in the art.

Accordingly, the present disclosure further pertains to methods of producing polypeptides of the disclosure (e.g., a variant ActRIIA, ActRIIB, ALK4, ALK7, or follistatin polypeptide). For example, a host cell transfected with an expression vector encoding polypeptides of the disclosure (e.g., a variant ActRIIA, ActRIIB, ALK4, ALK7, or follistatin polypeptide) can be cultured under appropriate conditions to allow expression of the polypeptides of the disclosure (e.g., a variant ActRIIA, ActRIIB, ALK4, ALK7, or follistatin polypeptide) to occur. The polypeptides of the disclosure (e.g., a variant ActRIIA, ActRIIB, ALK4, ALK7, or follistatin polypeptide) may be secreted and isolated from a mixture of cells and medium containing the polypeptides. Alternatively, the polypeptides of the disclosure (e.g., a variant ActRIIA, ActRIIB, ALK4, ALK7, or follistatin polypeptide) may be retained cytoplasmically or in a membrane fraction and the cells harvested, lysed and the protein isolated. A cell culture includes host cells, media and other byproducts. Suitable media for cell culture are well known in the art. The subject polypeptides of the disclosure (e.g., a variant ActRIIA, ActRIIB, ALK4, ALK7, or follistatin polypeptide) can be isolated from cell culture medium, host cells, or both, using techniques known in the art for purifying polypeptides, including ion-exchange chromatography, gel filtration chromatography, ultrafiltration, electrophoresis, and immunoaffinity purification with antibodies specific for particular epitopes of polypeptides of the disclosure (e.g., a variant ActRIIA, ActRIIB, ALK4, ALK7, or follistatin polypeptide). In one embodiment, the polypeptides of the disclosure (e.g., a variant ActRIIA, ActRIIB, ALK4, ALK7, or follistatin polypeptide) are fusion polypeptides containing a domain which facilitates purification.

In some embodiments, ActRII polypeptides, ALK4 polypeptides, ALK7 polypeptides, and ActRIIB-ALK4, ActRIIB-ALK7, ActRIIA-ALK4, and ActRIIA-ALK7 heteromultimers to be used in accordance with the methods described herein are isolated polypeptides. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of purity are well known in the art [see, e.g., Flatman et al., (2007) J. Chromatogr. B 848:79-87]. In some embodiments, ActRII polypeptides, ALK4 polypeptides, and ActRIIB-ALK4 heteromultimers to be used in accordance with the methods described herein are recombinant polypeptides.

In certain embodiments, ActRIIB or ActRIIA polypeptides of the disclosure can be produced by a variety of art-known techniques. For example, such ActRIIB or ActRIIA polypeptides can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the ActRIIB or ActRIIA polypeptides, fragments or variants thereof may be recombinantly produced using various expression systems (e.g., E. coli, Chinese Hamster Ovary cells, COS cells, baculovirus) as is well known in the art (also see above). In a further embodiment, the ActRIIB or ActRIIA polypeptides may be produced by digestion of naturally occurring or recombinantly produced full-length ActRIIB or ActRIIA polypeptides by using, for example, a protease, e.g., trypsin, thermolysin, chymotrypsin, pepsin, or paired basic amino acid converting enzyme (PACE). Computer analysis (using a commercially available software, e.g., MacVector, Omega, PCGene, Molecular Simulation, Inc.) can be used to identify proteolytic cleavage sites. Alternatively, such ActRIIB or ActRIIA polypeptides may be produced from naturally occurring or recombinantly produced full-length ActRIIB or ActRIIA polypeptides such as standard techniques known in the art, such as by chemical cleavage (e.g., cyanogen bromide, hydroxylamine).

In another embodiment, a fusion gene coding for a purification leader sequence, such as a poly-(His)/enterokinase cleavage site sequence at the N-terminus of the desired portion of the recombinant polypeptides of the disclosure (e.g., a variant ActRIIA, ActRIIB, ALK4, ALK7, or follistatin polypeptide), can allow purification of the expressed fusion polypeptide by affinity chromatography using a Ni²⁺ metal resin. The purification leader sequence can then be subsequently removed by treatment with enterokinase to provide the purified polypeptides of the disclosure (e.g., a variant ActRIIA, ActRIIB, ALK4, ALK7, or follistatin polypeptide) (e.g., see Hochuli et al., (1987) J. Chromatography 411:177; and Janknecht et al., Proc Natl Acad Sci USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

### 3. Antibody Antagonists

In certain aspects, an ActRII-ALK4 antagonist to be used in accordance with the methods and uses disclosed herein (e.g., treating, preventing, or reducing the progression rate and/or severity of heart failure associated with aging or one or more complications of heart failure associated with aging) is an antibody (ActRII-ALK4 antagonist antibody), or combination of antibodies. An ActRII-ALK4 antagonist antibody, or combination of antibodies, may bind to, for example, one or more ActRII ligands (e.g., activin A, activin B, GDF8, GDF11, BMP6, BMP10), ActRII receptor (ActRIIA and/or ActRIIB), and/or type I receptor (e.g., ALK4). As described herein, ActRII-ALK4 antagonist antibodies may be used, alone or in combination with one or more supportive therapies or active agents, to treat, prevent, or reduce the progression rate and/or severity of heart failure associated with aging, particularly treating, preventing or reducing the progression rate and/or severity of one or more heart failure-associated complications.

In certain aspects, an ActRII-ALK4 antagonist antibody, or combination of antibodies, is an antibody that inhibits at least activin (e.g., activin A, activin B, activin C, activin E, activin AB, activin AC, activin BC, activin AE, and/or activin BE). Therefore, in some embodiments, an ActRII-ALK4 antagonist antibody, or combination of antibodies, binds to at least activin. As used herein, an activin antibody (or anti-activin antibody) generally refers to an antibody that binds to activin with sufficient affinity such that the antibody is useful as a diagnostic and/or therapeutic agent in targeting activin. In certain embodiments, the extent of binding of an activin antibody to an unrelated, non-activin protein is less than about 10%, 9%, 8%, 7%, 6%, 5%, 4%, 3%, 2%, or less than about 1% of the binding of the antibody to activin as measured, for example, by a radioimmunoassay (RIA), Biacore, or other protein interaction or binding affinity assay. In certain embodiments, an activin antibody binds to an epitope of activin that is conserved among activin from different species. In certain embodiments, an anti-activin antibody binds to human activin. In some embodiments, an activin antibody may inhibit activin from binding to a type I and/or type II receptor (e.g., ActRIIA, ActRIIB, and/or ALK4,) and thus inhibit activin-mediated signaling (e.g., Smad signaling). It should be noted that activin A has similar sequence homology to activin B and therefore antibodies that bind to activin A, in some instances, may also bind to and/or inhibit activin B, which also applies to anti-activin B antibodies. In some embodiments, the disclosure relates to a multispecific antibody (e.g., bi-specific antibody), and uses thereof, that binds to activin and further binds to, for example, one or more additional ActRII-ALK4 ligands (e.g., activin A, activin B, GDF8, GDF11, BMP6, BMP10), one or more type I receptor and/or type II receptors (e.g., ActRIIA, ActRIIB, and/or ALK4). In some embodiments, a multispecific antibody that binds to activin does not bind or does not substantially bind to BMP9 (e.g., binds to BMP9 with a K_{D} of greater than 1×10⁻⁷ M or has relatively modest binding, e.g., about 1×10⁻⁸ M or about 1×10⁻⁹ M). In some embodiments, a multispecific antibody that binds to activin does not bind or does not substantially bind to activin A (e.g., binds to activin A with a K_{D} of greater than 1×10⁻⁷ M or has relatively modest binding, e.g., about 1×10⁻⁸ M or about 1×10⁻⁹ M). In some embodiments, the disclosure relates to combinations of antibodies, and uses thereof, wherein the combination of antibodies comprises an activin antibody and one or more additional antibodies that bind to, for example, one or more additional ActRII ligands (e.g., activin A, activin B, GDF8, GDF11, BMP6, BMP10), ActRII receptor (ActRIIA and/or ActRIIB), and/or type I receptor (e.g., ALK4). In some embodiments, a combination of antibodies that comprises an activin antibody does not comprise a BMP9 antibody.

In certain aspects, an ActRII-ALK4 antagonist antibody, or combination of antibodies, is an antibody that inhibits at least activin A. Therefore, in some embodiments, an ActRII-ALK4 antagonist antibody, or combination of antibodies, binds to at least activin A. As used herein, an activin A antibody (or anti-activin A antibody) generally refers to an antibody that binds to activin A with sufficient affinity such that the antibody is useful as a diagnostic and/or therapeutic agent in targeting activin A. In certain embodiments, the extent of binding of an activin A antibody to an unrelated, non-activin A protein is less than about 10%, 9%, 8%, 7%, 6%, 5%, 4%, 3%, 2%, or less than about 1% of the binding of the antibody to activin as measured, for example, by a radioimmunoassay (RIA), Biacore, or other protein interaction or binding affinity assay. In certain embodiments, an activin A antibody binds to an epitope of activin A that is conserved among activin A from different species. In certain embodiments, an anti-activin A antibody binds to human activin A. In some embodiments, an activin A antibody may inhibit activin A from binding to a type I and/or type II receptor (e.g., ActRIIA, ActRIIB, and/or ALK4) and thus inhibit activin A-mediated signaling (e.g., Smad signaling). In some embodiments, an activin A antibody may inhibit activin A from binding to a co-receptor and thus inhibit activin A-mediated signaling (e.g., Smad signaling). It should be noted that activin A has similar sequence homology to activin B and therefore antibodies that bind to activin A, in some instances, may also bind to and/or inhibit activin B. In some embodiments, the disclosure relates to a multispecific antibody (e.g., bi-specific antibody), and uses thereof, that binds to activin A and further binds to, for example, one or more additional ActRII ligands (e.g., activin A, activin B, GDF8, GDF11, BMP6, BMP10), ActRII receptor (ActRIIA and/or ActRIIB), and/or type I receptor (e.g., ALK4). In some embodiments, a multispecific antibody that binds to activin A does not bind or does not substantially bind to BMP9 (e.g., binds to BMP9 with a K_{D} of greater than 1×10⁻⁷ M or has relatively modest binding, e.g., about 1×10⁻⁸ M or about 1×10⁻⁹ M). In some embodiments, a multispecific antibody that binds to activin A does not bind or does not substantially bind to activin B (e.g., binds to activin B with a K_{D} of greater than 1 20×10⁻⁷ M or has relatively modest binding, e.g., about 1×10⁻⁸ M or about 1×10⁻⁹ M). In some embodiments, the disclosure relates to combinations of antibodies, and uses thereof, wherein the combination of antibodies comprises an activin A antibody and one or more additional antibodies that bind to, for example, one or more additional ActRII ligands (e.g., activin A, activin B, GDF8, GDF11, BMP6, BMP10), ActRII receptor (ActRIIA and/or ActRIIB), and/or type I receptor (e.g., ALK4). In some embodiments, a combination of antibodies that comprises an activin A antibody does not comprise a BMP9 antibody. In some embodiments, a combination of antibodies that comprises an activin A antibody does not comprise an activin B antibody. In some embodiments, an activin A antibody of the present disclosure comprises REGN-2477. In some embodiments, an activin A antibody of the present disclosure comprises garetosmab.

In certain aspects, an ActRII-ALK4 antagonist antibody, or combination of antibodies, is an antibody that inhibits at least activin B. Therefore, in some embodiments, an ActRII-ALK4 antagonist antibody, or combination of antibodies, binds to at least activin B. As used herein, an activin B antibody (or anti-activin B antibody) generally refers to an antibody that binds to activin B with sufficient affinity such that the antibody is useful as a diagnostic and/or therapeutic agent in targeting activin B. In certain embodiments, the extent of binding of an activin B antibody to an unrelated, non-activin B protein is less than about 10%, 9%, 8%, 7%, 6%, 5%, 4%, 3%, 2%, or less than about 1% of the binding of the antibody to activin as measured, for example, by a radioimmunoassay (RIA), Biacore, or other protein interaction or binding affinity assay. In certain embodiments, an activin B antibody binds to an epitope of activin B that is conserved among activin B from different species. In certain embodiments, an anti-activin B antibody binds to human activin B. In some embodiments, an activin B antibody may inhibit activin B from binding to a type I and/or type II receptor (e.g., ActRIIA, ActRIIB, and/or ALK4) and thus inhibit activin B-mediated signaling (e.g., Smad signaling). In some embodiments, an activin B antibody may inhibit activin B from binding to a co-receptor and thus inhibit activin B-mediated signaling (e.g., Smad signaling). It should be noted that activin B has similar sequence homology to activin A and therefore antibodies that bind to activin B, in some instances, may also bind to and/or inhibit activin A. In some embodiments, the disclosure relates to a multispecific antibody (e.g., bi-specific antibody), and uses thereof, that binds to activin B and further binds to, for example, one or more additional ActRII ligands (e.g., activin A, activin B, GDF8, GDF11, BMP6, BMP10), ActRII receptor (ActRIIA and/or ActRIIB), and/or type I receptor (e.g., ALK4). In some embodiments, a multispecific antibody that binds to activin B does not bind or does not substantially bind to BMP9 (e.g., binds to BMP9 with a K_{D} of greater than 1×10⁻⁷ M or has relatively modest binding, e.g., about 1×10⁻⁸ M or about 1×10⁻⁹ M). In some embodiments, a multispecific antibody that binds to activin B does not bind or does not substantially bind to activin A (e.g., binds to activin A with a K_{D} of greater than 1×10⁻⁷ M or has relatively modest binding, e.g., about 1×10⁻⁸ M or about 1×10⁻⁹ M). In some embodiments, the disclosure relates to combinations of antibodies, and uses thereof, wherein the combination of antibodies comprises an activin B antibody and one or more additional antibodies that bind to, for example, one or more additional ActRII ligands (e.g., activin A, activin B, GDF8, GDF11, BMP6, BMP10), ActRII receptor (ActRIIA and/or ActRIIB), and/or type I receptor (e.g., ALK4). In some embodiments, a combination of antibodies that comprises an activin B antibody does not comprise a BMP9 antibody. In some embodiments, a combination of antibodies that comprises an activin B antibody does not comprise an activin A antibody.

In certain aspects, an ActRII-ALK4 antagonist antibody, or combination of antibodies, is an antibody that inhibits at least GDF8. Therefore, in some embodiments, an ActRII-ALK4 antagonist antibody, or combination of antibodies, binds to at least GDF8. As used herein, a GDF8 antibody (or anti-GDF8 antibody) generally refers to an antibody that binds to GDF8 with sufficient affinity such that the antibody is useful as a diagnostic and/or therapeutic agent in targeting GDF8. In certain embodiments, the extent of binding of a GDF8 antibody to an unrelated, non-GDF8 protein is less than about 10%, 9%, 8%, 7%, 6%, 5%, 4%, 3%, 2%, or less than about 1% of the binding of the antibody to GDF8 as measured, for example, by a radioimmunoassay (RIA), Biacore, or other protein interaction or binding affinity assay. In certain embodiments, a GDF8 antibody binds to an epitope of GDF8 that is conserved among GDF8 from different species. In certain embodiments, an anti-GDF8 antibody binds to human GDF8. In some embodiments, a GDF8 antibody may inhibit GDF8 from binding to a type I and/or type II receptor (e.g., ActRIIA, ActRIIB, and/or ALK4) and thus inhibit GDF8-mediated signaling (e.g., Smad signaling). In some embodiments, a GDF8 antibody may inhibit GDF8 from binding to a co-receptor and thus inhibit GDF8-mediated signaling (e.g., Smad signaling). It should be noted that GDF8 has high sequence homology to GDF11 and therefore antibodies that bind to GDF8, in some instances, may also bind to and/or inhibit GDF11. In some embodiments, the disclosure relates to a multispecific antibody (e.g., bi-specific antibody), and uses thereof, that binds to GDF8 and further binds to, for example, one or more additional ActRII ligands (e.g., activin A, activin B, GDF8, GDF11, BMP6, BMP10), ActRII receptor (ActRIIA and/or ActRIIB), and/or type I receptor (e.g., ALK4). In some embodiments, a multispecific antibody that binds to GDF8 does not bind or does not substantially bind to BMP9 (e.g., binds to BMP9 with a K_{D} of greater than 1×10⁻⁷ M or has relatively modest binding, e.g., about 1×10⁻⁸ M or about 1×10⁻⁹ M). In some embodiments, a multispecific antibody that binds to GDF8 does not bind or does not substantially bind to activin A (e.g., binds to activin A with a K_{D} of greater than 1×10⁻⁷ M or has relatively modest binding, e.g., about 1×10⁻⁸ M or about 1×10⁻⁹ M). In some embodiments, the disclosure relates to combinations of antibodies, and uses thereof, wherein the combination of antibodies comprises a GDF8 antibody and one or more additional antibodies that bind to, for example, one or more additional ActRII ligands (e.g., activin A, activin B, GDF8, GDF11, BMP6, BMP10), ActRII receptor (ActRIIA and/or ActRIIB), and/or type I receptor (e.g., ALK4). In some embodiments, a combination of antibodies that comprises a GDF8 antibody does not comprise a BMP9 antibody. In some embodiments, a combination of antibodies that comprises a GDF8 antibody does not comprise an activin A antibody. In some embodiments, a GDF8 antibody of the present disclosure comprises REGN-1033. In some embodiments, a GDF8 antibody of the present disclosure comprises trevogumab. In some embodiments, a GDF8 antibody of the present disclosure comprises MYO-029. In some embodiments, a GDF8 antibody of the present disclosure comprises stamulumab. In some embodiments, a GDF8 antibody of the present disclosure comprises PF-06252616. In some embodiments, a GDF8 antibody of the present disclosure comprises domagrozumab. In some embodiments, a GDF8 antibody of the present disclosure comprises LY-2495655. In some embodiments, a GDF8 antibody of the present disclosure comprises landogrozumab. In some embodiments, a GDF8 antibody of the present disclosure comprises SRK-015.

In certain aspects, an ActRII-ALK4 antagonist antibody, or combination of antibodies, is an antibody that inhibits at least GDF11. Therefore, in some embodiments, an ActRII-ALK4 antagonist antibody, or combination of antibodies, binds to at least GDF11. As used herein, a GDF11 antibody (or anti-GDF11 antibody) generally refers to an antibody that binds to GDF11 with sufficient affinity such that the antibody is useful as a diagnostic and/or therapeutic agent in targeting GDF11. In certain embodiments, the extent of binding of a GDF11 antibody to an unrelated, non-GDF11 protein is less than about 10%, 9%, 8%, 7%, 6%, 5%, 4%, 3%, 2%, or less than about 1% of the binding of the antibody to GDF11 as measured, for example, by a radioimmunoassay (RIA), Biacore, or other protein interaction or binding affinity assay. In certain embodiments, a GDF11 antibody binds to an epitope of GDF11 that is conserved among GDF11 from different species. In certain embodiments, an anti-GDF11 antibody binds to human GDF11. In some embodiments, a GDF11 antibody may inhibit GDF11 from binding to a type I and/or type II receptor (e.g., ActRIIA, ActRIIB, and/or ALK4,) and thus inhibit GDF11-mediated signaling (e.g., Smad signaling). In some embodiments, a GDF11 antibody may inhibit GDF11 from binding to a co-receptor and thus inhibit GDF11-mediated signaling (e.g., Smad signaling). It should be noted that GDF11 has high sequence homology to GDF8 and therefore antibodies that bind to GDF11, in some instances, may also bind to and/or inhibit GDF8. In some embodiments, the disclosure relates to a multispecific antibody (e.g., bi-specific antibody), and uses thereof, that binds to GDF11 and further binds to, for example, one or more additional ActRII-ALK4 ligands (e.g., activin A, activin B, GDF8, GDF11, BMP6, BMP10), one or more type I receptor and/or type II receptors (e.g., ActRIIA, ActRIIB, and/or ALK4), and/or one or more co-receptors. In some embodiments, a multispecific antibody that binds to GDF11 does not bind or does not substantially bind to BMP9 (e.g., binds to BMP9 with a K_{D} of greater than 1×10⁻⁷ M or has relatively modest binding, e.g., about 1×10⁻⁸ M or about 1×10⁻⁹ M). In some embodiments, a multispecific antibody that binds to GDF11 does not bind or does not substantially bind to activin A (e.g., binds to activin A with a K_{D} of greater than 1×10⁻⁷ M or has relatively modest binding, e.g., about 1×10⁻⁸ M or about 1×10⁻⁹ M). In some embodiments, the disclosure relates to combinations of antibodies, and uses thereof, wherein the combination of antibodies comprises a GDF11 antibody and one or more additional antibodies that bind to, for example, one or more additional ActRII ligands (e.g., activin A, activin B, GDF8, GDF11, BMP6, BMP10), ActRII receptor (ActRIIA and/or ActRIIB), and/or type I receptor (e.g., ALK4). In some embodiments, a combination of antibodies that comprises a GDF11 antibody does not comprise a BMP9 antibody. In some embodiments, a combination of antibodies that comprises a GDF11 antibody does not comprise an activin A antibody.

In certain aspects, an ActRII-ALK4 antagonist antibody, or combination of antibodies, is an antibody that inhibits at least BMP6. Therefore, in some embodiments, an ActRII-ALK4 antagonist antibody, or combination of antibodies, binds to at least BMP6. As used herein, a BMP6 antibody (or anti-BMP6 antibody) generally refers to an antibody that can bind to BMP6 with sufficient affinity such that the antibody is useful as a diagnostic and/or therapeutic agent in targeting BMP6. In certain embodiments, the extent of binding of a BMP6 antibody to an unrelated, non-BMP6 protein is less than about 10%, 9%, 8%, 7%, 6%, 5%, 4%, 3%, 2%, or less than about 1% of the binding of the antibody to BMP6 as measured, for example, by a radioimmunoassay (RIA), Biacore, or other protein interaction or binding affinity assay. In certain embodiments, a BMP6 antibody binds to an epitope of BMP6 that is conserved among BMP6 from different species. In certain embodiments, an anti-BMP6 antibody binds to human BMP6. In some embodiments, a BMP6 antibody may inhibit BMP6 from binding to a type I and/or type II receptor (e.g., ActRIIA, ActRIIB, and/or ALK4) and thus inhibit BMP6-mediated signaling (e.g., Smad signaling). In some embodiments, a BMP6 antibody may inhibit BMP6 from binding to a co-receptor and thus inhibit BMP6-mediated signaling (e.g., Smad signaling). In some embodiments, the disclosure relates to a multispecific antibody (e.g., bi-specific antibody), and uses thereof, that binds to BMP6 and further binds to, for example, one or more additional ActRII ligands (e.g., activin A, activin B, GDF8, GDF11, BMP6, BMP10), ActRII receptor (ActRIIA and/or ActRIIB), and/or type I receptor (e.g., ALK4). In some embodiments, a multispecific antibody that binds to BMP6 does not bind or does not substantially bind to BMP9 (e.g., binds to BMP9 with a K_{D} of greater than 1×10⁻⁷ M or has relatively modest binding, e.g., about 1×10⁻⁸ M or about 1×10⁻⁹ M). In some embodiments, a multispecific antibody that binds to BMP6 does not bind or does not substantially bind to activin A (e.g., binds to activin A with a K_{D} of greater than 1×10⁻⁷ M or has relatively modest binding, e.g., about 1×10⁻⁸ M or about 1×10⁻⁹ M). In some embodiments, the disclosure relates to combinations of antibodies, and uses thereof, wherein the combination of antibodies comprises a BMP6 antibody and one or more additional antibodies that bind to, for example, one or more ActRII ligands (e.g., activin A, activin B, GDF8, GDF11, BMP6, BMP10), ActRII receptor (ActRIIA and/or ActRIIB), and/or type I receptor (e.g., ALK4). In some embodiments, a combination of antibodies that comprises a BMP6 antibody does not comprise a BMP9 antibody. In some embodiments, a combination of antibodies that comprises a BMP6 antibody does not comprise an activin A antibody.

In certain aspects, an ActRII-ALK4 antagonist antibody, or combination of antibodies, is an antibody that inhibits at least BMP10. Therefore, in some embodiments, an ActRII-ALK4 antagonist antibody, or combination of antibodies, binds to at least BMP10. As used herein, a BMP10 antibody (or anti-BMP10 antibody) generally refers to an antibody that can bind to BMP10 with sufficient affinity such that the antibody is useful as a diagnostic and/or therapeutic agent in targeting BMP10. In certain embodiments, the extent of binding of a BMP10 antibody to an unrelated, non-BMP10 protein is less than about 10%, 9%, 8%, 7%, 6%, 5%, 4%, 3%, 2%, or less than about 1% of the binding of the antibody to BMP10 as measured, for example, by a radioimmunoassay (RIA), Biacore, or other protein interaction or binding affinity assay. In certain embodiments, a BMP10 antibody binds to an epitope of BMP10 that is conserved among BMP10 from different species. In certain embodiments, an anti-BMP10 antibody binds to human BMP10. In some embodiments, a BMP10 antibody may inhibit BMP10 from binding to a type I and/or type II receptor (e.g., ActRIIA, ActRIIB, and/or ALK4) and thus inhibit BMP10-mediated signaling (e.g., Smad signaling). In some embodiments, a BMP10 antibody may inhibit BMP10 from binding to a co-receptor and thus inhibit BMP10-mediated signaling (e.g., Smad signaling). In some embodiments, the disclosure relates to a multispecific antibody (e.g., bi-specific antibody), and uses thereof, that binds to BMP10 and further binds to, for example, one or more additional ActRII ligands (e.g., activin A, activin B, GDF8, GDF11, BMP6, BMP10), ActRII receptor (ActRIIA and/or ActRIIB), and/or type I receptor (e.g., ALK4). In some embodiments, a multispecific antibody that binds to BMP10 does not bind or does not substantially bind to BMP9 (e.g., binds to BMP9 with a K_{D} of greater than 1×10⁻⁷ M or has relatively modest binding, e.g., about 1×10⁻⁸ M or about 1×10⁻⁹ M). In some embodiments, a multispecific antibody that binds to BMP10 does not bind or does not substantially bind to activin A (e.g., binds to activin A with a K_{D} of greater than 1×10⁻⁷ M or has relatively modest binding, e.g., about 1×10⁻⁸ M or about 1×10⁻⁹ M). In some embodiments, the disclosure relates to combinations of antibodies, and uses thereof, wherein the combination of antibodies comprises a BMP10 antibody and one or more additional antibodies that bind to, for example, one or more additional ActRII ligands (e.g., activin A, activin B, GDF8, GDF11, BMP6, BMP10), ActRII receptor (ActRIIA and/or ActRIIB), and/or type I receptor (e.g., ALK4). In some embodiments, a combination of antibodies that comprises a BMP10 antibody does not comprise a BMP9 antibody. In some embodiments, a combination of antibodies that comprises a BMP10 antibody does not comprise an activin A antibody.

In certain aspects, an ActRII-ALK4 antagonist antibody, or combination of antibodies, is an antibody that inhibits at least ActRIIB. Therefore, in some embodiments, an ActRII-ALK4 antagonist antibody, or combination of antibodies, binds to at least ActRIIB. As used herein, an ActRIIB antibody (anti-ActRIIB antibody) generally refers to an antibody that binds to ActRIIB with sufficient affinity such that the antibody is useful as a diagnostic and/or therapeutic agent in targeting ActRIIB. In certain embodiments, the extent of binding of an anti-ActRIIB antibody to an unrelated, non-ActRIIB protein is less than about 10%, 9%, 8%, 7%, 6%, 5%, 4%, 3%, 2%, or less than about 1% of the binding of the antibody to ActRIIB as measured, for example, by a radioimmunoassay (RIA), Biacore, or other protein-protein interaction or binding affinity assay. In certain embodiments, an anti-ActRIIB antibody binds to an epitope of ActRIIB that is conserved among ActRIIB from different species. In certain embodiments, an anti-ActRIIB antibody binds to human ActRIIB. In some embodiments, an anti-ActRIIB antibody may inhibit one or more ActRII-ALK4 ligands (e.g., activin A, activin B, GDF8, GDF11, BMP6, BMP10) from binding to ActRIIB. In some embodiments, an anti-ActRIIB antibody is a multispecific antibody (e.g., bi-specific antibody) that binds to ActRIIB and one or more ActRII ligands (e.g., activin A, activin B, GDF8, GDF11, BMP6, BMP10), ActRII receptor (e.g., ActRIIA), and/or type I receptor (e.g., ALK4). In some embodiments, the disclosure relates to combinations of antibodies, and uses thereof, wherein the combination of antibodies comprises an anti-ActRIIB antibody and one or more additional antibodies that bind to, for example, one or more ActRII-ALK4 ligands (e.g., activin A, activin B, GDF8, GDF11, BMP6, BMP10), type I receptors (e.g., ALK4), and/or additional type II receptors (e.g., ActRIIA). It should be noted that ActRIIB has sequence similarity to ActRIIA and therefore antibodies that bind to ActRIIB, in some instances, may also bind to and/or inhibit ActRIIA. In some embodiments, an anti-ActRII antibody of the present disclosure comprises bimagrumab (BYM338).

In certain aspects, an ActRII-ALK4 antagonist antibody, or combination of antibodies, is an antibody that inhibits at least ActRIIA. Therefore, in some embodiments, an ActRII-ALK4 antagonist antibody, or combination of antibodies, binds to at least ActRIIA. As used herein, an ActRIIA antibody (anti-ActRIIA antibody) generally refers to an antibody that binds to ActRIIA with sufficient affinity such that the antibody is useful as a diagnostic and/or therapeutic agent in targeting ActRIIA. In certain embodiments, the extent of binding of an anti-ActRIIA antibody to an unrelated, non-ActRIIA protein is less than about 10%, 9%, 8%, 7%, 6%, 5%, 4%, 3%, 2%, or less than about 1% of the binding of the antibody to ActRIIA as measured, for example, by a radioimmunoassay (RIA), Biacore, or other protein-protein interaction or binding affinity assay. In certain embodiments, an anti-ActRIIA antibody binds to an epitope of ActRIIA that is conserved among ActRIIA from different species. In certain embodiments, an anti-ActRIIA antibody binds to human ActRIIA. In some embodiments, an anti-ActRIIA antibody may inhibit one or more ActRII-ALK4 ligands (e.g., activin A, activin B, GDF8, GDF11, BMP6, BMP10) from binding to ActRIIA. In some embodiments, an anti-ActRIIA antibody is a multispecific antibody (e.g., bi-specific antibody) that binds to ActRIIA and one or more ActRII-ALK4 ligands (e.g., activin A, activin B, GDF8, GDF11, BMP6, BMP10), type I receptor (e.g., ALK4), and/or an additional type II receptor (e.g., ActRIIB). In some embodiments, the disclosure relates to combinations of antibodies, and uses thereof, wherein the combination of antibodies comprises an anti-ActRIIA antibody and one or more additional antibodies that bind to, for example, one or more ActRII-ALK4 ligands (e.g., activin A, activin B, GDF8, GDF11, BMP6, BMP10), type I receptors (e.g., ALK4), and/or additional type II receptors (e.g., ActRIIB). It should be noted that ActRIIA has sequence similarity to ActRIIB and therefore antibodies that bind to ActRIIA, in some instances, may also bind to and/or inhibit ActRIIB. In some embodiments, an anti-ActRII antibody of the present disclosure comprises bimagrumab (BYM338).

In certain aspects, an ActRII-ALK4 antagonist antibody, or combination of antibodies, is an antibody that inhibits at least ALK4. Therefore, in some embodiments, an ActRII-ALK4 antagonist antibody, or combination of antibodies, binds to at least ALK4. As used herein, an ALK4 antibody (anti-ALK4 antibody) generally refers to an antibody that binds to ALK4 with sufficient affinity such that the antibody is useful as a diagnostic and/or therapeutic agent in targeting ALK4. In certain embodiments, the extent of binding of an anti-ALK4 antibody to an unrelated, non-ALK4 protein is less than about 10%, 9%, 8%, 7%, 6%, 5%, 4%, 3%, 2%, or less than about 1% of the binding of the antibody to ALK4 as measured, for example, by a radioimmunoassay (RIA), Biacore, or other protein-protein interaction or binding affinity assay. In certain embodiments, an anti-ALK4 antibody binds to an epitope of ALK4 that is conserved among ALK4 from different species. In certain embodiments, an anti-ALK4 antibody binds to human ALK4. In some embodiments, an anti-ALK4 antibody may inhibit one or more ActRII-ALK4 ligands (e.g., activin A, activin B, GDF8, GDF11, BMP6, BMP10) from binding to ALK4. In some embodiments, an anti-ALK4 antibody is a multispecific antibody (e.g., bi-specific antibody) that binds to ALK4 and one or more ActRII-ALK4 ligands (e.g., activin A, activin B, GDF8, GDF11, BMP6, BMP10), and/or type II receptor (e.g., ActRIIA and/or ActRIIB). In some embodiments, the disclosure relates to combinations of antibodies, and uses thereof, wherein the combination of antibodies comprises an anti-ALK4 antibody and one or more additional antibodies that bind to, for example, one or more ActRII-ALK4 ligands (e.g., activin A, activin B, GDF8, GDF11, BMP6, BMP10), and/or type II receptors (e.g., ActRIIA and/or ActRIIB).

In certain aspects, an ActRII-ALK4 antagonist to be used in accordance with the methods and uses disclosed herein (e.g., treating, preventing, or reducing the progression rate and/or severity of heart failure associated with aging or one or more complications of heart failure associated with aging) is an antibody (ActRII-ALK4 antagonist antibody), or combination of antibodies. An ActRII-ALK4 antagonist antibody, or combination of antibodies, may bind to, for example, one or more ActRII ligands (e.g., activin A, activin B, GDF8, GDF11, BMP6, BMP10), ActRII receptor (ActRIIA and/or ActRIIB), and/or type I receptor (e.g., ALK4). As described herein, ActRII-ALK4 antagonist antibodies may be used, alone or in combination with one or more supportive therapies or active agents, to treat, prevent, or reduce the progression rate and/or severity of heart failure associated with aging, particularly treating, preventing or reducing the progression rate and/or severity of one or more heart failure-associated complications. In some embodiments, an ActRII-ALK4 antagonist antibody is selected from the group consisting of REGN-2477, garetosmab, REGN-1033, trevogumab, MYO-029, stamulumab, PF-06252616, domagrozumab, LY-2495655, landogrozumab, SRK-015, bimagrumab, and BYM338. In some embodiments, an ActRII-ALK4 antagonist antibody is selected from the group consisting of garetosmab, trevogumab, stamulumab, domagrozumab, landogrozumab, and bimagrumab.

The term antibody is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody fragment refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g., scFv); and multispecific antibodies formed from antibody fragments [see, e.g., Hudson et al. (2003) Nat. Med. 9:129-134; Plückthun, in The Pharmacology of Monoclonal Antibodies, vol. 113, Rosenburg and Moore eds., (Springer-Verlag, New York), pp. 269-315 (1994); WO 93/16185; and U.S. Pat. Nos. 5,571,894; 5,587,458; and 5,869,046]. Diabodies are antibody fragments with two antigen-binding sites that may be bivalent or bispecific [see, e.g., EP 404,097; WO 1993/01161; Hudson et al. (2003) Nat. Med. 9:129-134 (2003); and Hollinger et al. (1993) Proc. Natl. Acad. Sci. USA 90: 6444-6448]. Triabodies and tetrabodies are also described in Hudson et al. (2003) Nat. Med. 9:129-134. Single-domain antibodies are antibody fragments comprising all or a portion of the heavy-chain variable domain or all or a portion of the light-chain variable domain of an antibody. In certain embodiments, a single-domain antibody is a human single-domain antibody [see, e.g., U.S. Pat. No. 6,248,516]. Antibodies disclosed herein may be polyclonal antibodies or monoclonal antibodies. In certain embodiments, the antibodies of the present disclosure comprise a label attached thereto and able to be detected (e.g., the label can be a radioisotope, fluorescent compound, enzyme, or enzyme co-factor). In certain embodiments, the antibodies of the present disclosure are isolated antibodies. In certain embodiments, the antibodies of the present disclosure are recombinant antibodies.

The antibodies herein may be of any class. The class of an antibody refers to the type of constant domain or constant region possessed by its heavy chain. There are five major classes of antibodies: IgA, IgD, IgE, IgG, and IgM, and several of these may be further divided into subclasses (isotypes), for example, IgG₁, IgG₂, IgG₃, IgG₄, IgA₁, and IgA₂. The heavy chain constant domains that correspond to the different classes of immunoglobulins are called alpha, delta, epsilon, gamma, and mu.

In general, an antibody for use in the methods disclosed herein specifically binds to its target antigen, preferably with high binding affinity. Affinity may be expressed as a K_{D} value and reflects the intrinsic binding affinity (e.g., with minimized avidity effects). Typically, binding affinity is measured in vitro, whether in a cell-free or cell-associated setting. Any of a number of assays known in the art, including those disclosed herein, can be used to obtain binding affinity measurements including, for example, Biacore, radiolabeled antigen-binding assay (RIA), and ELISA. In some embodiments, antibodies of the present disclosure bind to their target antigens (e.g., ActRIIA, ActRIIB, activin A, activin B, GDF8, GDF11, BMP6, BMP10), with at least a K_{D} of 1×10⁻⁷ or stronger, 1×10⁻⁹ or stronger, 1×10⁻⁹ or stronger, 1×10⁻¹⁰ or stronger, 1×10⁻¹¹ or stronger, 1×10⁻¹² or stronger, 1×10⁻¹³ or stronger, or 1×10⁻¹⁴ or stronger.

In certain embodiments, K_{D} is measured by RIA performed with the Fab version of an antibody of interest and its target antigen as described by the following assay. Solution binding affinity of Fabs for the antigen is measured by equilibrating Fab with a minimal concentration of radiolabeled antigen (e.g., ¹²⁵I-labeled) in the presence of a titration series of unlabeled antigen, then capturing bound antigen with an anti-Fab antibody-coated plate [see, e.g., Chen et al. (1999) J. Mol. Biol. 293:865-881]. To establish conditions for the assay, multi-well plates (e.g., MICROTITER® from Thermo Scientific) are coated (e.g., overnight) with a capturing anti-Fab antibody (e.g., from Cappel Labs) and subsequently blocked with bovine serum albumin, preferably at room temperature (approximately 23° C.). In a non-adsorbent plate, radiolabeled antigen are mixed with serial dilutions of a Fab of interest [e.g., consistent with assessment of the anti-VEGF antibody, Fab-12, in Presta et al., (1997) Cancer Res. 57:4593-4599]. The Fab of interest is then incubated, preferably overnight but the incubation may continue for a longer period (e.g., about 65 hours) to ensure that equilibrium is reached. Thereafter, the mixtures are transferred to the capture plate for incubation, preferably at room temperature for about one hour. The solution is then removed and the plate is washed times several times, preferably with polysorbate 20 and PBS mixture. When the plates have dried, scintillant (e.g., MICROSCINT® from Packard) is added, and the plates are counted on a gamma counter (e.g., TOPCOUNT® from Packard).

According to another embodiment, K_{D} is measured using surface plasmon resonance assays using, for example a BIACORE® 2000 or a BIACORE® 3000 (BIAcore, Inc., Piscataway, N.J.) with immobilized antigen CM5 chips at about 10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. For example, an antigen can be diluted with 10 mM sodium acetate, pH 4.8, to 5 μg/ml (about 0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20®) surfactant (PBST) at a flow rate of approximately 25 μl/min. Association rates (kₒₙ.) and dissociation rates (kₒf) are calculated using, for example, a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ [see, e.g., Chen et al., (1999) J. Mol. Biol. 293:865-881]. If the on-rate exceeds, for example, 10⁶ M⁻¹ s⁻¹ by the surface plasmon resonance assay above, then the on-rate can be determined by using a fluorescent quenching technique that measures the increase or decrease in fluorescence emission intensity (e.g., excitation=295 nm; emission=340 nm, 16 nm band-pass) of a 20 nM anti-antigen antibody (Fab form) in PBS in the presence of increasing concentrations of antigen as measured in a spectrometer, such as a stop-flow equipped spectrophotometer (Aviv Instruments) or a 8000-series SLM-AMINCO® spectrophotometer (ThermoSpectronic) with a stirred cuvette.

Antibody fragments can be made by various techniques, including but not limited to proteolytic digestion of an intact antibody as well as production by recombinant host cells (e.g., E. coli or phage), as described herein. The nucleic acid and amino acid sequences of human ActRIIA, ActRIIB, ALK4, activin (activin A, activin B, activin C, and activin E), GDF11, GDF8, BMP10, and BMP6, are known in the art. In addition, numerous methods for generating antibodies are well known in the art, some of which are described herein. Therefore, antibody antagonists for use in accordance with this disclosure may be routinely made by the skilled person in the art based on the knowledge in the art and teachings provided herein.

In certain embodiments, an antibody provided herein is a chimeric antibody. A chimeric antibody refers to an antibody in which a portion of the heavy and/or light chain is derived from a particular source or species, while the remainder of the heavy and/or light chain is derived from a different source or species. Certain chimeric antibodies are described, for example, in U.S. Pat. No. 4,816,567; and Morrison et al., (1984) Proc. Natl. Acad. Sci. USA, 81:6851-6855. In some embodiments, a chimeric antibody comprises a non-human variable region (e.g., a variable region derived from a mouse, rat, hamster, rabbit, or non-human primate, such as a monkey) and a human constant region. In some embodiments, a chimeric antibody is a “class switched” antibody in which the class or subclass has been changed from that of the parent antibody. In general, chimeric antibodies include antigen-binding fragments thereof.

In certain embodiments, a chimeric antibody provided herein is a humanized antibody. A humanized antibody refers to a chimeric antibody comprising amino acid residues from non-human hypervariable regions (HVRs) and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Humanized antibodies and methods of making them are reviewed, for example, in Almagro and Fransson (2008) Front. Biosci. 13:1619-1633 and are further described, for example, in Riechmann et al., (1988) Nature 332:323-329; Queen et al. (1989) Proc. Nat'l Acad. Sci. USA 86:10029-10033; U.S. Pat. Nos. 5,821,337; 7,527,791; 6,982,321; and U.S. Pat. No. 7,087,409; Kashmiri et al., (2005) Methods 36:25-34 [describing SDR (a-CDR) grafting]; Padlan, Mol. Immunol. (1991) 28:489-498 (describing “resurfacing”); Dall'Acqua et al. (2005) Methods 36:43-60 (describing “FR shuffling”); Osbourn et al. (2005) Methods 36:61-68; and Klimka et al. Br. J. Cancer (2000) 83:252-260 (describing the “guided selection” approach to FR shuffling). Human framework regions that may be used for humanization include but are not limited to framework regions selected using the “best-fit” method [see, e.g., Sims et al. (1993) J. Immunol. 151:2296]; framework regions derived from the consensus sequence of human antibodies of a particular subgroup of light or heavy chain variable regions [see, e.g., Carter et al. (1992) Proc. Natl. Acad. Sci. USA, 89:4285; and Presta et al. (1993) J. Immunol., 151:2623]; human mature (somatically mutated) framework regions or human germline framework regions [see, e.g., Almagro and Fransson (2008) Front. Biosci. 13:1619-1633]; and framework regions derived from screening FR libraries [see, e.g., Baca et al., (1997) J. Biol. Chem. 272:10678-10684; and Rosok et al., (1996) J. Biol. Chem. 271:22611-22618].

In certain embodiments, an antibody provided herein is a human antibody. Human antibodies can be produced using various techniques known in the art. Human antibodies are described generally in van Dijk and van de Winkel (2008) Curr. Opin. Pharmacol. 5: 368-74 (2001) and Lonberg, Curr. Opin. Immunol. 20:450-459. For example, human antibodies may be prepared by administering an immunogen (e.g., ActRII-ALK4 ligands (e.g., activin A, activin B, GDF8, GDF11, BMP6, BMP10), ActRII receptor (ActRIIA and/or ActRIIB), and/or type I receptor (e.g., ALK4)) to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. For a review of methods for obtaining human antibodies from transgenic animals see, for example, Lonberg (2005) Nat. Biotech. 23:1117-1125; U.S. Pat. Nos. 6,075,181 and 6,150,584 (describing XENOMOUSE™ technology); U.S. Pat. No. 5,770,429 (describing HuMab® technology); U.S. Pat. No. 7,041,870 (describing K-M MOUSE® technology); and U.S. Patent Application Publication No. 2007/0061900 (describing VelociMouse® technology). Human variable regions from intact antibodies generated by such animals may be further modified, for example, by combining with a different human constant region.

Human antibodies provided herein can also be made by hybridoma-based methods. Human myeloma and mouse-human heteromyeloma cell lines for the production of human monoclonal antibodies have been described [see, e.g., Kozbor J. Immunol., (1984) 133: 3001; Brodeur et al. (1987) Monoclonal Antibody Production Techniques and Applications, pp. 51-63, Marcel Dekker, Inc., New York; and Boerner et al. (1991) J. Immunol., 147: 86]. Human antibodies generated via human B-cell hybridoma technology are also described in Li et al., (2006) Proc. Natl. Acad. Sci. USA, 103:3557-3562. Additional methods include those described, for example, in U.S. Pat. No. 7,189,826 (describing production of monoclonal human IgM antibodies from hybridoma cell lines) and Ni, Xiandai Mianyixue (2006) 26(4):265-268 (2006) (describing human-human hybridomas). Human hybridoma technology (Trioma technology) is also described in Vollmers and Brandlein (2005) Histol. Histopathol., 20(3):927-937 (2005) and Vollmers and Brandlein (2005) Methods Find Exp. Clin. Pharmacol., 27(3):185-91. Human antibodies provided herein may also be generated by isolating Fv clone variable-domain sequences selected from human-derived phage display libraries. Such variable-domain sequences may then be combined with a desired human constant domain. Techniques for selecting human antibodies from antibody libraries are known in the art and described herein.

For example, antibodies of the present disclosure may be isolated by screening combinatorial libraries for antibodies with the desired activity or activities. A variety of methods are known in the art for generating phage display libraries and screening such libraries for antibodies possessing the desired binding characteristics. Such methods are reviewed, for example, in Hoogenboom et al. (2001) in Methods in Molecular Biology 178:1-37, O'Brien et al., ed., Human Press, Totowa, N.J. and further described, for example, in the McCafferty et al. (1991) Nature 348:552-554; Clackson et al., (1991) Nature 352: 624-628; Marks et al. (1992) J. Mol. Biol. 222:581-597; Marks and Bradbury (2003) in Methods in Molecular Biology 248:161-175, Lo, ed., Human Press, Totowa, N.J.; Sidhu et al. (2004) J. Mol. Biol. 338(2):299-310; Lee et al. (2004) J. Mol. Biol. 340(5):1073-1093; Fellouse (2004) Proc. Natl. Acad. Sci. USA 101(34):12467-12472; and Lee et al. (2004) J. Immunol. Methods 284(1-2): 119-132.

In certain phage display methods, repertoires of VH and VL genes are separately cloned by polymerase chain reaction (PCR) and recombined randomly in phage libraries, which can then be screened for antigen-binding phage as described in Winter et al. (1994) Ann. Rev. Immunol., 12: 433-455. Phage typically display antibody fragments, either as single-chain Fv (scFv) fragments or as Fab fragments. Libraries from immunized sources provide high-affinity antibodies to the immunogen (e.g., ActRII ligands (e.g., activin A, activin B, GDF8, GDF11, BMP6, BMP10), ActRII receptor (ActRIIA and/or ActRIIB), and/or type I receptor (e.g., ALK4)) without the requirement of constructing hybridomas. Alternatively, the naive repertoire can be cloned (e.g., from human) to provide a single source of antibodies to a wide range of non-self and also self-antigens without any immunization as described by Griffiths et al. (1993) EMBO J, 12: 725-734. Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter (1992) J. Mol. Biol., 227: 381-388. Patent publications describing human antibody phage libraries include, for example: U.S. Pat. No. 5,750,373, and U.S. Patent Publication Nos. 2005/0079574, 2005/0119455, 2005/0266000, 2007/0117126, 2007/0160598, 2007/0237764, 2007/0292936, and 2009/0002360.

In certain embodiments, an antibody provided herein is a multispecific antibody, for example, a bispecific antibody. Multispecific antibodies (typically monoclonal antibodies) that have binding specificities for at least two different epitopes (e.g., two, three, four, five, or six or more) on one or more (e.g., two, three, four, five, six or more) antigens.

Techniques for making multispecific antibodies include, but are not limited to, recombinant co-expression of two immunoglobulin heavy-chain/light-chain pairs having different specificities [see, e.g., Milstein and Cuello (1983) Nature 305: 537; International patent publication no. WO 93/08829; and Traunecker et al. (1991) EMBO J. 10: 3655, and U.S. Pat. No. 5,731,168 (“knob-in-hole” engineering)]. Multispecific antibodies may also be made by engineering electrostatic steering effects for making antibody Fc-heterodimeric molecules (see, e.g., WO 2009/089004A1); cross-linking two or more antibodies or fragments [see, e.g., U.S. Pat. No. 4,676,980; and Brennan et al. (1985) Science, 229: 81]; using leucine zippers to produce bispecific antibodies [see, e.g., Kostelny et al. (1992) J. Immunol., 148(5):1547-1553]; using “diabody” technology for making bispecific antibody fragments [see, e.g., Hollinger et al. (1993) Proc. Natl. Acad. Sci. USA, 90:6444-6448]; using single-chain Fv (sFv) dimers [see, e.g., Gruber et al. (1994) J. Immunol., 152:5368]; and preparing trispecific antibodies (see, e.g., Tutt et al. (1991) J. Immunol. 147: 60. Multispecific antibodies can be prepared as full-length antibodies or antibody fragments. Engineered antibodies with three or more functional antigen-binding sites, including “Octopus antibodies,” are also included herein [see, e.g., US 2006/0025576A1].

In certain embodiments, an antibody disclosed herein is a monoclonal antibody. Monoclonal antibody refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present methods may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

For example, by using immunogens derived from activin, anti-protein/anti-peptide antisera or monoclonal antibodies can be made by standard protocols [see, e.g., Antibodies: A Laboratory Manual ed. by Harlow and Lane (1988) Cold Spring Harbor Press: 1988]. A mammal, such as a mouse, hamster, or rabbit, can be immunized with an immunogenic form of the activin polypeptide, an antigenic fragment which is capable of eliciting an antibody response, or a fusion protein. Techniques for conferring immunogenicity on a protein or peptide include conjugation to carriers or other techniques well known in the art. An immunogenic portion of an activin polypeptide can be administered in the presence of adjuvant. The progress of immunization can be monitored by detection of antibody titers in plasma or serum. Standard ELISA or other immunoassays can be used with the immunogen as antigen to assess the levels of antibody production and/or level of binding affinity.

Following immunization of an animal with an antigenic preparation of activin, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique [see, e.g., Kohler and Milstein (1975) Nature, 256: 495-497], the human B cell hybridoma technique [see, e.g., Kozbar et al. (1983) Immunology Today, 4:72], and the EBV-hybridoma technique to produce human monoclonal antibodies [Cole et al. (1985) Monoclonal Antibodies and Cancer Therapy, Alan R. Liss, Inc. pp. 77-96]. Hybridoma cells can be screened immunochemically for production of antibodies specifically reactive with an activin polypeptide, and monoclonal antibodies isolated from a culture comprising such hybridoma cells.

In certain embodiments, one or more amino acid modifications may be introduced into the Fc region of an antibody provided herein thereby generating an Fc region variant. The Fc region variant may comprise a human Fc region sequence (e.g., a human IgG1, IgG2, IgG3 or IgG4 Fc region) comprising an amino acid modification (e.g., a substitution, deletion, and/or addition) at one or more amino acid positions.

For example, the present disclosure contemplates an antibody variant that possesses some but not all effector functions, which make it a desirable candidate for applications in which the half-life of the antibody in vivo is important yet certain effector functions [e.g., complement-dependent cytotoxicity (CDC) and antibody-dependent cellular cytotoxicity (ADCC)] are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in, for example, Ravetch and Kinet (1991) Annu. Rev. Immunol. 9:457-492. Non-limiting examples of in vitro assays to assess ADCC activity of a molecule of interest are described in U.S. Pat. No. 5,500,362; Hellstrom, I. et al. (1986) Proc. Natl. Acad. Sci. USA 83:7059-7063]; Hellstrom, I et al. (1985) Proc. Natl. Acad. Sci. USA 82:1499-1502; U.S. Pat. No. 5,821,337; Bruggemann, M. et al. (1987) J. Exp. Med. 166:1351-1361. Alternatively, non-radioactive assays methods may be employed (e.g., ACTI™, non-radioactive cytotoxicity assay for flow cytometry; CellTechnology, Inc. Mountain View, Calif.; and CytoTox 96® non-radioactive cytotoxicity assay, Promega, Madison, Wis.). Useful effector cells for such assays include peripheral blood mononuclear cells (PBMC) and natural killer (NK) cells. Alternatively, or additionally, ADCC activity of the molecule of interest may be assessed in vivo, for example, in an animal model such as that disclosed in Clynes et al. (1998) Proc. Natl. Acad. Sci. USA 95:652-656. Clq binding assays may also be carried out to confirm that the antibody is unable to bind Clq and hence lacks CDC activity [see, e.g., Clq and C3c binding ELISA in WO 2006/029879 and WO 2005/100402]. To assess complement activation, a CDC assay may be performed [see, e.g., Gazzano-Santoro et al. (1996) J. Immunol. Methods 202:163; Cragg, M. S. et al. (2003) Blood 101:1045-1052; and Cragg, M. S, and M. J. Glennie (2004) Blood 103:2738-2743]. FcRn binding and in vivo clearance/half-life determinations can also be performed using methods known in the art [see, e.g., Petkova, S. B. et al. (2006) Intl. Immunol. 18(12):1759-1769]. Antibodies of the present disclosure with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

In addition, the techniques used to screen antibodies in order to identify a desirable antibody may influence the properties of the antibody obtained. For example, if an antibody is to be used for binding an antigen in solution, it may be desirable to test solution binding. A variety of different techniques are available for testing interactions between antibodies and antigens to identify particularly desirable antibodies. Such techniques include ELISAs, surface plasmon resonance binding assays (e.g., the Biacore binding assay, Biacore AB, Uppsala, Sweden), sandwich assays (e.g., the paramagnetic bead system of IGEN International, Inc., Gaithersburg, Maryland), western blots, immunoprecipitation assays, and immunohistochemistry.

In certain embodiments, amino acid sequence variants of the antibodies and/or the binding polypeptides provided herein are contemplated. For example, it may be desirable to improve the binding affinity and/or other biological properties of the antibody and/or binding polypeptide. Amino acid sequence variants of an antibody and/or binding polypeptides may be prepared by introducing appropriate modifications into the nucleotide sequence encoding the antibody and/or binding polypeptide, or by peptide synthesis. Such modifications include, for example, deletions from, and/or insertions into and/or substitutions of residues within the amino acid sequences of the antibody and/or binding polypeptide. Any combination of deletion, insertion, and substitution can be made to arrive at the final construct, provided that the final construct possesses the desired characteristics, e.g., target-binding (e.g., and activin such as activin E and/or activin C binding).

Alterations (e.g., substitutions) may be made in HVRs, for example, to improve antibody affinity. Such alterations may be made in HVR “hotspots,” i.e., residues encoded by codons that undergo mutation at high frequency during the somatic maturation process [see, e.g., Chowdhury (2008) Methods Mol. Biol. 207:179-196 (2008)], and/or SDRs (a-CDRs), with the resulting variant VH or VL being tested for binding affinity. Affinity maturation by constructing and reselecting from secondary libraries has been described in the art [see, e.g., Hoogenboom et al., in Methods in Molecular Biology 178:1-37, O'Brien et al., ed., Human Press, Totowa, N.J., (2001). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind to the antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of the antibody and/or the binding polypeptide that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody-antigen is affected. Further substitutions may be introduced at the amino acid locations demonstrating functional sensitivity to the initial substitutions. Alternatively, or additionally, a crystal structure of an antigen-antibody complex is determined to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion of the N- or C-terminus of the antibody to an enzyme (e.g., for ADEPT) or a polypeptide which increases the serum half-life of the antibody.

In certain embodiments, an antibody and/or binding polypeptide provided herein may be further modified to contain additional nonproteinaceous moieties that are known in the art and readily available. The moieties suitable for derivatization of the antibody and/or binding polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody and/or binding polypeptide may vary, and if more than one polymer are attached, they can be the same or different molecules. In general, the number and/or type of polymers used for derivatization can be determined based on considerations including, but not limited to, the particular properties or functions of the antibody and/or binding polypeptide to be improved, whether the antibody derivative and/or binding polypeptide derivative will be used in a therapy under defined conditions.

### 4. Small Molecule Antagonists

In certain aspects, an ActRII-ALK4 antagonist to be used in accordance with the methods and uses disclosed herein (e.g., treating, preventing, or reducing the progression rate and/or severity of heart failure associated with aging, or one or more complications of heart failure associated with aging, is a small molecule (ActRII-ALK4 small molecule antagonist), or combination of small molecule antagonists. An ActRII-ALK4 small molecule antagonist, or combination of small molecule antagonists, may inhibit, for example, one or more ActRII ligands (e.g., activin A, activin B, GDF8, GDF11, BMP6, BMP10), ActRII receptor (ActRIIA and/or ActRIIB), type I receptor (e.g., ALK4), a type II receptor (e.g., ActRIIB and/or ActRIIA), and/or one or more signaling factors. In some embodiments, an ActRII-ALK4 small molecule antagonist, or combination of small molecule antagonists, inhibits signaling mediated by one or more ActRII-ALK4 ligands, for example, as determined in a cell-based assay such as those described herein. As described herein, ActRII-ALK4 small molecule antagonists may be used, alone or in combination with one or more supportive therapies or active agents, to treat, prevent, or reduce the progression rate and/or severity of heart failure), particularly treating, preventing or reducing the progression rate and/or severity of one or more heart failure-associated complications.

In some embodiments, a ActRII-ALK4 small molecule antagonist, or combination of small molecule antagonists, inhibits at least GDF11, optionally further inhibiting one or more of GDF8, activin (e.g., activin A, activin B, activin C, activin E, activin AB, activin AC, activin BC, activin AE and/or activin BE), BMP6, BMP10, ActRIIA, ActRIIB, ALK4, and one or more Smad signaling factors. In some embodiments, a ActRII-ALK4 small molecule antagonist, or combination of small molecule antagonists, inhibits at least GDF8, optionally further inhibiting one or more of GDF11, activin (e.g., activin A, activin B, activin C, activin E, activin AB, activin AC, activin BC, activin AE and/or activin BE), BMP6, BMP10, ActRIIA, ActRIIB, ALK4, and one or more Smad signaling factors. In some embodiments, a ActRII-ALK4 small molecule antagonist, or combination of small molecule antagonists, inhibits at least activin (activin A, activin B, activin C, activin E, activin AB, activin AC, activin BC, activin AE and/or activin BE), optionally further inhibiting one or more of GDF8, GDF11, BMP6, BMP10, ActRIIA, ActRIIB, ALK4, and one or more Smad signaling factors. In some embodiments, an ActRII-ALK4 small molecule antagonist, or combination of small molecule antagonists, inhibits at least activin B, optionally further inhibiting one or more of GDF8, GDF11, BMP6, BMP10, ActRIIA, ActRIIB, ALK4, and one or more Smad signaling factors. In some embodiments, a ActRII-ALK4 small molecule antagonist, or combination of small molecule antagonists, inhibits at least BMP6, optionally further inhibiting one or more of GDF8, activin (e.g., activin A, activin B, activin C, activin E, activin AB, activin AC, activin BC, activin AE and/or activin BE), GDF11, BMP10, ActRIIA, ActRIIB, ALK4, and one or more Smad proteins (e.g., Smads 2 and 3). In some embodiments, an ActRII-ALK4 small molecule antagonist, or combination of small molecule antagonists, inhibits at least BMP10, optionally further inhibiting one or more of GDF8, activin (e.g., activin A, activin B, activin C, activin E, activin AB, activin AC, activin BC, activin AE and/or activin BE), BMP6, GDF11, ActRIIA, ActRIIB, ALK4, and one or more Smad signaling factors. In some embodiments, an ActRII-ALK4 small molecule antagonist, or combination of small molecule antagonists, inhibits at least ActRIIA, optionally further inhibiting one or more of GDF8, activin (e.g., activin A, activin B, activin C, activin E, activin AB, activin AC, activin BC, activin AE and/or activin BE), BMP6, GDF11, BMP10, ActRIIB, ALK4, and one or more Smad signaling factors. In some embodiments, an ActRII-ALK4 small molecule antagonist, or combination of small molecule antagonists, inhibits at least ActRIIB, optionally further inhibiting one or more of GDF8, activin (e.g., activin A, activin B, activin C, activin E, activin AB, activin AC, activin BC, activin AE and/or activin BE), BMP6, GDF11, BMP10, ActRIIA, ALK4, and one or more Smad signaling factors. In some embodiments, an ActRII-ALK4 small molecule antagonist, or combination of small molecule antagonists, inhibits at least ALK4, optionally further inhibiting one or more of GDF8, activin (e.g., activin A, activin B, activin C, activin E, activin AB, activin AC, activin BC, activin AE and/or activin BE), BMP6, GDF11, BMP10, ActRIIA, ActRIIB, and one or more Smad signaling factors. In some embodiments, an ActRII-ALK4 small molecule antagonist, or combination of small molecule antagonists, as disclosed herein does not inhibit or does not substantially inhibit BMP9. In some embodiments, an ActRII-ALK4 small molecule antagonist, or combination of small molecule antagonists, as disclosed herein does not inhibit or does not substantially inhibit activin A.

ActRII-ALK4 small molecule antagonists can be direct or indirect inhibitors. For example, an indirect small molecule antagonist, or combination of small molecule antagonists, may inhibit the expression (e.g., transcription, translation, cellular secretion, or combinations thereof) of at least one or more ActRII-ALK4 ligands (e.g., activin A, activin B, GDF8, GDF11, BMP6, BMP10), type I receptor (e.g., ALK4), type II receptors (e.g., ActRIIA and/or ActRIIB), and/or one or more downstream signaling components (e.g., Smads). Alternatively, a direct small molecule antagonist, or combination of small molecule antagonists, may directly bind to and inhibit, for example, one or more one or more ActRII-ALK4 ligands (e.g., activin A, activin B, GDF8, GDF11, BMP6, BMP10), type I receptor (e.g., ALK4), type II receptors (e.g., ActRIIA and/or ActRIIB), and/or one or more downstream signaling components (e.g., Smads). Combinations of one or more indirect and one or more direct ActRII-ALK4 small molecule antagonists may be used in accordance with the methods disclosed herein.

Binding small-molecule antagonists of the present disclosure may be identified and chemically synthesized using known methodology (see, e.g., PCT Publication Nos. WO 00/00823 and WO 00/39585). In general, small molecule antagonists of the disclosure are usually less than about 2000 daltons in size, alternatively less than about 1500, 750, 500, 250 or 200 daltons in size, wherein such organic small molecules that are capable of binding, preferably specifically, to a polypeptide as described herein. These small molecule antagonists may be identified without undue experimentation using well-known techniques. In this regard, it is noted that techniques for screening organic small-molecule libraries for molecules that are capable of binding to a polypeptide target are well known in the art (see, e.g., international patent publication Nos. WO00/00823 and WO00/39585).

Binding organic small molecules of the present disclosure may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, allyl sulfonates, aromatic compounds, heterocyclic compounds, anilines, allenes, alkynes, diols, amino alcohols, oxazolidines, oxazolines, thiazolidines, thiazolines, enamines, sulfonamides, epoxides, aziridines, isocyanates, sulfonyl chlorides, diazo compounds, and acid chlorides.

### 5. Polynucleotide Antagonists

In certain aspects, an ActRII-ALK4 antagonist to be used in accordance with the methods and uses disclosed herein (e.g., treating, preventing, or reducing the progression rate and/or severity of heart failure associated with aging, or one or more complications of heart failure associated with aging) is a polynucleotide (ActRII-ALK4 polynucleotide antagonist), or combination of polynucleotides. An ActRII-ALK4 polynucleotide antagonist, or combination of polynucleotide antagonists, may inhibit, for example, one or more ActRII-ALK4 ligands (e.g., activin A, activin B, GDF8, GDF11, BMP6, BMP10), type I receptors (e.g., ALK4), type II receptors (e.g., ActRIIA and/or ActRIIB), and/or downstream signaling component (e.g., Smads). In some embodiments, an ActRII-ALK4 polynucleotide antagonist, or combination of polynucleotide antagonists, inhibits signaling mediated by one or more ActRII-ALK4 ligands, for example, as determined in a cell-based assay such as those described herein. As described herein, ActRII-ALK4 polynucleotide antagonists may be used, alone or in combination with one or more supportive therapies or active agents, to treat, prevent, or reduce the progression rate and/or severity of heart failure associated with aging, particularly treating, preventing or reducing the progression rate and/or severity of one or more heart failure-associated complications.

In some embodiments, an ActRII-ALK4 polynucleotide antagonist, or combination of polynucleotide antagonists, inhibits at least GDF11, optionally further inhibiting one or more of GDF8, activin (e.g., activin A, activin B, activin C, activin E, activin AB, activin AC, activin BC, activin AE and/or activin BE), BMP6, BMP10, ActRIIA ActRIIB, ALK4, and one or more Smad signaling factors. In some embodiments, an ActRII-ALK4 polynucleotide antagonist, or combination of polynucleotide antagonists, inhibits at least GDF8, optionally further inhibiting one or more of GDF11, activin (e.g., activin A, activin B, activin C, activin E, activin AB, activin AC, activin BC, activin AE and/or activin BE), BMP6, BMP10, ActRIIA, ActRIIB, ALK4, and one or more Smad signaling factors. In some embodiments, an ActRII-ALK4 polynucleotide antagonist, or combination of polynucleotide antagonists, inhibits at least activin (activin A, activin B, activin C, activin E, activin AB, activin AC, activin BC, activin AE and/or activin BE), optionally further inhibiting one or more of GDF8, GDF11, BMP6, BMP10, ActRIIA, ActRIIB, ALK4, and one or more Smad signaling factors. In some embodiments, an ActRII-ALK4 polynucleotide antagonist, or combination of polynucleotide antagonists, inhibits at least activin B, optionally further inhibiting one or more of GDF8, GDF11, BMP6, BMP10, ActRIIA, ActRIIB, ALK4, and one or more Smad signaling factors. In some embodiments, an ActRII-ALK4 polynucleotide antagonist, or combination of polynucleotide antagonists, inhibits at least BMP6, optionally further inhibiting one or more of GDF8, activin (e.g., activin A, activin B, activin C, activin E, activin AB, activin AC, activin BC, activin AE and/or activin BE), GDF11, BMP10, ActRIIA, ActRIIB, ALK4, and one or more Smad proteins signaling factors. In some embodiments, an ActRII-ALK4 polynucleotide antagonist, or combination of polynucleotide antagonists, inhibits at least BMP10, optionally further inhibiting one or more of GDF8, activin (e.g., activin A, activin B, activin C, activin E, activin AB, activin AC, activin BC, activin AE and/or activin BE), BMP6, GDF11, ActRIIA, ActRIIB, ALK4, and one or more Smad signaling factors. In some embodiments, an ActRII-ALK4 polynucleotide antagonist, or combination of polynucleotide antagonists, inhibits at least ActRIIA, optionally further inhibiting one or more of GDF8, activin (e.g., activin A, activin B, activin C, activin E, activin AB, activin AC, activin BC, activin AE and/or activin BE), BMP6, GDF11, BMP10, ActRIIB, ALK4, and one or more Smad signaling factors. In some embodiments, an ActRII-ALK4 polynucleotide antagonist, or combination of polynucleotide antagonists, inhibits at least ActRIIB, optionally further inhibiting one or more of GDF8, activin (e.g., activin A, activin B, activin C, activin E, activin AB, activin AC, activin BC, activin AE and/or activin BE), BMP6, GDF11, ActRIIA, BMP10, ALK4, and one or more Smad signaling factors. In some embodiments, an ActRII-ALK4 polynucleotide antagonist, or combination of polynucleotide antagonists, inhibits at least ALK4, optionally further inhibiting one or more of GDF8, activin (e.g., activin A, activin B, activin C, activin E, activin AB, activin AC, activin BC, activin AE and/or activin BE), BMP6, GDF11, ActRIIA, ActRIIB, BMP10, and one or more Smad signaling factors. In some embodiments, an ActRII-ALK4 polynucleotide antagonist, or combination of polynucleotide antagonists, as disclosed herein does not inhibit or does not substantially inhibit BMP9. In some embodiments, an ActRII-ALK4 polynucleotide antagonist, or combination of polynucleotide antagonists, as disclosed herein does not inhibit or does not substantially inhibit activin A.

In some embodiments, the polynucleotide antagonists of the disclosure may be an antisense nucleic acid, an RNAi molecule [e.g., small interfering RNA (siRNA), small-hairpin RNA (shRNA), microRNA (miRNA)], an aptamer and/or a ribozyme. The nucleic acid and amino acid sequences of human GDF11, GDF8, activin (activin A, activin B, activin C, and activin E), BMP6, ActRIIA, ActRIIB, BMP10, ALK4, and Smad signaling factors are known in the art. In addition, many different methods of generating polynucleotide antagonists are well known in the art. Therefore, polynucleotide antagonists for use in accordance with this disclosure may be routinely made by the skilled person in the art based on the knowledge in the art and teachings provided herein.

Antisense technology can be used to control gene expression through antisense DNA or RNA, or through triple-helix formation. Antisense techniques are discussed, for example, in Okano (1991) J. Neurochem. 56:560; Oligodeoxynucleotides as Antisense Inhibitors of Gene Expression, CRC Press, Boca Raton, Fla. (1988). Triple-helix formation is discussed in, for instance, Cooney et al. (1988) Science 241:456; and Dervan et al., (1991) Science 251:1300. The methods are based on binding of a polynucleotide to a complementary DNA or RNA. In some embodiments, the antisense nucleic acids comprise a single-stranded RNA or DNA sequence that is complementary to at least a portion of an RNA transcript of a gene disclosed herein. However, absolute complementarity, although preferred, is not required.

A sequence “complementary to at least a portion of an RNA,” referred to herein, means a sequence having sufficient complementarity to be able to hybridize with the RNA, forming a stable duplex; in the case of double-stranded antisense nucleic acids of a gene disclosed herein, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with an RNA it may contain and still form a stable duplex (or triplex as the case may be). One skilled in the art can ascertain a tolerable degree of mismatch by use of standard procedures to determine the melting point of the hybridized complex.

Polynucleotides that are complementary to the 5′ end of the message, for example, the 5′-untranslated sequence up to and including the AUG initiation codon, should work most efficiently at inhibiting translation. However, sequences complementary to the 3′-untranslated sequences of mRNAs have been shown to be effective at inhibiting translation of mRNAs as well [see, e.g., Wagner, R., (1994) Nature 372:333-335]. Thus, oligonucleotides complementary to either the 5′- or 3′-non-translated, non-coding regions of a gene of the disclosure, could be used in an antisense approach to inhibit translation of an endogenous mRNA. Polynucleotides complementary to the 5′-untranslated region of the mRNA should include the complement of the AUG start codon. Antisense polynucleotides complementary to mRNA coding regions are less efficient inhibitors of translation but could be used in accordance with the methods of the present disclosure. Whether designed to hybridize to the 5′-, 3′- or coding region of an mRNA of the disclosure, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

In one embodiment, the antisense nucleic acid of the present disclosure is produced intracellularly by transcription from an exogenous sequence. For example, a vector or a portion thereof is transcribed, producing an antisense nucleic acid (RNA) of a gene of the disclosure. Such a vector contains a sequence encoding the desired antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the sequence encoding desired genes of the instant disclosure, or fragments thereof, can be by any promoter known in the art to act in vertebrate, preferably human cells. Such promoters can be inducible or constitutive. Such promoters include, but are not limited to, the SV40 early promoter region [see, e.g., Benoist and Chambon (1981) Nature 290:304-310], the promoter contained in the 3′ long-terminal repeat of Rous sarcoma virus [see, e.g., Yamamoto et al. (1980) Cell 22:787-797], the herpes thymidine promoter [see, e.g., Wagner et al. (1981) Proc. Natl. Acad. Sci. U.S.A. 78:1441-1445], and the regulatory sequences of the metallothionein gene [see, e.g., Brinster, et al. (1982) Nature 296:39-42].

In some embodiments, the polynucleotide antagonists are interfering RNA (RNAi) molecules that target the expression of one or more of: GDF11, GDF8, activin (activin A, activin B, activin C, and activin E), BMP6, ActRIIA, ActRIIB, BMP10, ALK4, and Smad signaling factors. RNAi refers to the expression of an RNA which interferes with the expression of the targeted mRNA. Specifically, RNAi silences a targeted gene via interacting with the specific mRNA through a siRNA (small interfering RNA). The ds RNA complex is then targeted for degradation by the cell. An siRNA molecule is a double-stranded RNA duplex of 10 to 50 nucleotides in length, which interferes with the expression of a target gene which is sufficiently complementary (e.g. at least 80% identity to the gene). In some embodiments, the siRNA molecule comprises a nucleotide sequence that is at least 85, 90, 95, 96, 97, 98, 99, or 100% identical to the nucleotide sequence of the target gene.

Additional RNAi molecules include short-hairpin RNA (shRNA); also, short-interfering hairpin and microRNA (miRNA). The shRNA molecule contains sense and antisense sequences from a target gene connected by a loop. The shRNA is transported from the nucleus into the cytoplasm, and it is degraded along with the mRNA. Pol III or U6 promoters can be used to express RNAs for RNAi. Paddison et al. [Genes & Dev. (2002) 16:948-958, 2002] have used small RNA molecules folded into hairpins as a means to affect RNAi. Accordingly, such short-hairpin RNA (shRNA) molecules are also advantageously used in the methods described herein. The length of the stem and loop of functional shRNAs varies; stem lengths can range anywhere from about 25 to about 30 nt, and loop size can range between 4 to about 25 nt without affecting silencing activity. While not wishing to be bound by any particular theory, it is believed that these shRNAs resemble the double-stranded RNA (dsRNA) products of the DICER RNase and, in any event, have the same capacity for inhibiting expression of a specific gene. The shRNA can be expressed from a lentiviral vector. An miRNA is a single-stranded RNA of about 10 to 70 nucleotides in length that are initially transcribed as pre-miRNA characterized by a “stem-loop” structure, which are subsequently processed into mature miRNA after further processing through the RISC.

Molecules that mediate RNAi, including without limitation siRNA, can be produced in vitro by chemical synthesis (Hohjoh, FEBS Lett 521:195-199, 2002), hydrolysis of dsRNA (Yang et al., Proc Natl Acad Sci USA 99:9942-9947, 2002), by in vitro transcription with T7 RNA polymerase (Donzeet et al., Nucleic Acids Res 30:e46, 2002; Yu et al., Proc Natl Acad Sci USA 99:6047-6052, 2002), and by hydrolysis of double-stranded RNA using a nuclease such as E. coli RNase III (Yang et al., Proc Natl Acad Sci USA 99:9942-9947, 2002).

According to another aspect, the disclosure provides polynucleotide antagonists including but not limited to, a decoy DNA, a double-stranded DNA, a single-stranded DNA, a complexed DNA, an encapsulated DNA, a viral DNA, a plasmid DNA, a naked RNA, an encapsulated RNA, a viral RNA, a double-stranded RNA, a molecule capable of generating RNA interference, or combinations thereof.

In some embodiments, the polynucleotide antagonists of the disclosure are aptamers. Aptamers are nucleic acid molecules, including double-stranded DNA and single-stranded RNA molecules, which bind to and from tertiary structures that specifically bind to a target molecule. The generation and therapeutic use of aptamers are well established in the art (see, e.g., U.S. Pat. No. 5,475,096). Additional information on aptamers can be found in U.S. Patent Application Publication No. 20060148748. Nucleic acid aptamers are selected using methods known in the art, for example via the Systematic Evolution of Ligands by Exponential Enrichment (SELEX) process. SELEX is a method for the in vitro evolution of nucleic acid molecules with highly specific binding to target molecules as described in, e.g., U.S. Pat. Nos. 5,475,096; 5,580,737; 5,567,588; 5,707,796; 5,763,177; 6,011,577; and 6,699,843. Another screening method to identify aptamers is described in U.S. Pat. No. 5,270,163. The SELEX process is based on the capacity of nucleic acids for forming a variety of two- and three-dimensional structures, as well as the chemical versatility available within the nucleotide monomers to act as ligands (form specific binding pairs) with virtually any chemical compound, whether monomeric or polymeric, including other nucleic acid molecules and polypeptides. Molecules of any size or composition can serve as targets. The SELEX method involves selection from a mixture of candidate oligonucleotides and stepwise iterations of binding, partitioning and amplification, using the same general selection scheme, to achieve desired binding affinity and selectivity. Starting from a mixture of nucleic acids, which can comprise a segment of randomized sequence, the SELEX method includes steps of contacting the mixture with the target under conditions favorable for binding; partitioning unbound nucleic acids from those nucleic acids which have bound specifically to target molecules; dissociating the nucleic acid-target complexes; amplifying the nucleic acids dissociated from the nucleic acid-target complexes to yield a ligand enriched mixture of nucleic acids. The steps of binding, partitioning, dissociating and amplifying are repeated through as many cycles as desired to yield nucleic acid ligands which bind with high affinity and specificity to the target molecule.

Typically, such binding molecules are separately administered to the animal [see, e.g., O'Connor (1991) J. Neurochem. 56:560], but such binding molecules can also be expressed in vivo from polynucleotides taken up by a host cell and expressed in vivo [see, e.g., Oligodeoxynucleotides as Antisense Inhibitors of Gene Expression, CRC Press, Boca Raton, Fla. (1988)].

### 6. Heart Failure

In part, the present disclosure relates to a method of treating heart failure comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the disclosure relates to a method of treating, preventing, or reducing the progression rate and/or severity of one or more comorbidities of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the disclosure relates to treating heart failure associated with aging, wherein the patient has heart failure with preserved ejection fraction (HFpEF). In some embodiments, the disclosure relates to a method of treating HFpEF. In some embodiments, the disclosure relates to a method of treating a patient with diastolic dysfunction. In some embodiments, the disclosure relates to a method of treating a patient with no reduction in left ventricular ejection fraction (LVEF). In some embodiments, the disclosure relates to treating a patient with an increase in left ventricular wall thickness. In some embodiments, the disclosure relates to treating a patient with an increase in left atrial size.

These methods are particularly aimed at therapeutic and prophylactic treatments of animals, and more particularly, humans. The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The terms “treatment”, “treating”, “alleviating” and the like are used herein to generally mean obtaining a desired pharmacologic and/or physiologic effect, and may also be used to refer to improving, alleviating, and/or decreasing the severity of one or more clinical complication of a condition being treated (e.g., heart failure). The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample. In some embodiments, the disclosure relates to methods of administering an ActRII-ALK4 antagonist to a patient in need of treatment (e.g., a “patient in need thereof”). Such patients in need of treatment with an ActRII-ALK4 antagonist are patients having a disorder or condition disclosed in the instant application including, but not limited to, heart failure associated with aging.

In general, treatment or prevention of a disease or condition as described in the present disclosure (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) is achieved by administering one or more ActRII-ALK4 antagonists of the disclosure in an “effective amount”. An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

The main terminology used to describe HF is based on measurement of left ventricular ejection fraction (LVEF). HF comprises a wide range of patients (Table 1). Some patients have normal LVEF, which is typically considered as ≥50% and is referred to as HF with preserved ejection fraction (HFpEF). Other patients have HF with reduced LVEF (HFrEF), which is typically considered to be <40%. Patients with an LVEF in the range of between about 40% and about 49% represent a “grey area”, which is sometimes defined as HF with mid-range ejection fraction (HFmrEF). Sometimes these patients in the “grey area” are identified as having HFrEF, depending on the clinician. Differentiation of patients with HF based on LVEF is important due to different underlying etiologies, demographics, co-morbidities and response to therapies. Most clinical trials published after 1990 selected patients based on LVEF (usually measured using echocardiography, a radionuclide technique or cardiac magnetic resonance (CMR)), and to the best of our knowledge, it is only in patients with HFrEF that therapies have been shown to reduce both morbidity and mortality. Typically, patients with heart failure associated with aging have normal LVEF (e.g., HFpEF).

TABLE 1
Definition of heart failure by left ventricular ejection fraction analysis

| Type of HF | | HFrEF | HFmrEF | HFpEF |
|---|---|---|---|---|
| Criteria | 1 | Symptoms ± Signs | Symptoms ± Signs | Symptoms ± Signs |
| | 2 | LVEF <40% | LVEF 40-49% | LVEF ≥50% |
| | 3 | — | 1. Elevated levels of | 1. Elevated levels of |
| | | | natriuretic peptides | natriuretic peptides |
| | | | 2. At least one additional | 2. At least one additional |
| | | | criterion: | criterion: |
| | | | a. relevant structural heart | a. relevant structural heart |
| | | | disease (LVH and/or LAE) | disease (LVH and/or LAE) |
| | | | b. diastolic dysfunction | b. diastolic dysfunction |

Symptoms: e.g., breathlessness, ankle swelling and fatigue
Signs: e.g., elevated jugular venous pressure, pulmonary crackles and peripheral edema. Signs may not be present in the early stages of HF (especially in HFpEF) and in patients treated with diuretics.
Symptoms and signs are caused by a structural and/or functional cardiac abnormality.
HF = heart failure; HFmrEF = heart failure with mid-range ejection fraction; HFpEF = heart failure with preserved ejection fraction; HFrEF = heart failure with reduced ejection fraction; LAE = left atrial enlargement; LVEF = left ventricular ejection fraction; LVH = left ventricular hypertrophy;

In certain aspects, the disclosure relates to a method of treating, preventing, or reducing the progression rate and/or severity of HFpEF comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the disclosure relates to a method of treating a patient that has normal LVEF. In some embodiments, the disclosure relates to a method of treating a patient having normal LVEF. In some embodiments, normal LVEF is an LVEF of ≥50%. In some embodiments, the disclosure relates to a method of treating a patient with HFpEF. In some embodiments, the disclosure relates to a method of treating a patient having HFpEF and elevated levels of natriuretic peptides. In some embodiments, the disclosure relates to treating a patient having HFpEF, elevated levels of natriuretic peptides, and a structural heart disease and/or diastolic dysfunction. In some embodiments, the disclosure relates to treating a patient with heart failure associated with aging, wherein the patient has HFpEF. In some embodiments, the disclosure relates to treating a patient with heart failure associated with aging, wherein the patient has normal LVEF. In some embodiments, the disclosure relates to treating a patient with heart failure associated with aging, wherein the patient has an LVEF of ≥50%. In some embodiments, the disclosure relates to treating a patient with heart failure associated with aging, wherein the patient has elevated levels of natriuretic peptides. In certain aspects, the disclosure relates to a method of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient has HFpEF.

In certain aspects, the disclosure relates to a method of treating, preventing, or reducing the progression rate and/or severity of heart failure with reduced ejection fraction (HFrEF) comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the disclosure relates to a method of treating a patient having reduced LVEF. In some embodiments, the disclosure relates to a method of treating a patient with reduced LVEF and an LVEF of <40%. In some embodiments, the disclosure relates to a method of treating a patient with reduced LVEF and HF associated with reduced ejection fraction (HFrEF).

In certain aspects, the disclosure relates to a method of treating, preventing, or reducing the progression rate and/or severity of heart failure with mid-range ejection fraction (HFmrEF) comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the disclosure relates to a method of treating a patient has mid-range LVEF. In some embodiments, the disclosure relates to a method of treating a patient with mid-range LVEF and an LVEF of between about 40% and about 49%. In some embodiments, the disclosure relates to treating a patient with mid-range LVEF and HF associated with mid-range ejection fraction (HFmrEF). In some embodiments, the disclosure relates to a method of treating a patient having HFmrEF and elevated levels of natriuretic peptides. In some embodiments, the disclosure relates to a method of treating a patient having HFmrEF and elevated levels of natriuretic peptides, and a structural heart disease and/or diastolic dysfunction.

Diagnosis of HFpEF can be more challenging than a diagnosis of HFrEF. Patients with HFpEF generally do not have a dilated LV, but instead often have an increase in LV wall thickness and/or increased left atrial (LA) size as a sign of increased filling pressures. Most have additional ‘evidence’ of impaired LV filling or suction capacity, also classified as diastolic dysfunction, which is generally accepted as the likely cause of HF in these patients (hence the term ‘diastolic HF’). However, most patients with HFrEF (previously referred to as ‘systolic HF’) also have diastolic dysfunction, and subtle abnormalities of systolic function have been shown in patients with HFpEF. Hence the preference for stating preserved or reduced LVEF over preserved or reduced ‘systolic function’.

In previous guidelines it was acknowledged that a grey area exists between HFrEF and HFpEF. These patients have an LVEF that ranges from 40 to 49%, hence the term HFmrEF. Patients with HFmrEF most likely have primarily mild systolic dysfunction, but with features of diastolic dysfunction.

Patients without detectable LV myocardial disease may have other cardiovascular causes for HF (e.g., pulmonary hypertension, valvular heart disease, etc.). Patients with non-cardiovascular pathologies (e.g., anemia, pulmonary, renal or hepatic disease) may have symptoms similar or identical to those of HF and each may complicate or exacerbate the HF syndrome.

The NYHA functional classification (Table 2) has been used to describe the severity of symptoms and exercise intolerance. However, symptom severity correlates poorly with many measures of LV function; although there is a clear relationship between the severity of symptoms and survival, patients with mild symptoms may still have an increased risk of hospitalization and death. Sometimes the term ‘advanced HF’ is used to characterize patients with severe symptoms, recurrent decompensation and severe cardiac dysfunction.

TABLE 2
New York Heart Association (NYHA) functional classification
of HF based on severity of symptoms and physical activity

| | |
|---|---|
| Class I | No limitation of physical activity. Ordinary physical activity |
| | does not cause undue breathlessness, fatigue, or palpitations. |
| Class II | Slight limitation of physical activity. Comfortable at rest, |
| | but ordinary physical activity results in undue breathlessness, |
| | fatigue, or palpitations. |
| Class III | Marked limitation of physical activity. Comfortable at rest, |
| | but less than ordinary physical activity results in undue |
| | breathlessness, fatigue, or palpitations. |
| Class IV | Unable to carry on any physical activity without discomfort. |
| | Symptoms at rest can be present If any physical activity is |
| | undertaken, discomfort is increased. |

In some embodiments, the disclosure relates to a method of treating a patient having NYHA Class I HF. In some embodiments, a patient with NYHA Class I HF has no limitation of physical activity. In some embodiments, a patient with NYHA Class I HF experiences physical activity that does not cause undue breathlessness, fatigue, and/or palpitations. In some embodiments, the disclosure relates to a method of treating a patient having NYHA Class II HF. In some embodiments, a patient with NYHA Class II HF has slight limitation of physical activity. In some embodiments, a patient with NYHA Class II HF experiences ordinary physical activity resulting in undue breathlessness, fatigue, or palpitations. In some embodiments, the disclosure relates to a method of treating a patient having NYHA Class III HF. In some embodiments, a patient with NYHA Class III HF has marked limitation of physical activity. In some embodiments, a patient with NYHA Class III HF experiences less than ordinary physical activity resulting in undue breathlessness, fatigue, or palpitations. In some embodiments, the disclosure relates to a method of treating a patient having NYHA Class IV HF. In some embodiments, a patient with NYHA Class IV HF is unable to carry on any physical activity without discomfort. In some embodiments, a patient with NYHA Class IV HF experiences symptoms at rest, as well as when any physical activity is undertaken, discomfort is increased.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves the patient's NYHA functional heart failure Class. In some embodiments, the method relates to reducing the patient's NYHA Class from Class IV to Class III. In some embodiments, the method relates to reducing the patient's NYHA Class from Class IV to Class II. In some embodiments, the method relates to reducing the patient's NYHA Class from Class IV to Class I. In some embodiments, the method relates to reducing the patient's NYHA Class from Class III to Class II. In some embodiments, the method relates to reducing the patient's NYHA Class from Class III to Class I. In some embodiments, the method relates to reducing the patient's NYHA Class from Class II to Class I.

The American College of Cardiology Foundation/American Heart Association (ACCF/AHA) classification describes stages of HF development based on structural changes and symptoms (Table 3). The ACC/AHA classification system places emphasis on staging and development of disease, similar to the approach commonly used in oncology. These HF stages progress from antecedent risk factors (stage A) to the development of subclinical cardiac dysfunction (stage B), then symptomatic HF (stage C), and finally, end-stage refractory disease (stage D). ACC/AHA stages are progressive from stage A to stage D.

TABLE 3
American College of Cardiology Foundation/American Heart
Association (ACCF/AHA) stages of heart failure

| | |
|---|---|
| A | At high risk for HF but without structural heart disease or symptoms |
| | of HF. |
| B | Structural heart disease but without signs or symptoms of HF. |
| C | Structural heart disease with prior or current symptoms of HF. |
| D | Refractory HF requiring specialized interventions. |

In some embodiments, the disclosure relates to a method of treating a patient having ACCF/AHA Stage A HF. In some embodiments, a patient with ACCF/AHA Stage A HF is at high risk for HF but without structural heart disease or symptoms of HF. In some embodiments, the disclosure relates to a method of treating a patient having ACCF/AHA Stage B HF. In some embodiments, a patient with Stage B HF has structural heart disease but without known signs or symptoms of HF. In some embodiments, the disclosure relates to a method of treating a patient having ACCF/AHA Stage C HF. In some embodiments, a patient with ACCF/AHA Stage C HF has structural heart disease with prior or current symptoms of HF. In some embodiments, the disclosure relates to a method of treating a patient having ACCF/AHA Stage D HF. In some embodiments, a patient with ACCF/AHA Stage D HF has refractory HF requiring specialized interventions.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves the patient's ACCF/AHA stage of heart failure. In some embodiments, the method relates to reducing the patient's ACCF/AHA Stage from Stage D to Stage C. In some embodiments, the method relates to reducing the patient's ACCF/AHA Stage from Stage D to Stage B. In some embodiments, the method relates to reducing the patient's ACCF/AHA Stage from Stage D to Stage A. In some embodiments, the method relates to reducing the patient's ACCF/AHA Stage from Stage C to Stage B. In some embodiments, the method relates to reducing the patient's ACCF/AHA Stage from Stage C to Stage A. In some embodiments, the method relates to reducing the patient's ACCF/AHA Stage from Stage B to Stage A.

The Killip classification may be used to describe the severity of the patient's condition in the acute setting after myocardial infarction. Patients with HF complicating acute myocardial infarction (AMI) can be classified according to Killip and Kimball into the classes shown in Table 4.

TABLE 4
Killip Classification of HF complicating AMI

| | |
|---|---|
| Class I | No clinical signs of HF |
| Class II | HF with rales and S3 gallop |
| Class III | With frank acute pulmonary edema |
| Class IV | Cardiogenic shock, hypotension (SBP, 90 mmHg) and |
| | evidence of peripheral vasoconstriction such as oliguria, |
| | cyanosis and diaphoresis |

In some embodiments, the disclosure relates to a method of treating a patient having Killip Class I HF complicating AMI. In some embodiments, a patient with Killip Class I HF complicating AMI has no clinical signs of HF. In some embodiments, the disclosure relates to a method of treating a patient having Killip Class II HF complicating AMI. In some embodiments, a patient with Killip Class II HF complicating AMI has HF with rales and S3 gallop. In some embodiments, the disclosure relates to a method of treating a patient having Killip Class III HF complicating AMI. In some embodiments, a patient with Killip Class III HF complicating AMI has frank acute pulmonary edema. In some embodiments, the disclosure relates to a methods of treating a patient having Killip Class IV HF complicating AMI. In some embodiments, a patient with Killip Class IV HF complicating AMI has cardiogenic shock, hypotension (e.g., SBP, 90 mmHg) and evidence of peripheral vasoconstriction such as oliguria, cyanosis and diaphoresis.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves the patient's Killip HF Classification. In some embodiments, the method relates to reducing the patient's Killip Class from Class IV to Class III. In some embodiments, the method relates to reducing the patient's Killip Class from Class IV to Class II. In some embodiments, the method relates to reducing the patient's Killip Class from Class IV to Class I. In some embodiments, the method relates to reducing the patient's Killip Class from Class III to Class II. In some embodiments, the method relates to reducing the patient's Killip Class from Class III to Class I. In some embodiments, the method relates to reducing the patient's Killip Class from Class II to Class I.

The Framingham criteria for diagnosis of heart failure (Table 5) requires presence of at least two major criteria, or at least one major and two minor criteria. Although these criteria have served as a gold reference standard for decades, they are largely predicated on the presence of congestion at rest. Importantly, this clinical feature is often absent in ambulatory patients who have well-compensated HF, or in patients with HF who develop abnormal hemodynamics exclusively during exercise. Therefore, despite being highly specific, the Framingham criteria tend to have a poor sensitivity for the diagnosis of HF.

TABLE 5
Framingham criteria for diagnosis of heart failure

| | |
|---|---|
| Major criteria | Paroxysmal nocturnal dyspnea or orthopnea |
| | Jugular vein distension |
| | Rales |
| | Radiographic cardiomegaly |
| | Acute pulmonary edema |
| | S3 gallop |
| | Increased venous pressure greater than 16 cm of water |
| | Circulation time greater than or equal to 25 seconds |
| | Hepatojugular reflex |
| | Weight loss greater than or equal to 4.5 kg in 5 days in |
| | response to treatment |
| Minor criteria | Bilateral ankle edema |
| | Nocturnal cough |
| | Dyspnea on ordinary exertion |
| | Hepatomegaly |
| | Pleural effusion |
| | Decrease in vital capacity by 1/3 from maximum |
| | recorded |
| | Tachycardia (heart rate greater than 120/min) |

In some embodiments, the disclosure relates to a methods of treating a patient having one or more major Framingham criteria for diagnosis of HF. In some embodiments, a patient has one or more of paroxysmal nocturnal dyspnea or orthopnea, jugular vein distension, rales, radiographic cardiomegaly, acute pulmonary edema, S3 gallop, increased venous pressure greater than 16 cm of water, circulation time greater than or equal to 25 seconds, hepatojugular reflex, and weight loss greater than or equal to 4.5 kg in 5 days in response to treatment. In some embodiments, the disclosure relates to a methods of treating a patient having one or more minor Framingham criteria for diagnosis of HF. In some embodiments, a patient has one or more of bilateral ankle edema, nocturnal cough, dyspnea on ordinary exertion, hepatomegaly, pleural effusion, decrease in vital capacity by 1/3 from maximum recorded, and tachycardia (heart rate greater than 120/min). In some embodiments, a patient has at least two Framingham major criteria. In some embodiments, a patient has at least one major Framingham criteria and at least two minor Framingham criteria.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method reduces the number of Framingham criteria for heart failure that the patient has. In some embodiments, the method relates to decreasing the number of major Framingham criteria for heart failure that the patient has. In some embodiments, the method relates to decreasing the number of minor Framingham criteria for heart failure that the patient has.

There are many known symptoms and signs of heart failure that a medical professional may look for regarding a diagnosis of heart failure. Some symptoms may be non-specific and do not, therefore, help discriminate between HF and other problems. Symptoms and signs of HF due to fluid retention may resolve quickly with diuretic therapy. Signs, such as elevated jugular venous pressure and displacement of the apical impulse, may be more specific, but are harder to detect and have poor reproducibility. HF is unusual in an individual with no relevant medical history (e.g., a potential cause of cardiac damage), whereas certain features, particularly previous myocardial infarction, greatly increase the likelihood of HF in a patient with appropriate symptoms and signs. Symptoms and signs are important in monitoring a patient's response to treatment and stability over time. Persistence of symptoms despite treatment usually indicates the need for additional therapy, and worsening of symptoms is a serious development (placing the patient at risk of urgent hospital admission and death) and merits prompt medical attention.

TABLE 6
Signs and Symptoms of Heart Failure

| Symptoms | Signs |
|---|---|
| Typical | More specific |
| Breathlessness | Elevated jugular venous pressure |
| Orthopnea | Hepatojugular reflux |
| Paroxysmal nocturnal dyspnea | Third heart sound (gallop rhythm) |
| Reduced exercise tolerance | Laterally displaced apical impulse |
| Fatigue, tiredness, increased time to recover | |
| after exercise | |
| Ankle swelling | |
| Less typical | Less specific |
| Nocturnal cough | Weight gain (>2 kg/week) |
| Wheezing | Weight loss (in advanced HF) |
| Bloated feeling | Tissue wasting (cachexia) |
| Loss of appetite | Cardiac murmur |
| Confusion (especially in the elderly) | Peripheral edema (ankle, sacral, scrotal) |
| Depression | Pulmonary crepitations |
| Palpitations | Reduced air entry and dullness to |
| Dizziness | percussion at lung bases (pleural effusion) |
| Syncope | Tachycardia |
| Bendopnea | Irregular pulse |
| | Tachypnoea |
| | Cheyne Stokes respiration |
| | Hepatomegaly |
| | Ascites |
| | Cold extremities |
| | Oliguria |
| | Narrow pulse pressure |

In some embodiments, the disclosure relates to a method of treating a patient having one or more typical and/or less typical symptoms of HF. In some embodiments, the disclosure relates to a method of treating a patient having one or more specific and/or less specific signs of HF. In some embodiments, the disclosure relates to treating a patient having one or more typical symptoms, less typical symptoms, specific signs, and/or less specific signs of HF. In some embodiments, the disclosure relates to a method treating a patient having one or more typical symptoms of HF. In some embodiments, a patient has one or more symptoms selected from the group consisting of breathlessness, orthopnea, paroxysmal nocturnal dyspnea, reduced exercise tolerance, fatigue, tiredness, increased time to recover after exercise, and ankle swelling. In some embodiments, a patient has one or more less typical symptoms of HF. In some embodiments, a patient has one or more less typical symptoms selected from the group consisting of nocturnal cough, wheezing, bloated feeling, loss of appetite, confusion (especially in the elderly), depression, palpitations, dizziness, syncope, and bendopnea. In some embodiments, a patient has one or more signs of HF. In some embodiments, a patient has one or more signs of HF selected from the group consisting of elevated jugular venous pressure, hepatojugular reflux, third heart sound (gallop rhythm), and laterally displaced apical impulse. In some embodiments, a patient has one or more less specific signs of HF. In some embodiments, a patient has one or more less specific signs of HF selected from the group consisting of weight gain (>2 kg/week), weight loss (in advanced HF), tissue wasting (cachexia), cardiac murmur, peripheral edema (ankle, sacral, scrotal), pulmonary crepitations, reduced air entry and dullness to percussion at lung bases (pleural effusion), tachycardia, irregular pulse, tachypnoea, Cheyne Stokes respiration, hepatomegaly, ascites, cold extremities, oliguria, and narrow pulse pressure.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method reduces the number of signs and/or symptoms of heart failure that the patient has. In some embodiments, the method relates to decreasing the number of signs of heart failure that the patient has. In some embodiments, the method relates to decreasing the number of symptoms of heart failure that the patient has.

Heart Failure Associated with Aging

With age being one of the dominant risk factors for development of cardiovascular diseases, prevalence increases dramatically as a patient's age increases. The prevalence of heart failure in the adult population in developed countries is 1-2%, which rises to >10% among persons 70 years or older (McMurray et al. Eur. J. Heart Fail, 2012, 14:803-869). The Framingham Study indicated that the incidence of heart failure approximately doubled over each successive decade of life, rising more steeply with age in women than in men. The annual incidence in men rose from 2 per 1000 at age 35 to 64 years to 12 per 1000 at age 65 to 94 years. Because the increase in risk with age is balanced by decreased life expectancy with older age, the lifetime likelihood of developing HF is approximately 20 percent at all ages above 40. This population can be divided into patients with a preserved ejection fraction (HFpEF) and patients with a reduced ejection fraction (HFrEF). Nearly half of these heart failure patients have HFpEF. As the heart ages, changes can occur at many different levels: structural, functional, cellular, and molecular, all of which can lead to heart failure. Commonly, heart failure associated with aging is characterized by increased left ventricular (LV) wall thickness (i.e., LV hypertrophy) and diastolic dysfunction, with no reduction in left ventricular ejection fraction (LVEF) (i.e., HFpEF). While ejection fraction by definition in HFpEF patients is normal, LV contractility is impaired. To avoid a low specificity when diagnosing HFpEF, exertional dyspnea and a normal LVEF can be coupled with objective measures of diastolic LV dysfunction, LV hypertrophy, left atrial (LA) enlargement, and/or plasma levels of natriuretic peptides (NP).

Diagnosis of HFpEF remains challenging. In an HFpEF patient, LVEF is normal and signs and symptoms for HF are often non-specific and do not discriminate well between HF and other clinical conditions. Diagnosis of chronic HFpEF, especially in a typical elderly patient with co-morbidities and no obvious signs of central fluid overload, is cumbersome and a validated gold standard is elusive. To improve the specificity of diagnosing HFpEF, a clinical diagnosis should be supported by objective measures of cardiac dysfunction at rest or during exercise. A diagnosis of HFpEF typically requires the following: presence of symptoms and/or signs of HF; a ‘preserved’ EF (defined as LVEF≥50% or sometimes as 40-49% for HFmrEF); elevated levels of NPs (BNP>35 pg/mL and/or NT-proBNP>125 pg/mL); objective evidence of other cardiac functional and structural alterations underlying HF; and in case of uncertainty, a stress test or invasively measured elevated LV filling pressure may be needed to confirm the diagnosis of HFpEF.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the patient is at least 40 years old. In some embodiments, the patient is at least 45 years old. In some embodiments, the patient is at least 50 years old. In some embodiments, the patient is at least 55 years old. In some embodiments, the patient is at least 60 years old. In some embodiments, the patient is at least 65 years old. In some embodiments, the patient is at least 70 years old. In some embodiments, the patient is at least 75 years old. In some embodiments, the patient is at least 80 years old. In some embodiments, the patient is at least 85 years old. In some embodiments, the patient is at least 90 years old. In some embodiments, the patient is at least 95 years old. In some embodiments, the patient is at least 100 years old. In some embodiments, the patient is between about 40 and about 100 years old. In some embodiments, the patient has heart failure with preserved ejection fraction (HFpEF). In some embodiments, the heart failure is heart failure associated with preserved ejection fraction (HFpEF). In some embodiments, the patient has dyspnea. In some embodiments, the patient has exertional dyspnea. In some embodiments, the patient has increased left ventricular wall thickness. In some embodiments, the patient has LV hypertrophy. In some embodiments, the patient has diastolic dysfunction. In some embodiments, the patient has LV diastolic dysfunction. In some embodiments, the patient has left atrial enlargement. In some embodiments, the patient has no reduction in left ventricular ejection fraction. In some embodiments, the patient has a left ventricular ejection fraction of ≥50%. In some embodiments, the patient has increased levels of natriuretic peptides.

### Structural Changes

As a patient ages, significant structural changes in the heart and vasculature occur. Some examples of this include, but are not limited to, increased vascular intimal thickness, increased vascular stiffening, increased left ventricular wall thickness (within normal limits) and increased left atrial size (Table 7). Overall changes in thickness and shape of the heart have important implications for cardiac wall stress and overall contractile efficiency.

In certain aspects, the disclosure relates to methods of treating, preventing, or reducing the progression rate and/or severity of heart failure associated with aging, or one or more complications of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient has cardiovascular structural remodeling. In some embodiments a patient has cardiovascular structural remodeling selected from the group consisting of an increase in vascular intimal thickness, an increase in vascular stiffness, an increase in LV hypertrophy (e.g., increase in LV wall thickness), and an increase in left atrial enlargement (e.g., increase in left atrial wall size). In some embodiments, the patient has an increase in vascular intimal thickness. In some embodiments, the patient has an increase in vascular stiffness. In some embodiments, the patient has an increase in LV hypertrophy. In some embodiments, the patient has an increase in LV wall thickness. In some embodiments, the patient has systolic hypertension. In some embodiments, the patient has retarded early diastolic cardiac filling. In some embodiments, the patient has increased cardiac filling pressure. In some embodiments, the patient has a lower threshold for dyspnea. In some embodiments, the patient has an increased likelihood of heart failure with relatively normal systolic function. In some embodiments, the patient has left atrial enlargement. In some embodiments, the patient has an increase in left atrial size. In some embodiments, the patient has an increased prevalence of lone atrial fibrillation and/or other atrial arrhythmias.

In certain aspects, the disclosure relates to methods of treating, preventing, or reducing the progression rate and/or severity of heart failure associated with aging, or one or more complications of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves cardiovascular structural remodeling in the patient. In some embodiments is the method improves cardiovascular structural remodeling selected from the group consisting of an increase in vascular intimal thickness, an increase in vascular stiffness, an increase in LV hypertrophy (e.g., increase in LV wall thickness), and an increase in left atrial enlargement (e.g., increase in left atrial wall size). In some embodiments, the method decreases vascular intimal thickness in the patient. In some embodiments, the method decreases vascular stiffness in the patient. In some embodiments, the method decreases LV hypertrophy in the patient. In some embodiments, the decreases LV wall thickness in the patient. In some embodiments, the method improves systolic hypertension in the patient. In some embodiments, the method improves early diastolic cardiac filling in the patient. In some embodiments, the method decreases cardiac filling pressure in the patient. In some embodiments, the method improves left atrial enlargement in the patient. In some embodiments, the method decreases left atrial size in the patient. In some embodiments, the method decreases prevalence of lone atrial fibrillation and/or other atrial arrhythmias in the patient.

TABLE 7
Relationship of Cardiovascular Human Structural
Changes to Cardiovascular Disease

| | | |
|---|---|---|
| | Plausible | Possible Relation |
| Changes | Mechanism(s) | to Human Disease |

Cardiovascular structural remodeling

| | | |
|---|---|---|
| Increase in vascular | Increased migration of, | Early stages of atherosclerosis |
| intimal thickness | and increased matrix | |
| | production by VSMC; | |
| | possible derivation of | |
| | intimal cells from other | |
| | sources | |
| Increase in vascular | Elastin fragmentation, | Systolic hypertension; |
| stiffness | increase in elastase | LV wall thickening; |
| | activity, increase in | Stroke; |
| | collagen production by | Atherosclerosis |
| | VSMC and increase in | |
| | cross-linking of | |
| | collagen, and altered | |
| | growth factor | |
| | regulation/tissue repair | |
| | mechanisms | |
| Increase in LV wall | Increase in LV myocyte | Retarded early diastolic cardiac |
| thickness | size with altered Ca²⁺ | filling; |
| “LV hypertrophy” | handling, increase in | Increased cardiac filling pressure; |
| | myocyte number | Lower threshold for dyspnea; |
| | (necrotic and apoptotic | Increased likelihood of heart failure |
| | death), altered growth | with relatively normal systolic |
| | factor regulation, focal | function |
| | matrix collagen | |
| | deposition | |
| Increase in left atrial | Increase in left atrial | Increased prevalence of lone atrial |
| size | pressure/volume | fibrillation and other atrial |
| “LA enlargement” | | arrhythmias |

Abbreviations: VSMC = vascular smooth muscle cells; LV = left ventricle; PUFA = polyunsaturated fatty acids (Strait and Lakatta, Heart Fail Clin, 2012, 8: 143-164).

Ventricular Structure

On a structural level, one of the most striking phenomenon seen with age is an increase in the thickness of the LV wall as a result of increased cardiomyocyte size (i.e., LV hypertrophy). LV hypertrophy is mostly seen as a compensatory response by the body after the loss of cardiomyocytes that occurs with aging, causing the left ventricle to work harder. As the workload increases, muscle tissue in the chamber wall thickens, and sometimes the size of the chamber itself also increases. The enlarged heart muscle loses elasticity and eventually may fail to pump with as much force as needed. There have been conflicting data concerning the evolution of LV mass with age, with recent analyses trending towards little to no effect on mass (Akasheva et al., PLoS One, 2015, 10:e0135883) or there may be a slight sex-specific decrease in men only (Strait and Lakatta, Heart Fail Clin, 2012, 8:143-164). LV dimension decreases with age, reflected by an increase in the mass/volume ratio and a decrease in LV end-diastolic volume. Therefore, aging is typically associated with LV hypertrophy. Such hypertrophy affects the LV in an asymmetrical way, mostly affecting the interventricular septum and leading to a redistribution of cardiac muscle, explaining the possible lack of effect on total cardiac mass. LV hypertrophy and a decreased LV cavity volume are some of the hallmarks of HFpEF, which is common in aging patients with heart failure.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient has a change in ventricular structure in the heart. In some embodiments, a change in ventricular structure in the heart is selected from the group consisting of LV hypertrophy, an increase in cardiomyocyte size, a loss of cardiomyocytes, little to no change in LV mass, and a decrease in LV end-diastolic volume. In some embodiments, the patient has LV hypertrophy. In some embodiments, the patient has an increase in thickness of the LV wall. In some embodiments, the patient has increased cardiomyocyte size. In some embodiments, the patient has a loss of cardiomyocytes. In some embodiments, the patient has a decrease in LV end-diastolic volume.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves one or more changes in ventricular structure in the heart. In some embodiments, the method improves ventricular structure in the heart selected from the group consisting of LV hypertrophy, an increase in cardiomyocyte size, a loss of cardiomyocytes, little to no change in LV mass, and a decrease in LV end-diastolic volume. In some embodiments, the method decreases LV hypertrophy. In some embodiments, the method prevents LV hypertrophy from worsening. In some embodiments, the method repairs LV hypertrophy. In some embodiments, the method decreases thickness of the LV wall. In some embodiments, the method decreases cardiomyocyte size. In some embodiments, the method improves the loss of cardiomyocytes. In some embodiments, the method prevents the loss of cardiomyocytes from worsening. In some embodiments, the method increases LV end-diastolic volume.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient has left ventricular hypertrophy. In some embodiments, the disclosure relates to methods of adjusting one or more parameters in the heart failure patient toward a more normal level (e.g., normal as compared to healthy people of similar age and sex), comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the method relates to decreasing the patient's left ventricular hypertrophy by least 1% (e.g., 1, 5, 10, 15, 20, 25, 30, 35, 40, 45, 50, 55, 60, 65, 70, 75, 80, 85, 90, 95, or 100%). In some embodiments, the method relates to decreasing the patient's left ventricular hypertrophy by at least 1%. In some embodiments, the method relates to decreasing the patient's left ventricular hypertrophy by at least 5%. In some embodiments, the method relates to decreasing the patient's left ventricular hypertrophy by at least 10%. In some embodiments, the method relates to decreasing the patient's left ventricular hypertrophy by at least 15%. In some embodiments, the method relates to decreasing the patient's left ventricular hypertrophy by at least 20%. In some embodiments, the method relates to decreasing the patient's left ventricular hypertrophy by at least 25%. In some embodiments, the method relates to decreasing the patient's left ventricular hypertrophy by at least 30%. In some embodiments, the method relates to decreasing the patient's left ventricular hypertrophy by at least 35%. In some embodiments, the method relates to decreasing the patient's left ventricular hypertrophy by at least 40%. In some embodiments, the method relates to decreasing the patient's left ventricular hypertrophy by at least 45%. In some embodiments, the method relates to decreasing the patient's left ventricular hypertrophy by at least 50%. In some embodiments, the method relates to decreasing the patient's left ventricular hypertrophy by at least 55%. In some embodiments, the method relates to decreasing the patient's left ventricular hypertrophy by at least 60%. In some embodiments, the method relates to decreasing the patient's left ventricular hypertrophy by at least 65%. In some embodiments, the method relates to decreasing the patient's left ventricular hypertrophy by at least 70%. In some embodiments, the method relates to decreasing the patient's left ventricular hypertrophy by at least 75%. In some embodiments, the method relates to decreasing the patient's left ventricular hypertrophy by at least 80%. In some embodiments, the method relates to decreasing the patient's left ventricular hypertrophy by at least 85%. In some embodiments, the method relates to decreasing the patient's left ventricular hypertrophy by at least 90%. In some embodiments, the method relates to decreasing the patient's left ventricular hypertrophy by at least 95%. In some embodiments, the method relates to decreasing the patient's left ventricular hypertrophy by 100%.

Atrial Structure

In the elderly, atrial contraction plays a much greater role in LV filling during diastole than in the young population. This change in function is associated with the development of atrial hypertrophy (thickening) and dilation. Left atrial size has been associated with the presence of atrial fibrillation. Two important aspects of age-related structural remodeling of the heart—LV hypertrophy and atrial dilation—are therefore associated with the two main cardiac pathologies of old age: HFpEF and atrial fibrillation. These two pathologies often occur together, with two-thirds of HFpEF patients at some point presenting with atrial fibrillation and with most patients first developing atrial fibrillation and then heart failure. Echocardiographic studies show that the aortic root dilates modestly with age, approximating 6% between the fourth and eighth decades. In normal aging, however, such aortic root dilation provides an additional stimulus for LV hypertrophy because the larger volume of blood in the proximal aorta leads to a greater inertial load against which the senescent heart must pump.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) wherein the patient has a change in atrial structure in the heart. In some embodiments, a change in atrial structure in the heart is selected from the group consisting of left atrial hypertrophy, arrhythmia, atrial dilation, aortic root dilation, and atrial fibrillation. In some embodiments, the patient has atrial hypertrophy. In some embodiments, the patient has left atrial hypertrophy. In some embodiments, the patient has left atrial enlargement. In some embodiments, the patient has arrhythmia. In some embodiments, the patient has atrial dilation. In some embodiments, the patient has aortic root dilation. In some embodiments, the patient has atrial fibrillation.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) wherein the method improves a change in atrial structure in the heart. In some embodiments, the method improves a change in atrial structure in the heart is selected from the group consisting of left atrial hypertrophy, arrhythmia, atrial dilation, aortic root dilation, and atrial fibrillation. In some embodiments, the method improves atrial hypertrophy. In some embodiments, the method improves left atrial hypertrophy. In some embodiments, the method improves left atrial enlargement. In some embodiments, the method decreases arrhythmia in the patient. In some embodiments, the method improves atrial dilation. In some embodiments, the improves aortic root dilation. In some embodiments, the method improves atrial fibrillation

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient has left atrial enlargement. In some embodiments, the disclosure relates to methods of adjusting one or more parameters in the heart failure patient toward a more normal level (e.g., normal as compared to healthy people of similar age and sex), comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the method relates to decreasing the patient's left atrial enlargement by least 1% (e.g., 1, 5, 10, 15, 20, 25, 30, 35, 40, 45, 50, 55, 60, 65, 70, 75, 80, 85, 90, 95, or 100%). In some embodiments, the method relates to decreasing the patient's left atrial enlargement by at least 1%. In some embodiments, the method relates to decreasing the patient's left atrial enlargement by at least 5%. In some embodiments, the method relates to decreasing the patient's left atrial enlargement by at least 10%. In some embodiments, the method relates to decreasing the patient's left atrial enlargement by at least 15%. In some embodiments, the method relates to decreasing the patient's left atrial enlargement by at least 20%. In some embodiments, the method relates to decreasing the patient's left atrial enlargement by at least 25%. In some embodiments, the method relates to decreasing the patient's left atrial enlargement by at least 30%. In some embodiments, the method relates to decreasing the patient's left atrial enlargement by at least 35%. In some embodiments, the method relates to decreasing the patient's left atrial enlargement by at least 40%. In some embodiments, the method relates to decreasing the patient's left atrial enlargement by at least 45%. In some embodiments, the method relates to decreasing the patient's left atrial enlargement by at least 50%. In some embodiments, the method relates to decreasing the patient's left atrial enlargement by at least 55%. In some embodiments, the method relates to decreasing the patient's left atrial enlargement by at least 60%. In some embodiments, the method relates to decreasing the patient's left atrial enlargement by at least 65%. In some embodiments, the method relates to decreasing the patient's left atrial enlargement by at least 70%. In some embodiments, the method relates to decreasing the patient's left atrial enlargement by at least 75%. In some embodiments, the method relates to decreasing the patient's left atrial enlargement by at least 80%. In some embodiments, the method relates to decreasing the patient's left atrial enlargement by at least 85%. In some embodiments, the method relates to decreasing the patient's left atrial enlargement by at least 90%. In some embodiments, the method relates to decreasing the patient's left atrial enlargement by at least 95%. In some embodiments, the method relates to decreasing the patient's left atrial enlargement by 100%.

### Functional Changes

There are a number of functional changes and compensatory responses that the aged heart undergoes that diminish its ability to respond to increased workload, and also that decrease its reserve capacity. Aging affects the diastolic, systolic, as well as electrical function of the heart. Changes in maximal heart rate, end-systolic volume (ESV), end-diastolic volume (EDV), contractility, prolonged systolic contraction, prolonged diastolic relaxation, and sympathetic signaling are examples of functional changes as a subject ages. Aging patients may have altered regulation of vascular tone, a reduced threshold for cell Ca²⁺ overload, increased cardiovascular reserve, and reduced physical activity. Such functional changes may lead to vascular stiffening, hypertension, early atherosclerosis, a lower threshold for atrial and ventricular arrhythmia, increased myocyte death, increased fibrosis, and a lower threshold for and increased severity of heart failure (Table 8).

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) wherein the patient has a functional change in the heart. In some embodiments, a functional change in the heart is selected from the group consisting of changes in diastolic heart function, changes in systolic heart function, and changes in electrical heart function. In some embodiments, the patient has changes in diastolic heart function. In some embodiments, the patient has changes in systolic heart function. In some embodiments, the patient has changes in electrical heart function.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) wherein the method improves a functional change in the heart. In some embodiments, the method improves a functional change in the heart selected from the group consisting of changes in diastolic heart function, changes in systolic heart function, and changes in electrical heart function. In some embodiments, the improves changes in diastolic heart function. In some embodiments, the method improves changes in systolic heart function. In some embodiments, method improves changes in electrical heart function.

TABLE 8
Relationship of Cardiovascular Human Functional
Changes to Cardiovascular Disease

| | | |
|---|---|---|
| | Plausible | Possible Relation |
| Changes | Mechanism(s) | to Human Disease |

Cardiovascular functional changes

| | | |
|---|---|---|
| Altered regulation of | Decrease in NO | Vascular stiffening; |
| vascular tone | production/effects | Hypertension; |
| | | Early atherosclerosis |
| Reduced threshold for | Changes in gene | Lower threshold for atrial and |
| cell Ca²⁺ overload | expression of proteins | ventricular arrhythmia; |
| | that regulate Ca²⁺ | Increased myocyte death; |
| | handling; increased | Increased fibrosis |
| | ω6ω3 PUFA ratio in | |
| | cardiac membranes | |
| Increased cardiovascular | N/A | Lower threshold for, and increased |
| reserve | | severity of heart failure |
| Reduced physical | Learned lifestyle | Exaggerate age changes in some |
| activity | | aspects of CV structure and |
| | | function; |
| | | Negative impact on atherosclerotic |
| | | vascular disease, hypertension, and |
| | | heart failure |

Abbreviations: VSMC = vascular smooth muscle cells; LV = left ventricle; PUFA = polyunsaturated fatty acids (Strait and Lakatta, Heart Fail Clin, 2012, 8: 143-164).

Diastolic Function

Diastolic function refers to several different physiological processes that allow the left ventricle (LV) to fill with sufficient blood for the body's current needs at a low enough pressure to prevent pulmonary congestion. The normal LV functions as a suction pump, with the degree of early diastolic suction being related to the extent of shortening in the previous beat and the pressure in the left atrium at the time of mitral valve in addition to LV relaxation. A hallmark of cardiac aging is a decrease in LV diastolic function (e.g., diastolic dysfunction), in which the heart experiences impaired ventricular relaxation, and increased filling pressures. Normal diastolic filling can be divided into two phases: passive filling early during diastole (‘E’), known as early diastolic transmitral flow velocity, and active filling late during diastole by atrial contraction (‘A’), known as late diastolic transmitral flow velocity. At the early stage of impaired diastolic function, the rate of the heart filling with blood declines (e.g., smaller E), the bull of ventricular filling shifts to later in diastole, and there is significant atrial enlargement and a larger blood volume for the atrium to eject during contraction (e.g., larger A). Therefore, the atrium assumes a greater portion of the total end diastolic volume and the E/A ratio decreases, which is a hallmark of diastolic dysfunction at early stages of HFpEF. The E/A ratio in healthy young adults is typically >1. Diastolic dysfunction is linked to HFpEF (heart failure with preserved ejection fraction). Diastolic dysfunction represents a combination of impaired left ventricular (LV) relaxation, restoration forces, myocyte lengthening load, and atrial function, all culminating in increased LV filling pressures. Ratios of early to late diastolic transmitral flow velocity (E/A) can be used to assess diastolic function.

There are other ways to estimate diastolic dysfunction aside from measuring (E/A). One measurement to use is the ratio of early diastolic transmitral flow to early diastolic mitral annular tissue velocity (E/e′), which estimates LV filling pressures. A normal (E/e′) is typically <15, and values greater than 15 suggest elevated LV filling pressure and HFpEF. The ratio (E/e′) can therefore also be used to assess diastolic function and is clinically preferred, as diastolic dysfunction leads to a larger E/e′ ratio due to impaired ventricular relaxation and thus a smaller e′ measurement and larger E measurement. A ratio of early diastolic mitral annular tissue velocity to late diastolic mitral annular tissue velocity (e′/a′) can also be measured.

Finally, deceleration time (DT, also referred to as E deceleration time) can be used to estimate diastolic dysfunction. DT is the interval of time from the peak of the E-wave in an echocardiogram to its projected baseline. E-wave deceleration time in a normal patient is typically between 150 ms and 240 ms. DT indicates the duration for equalizing the pressure difference between the left atrium and the left ventricle.

Although these measurements of diastolic dysfunction have important diagnostic and prognostic implications, they should be interpreted in the context of a patient's age and the rest of the echocardiogram to describe diastolic function and guide patient management. In healthy hearts, a significant amount of LV ejection and LA filling results from descent of the mitral annulus toward the apex. This longitudinal motion normally precedes filling. This motion can be both decreased and delayed in either the setting of global dysfunction (all motion is reduced) or in various settings associated with LV hypertrophy (contraction shifts from longitudinal shortening to radial thickening).

In the absence of endocardial or pericardial disease, diastolic LV dysfunction results from increased myocardial stiffness. Two compartments within the myocardium regulate its diastolic stiffness. These compartments are the extracellular matrix and cardiomyocytes. A stiffness change within one compartment is also transmitted to the other compartment via matricellular proteins. Stiffness of the extracellular matrix is largely determined by collagen through regulation of its total amount, relative abundance of collagen type I, and degree of collagen cross-linking, which are all thought to play a role in HFpEF. In addition to collagen deposition, intrinsic cardiomyocyte stiffness also contributes to diastolic LV dysfunction in HFpEF.

TABLE 9
Variables Used to Assess LV Diastolic Function

| Variable | Name | Utility | Limitations |
|---|---|---|---|
| E | Mitral E velocity | E-wave velocity | 1. In patients with coronary artery |
| | | reflects the LA-LV | disease and patients with HCM in whom |
| | | pressure gradient | LVEF is >50%, mitral velocities |
| | | during early diastole | correlate poorly with LV filling |
| | | and is affected by | pressures |
| | | alterations in the rate | 2. More challenging to apply in patients |
| | | of LV relaxation and | with arrhythmias. |
| | | LAP. | 3. Directly affected by alterations in LV |
| | | | volumes and elastic recoil. |
| | | | 4. Age dependent (decreasing with age). |
| A | Mitral A velocity | A-wave velocity | 1. Sinus tachycardia, first-degree AV |
| | | reflects the LA-LV | block and paced rhythm can result in |
| | | pressure gradient | fusion of the E and A waves. If mitral |
| | | during late diastole, | flow velocity at the start of atrial |
| | | which is affected by | contraction is >20 cm/sec, A velocity |
| | | LV compliance and | may be increased. |
| | | LA contractile | 2. Not applicable in AF/atrial flutter |
| | | function. | patients. |
| | | | 3. Age dependent (increases with |
| | | | aging). |
| E/A ratio | Mitral E/A ratio | Mitral inflow E/A | 1. The U-shaped relation with LV |
| | | ratio and DT are used | diastolic function makes it difficult to |
| | | to identify the filling | differentiate normal from PN filling, |
| | | patterns: normal, | particularly with normal LVEF, without |
| | | impaired relaxation, | additional variables. |
| | | PN, and restrictive | 2. If mitral flow velocity at the start of |
| | | filling. | atrial contraction is >20 cm/sec, E/A |
| | | | ratio will be reduced due to fusion. |
| | | | 3. Not applicable in AF/atrial flutter |
| | | | patients. |
| | | | 4. Age dependent (decreases with |
| | | | aging). |
| DT | Mitral E-velocity | DT is influenced by | 1. DT does not relate to LVEDP in |
| deceleration | DT | LV relaxation, LV | normal LVEF |
| time | | diastolic pressures | 2. Should not be measured with E and A |
| | | following mitral | fusion due to potential inaccuracy. |
| | | valve opening, and | 3. Age dependent (increases with |
| | | LV stiffness. | aging). |
| | | | 4. Not applied in atrial flutter. |
| E/e′ | Mitral E/e′ ratio | e′ velocity can be | 1. E/e′ ratio is not accurate in normal |
| | | used to correct for | subjects, patients with heavy annular |
| | | the effect of LV | calcification, mitral valve and |
| | | relaxation on mitral | pericardial disease. |
| | | E velocity, and E/e′ | 2. “Gray zone” of values in which LV |
| | | ratio can be used to | filling pressures are indeterminate. |
| | | predict LV filling | 3. Accuracy is reduced in patients with |
| | | pressures. | CAD and regional dysfunction at the |
| | | | sampled segments. |
| | | | 4. Different cutoff values depending on |
| | | | the site used for measurement. |
| LAVI | Left atrium | LA volume reflects | 1. LA dilation is seen in bradycardia, |
| | maximum volume | the cumulative | high-output states, heart transplants with |
| | index | effects of increased | biatrial technique, atrial |
| | | LV filling pressures | flutter/fibrillation, significant mitral |
| | | over time. Increased | valve disease, despite normal LV |
| | | LA volume is an | diastolic function. |
| | | independent | 2. LA dilatation occurs in well-trained |
| | | predictor of death, | athletes who have bradycardia and are |
| | | heart failure, AF, and | well hydrated. |
| | | ischemic stroke. | 3. Suboptimal image quality, including |
| | | | LA foreshortening, in technically |
| | | | challenging studies precludes accurate |
| | | | tracings. |
| | | | 4. It can be difficult to measure LA |
| | | | volumes in patients with ascending and |
| | | | descending aortic aneurysms as well as |
| | | | in patients with large interatrial septal |
| | | | aneurysms. |

Abbreviations: A = late (atrial) transmitral pulse-wave Doppler flow; AF = atrial fibrillation; DT = deceleration time; E = early transmitral pulsed-wave Doppler flow; e′ = early mitral annular tissue Doppler velocity; LA = left atrium; LAP = left atrial pressure; LV = left ventricle; LAVI = left atrial volume indexed to body surface area. TR = tricuspid regurgitation (Nagueh, S. F. et al., J Am Soc Echocardiogr., 2016, 29: 277-314).

There are multiple sets of guidelines published for diagnosing diastolic dysfunction. While parameters may differ, all guidelines require the presence of signs or symptoms of HF, evidence of normal systolic LV function, and evidence of diastolic dysfunction or surrogate markers that include LV hypertrophy, LA enlargement, atrial fibrillation or elevated BNP levels. According to American Society of Echocardiography and the European Association of Cardiovascular Imaging, diastolic dysfunction can be divided into four grades or stages, based on the above measurements, among others. Table 10 presents a summary of the expected findings for the different grades of diastolic dysfunction. (Nagueh, S. F. et al., J Am Soc Echocardiogr., 2016, 29:277-314). Importantly, E/e′ ratio can be measured to determine grade of diastolic dysfunction in a patient suspected of HFpEF. An E/e′ value in a patient with Grade 1 diastolic dysfunction is less than 8. An E/e′ value in a patient with Grade 2 diastolic dysfunction is between 8 and 15. An E/e′ value in a patient with Grade 3 diastolic dysfunction is above 15.

TABLE 10
Stages/Grades of Diastolic Dysfunction

| | | | | | | | | | LA |
|---|---|---|---|---|---|---|---|---|---|
| Stage of | | | | | Mitral | Average | | Peak TR | Volume |
| Diastolic | LV | | | | E/A | E/e′ | | velocity | Index |
| Dysfunction | Relaxation | LAP | E | A | ratio | ratio | DT | (m/sec) | (LAVI) |
| Normal | Normal | Normal | 60-100 cm/s | 40-85 cm/s | 1-2 | <8 | <160 ms | <2.8 | Normal |
| Grade 1 | Impaired | Low or | decrease | increase | <1 | <8 | >160 ms | <2.8 | Normal or |
| | | normal | | | | | | | increased |
| Grade 2 | Impaired | Elevated | increase | decrease | 1-2 | 8-15 | <160 ms | >2.8 | Increased |
| Grade 3 | Impaired | Elevated | increase | decrease | >2 | >15 | <160 ms | Increased | Increased |

Abbreviations: A = late (atrial) transmitral pulse-wave Doppler flow; AF = atrial fibrillation; DT = deceleration time; E = early transmitral pulsed-wave Doppler flow; e′ = early mitral annular tissue Doppler velocity; LA = left atrium; LAP = left atrial pressure; LV = left ventricle; LAVI = left atrial volume indexed to body surface area; TR velocity = tricuspid 5 regurgitation velocity. (Nagueh, S. F. et al., J Am Soc Echocardiogr., 2016, 29: 277-314) and (Lekavich C. L. et al., Heart Fail Rev, 2015, 20: 643-653).

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient has diastolic dysfunction. In some embodiments, the patient has a decrease in left ventricle diastolic function in comparison to healthy people of similar age and sex. In some embodiments, the patient has decreased left ventricular relaxation in comparison to healthy people of similar age and sex. In some embodiments, a patient's E/A ratio is measured. In some embodiments, a patient's ratio of early diastolic transmitral flow velocity to late diastolic transmitral flow velocity (E/A) is measured. In some embodiments, the patient's rate of filling of blood in the heart is decreased in comparison to healthy people of similar age and sex. In some embodiments, the patient has an increased amount of blood volume for the atrium of the heart to eject during contraction. In some embodiments, the patient has atrial enlargement. In some embodiments, the patient has a decrease in E/A ratio in comparison to healthy people of similar age and sex. In some embodiments, the patient has increased left atrial pressure in comparison to people of similar age and sex. In some embodiments, the patient has decreased LV filling pressure in comparison to healthy people of similar age and sex. In some embodiments, the patient's ratio of early diastolic transmitral flow to early diastolic mitral annular tissue velocity (E/e′) is measured. In some embodiments, a patient's E/e′ ratio is increased in comparison to healthy people of similar age and sex. In some embodiments, the patient's E/e′ ratio is less than 8. In some embodiments, the patient's E/e′ ratio is between 8 and 15. In some embodiments, the patient's E/e′ ratio is greater than 15. In some embodiments, a patient's ratio of early diastolic mitral annular tissue velocity to late diastolic mitral annular tissue velocity (e′/a′) is measured. In some embodiments, a patient's deceleration time (DT) is measured. In some embodiments, a patient's deceleration time is reduced compared to healthy people of similar age and sex. In some embodiments, a patient's deceleration time is less than 160 ms. In some embodiments, a patient's tricuspid regurgitation velocity (TR velocity) is measured. In some embodiments, a patient's TR velocity is generally increased. In some embodiments, a patient's TR velocity is generally greater than 2.8 m/sec. In some embodiments, a patient's left atrial volume index (LAVI) is measured. In some embodiments, a patient's LAVI is increased compared to healthy people of similar age and sex.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves diastolic dysfunction. In some embodiments, the method increases left ventricular diastolic function. In some embodiments, the method improves left ventricular relaxation. In some embodiments, the method improves a patient's ratio of early diastolic transmitral flow velocity to late diastolic transmitral flow velocity (E/A). In some embodiments, the method generally decreases a patient's E/A ratio. In some embodiments, the method improves a patient's ratio of early diastolic mitral annular tissue velocity to late diastolic mitral annular tissue velocity (e′/a′). In some embodiments, the method generally decreases a patient's e′/a′ ratio. In some embodiments, the method improves a patient's deceleration time (DT) in the heart. In some embodiments, the method generally increases a patient's deceleration time (DT) in the heart. In some embodiments, the method generally decreases a patient's DT to below 160 ms. In some embodiments, the method increases a patient's rate of filling of blood in the heart. In some embodiments, the method decreases the patient's amount of blood volume for the atrium of the heart to eject during contraction. In some embodiments, the method increases left ventricular relaxation. In some embodiments, the method decreases left atrial pressure. In some embodiments, the method improves atrial enlargement. In some embodiments, the method increases LV filling pressure. In some embodiments, the method generally decreases a patient's TR velocity. In some embodiments, the method generally decreases a patient's TR velocity to below 2.8 m/sec. In some embodiments, the method decreases a patient's left atrial volume index (LAVI) measurement.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method decreases a patient's ratio of early diastolic transmitral flow to early diastolic mitral annular tissue velocity (E/e′) (e.g., by at least 5, 10, 15, 20, 25, 30, 35, 40, 45, 50, 55, 60, 65, 70, 75, 80, 85, 90, 95, or 100%). In some embodiments, the method relates to decreasing the patient's E/e′ ratio by at least 5%. In some embodiments, the method relates to decreasing the patient's E/e′ ratio by at least 10%. In some embodiments, the method relates to decreasing the patient's E/e′ ratio by at least 15%. In some embodiments, the method relates to decreasing the patient's E/e′ ratio by at least 20%. In some embodiments, the method relates to decreasing the patient's E/e′ ratio by at least 25%. In some embodiments, the method relates to decreasing the patient's E/e′ ratio by at least 30%. In some embodiments, the method relates to decreasing the patient's E/e′ ratio by at least 35%. In some embodiments, the method relates to decreasing the patient's E/e′ ratio by at least 40%. In some embodiments, the method relates to decreasing the patient's E/e′ ratio by at least 45%. In some embodiments, the method relates to decreasing the patient's E/e′ ratio by at least 50%. In some embodiments, the method relates to decreasing the patient's E/e′ ratio by at least 55%. In some embodiments, the method relates to decreasing the patient's E/e′ ratio by at least 60%. In some embodiments, the method relates to decreasing the patient's E/e′ ratio by at least 65%. In some embodiments, the method relates to decreasing the patient's E/e′ ratio by at least 70%. In some embodiments, the method relates to decreasing the patient's E/e′ ratio by at least 75%. In some embodiments, the method relates to decreasing the patient's E/e′ ratio by at least 80%. In some embodiments, the method relates to decreasing the patient's E/e′ ratio by at least 85%. In some embodiments, the method relates to decreasing the patient's E/e′ ratio by at least 90%. In some embodiments, the method relates to decreasing the patient's E/e′ ratio by at least 95%. In some embodiments, the method relates to decreasing the patient's E/e′ ratio by 100%.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method decreases a patient's ratio of early diastolic transmitral flow to early diastolic mitral annular tissue velocity (E/e′) (e.g., by at least 1, 2, 3, 4, 5, 6, 7, 8, 9, 10, 11, 12, 13, 14, 15, 16, 17, 18, 19, 20, 25, 35, 40, 45, or 50) In some embodiments, the method relates to decreasing the patient's E/e′ ratio by at least 1. In some embodiments, the method relates to decreasing the patient's E/e′ ratio by at least 2. In some embodiments, the method relates to decreasing the patient's E/e′ ratio by at least 3. In some embodiments, the method relates to decreasing the patient's E/e′ ratio by at least 4. In some embodiments, the method relates to decreasing the patient's E/e′ ratio by at least 5. In some embodiments, the method relates to decreasing the patient's E/e′ ratio by at least 6. In some embodiments, the method relates to decreasing the patient's E/e′ ratio by at least 7. In some embodiments, the method relates to decreasing the patient's E/e′ ratio by at least 8. In some embodiments, the method relates to decreasing the patient's E/e′ ratio by at least 9. In some embodiments, the method relates to decreasing the patient's E/e′ ratio by at least 10. In some embodiments, the method relates to decreasing the patient's E/e′ ratio by at least 11. In some embodiments, the method relates to decreasing the patient's E/e′ ratio by at least 12. In some embodiments, the method relates to decreasing the patient's E/e′ ratio by at least 13. In some embodiments, the method relates to decreasing the patient's E/e′ ratio by at least 14. In some embodiments, the method relates to decreasing the patient's E/e′ ratio by at least 15. In some embodiments, the method relates to decreasing the patient's E/e′ ratio by at least 16. In some embodiments, the method relates to decreasing the patient's E/e′ ratio by at least 17. In some embodiments, the method relates to decreasing the patient's E/e′ ratio by at least 18. In some embodiments, the method relates to decreasing the patient's E/e′ ratio by at least 19. In some embodiments, the method relates to decreasing the patient's E/e′ ratio by at least 20. In some embodiments, the method relates to decreasing the patient's E/e′ ratio by at least 25. In some embodiments, the method relates to decreasing the patient's E/e′ ratio by at least 30. In some embodiments, the method relates to decreasing the patient's E/e′ ratio by at least 35. In some embodiments, the method relates to decreasing the patient's E/e′ ratio by at least 40. In some embodiments, the method relates to decreasing the patient's E/e′ ratio by at least 45. In some embodiments, the method relates to decreasing the patient's E/e′ ratio by at least 50.

In some embodiments, a patient's diastolic dysfunction grade is normal. In some embodiments, a normal grade of diastolic dysfunction comprises an E/A between 1 and 2, an E/e′ of <8, a normal left atrial volume index (LAVI), and a deceleration time (DT) of <160 ms, wherein normal refers to a healthy person of similar age and sex to the patient. In some embodiments, a patient's diastolic dysfunction stage is Grade 1. In some embodiments, Grade 1 diastolic dysfunction comprises an E/A<1 due to impaired relaxation, an E/e′ of <8, a normal or increased LAVI, and an increased deceleration time relative to a healthy person of similar age and sex. In some embodiments, a patient's diastolic dysfunction stage is Grade 2. In some embodiments, Grade 2 diastolic dysfunction comprises an E/A between 1 and 2, an E/e′ of between 8 and 15, an increased LAVI, and a decreased deceleration time relative to a healthy person of similar age and sex. In some embodiments, an increased E/e′ and/or increased LA size corroborates a diagnosis of Grade 2 from Grade 1. In some embodiments, a patient's diastolic dysfunction stage is Grade 3. In some embodiments, Grade 3 diastolic dysfunction comprises an E/A>2, an E/e′ of greater than 15, an increased LAVI, and a very short E deceleration time (<140 ms) due to severely reduced LV compliance and high LV filling pressure relative to a healthy person of similar age and sex.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves the patient's diastolic dysfunction grade. In some embodiments, the method relates to improving the patient's diastolic dysfunction grade from Grade 3 to Grade 2. In some embodiments, the method relates to improving the patient's diastolic dysfunction grade from Grade 3 to Grade 1. In some embodiments, the method relates to improving the patient's diastolic dysfunction grade from Grade 3 to normal. In some embodiments, the method relates to improving the patient's diastolic dysfunction grade from Grade 2 to Grade 1. In some embodiments, the method relates to improving the patient's diastolic dysfunction grade from Grade 2 to normal. In some embodiments, the method relates to improving the patient's diastolic dysfunction grade from Grade 1 to normal.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method increases the patient's LV diastolic function (e.g., by at least 5, 10, 15, 20, 25, 30, 35, 40, 45, 50, 55, 60, 65, 70, 75, 80, 85, 90, 95, or 100%). In some embodiments, the method relates to increasing the patient's LV diastolic function by at least 5%. In some embodiments, the method relates to increasing the patient's LV diastolic function by at least 10%. In some embodiments, the method relates to increasing the patient's LV diastolic function by at least 15%. In some embodiments, the method relates to increasing the patient's LV diastolic function by at least 20%. In some embodiments, the method relates to increasing the patient's LV diastolic function by at least 25%. In some embodiments, the method relates to increasing the patient's LV diastolic function by at least 30%. In some embodiments, the method relates to increasing the patient's LV diastolic function by at least 35%. In some embodiments, the method relates to increasing the patient's LV diastolic function by at least 40%. In some embodiments, the method relates to increasing the patient's LV diastolic function by at least 45%. In some embodiments, the method relates to increasing the patient's LV diastolic function by at least 50%. In some embodiments, the method relates to increasing the patient's LV diastolic function by at least 55%. In some embodiments, the method relates to increasing the patient's LV diastolic function by at least 60%. In some embodiments, the method relates to increasing the patient's LV diastolic function by at least 65%. In some embodiments, the method relates to increasing the patient's LV diastolic function by at least 70%. In some embodiments, the method relates to increasing the patient's LV diastolic function by at least 75%. In some embodiments, the method relates to increasing the patient's LV diastolic function by at least 80%. In some embodiments, the method relates to increasing the patient's LV diastolic function by at least 85%. In some embodiments, the method relates to increasing the patient's LV diastolic function by at least 90%. In some embodiments, the method relates to increasing the patient's LV diastolic function by at least 95%. In some embodiments, the method relates to increasing the patient's LV diastolic function by 100%.

Systolic Function

The overall resting systolic function of cardiac muscle does not typically change with healthy aging. LV ejection fraction, which is generally the most commonly used measure of LV systolic performance, is typically preserved during aging (i.e., HFpEF). Effects on systolic function are usually reflected by an age-associated reduction in cardiac reserve observable during exercise. Studies have shown that even mild limitations in basal contractility in HFpEF may become more problematic in the setting of exercise stress, where an inability to enhance contractility may be associated with impaired cardiac output reserve, more severe symptoms of exercise intolerance, and reduced aerobic capacity. Factors involved in this reduction include a decrease of myocardial contractility, and a decrease in maximum heart rate and maximum ejection fraction achieved during exercise. Decreased cardiac functional reserve is associated with heart failure in general.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient has normal systolic function. In some embodiments, a patient has no change in systolic function. In some embodiments, a patient an age-associated reduction in cardiac reserve observable during exercise.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves systolic function.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient has an ejection fraction of at least 50% (e.g., 50, 55, 60, 65, 70, 75, 80, 85, 90, 95, or 100%). In some embodiments, the method relates to patient's having an ejection fraction of at least 50%. In some embodiments, the method relates to patient's having an ejection fraction of at least 55%. In some embodiments, the method relates to patient's having an ejection fraction of at least 60%. In some embodiments, the method relates to patient's having an ejection fraction of at least 65%. In some embodiments, the method relates to patient's having an ejection fraction of at least 70%. In some embodiments, the method relates to patient's having an ejection fraction of at least 75%. In some embodiments, the method relates to patient's having an ejection fraction of at least 80%. In some embodiments, the method relates to patient's having an ejection fraction of at least 85%. In some embodiments, the method relates to patient's having an ejection fraction of at least 90%. In some embodiments, the method relates to patient's having an ejection fraction of at least 95%. In some embodiments, the method relates to patient's having an ejection fraction of 100%. In some embodiments, the ejection fraction is the right ventricular ejection fraction. In some embodiments, the ejection fraction is the left ventricular ejection fraction. In some embodiments, the ejection fraction is measured using an echocardiogram. In some embodiments, the patient has a preserved left ventricular ejection fraction.

Cardiac Output

In general, normal cardiac output at rest is about 2.5-4.2 L/min/m², and cardiac output can decline by almost 40% without deviating from the normal limits. A low cardiac index of less than about 2.5 L/min/m² usually indicates a disturbance in cardiovascular performance. In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method increases the patient's cardiac output (e.g., by at least 5, 10, 15, 20, 25, 30, 35, 40, 45, 50, 55, 60, 65, 70, 75, 80, 85, 90, 95, or 100%). In some embodiments, the method relates to increasing the patient's cardiac output by at least 5%. In some embodiments, the method relates to increasing the patient's cardiac output by at least 10%. In some embodiments, the method relates to increasing the patient's cardiac output by at least 15%. In some embodiments, the method relates to increasing the patient's cardiac output by at least 20%. In some embodiments, the method relates to increasing the patient's cardiac output by at least 25%. In some embodiments, the method relates to increasing the patient's cardiac output by at least 30%. In some embodiments, the method relates to increasing the patient's cardiac output by at least 35%. In some embodiments, the method relates to increasing the patient's cardiac output by at least 40%. In some embodiments, the method relates to increasing the patient's cardiac output by at least 45%. In some embodiments, the method relates to increasing the patient's cardiac output by at least 50%. In some embodiments, the method relates to increasing the patient's cardiac output by at least 55%. In some embodiments, the method relates to increasing the patient's cardiac output by at least 60%. In some embodiments, the method relates to increasing the patient's cardiac output by at least 65%. In some embodiments, the method relates to increasing the patient's cardiac output by at least 70%. In some embodiments, the method relates to increasing the patient's cardiac output by at least 75%. In some embodiments, the method relates to increasing the patient's cardiac output by at least 80%. In some embodiments, the method relates to increasing the patient's cardiac output by at least 85%. In some embodiments, the method relates to increasing the patient's cardiac output by at least 90%. In some embodiments, the method relates to increasing the patient's cardiac output by at least 95%. In some embodiments, the method relates to increasing the patient's cardiac output by 100%. In some embodiments, the method relates to increasing the patient's cardiac output to at least 4.2 L/min/m2. In some embodiments, the cardiac output is measured at rest. In some embodiments, the cardiac output is measured using a right heart catheter.

Electrical Function

In the cardiac conduction system, aging is associated with a vital reduction of pacemaker cells in the sinoatrial node. A pronounced decline in the number of pacemaker cells occurs after age 60. By age 75 less than 10% of the number of pacemaker cells seen in young adults remain. A variable degree of calcification on the left side of the cardiac skeleton also occurs with aging. These conduction system changes are reflected by an increased incidence of sinus dysfunction in the elderly and manifests itself by palpitations, dizziness, syncope with persistent fatigue and confusion. In addition, tissue remodeling affects the functioning of the atrioventricular node, the bundle of His and the bundle branches. The resulting changes in depolarization and repolarization of the atria and the ventricles are reflected by age-associated changes in electrocardiogram (ECG) measurements. Changes in echocardiogram measurements include an increase in P-wave duration, P-R interval and Q-T interval, and T-wave voltage and a leftward shift of the QRS axis. The P-R interval, representing atrioventricular conduction, generally increases from 159 ms at ages 20-35 to 172 ms beyond age 60. The QRS axis shifts leftward, possibly due to increases in LV wall thickness, with 20% of healthy subjects having a left axis deviation by age 100. Interestingly, despite increased LV thickness, there is a decline in the R- and S-wave amplitudes with aging evident by age 40. In addition, the prevalence of both atrial and ventricular ectopic beats increases.

TABLE 11
Normal Age-Associated Changes in Resting ECG Measurements

| Measurement | Change with Age | Effect on Mortality |
|---|---|---|
| R-R Interval | No Change | N/A |
| P-wave Duration | Minor Increase | None |
| P-R Interval | Increase | None |
| QRS Duration | No Change | N/A |
| QRS Axis | Leftward Shift | None |
| Q-T Interval | Minor Increase | Probable Increase |
| T-wave Voltage | Decrease | None |

(Strait and Lakatta, Heart Fail Clin, 2012, 8: 143-164).

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient is evaluated for heart failure using electrocardiography. In some embodiments, a patient has a reduction in number of pacemaker cells. In some embodiments, a patient has an increase in P-wave duration on an electrocardiogram. In some embodiments, a patient has an increase in P-R interval on an electrocardiogram. In some embodiments, a patient has an increase in Q-T interval on an electrocardiogram. In some embodiments, a patient has a decrease in T-wave voltage on an electrocardiogram. In some embodiments, a patient has a leftward shift of the QRS axis on an electrocardiogram.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves electrocardiography measurements. In some embodiments, the method increases the number of pacemaker cells present in a patient. In some embodiments, the method decreases P-wave duration on an electrocardiogram. In some embodiments, the method decreases P-R interval on an electrocardiogram. In some embodiments, the method decreases Q-T interval on an electrocardiogram. In some embodiments, the method increases T-wave voltage on an electrocardiogram. In some embodiments, the method shifts the QRS axis to a normal position on an electrocardiogram.

### Natriuretic Peptides

Plasma concentration of natriuretic peptides (NPs), including BNP and NT-proBNP, can be used as an initial diagnostic test, especially in a non-acute setting when echocardiography is not immediately available. Elevated NPs help establish an initial working diagnosis, identifying those who require further cardiac investigation. Patients with values below the cutoff point for the exclusion of important cardiac dysfunction typically do not require echocardiography. In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient has elevated level of one or more natriuretic peptides. In some embodiments, the method relates to treating a patient having heart failure wherein the patient has elevated levels of BNP. In some embodiments, the method relates to treating a patient having heart failure wherein the patient has elevated levels of NT-proBNP. In some embodiments, the patients NP (e.g., BNP and/or NT-proBNP) is elevated compared to healthy people of similar age and sex.

Both BNP and NT-proBNP are markers of atrial and ventricular distension due to increased intracardiac pressure. The New York Heart Association (NYHA) developed a 4-stage functional classification system for congestive heart failure (CHF) based on the severity of symptoms. Studies have demonstrated that the measured concentrations of circulating BNP and NT-proBNP increase with the severity of CHF based on the NYHA classification.

Patients with normal plasma NP concentrations are unlikely to have HF. The upper limit of normal in the non-acute setting for B-type natriuretic peptide (BNP) is 35 pg/mL, and for N-terminal pro-BNP (NT-proBNP) it is 125 pg/mL; in the acute setting, higher values should be used [e.g., BNP, 100 pg/mL; NT-proBNP, 300 pg/mL; and mid-regional pro A-type natriuretic peptide (MR-proANP), 120 pmol/L]. Diagnostic values apply similarly to HFrEF and HFpEF. On average, values are typically lower for HFpEF than for HFrEF.

There are numerous cardiovascular and non-cardiovascular causes of elevated NPs that may weaken their diagnostic utility in HF. Among them, AF, age and renal failure are the most important factors impeding the interpretation of NP measurements. On the other hand, NP levels may be disproportionally low in obese patients.

BNP

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient has elevated levels of BNP. In some embodiments, the method relates to patients having a BNP level of at least 35 pg/mL. In some embodiments, the method relates to patients having a BNP level of at least 40 pg/mL. In some embodiments, the method relates to patients having a BNP level of at least 50 pg/mL. In some embodiments, the method relates to patients having a BNP level of at least 60 pg/mL. In some embodiments, the method relates to patients having a BNP level of at least 70 pg/mL. In some embodiments, the method relates to patients having a BNP level of at least 80 pg/mL. In some embodiments, the method relates to patients having a BNP level of at least 90 pg/mL. In some embodiments, the method relates to patients having a BNP level of at least 100 pg/mL. In some embodiments, the method relates to patients having a BNP level of at least 150 pg/mL. In some embodiments, the method relates to patients having a BNP level of at least 200 pg/mL. In some embodiments, the method relates to patients having a BNP level of at least 300 pg/mL. In some embodiments, the method relates to patients having a BNP level of at least 400 pg/mL. In some embodiments, the method relates to patients having a BNP level of at least 500 pg/mL. In some embodiments, the method relates to patients having a BNP level of at least 1000 pg/mL. In some embodiments, the method relates to patients having a BNP level of at least 5000 pg/mL. In some embodiments, the method relates to patients having a BNP level of at least 10,000 pg/mL. In some embodiments, the method relates to patients having a BNP level of at least 15,000 pg/mL. In some embodiments, the method relates to patients having a BNP level of at least 20,000 pg/mL.

In some embodiments, the disclosure relates to methods of adjusting one or more natriuretic peptides in the heart failure patient toward a more normal level (e.g., normal as compared to healthy people of similar age and sex), comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the method relates to reducing the patient's BNP by at least 5 pg/mL. In some embodiments, the method relates to reducing the patient's BNP by at least 10 pg/mL. In some embodiments, the method relates to reducing the patient's BNP by at least 50 pg/mL. In some embodiments, the method relates to reducing the patient's BNP by at least 100 pg/mL. In some embodiments, the method relates to reducing the patient's BNP by at least 200 pg/mL. In some embodiments, the method relates to reducing the patient's BNP by at least 500 pg/mL. In some embodiments, the method relates to reducing the patient's BNP by at least 1000 pg/mL. In some embodiments, the method relates to reducing the patient's BNP by at least 5000 pg/mL.

In some embodiments, the method relates to reducing the patient's BNP by at least 5% (e.g., 5, 10, 15, 20, 25, 30, 35, 40, 45, 50, 55, 60, 65, 70, 75, 80, 85, 90, 95, or 100%). In some embodiments, the method relates to reducing the patient's BNP by at least 5%. In some embodiments, the method relates to reducing the patient's BNP by at least 10%. In some embodiments, the method relates to reducing the patient's BNP by at least 15%. In some embodiments, the method relates to reducing the patient's BNP by at least 20%. In some embodiments, the method relates to reducing the patient's BNP by at least 25%. In some embodiments, the method relates to reducing the patient's BNP by at least 30%. In some embodiments, the method relates to reducing the patient's BNP by at least 35%. In some embodiments, the method relates to reducing the patient's BNP by at least 40%. In some embodiments, the method relates to reducing the patient's BNP by at least 45%. In some embodiments, the method relates to reducing the patient's BNP by at least 50%. In some embodiments, the method relates to reducing the patient's BNP by at least 55%. In some embodiments, the method relates to reducing the patient's BNP by at least 60%. In some embodiments, the method relates to reducing the patient's BNP by at least 65%. In some embodiments, the method relates to reducing the patient's BNP by at least 70%. In some embodiments, the method relates to reducing the patient's BNP by at least 75%. In some embodiments, the method relates to reducing the patient's BNP by at least 80%. In some embodiments, the method relates to reducing the patient's BNP by at least 85%. In some embodiments, the method relates to reducing the patient's BNP by at least 90%. In some embodiments, the method relates to reducing the patient's BNP by at least 95%. In some embodiments, the method relates to reducing the patient's BNP by 100%.

NT-proBNP

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient has a NT-proBNP level of at least 100 pg/mL (e.g., 100, 125, 150, 200, 300, 400, 500, 1000, 3000, 5000, 10,000, 15,000, 20,000, 25,000, or 30,000 pg/mL). In some embodiments, the method relates to patient's having a NT-proBNP level of at least 100 pg/mL. In some embodiments, the method relates to patients having a NT-proBNP level of at least 125 pg/mL. In some embodiments, the method relates to patients having a NT-proBNP level of at least 150 pg/mL. In some embodiments, the method relates to patients having a NT-proBNP level of at least 200 pg/mL. In some embodiments, the method relates to patients having a NT-proBNP level of at least 300 pg/mL. In some embodiments, the method relates to patients having a NT-proBNP level of at least 400 pg/mL. In some embodiments, the method relates to patients having a NT-proBNP level of at least 500 pg/mL. In some embodiments, the method relates to patients having a NT-proBNP level of at least 1000 pg/mL. In some embodiments, the method relates to patients having a NT-proBNP level of at least 5000 pg/mL. In some embodiments, the method relates to patients having a NT-proBNP level of at least 10,000 pg/mL. In some embodiments, the method relates to patients having a NT-proBNP level of at least 15,000 pg/mL. In some embodiments, the method relates to patients having a NT-proBNP level of at least 20,000 pg/mL. In some embodiments, the method relates to patients having a NT-proBNP level of at least 25,000 pg/mL. In some embodiments, the method relates to patients having a NT-proBNP level of at least 30,000 pg/mL.

In some embodiments, the disclosure relates to methods of adjusting one or more natriuretic peptides in the heart failure patient toward a more normal level (e.g., normal as compared to healthy people of similar age and sex), comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the method relates to reducing the patient's NT-proBNP by at least 10 pg/mL. In some embodiments, the method relates to reducing the patient's NT-proBNP by at least 25 pg/mL. In some embodiments, the method relates to reducing the patient's NT-proBNP by at least 50 pg/mL. In some embodiments, the method relates to reducing the patient's NT-proBNP by at least 100 pg/mL. In some embodiments, the method relates to reducing the patient's NT-proBNP by at least 200 pg/mL. In some embodiments, the method relates to reducing the patient's NT-proBNP by at least 500 pg/mL. In some embodiments, the method relates to reducing the patient's NT-proBNP by at least 1000 pg/mL. In some embodiments, the method relates to reducing the patient's NT-proBNP by at least 5000 pg/mL. In some embodiments, the method relates to reducing the patient's NT-proBNP by at least 10,000 pg/mL. In some embodiments, the method relates to reducing the patient's NT-proBNP by at least 15,000 pg/mL. In some embodiments, the method relates to reducing the patient's NT-proBNP by at least 20,000 pg/mL. In some embodiments, the method relates to reducing the patient's NT-proBNP by at least 25,000 pg/mL.

In some embodiments, the method relates to reducing the patient's NT-proBNP by at least 5% (e.g., 5, 10, 15, 20, 25, 30, 35, 40, 45, 50, 55, 60, 65, 70, 75, 80, 85, 90, 95, or 100%). In some embodiments, the method relates to reducing the patient's NT-proBNP by at least 5%. In some embodiments, the method relates to reducing the patient's NT-proBNP by at least 10%. In some embodiments, the method relates to reducing the patient's NT-proBNP by at least 15%. In some embodiments, the method relates to reducing the patient's NT-proBNP by at least 20%. In some embodiments, the method relates to reducing the patient's NT-proBNP by at least 25%. In some embodiments, the method relates to reducing the patient's NT-proBNP by at least 30%. In some embodiments, the method relates to reducing the patient's NT-proBNP by at least 35%. In some embodiments, the method relates to reducing the patient's NT-proBNP by at least 40%. In some embodiments, the method relates to reducing the patient's NT-proBNP by at least 45%. In some embodiments, the method relates to reducing the patient's NT-proBNP by at least 50%. In some embodiments, the method relates to reducing the patient's NT-proBNP by at least 55%. In some embodiments, the method relates to reducing the patient's NT-proBNP by at least 60%. In some embodiments, the method relates to reducing the patient's NT-proBNP by at least 65%. In some embodiments, the method relates to reducing the patient's NT-proBNP by at least 70%. In some embodiments, the method relates to reducing the patient's NT-proBNP by at least 75%. In some embodiments, the method relates to reducing the patient's NT-proBNP by at least 80%. In some embodiments, the method relates to reducing the patient's NT-proBNP by at least 85%. In some embodiments, the method relates to reducing the patient's NT-proBNP by at least 90%. In some embodiments, the method relates to reducing the patient's NT-proBNP by at least 95%. In some embodiments, the method relates to reducing the patient's NT-proBNP by 100%.

### Troponin Levels

Troponin, or the troponin complex, is a complex of three regulatory proteins (troponin C, troponin I, and troponin T) that is integral to muscle contraction in skeletal muscle and cardiac muscle, but not smooth muscle. Blood troponin levels may be used as a diagnostic marker for stroke, although the sensitivity of this measurement is low. Measurements of cardiac-specific troponins I and T are extensively used as diagnostic and prognostic indicators in the management of myocardial infarction and acute coronary syndrome.

Certain subtypes of troponin (cardiac I and T) are sensitive and specific indicators of damage to the heart muscle (myocardium). They are measured in the blood to differentiate between unstable angina and myocardial infarction (heart attack) in people with chest pain or acute coronary syndrome. A person who recently had a myocardial infarction would have an area of damaged heart muscle and elevated cardiac troponin levels in the blood. This can also occur in people with coronary vasospasm, a type of myocardial infarction involving severe constriction of the cardiac blood vessels. After a myocardial infarction troponins may remain high for up to 2 weeks.

Cardiac troponins are a marker of heart muscle damage. Diagnostic criteria for raised troponin indicating myocardial infarction is currently set by the WHO at a threshold of 2 pg or higher. Critical levels of other cardiac biomarkers are also relevant, such as creatine kinase. Other conditions that directly or indirectly lead to heart muscle damage and death can also increase troponin levels, such as kidney failure. Severe tachycardia (for example due to supraventricular tachycardia) in an individual with normal coronary arteries can also lead to increased troponins for example, it is presumed due to increased oxygen demand and inadequate supply to the heart muscle.

Troponins are increased in patients with heart failure, where they also predict mortality and ventricular rhythm abnormalities. They can rise in inflammatory conditions such as myocarditis and pericarditis with heart muscle involvement (which is then termed myopericarditis).

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient has elevated levels of troponin. In some embodiments, the disclosure relates to methods of adjusting one or more parameters in the heart failure patient toward a more normal level (e.g., normal as compared to healthy people of similar age and sex), comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the method relates to decreasing the patient's troponin levels by least 1% (e.g., 1, 5, 10, 15, 20, 25, 30, 35, 40, 45, 50, 55, 60, 65, 70, 75, 80, 85, 90, 95, or 100%). In some embodiments, the method relates to decreasing the patient's troponin levels by at least 1%. In some embodiments, the method relates to decreasing the patient's troponin levels by at least 5%. In some embodiments, the method relates to decreasing the patient's troponin levels by at least 10%. In some embodiments, the method relates to decreasing the patient's troponin levels by at least 15%. In some embodiments, the method relates to decreasing the patient's troponin levels by at least 20%. In some embodiments, the method relates to decreasing the patient's troponin levels by at least 25%. In some embodiments, the method relates to decreasing the patient's troponin levels by at least 30%. In some embodiments, the method relates to decreasing the patient's troponin levels by at least 35%. In some embodiments, the method relates to decreasing the patient's troponin levels by at least 40%. In some embodiments, the method relates to decreasing the patient's troponin levels by at least 45%. In some embodiments, the method relates to decreasing the patient's troponin levels by at least 50%. In some embodiments, the method relates to decreasing the patient's troponin levels by at least 55%. In some embodiments, the method relates to decreasing the patient's troponin levels by at least 60%. In some embodiments, the method relates to decreasing the patient's troponin levels by at least 65%. In some embodiments, the method relates to decreasing the patient's troponin levels by at least 70%. In some embodiments, the method relates to decreasing the patient's troponin levels by at least 75%. In some embodiments, the method relates to decreasing the patient's troponin levels by at least 80%. In some embodiments, the method relates to decreasing the patient's troponin levels by at least 85%. In some embodiments, the method relates to decreasing the patient's troponin levels by at least 90%. In some embodiments, the method relates to decreasing the patient's troponin levels by at least 95%. In some embodiments, the method relates to decreasing the patient's troponin levels by 100%.

### Exercise Capacity (6MWD AND BDI)

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). Any suitable measure of exercise capacity can be used. For example, exercise capacity in a 6-minute wall test (6MWT), which measures how far the patient can wall in 6 minutes, i.e., the 6-minute wall distance (6MWD), is frequently used to assess heart failure severity and disease progression. The Borg dyspnea index (BDI) is a numerical scale for assessing perceived dyspnea (breathing discomfort). It measures the degree of breathlessness, for example, after completion of the 6MWT, where a BDI of 0 indicates no breathlessness and 10 indicates maximum breathlessness. In some embodiments, the method relates to increasing 6MWD by at least 10 meters in the patient having heart failure (e.g., heart failure associated with aging). In some embodiments, the method relates to increasing 6MWD by at least 30 meters in the patient having heart failure (e.g., heart failure associated with aging). In some embodiments, the method relates to increasing 6MWD by at least 40 meters in the patient having heart failure (e.g., heart failure associated with aging). In some embodiments, the method relates to increasing 6MWD by at least 60 meters in the patient having heart failure (e.g., heart failure associated with aging). In some embodiments, the method relates to increasing 6MWD by at least 70 meters in the patient having heart failure (e.g., heart failure associated with aging). In some embodiments, the method relates to increasing 6MWD by at least 80 meters in the patient having heart failure (e.g., heart failure associated with aging). In some embodiments, the method relates to increasing 6MWD by at least 90 meters in the patient having heart failure (e.g., heart failure associated with aging). In some embodiments, the method relates to increasing 6MWD by at least 100 meters in the patient having heart failure (e.g., heart failure associated with aging). In some embodiments, the 6MWD is tested after the patient has received 4 weeks of treatment utilizing disclosed herein. In some embodiments, the 6MWD is tested after the patient has received 8 weeks of treatment utilizing disclosed herein. In some embodiments, the 6MWD is tested after the patient has received 12 weeks of treatment utilizing disclosed herein. In some embodiments, the 6MWD is tested after the patient has received 16 weeks of treatment utilizing de disclosed herein. In some embodiments, the 6MWD is tested after the patient has received 20 weeks of treatment utilizing disclosed herein. In some embodiments, the 6MWD is tested after the patient has received 22 weeks of treatment utilizing disclosed herein. In some embodiments, the 6MWD is tested after the patient has received 24 weeks of treatment utilizing disclosed herein. In some embodiments, the 6MWD is tested after the patient has received 26 weeks of treatment utilizing disclosed herein. In some embodiments, the 6MWD is tested after the patient has received 28 weeks of treatment utilizing disclosed herein.

In some embodiments, the method relate to lowering BDI by at least 0.5 index points in the patient having heart failure associated with aging. In some embodiments, the method relate to lowering BDI by at least 1 index points in the patient having heart failure associated with aging. In some embodiments, the method relate to lowering BDI by at least 1.5 index points in the patient having heart failure associated with aging. In some embodiments, the method relate to lowering BDI by at least 2 index points in the patient having heart failure associated with aging. In some embodiments, the method relate to lowering BDI by at least 2.5 index points in the patient having heart failure associated with aging. In some embodiments, the method relate to lowering BDI by at least 3 index points in the patient having heart failure associated with aging. In some embodiments, the method relate to lowering BDI by at least 3.5 index points in the patient having heart failure associated with aging. In some embodiments, the method relate to lowering BDI by at least 4 index points in the patient having heart failure associated with aging. In some embodiments, the method relate to lowering BDI by at least 4.5 index points in the patient having heart failure associated with aging. In some embodiments, the method relate to lowering BDI by at least 5 index points in the patient having heart failure associated with aging. In some embodiments, the method relate to lowering BDI by at least 5.5 index points in the patient having heart failure associated with aging. In some embodiments, the method relate to lowering BDI by at least 6 index points in the patient having heart failure associated with aging. In some embodiments, the method relate to lowering BDI by at least 6.5 index points in the patient having heart failure associated with aging. In some embodiments, the method relate to lowering BDI by at least 7 index points in the patient having heart failure associated with aging. In some embodiments, the method relate to lowering BDI by at least 7.5 index points in the patient having heart failure associated with aging. In some embodiments, the method relate to lowering BDI by at least 8 index points in the patient having heart failure associated with aging. In some embodiments, the method relate to lowering BDI by at least 8.5 index points in the patient having heart failure associated with aging. In some embodiments, the method relate to lowering BDI by at least 9 index points in the patient having heart failure associated with aging. In some embodiments, the method relate to lowering BDI by at least 9.5 index points in the patient having heart failure associated with aging. In some embodiments, the method relate to lowering BDI by at least 3 index points in the patient having heart failure associated with aging. In some embodiments, the method relate to lowering BDI by 10 index points in the patient having heart failure associated with aging.

### Stress Diastolic Testing

In patients with exertional dyspnea, exercise hemodynamic response provides more physiological and diagnostic information than assessment of LV diastolic function at rest. Therefore, it is helpful to assess hemodynamic response to exercise to confirm that dyspnea is a consequence of left ventricular diastolic dysfunction. There are two types of diastolic stress tests-invasive and echocardiographic. An invasive diastolic stress test is performed while the patient is doing exercise on a bicycle, which is fixed at a catheterization table. Changes of pulmonary capillary wedge pressure, an indirect parameter of LV filling pressure, during exercise is evaluated by right heart catheterization through the right internal jugular vein or by introducing a pigtail catheter into the LV from a radial arterial access site. LV systolic pressure, minimal LV pressure, LV end-diastolic pressure, and mean LV diastolic pressures are measured. A non-invasive measure comprises the combination of pulsed and tissue Doppler parameters, E/e′, which is typically measured to determine LV filling pressures. The American Society of Echocardiography, among others, has proposed that diastolic stress test should be considered abnormal in presence of these parameters: (i) septal e′ velocity <7 cm/s or lateral e′ velocity <10 cm/s at rest; (ii) average E/e′>14 or septal E/e′ ratio >15 with exercise; (iii) peak tricuspid regurgitation (TR) velocity >2.8 m/s with exercise, and (iv) left atrium volume index (LAVI) of >34 mL/m². The combination of E/e′ and TR>2.8 m/s during exercise has been shown to be sensitive for detection of HFpEF. It has also been shown that elevation of E/e′ is related to reduced oxygen consumption, whereas the combination of increased E/e′ and TR velocity was associated with elevated NT-proBNP values during exercise.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient is assessed for diastolic dysfunction using stress diastolic testing. In some embodiments, the diastolic stress test is performed on a bicycle fixed to a catheterization table. In some embodiments, the diastolic stress test is performed using echocardiography. In some embodiments, a patient with an abnormal diastolic stress test has parameters comprising a septal e′ velocity <7 cm/s or lateral e′ velocity <10 cm/s at rest, an average E/e′>14 or septal E/e′ ratio >15 with exercise, a peak tricuspid regurgitation (TR) velocity >2.8 m/s with exercise, and an left atrium volume index (LAVI) of >34 mL/m².

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves the patient's stress diastolic test result. In some embodiments, the method improves the patient's diastolic function as reported by the diastolic stress test. In some embodiments, the method increases septal e′ velocity to >7 cm/s or lateral e′ velocity to >10 cm/s at rest, decreases average E/e′ to below 14 or septal E/e′ ratio to below 15 with exercise, decreases peak tricuspid regurgitation (TR) velocity to <2.8 m/s with exercise, and decreases left atrium volume index (LAVI) to <34 mL/m².

### H₂FPEF Score

In patients with suspected HFpEF, including heart failure associated with aging, an H₂FPEF score can be used to estimate the probability of HFpEF versus noncardiac causes of dyspnea. Dyspnea is a common sign of heart failure in elderly heart failure patients. A group at the Mayo Clinic developed and clinically validated the H₂FPEF score, which is a sum of points assigned to the following clinical variables: Heavy (e.g., body mass index of >30 kg/m²=two points); Hypertensive (e.g., the patient is taking two or more antihypertensive medicines=one point); Arterial Fibrillation (AF) (e.g., paroxysmal or persistent=three points); Pulmonary hypertension (PH) (e.g., pulmonary artery systolic pressure of >35 mm Hg by echocardiography=one point); Elder (e.g., the age of the patient is >60 years=one point); and Filling pressure (e.g., echocardiography measuring E/e′ of >9=one point). The probability that HFpEF is the cause of symptoms in a patient increases with increasing total H₂FPEF score (ranging from lowest of 0 to highest of 9). The factor of elderly age (e.g., the patient is 60 years or older) alone is one point out of the total 9 points that comprise the H₂FPEF score. A low H₂FPEF score of 0 or 1 is associated with a low (e.g., <25 percent) probability of HFpEF in the patient. A low score suggests that symptoms are most likely due to a noncardiac cause. However, if the cause of symptoms remains uncertain after evaluation for noncardiac causes, a cardiology consultation and right heart catheterization is suggested to determine if HFpEF is present. An intermediate H₂FPEF score of 2 to 5 is associated with an intermediate (e.g., 40 to 80 percent) probability of HFpEF. In intermediate scoring patients, an assessment is done to determine if the natriuretic peptide level high (e.g., brain natriuretic peptide (BNP)>100 pg/mL or N-terminal proBNP (NT-proBNP)>300 pg/mL), and if there is an absence significant lung disease. If both criteria are met, the clinical findings are diagnostic for HFpEF. If one or both of criteria are not satisfied, a cardiology consultation and right heart catheterization are typically performed to gather more information. In right heart catheterization, a pulmonary capillary wedge pressure (PCWP) of ≥15 mmHg at rest or ≥25 mmHg during exercise is diagnostic for HFpEF. An H₂FPEF score of 6 or greater is associated with a greater than 90 percent probability of HFpEF and is thus considered diagnostic for HFpEF. Two components of the H₂FPEF score are derived from Doppler echocardiography: the estimated pulmonary artery systolic pressure (PASP) and E/e′ ratio. Elevation in estimated PASP by echocardiography is very common in patients with HFpEF, and identification of an elevated PASP in an older patient with dyspnea should trigger consideration for the diagnosis of HFpEF.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient is evaluated for HFpEF using an H₂FPEF score. In some embodiments, a patient has an H₂FPEF score of 0. In some embodiments, a patient has an H₂FPEF score of 1. In some embodiments, a patient has an H₂FPEF score of 2. In some embodiments, a patient has an H₂FPEF score of 3. In some embodiments, a patient has an H₂FPEF score of 4. In some embodiments, a patient has an H₂FPEF score of 5. In some embodiments, a patient has an H₂FPEF score of 6. In some embodiments, a patient has an H₂FPEF score of 7. In some embodiments, a patient has an H₂FPEF score of 8. In some embodiments, a patient has an H₂FPEF score of 9. In some embodiments, a patient has an H₂FPEF score of between about 0 and about 1. In some embodiments, a patient has an H₂FPEF score of between about 2 and about 5. In some embodiments, a patient has an H₂FPEF score of between about 6 and about 9.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) wherein the method decreases a patient's H₂FPEF score (e.g., by 1, 2, 3, 4, 5, 6, 7, 8, 9 points). In some embodiments, the method relates to decreasing a patient's H₂FPEF score by at least 1 point. In some embodiments, the method relates to decreasing a patient's H₂FPEF score by at least 2 points. In some embodiments, the method relates to decreasing a patient's H₂FPEF score by at least 3 points. In some embodiments, the method relates to decreasing a patient's H₂FPEF score by at least 4 points. In some embodiments, the method relates to decreasing a patient's H₂FPEF score by at least 5 points. In some embodiments, the method relates to decreasing a patient's H₂FPEF score by at least 6 points. In some embodiments, the method relates to decreasing a patient's H₂FPEF score by at least 7 points. In some embodiments, the method relates to decreasing a patient's H₂FPEF score by at least 8 points. In some embodiments, the method relates to decreasing a patient's H₂FPEF score by at least 9 points.

### Right Heart Catheterization

Right heart catheterization (sometimes called pulmonary catheterization) is not universally required for diagnosis and evaluation of HFpEF. However, in selected patients with intermediate H₂FPEF scores (and selected patients with low H₂FPEF scores with undetermined causes of symptoms), right heart catheterization is useful for assessment of cardiac filling pressures at rest and during exercise, to help make or exclude a diagnosis of HFpEF. Right heart catheterization is a test used to see how well the heart is pumping (e.g., how much it pumps per minute) and to measure the blood pressure in the heart and the main blood vessels in the lungs. Right heart catheterization is different than a left heart catheterization (coronary angiography), which is used to check for blockages in the arteries. In right heart catheterization, a pulmonary artery (PA) catheter is guided to the right side of the heart and into the pulmonary artery, which is the main artery that carries blood to the lungs. Blood flow through the heart can be observed and pressures inside the heart and lungs and measured. As the catheter advances toward the pulmonary artery, pressures are measured along the way, inside the chambers on the right side of the heart, including in the right atrium and right ventricle. Indirect measurements of pressures on the left side of the heart can also be measured. Cardiac output (e.g., the amount of blood the heart pumps per minute) is also determined. A pulmonary capillary wedge pressure (PCWP) of ≥15 mmHg at rest or ≥25 mmHg during exercise is diagnostic of HFpEF. In some embodiments, a patient is assessed for heart failure using right heart catheterization. In some embodiments, a patient is diagnosed with HFpEF using right heart catheterization. In some embodiments, a subject with a PCWP of ≥15 mmHg at rest measured with right heart catheterization has HFpEF. In some embodiments, a subject with a PCWP of ≥25 mmHg during exercise measured with right heart catheterization has HFpEF.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves pulmonary capillary wedge pressure (PCWP). In some embodiments, the method decreases PCWP at rest to at least below 15 mm Hg. In some embodiments, the method decreases PCWP during exercise to at least below 25 mm Hg.

### Heart Failure Association (HFA) of the European Society of Cardiology (ESC) Criteria for Diagnosing HFpEF

The European Heart Failure Association recently published consensus and proposed criteria for diagnosis of HFpEF (Table 12). This consensus was aimed to provide stepwise diagnostic approach from clinical assessment to more specific tests. The criteria were separated into 3 groups: functional, morphological, and biomarker. Major functional criteria included echocardiographic parameters that were proposed in the guidelines for assessment of LV diastolic dysfunction (reduced septal e′, increased E/e′, and increased TR) (see also Table 10). Minor functional criteria included intermediate values of E/e′ and reduced LV global longitudinal strain (<−16%). Major morphological criteria include dilated left atrial volume index (LAVI≥34 ml/m² in sinus rhythm and ≥40 ml/m² in atrial fibrillation) or left ventricle hypertrophy defined as LV mass index (LVMI)≥149 g/m² in men or ≥122 g/m² in women together with increased relative wall thickness ≥0.42. Interestingly, minor morphological criteria were high normal values of LA volume index (29-34 ml/m2 in sinus rhythm and ≥34-40 ml/m² in atrial fibrillation), increased LV mass index defined by current echocardiographic guidelines (≥115 g/m2 in men or ≥95 g/m2 in women), or relative wall thickness ≥0.42 or LV wall thickness ≥12 mm. Major and minor biomarker criteria refer to different levels of BNP and pro-BNP with various cutoff values for patients with sinus rhythm and atrial fibrillation (values are 3 times higher in the atrial fibrillation group). Only one criterion from each group can be included in the score. A score of ≥5 points indicates HFpEF. A score of 2-4 points indicates a diastolic stress test or invasive hemodynamic measurements should be pursued. A score of 1 point or less indicates that a diagnosis of HFpEF is unlikely. (Pieske B., et al., Eur Heart J, 2019, 40:3297-3317) and (Tadic M. et al., Heart Failure Reviews, 2020, 10.1007/s10741-020-09966-4).

TABLE 12
Summary of European Heart Failure Association Criteria for Diagnosing HFpEF

| | | | Biomarkers | |
|---|---|---|---|---|
| | | | | Atrial |
| Criteria | Functional | Morphological | Sinus Rhythm | Fibrillation |
| Major Criteria | Septal e′ | LAVI >34 | NT-proBNP >220 | NT-proBNP >660 |
| (each worth 2 | velocity <7 | mL/m² | pg/mL or | pg/mL or |
| points) | cm/s | | BNP >80 pg/mL | BNP >240 pg/mL |
| | Lateral e′ | LVMI ≥149 | — | — |
| | velocity <10 | g/m² for men | | |
| | cm/s at rest | and ≥122 g/m² | | |
| | | for women and | | |
| | | RWT >0.42 | | |
| | Average E/e′ >14 | — | — | — |
| | or septal E/e′ | | | |
| | ratio >15 with | | | |
| | exercise TR | | | |
| | velocity >2.8 | — | — | — |
| | m/s with | | | |
| | exercise | | | |
| Minor Criteria | Average E/e′ | LAVI 29-34 | 5-NT-proBNP | NT-proBNP |
| (each worth 1 | 9-14 | mL/m² | 125-220 pg/mL | 365-660 pg/mL |
| point) | | | or BNP 35-80 | or BNP 105-240 |
| | | | pg/mL | pg/mL |
| | GLS <16% | LVMI >115 | — | — |
| | | g/m2 for men | | |
| | | and 95 g/m² for | | |
| | | women; | | |
| | | RWT >0.42 | | |
| | — | LV wall | — | — |
| | | thickness ≥12 | | |
| | | mm | | |

Abbreviations: BNP brain natriuretic peptide, HFpEF heart failure with preserved ejection fraction, E and e′ early diastolic mitral flow velocity measured by pulsed and tissue Doppler, GLS left ventricular global longitudinal strain, LAVI left atrial volume index, LVMI left ventricular mass index, RWT relative wall thickness. (Pieske B., et al., Eur Heart J, 2019, 40: 3297-3317) and (Tadic M. et al., Heart Failure Reviews, 2020, 10.1007/s10741-020-09966-4).

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient is evaluated for HFpEF using the European Heart Failure Association (EHFA) criteria.

In some embodiments, a patient has a European Heart Failure Association (EHFA) score of 0. In some embodiments, a patient has an EHFA score of 1. In some embodiments, a patient has an EHFA score of 2. In some embodiments, a patient has an EHFA score of 3. In some embodiments, a patient has an EHFA score of 4. In some embodiments, a patient has an EHFA score of 5. In some embodiments, a patient has an EHFA score of 6. In some embodiments, a patient has an EHFA score of 7. In some embodiments, a patient has an EHFA score of 8. In some embodiments, a patient with an EHFA score of ≥5 points is diagnosed with HFpEF. In some embodiments, a patient with an EHFA score of between 2 and 4 points may have HFpEF and requires a diastolic stress test or invasive hemodynamic measurements to confirm. In some embodiments, a patient with an EHFA score of 1 point or less does not likely have HFpEF.

In some embodiments, a patient has one or more major EHFA criteria for HFpEF. In some embodiments, a patient has one or more major functional EHFA criteria for HFpEF. In some embodiments, a major functional criterion is selected from the group consisting of a septal e′ velocity <7 cm/s, a lateral e′ velocity <10 cm/s at rest, an average E/e′>14 or septal E/e′ ratio >15 with exercise and a TR velocity >2.8 m/s with exercise. In some embodiments, a patient has a septal e′ velocity <7 cm/s. In some embodiments, a patient has a lateral e′ velocity <10 cm/s at rest. In some embodiments, a patient has an average E/e′>14 or septal E/e′ ratio >15 with exercise. In some embodiments, a patient has a TR velocity >2.8 m/s with exercise. In some embodiments, a patient has one or more major morphological EHFA criteria for HFpEF. In some embodiments, a major morphological criterion is selected from the group consisting of a LAVI>34 mL/m² and an LVMI≥149 g/m² for men and >122 g/m² for women and RWT>0.42. In some embodiments, a patient has an LAVI>34 mL/m². In some embodiments, a male patient has an LVMI≥149 g/m². In some embodiments, a female patient has an LAVI≥122 g/m². In some embodiments, a patient has a RWT>0.42. In some embodiments, a patient has one or more major biomarker EHFA criteria for HFpEF. In some embodiments, a major biomarker criterion is sinus rhythm, with NT-proBNP>220 pg/mL and/or BNP>80 pg/mL. In some embodiments, a patient has an NT-proBNP>220 pg/mL and/or BNP>80 pg/mL. In some embodiments, a major biomarker criterion is atrial fibrillation, with NT-proBNP>660 pg/mL and/or BNP>240 pg/mL. In some embodiments, a patient has an NT-proBNP>660 pg/mL or BNP>240 pg/mL.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves one or more major functional EHFA criteria. In some embodiments, the method improves one or more major functional criterion selected from the group consisting of increasing septal e′ velocity to >7 cm/s, increasing lateral e′ velocity to >10 cm/s at rest, decreasing E/e′ to <14 or septal E/e′ ratio to <15 with exercise and decreasing TR velocity to <2.8 m/s with exercise.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves one or more major morphological EHFA criteria. In some embodiments, the method improves one or more major morphological criterion selected from the group consisting of decreasing LAVI to <34 mL/m² and decreasing LVMI to <149 g/m² for men and <122 g/m² for women, and decreasing RWT to <0.42.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves one or more major biomarker EHFA criteria. In some embodiments, the method improves sinus rhythm, comprising decreasing NT-proBNP to <220 pg/mL and/or decreasing BNP to <80 pg/mL. In some embodiments, the method improves atrial fibrillation, comprising decreasing NT-proBNP to <660 pg/mL and/or decreasing BNP to <240 pg/mL.

In some embodiments, a patient has one or more minor EHFA criteria for HFpEF. In some embodiments, a patient has one or more minor functional EHFA criteria for HFpEF. In some embodiments, a minor functional criterion is selected from the group consisting of an average E/e′ 9-14 and a GLS<16%. In some embodiments, a patient has an average E/e′ 9-14. In some embodiments, a patient has a GLS<16%. In some embodiments, a patient has one or more minor morphological EHFA criteria for HFpEF. In some embodiments, a minor morphological criterion is selected from the group consisting of a LAVI 29-34 mL/m², an LVMI>115 g/m² for men, an LVMI of 95 g/m² for women, a RWT>0.42, and an LV wall thickness >12 mm. In some embodiments, a patient has an LAVI 29-34 mL/m2. In some embodiments, a male patient has an LVMI>115 g/m2. In some embodiments, a female patient has an LVMI of 95 g/m2. In some embodiments, a patient has a RWT>0.42. In some embodiments, a patient has one or more minor biomarker EHFA criteria for HFpEF. In some embodiments, a patient has an LV wall thickness >12 mm. In some embodiments, a minor biomarker criterion is sinus rhythm, with 5-NT-proBNP 125-220 pg/mL and/or BNP 35-80 pg/mL. In some embodiments, a patient has an 5-NT-proBNP 125-220 pg/mL and/or BNP 35-80 pg/mL. In some embodiments, a minor biomarker criterion is atrial fibrillation, with NT-proBNP 365-660 pg/mL and/or BNP 105-240 pg/mL. In some embodiments, a patient has an NT-proBNP 365-660 pg/mL and/or BNP 105-240 pg/mL.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves one or more minor functional EHFA criteria. In some embodiments, the method improves minor functional criteria, comprising decreasing E/e′ to 8 or below and increasing GLS to >16%.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves one or more minor morphological EHFA criteria. In some embodiments, the method improves one or more minor morphological criterion selected from the group consisting of decreasing LAVI to <34 mL/m², decreasing LVMI to <115 g/m² for men, decreasing LVMI to below 95 g/m² for women, decreasing RWT to <0.42, and decreasing LV wall thickness to <12 mm.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves one or more minor biomarker EHFA criteria. In some embodiments, the method improves sinus rhythm, comprising decreasing 5-NT-proBNP to <220 pg/mL and/or decreasing BNP to <80 pg/mL. In some embodiments, the method improves atrial fibrillation, comprising decreasing NT-proBNP to <660 pg/mL and/or decreasing BNP to <240 pg/mL.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method decreases the patient's EHFA score (e.g., by 1, 2, 3, 4, 5, 6, 7, or 8 points). In some embodiments, the method relates to decreasing a patient's EHFA score by at least 1 point. In some embodiments, the method relates to decreasing a patient's EHFA score by at least 2 points. In some embodiments, the method relates to decreasing a patient's EHFA score by at least 3 points. In some embodiments, the method relates to decreasing a patient's EHFA score by at least 4 points. In some embodiments, the method relates to decreasing a patient's EHFA score by at least 5 points. In some embodiments, the method relates to decreasing a patient's EHFA score by at least 6 points. In some embodiments, the method relates to decreasing a patient's EHFA score by at least 7 points. In some embodiments, the method relates to decreasing a patient's EHFA score by at least 8 points.

### Rate of Hospitalization

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method reduces the patient's hospitalization rate (e.g., by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, the method relates to reducing the patient's hospitalization rate by at least 1%. In some embodiments, the method relates to reducing the patient's hospitalization rate by at least 2%. In some embodiments, the method relates to reducing the patient's hospitalization rate by at least 3%. In some embodiments, the method relates to reducing the patient's hospitalization rate by at least 4%. In some embodiments, the method relates to reducing the patient's hospitalization rate by at least 5%. In some embodiments, the method relates to reducing the patient's hospitalization rate by at least 10%. In some embodiments, the method relates to reducing the patient's hospitalization rate by at least 15%. In some embodiments, the method relates to reducing the patient's hospitalization rate by at least 20%. In some embodiments, the method relates to reducing the patient's hospitalization rate by at least 25%. In some embodiments, the method relates to reducing the patient's hospitalization rate by at least 30%. In some embodiments, the method relates to reducing the patient's hospitalization rate by at least 35%. In some embodiments, the method relates to reducing the patient's hospitalization rate by at least 40%. In some embodiments, the method relates to reducing the patient's hospitalization rate by at least 45%. In some embodiments, the method relates to reducing the patient's hospitalization rate by at least 50%. In some embodiments, the method relates to reducing the patient's hospitalization rate by at least 55%. In some embodiments, the method relates to reducing the patient's hospitalization rate by at least 60%. In some embodiments, the method relates to reducing the patient's hospitalization rate by at least 65%. In some embodiments, the method relates to reducing the patient's hospitalization rate by at least 70%. In some embodiments, the method relates to reducing the patient's hospitalization rate by at least 75%. In some embodiments, the method relates to reducing the patient's hospitalization rate by at least 80%. In some embodiments, the method relates to reducing the patient's hospitalization rate by at least 85%. In some embodiments, the method relates to reducing the patient's hospitalization rate by at least 90%. In some embodiments, the method relates to reducing the patient's hospitalization rate by at least 95%. In some embodiments, the method relates to reducing the patient's hospitalization rate by 100%.

In some embodiments, reducing a patient's hospitalization rate comprises reducing the need to for the patient to stay at the hospital. In some embodiments, reducing a patient's hospitalization rate comprises reducing the number of total patient hospital visits. In some embodiments, reducing a patient's hospitalization rate comprises increasing the time to initial hospitalization of the patient. In some embodiments, reducing a patient's hospitalization rate comprises increasing the length of life of the patient. In some embodiments, reducing a patient's hospitalization rate comprises increasing the time between patient hospital visits. In some embodiments, reducing a patient's hospitalization rate comprises decreasing the number of recurrent patient hospital visits.

In certain aspects, the disclosure relates to methods of treating, preventing, or reducing the progression rate and/or severity of heart failure associated with aging comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method reduces the patient's hospitalization rate. In some embodiments, the method reduces the need to for the patient to stay at the hospital. In some embodiments, the method reduces the number of total patient hospital visits. In some embodiments, the method increases the time to initial hospitalization of the patient. In some embodiments, the method increases the length of life of the patient. In some embodiments, the method increases the time between hospital visits. In some embodiments, the method decreases the number of recurrent hospital visits.

### Rate of Worsening of Heart Failure

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method reduces the patient's rate of worsening of heart failure (e.g., by at least 1%, 2%, 3%, 4%, 5%, 10%, 15%, 20%, 25%, 30%, 35%, 40%, 45%, 50%, 55%, 60%, 65% 70%, 75%, 80%, 85%, 90%, 95%, or 100%). In some embodiments, the method relates to reducing the patient's rate of worsening of heart failure by at least 10%. In some embodiments, the method relates to reducing the patient's rate of worsening of heart failure by at least 2%. In some embodiments, the method relates to reducing the patient's rate of worsening of heart failure by at least 3%. In some embodiments, the method relates to reducing the patient's rate of worsening of heart failure by at least 4%. In some embodiments, the method relates to reducing the patient's rate of worsening of heart failure by at least 5%. In some embodiments, the method relates to reducing the patient's rate of worsening of heart failure by at least 10%. In some embodiments, the method relates to reducing the patient's rate of worsening of heart failure by at least 15%. In some embodiments, the method relates to reducing the patient's rate of worsening of heart failure by at least 20%. In some embodiments, the method relates to reducing the patient's rate of worsening of heart failure by at least 25%. In some embodiments, the method relates to reducing the patient's rate of worsening of heart failure by at least 30%. In some embodiments, the method relates to reducing the patient's rate of worsening of heart failure by at least 35%. In some embodiments, the method relates to reducing the patient's rate of worsening of heart failure by at least 40%. In some embodiments, the method relates to reducing the patient's rate of worsening of heart failure by at least 45%. In some embodiments, the method relates to reducing the patient's rate of worsening of heart failure by at least 50%. In some embodiments, the method relates to reducing the patient's rate of worsening of heart failure by at least 55%. In some embodiments, the method relates to reducing the patient's rate of worsening of heart failure by at least 60%. In some embodiments, the method relates to reducing the patient's rate of worsening of heart failure by at least 65%. In some embodiments, the method relates to reducing the patient's rate of worsening of heart failure by at least 70%. In some embodiments, the method relates to reducing the patient's rate of worsening of heart failure by at least 75%. In some embodiments, the method relates to reducing the patient's rate of worsening of heart failure by at least 80%. In some embodiments, the method relates to reducing the patient's rate of worsening of heart failure by at least 85%. In some embodiments, the method relates to reducing the patient's rate of worsening of heart failure by at least 90%. In some embodiments, the method relates to reducing the patient's rate of worsening of heart failure by at least 95%. In some embodiments, the method relates to reducing the patient's rate of worsening of heart failure by 100%.

### Cardiac Imaging

Echocardiogram

The term “echocardiography” as used herein refers to two-dimensional/three-dimensional echocardiography, pulsed and continuous wave Doppler, color flow Doppler, tissue Doppler imaging (TDI) contrast echocardiography, deformation imaging (strain and strain rate), and transthoracic echocardiography (TTE, or two-dimensional echocardiography). TTE is typically the method of choice for assessment of myocardial systolic and diastolic function of both left and right ventricles. In some embodiments, a patient is assessed for heart failure using echocardiography. In some embodiments, a patient is assessed for heart failure using two-dimensional echocardiography. In some embodiments, a patient is assessed for heart failure using three-dimensional echocardiography. In some embodiments, a patient is assessed for heart failure using pulsed and continuous wave Doppler echocardiography. In some embodiments, a patient is assessed for heart failure using echocardiography. In some embodiments, a patient is assessed for heart failure using, color flow Doppler echocardiography. In some embodiments, a patient is assessed for heart failure using tissue Doppler imaging (TDI) contrast echocardiography. In some embodiments, a patient is assessed for heart failure using deformation imaging (strain and strain rate) echocardiography. In some embodiments, a patient is assessed for heart failure using transthoracic echocardiography (TTE).

Echocardiography is a useful and widely available test in patients with suspected HF to establish a diagnosis. It provides information on LV structure and systolic function (e.g., measured by M-mode in a parasternal short axis view at the papillary muscle level), including, but not limited to LV wall thickness (LVWT), LV mass (LVM), LV end diastolic diameter (LVEDD), LV end systolic diameter (LVESD), fractional shortening (FS) (calculated using the equation FS=100%×[(EDD−ESD)/EDD]), LV end diastolic volume (LVEDV), LV end systolic volume (LVESV), ejection fraction (calculated using the equation EF=100%×[(EDV−ESV)/EDV]), Hypertrophy index (calculated as the ratio of LVM to LVESV), and relative wall thickness (calculated as the ratio of LVWT to LVESD). This information is crucial in establishing a diagnosis and in determining appropriate treatment. In some embodiments, a patient's LV wall thickness (LVWT) is measured using echocardiography. In some embodiments, a patient's LV mass (LVM) is measured using echocardiography. In some embodiments, a patient's LV end diastolic diameter (LVEDD) is measured using echocardiography. In some embodiments, a patient's LV end systolic diameter (LVESD) is measured using echocardiography. In some embodiments, a patient's fractional shortening (FS) is measured using echocardiography. In some embodiments, a patient's LV end diastolic volume (LVEDV) is measured using echocardiography. In some embodiments, a patient's LV end systolic volume (LVESV) is measured using echocardiography. In some embodiments, a patient's ejection fraction is measured using echocardiography. In some embodiments, a patient's hypertrophy index is measured using echocardiography. In some embodiments, a patient's relative wall thickness is measured using echocardiography. There are numerous clinical presentation factors, echocardiography features, and other features that could be indicative of heart failure associated with aging. In some embodiments, an echocardiogram performed on a patient shows structural left heart abnormalities. In some embodiments, the structural left heart abnormality is a disease of the left heart valves. In some embodiments, the structural left heart abnormality is left atrium enlargement (e.g., >4.2 cm).

In a patient that has symptoms of left heart failure, an echocardiogram may be performed to evaluate various parameters. For instance, in some embodiments, an echocardiogram using Doppler performed on a patient may show indices of increased filling pressures and/or diastolic dysfunction (e.g., increased E/e′ or >Type 2-3 mitral flow abnormality, See Tables 9, 10, 12). In some embodiments, imaging (e.g. echocardiogram, CT scan, chest X-ray, or cardiac MRI) performed on a patient shows Kerley B lines. In some embodiments, imaging (e.g. echocardiogram, CT scan, chest X-ray, or cardiac MRI) performed on a patient shows pleural effusion. In some embodiments, imaging (e.g. echocardiogram, CT scan, chest X-ray, or cardiac MRI) performed on a patient shows pulmonary edema. In some embodiments, imaging (e.g., echocardiogram, CT scan, chest X-ray, or cardiac MRI) performed on a patient shows left atrium enlargement. Id.

Key functional alterations of HFpEF/HFmrEF heart failure comprise an E/e′>13 and a mean e′ septal and lateral wall <9 cm/s. Other (indirect) echocardiographically derived measurements are longitudinal strain or tricuspid regurgitation velocity (TRV). In identifying patients with suspected HFpEF, echocardiography is helpful in demonstrating that LVEF is preserved (e.g., >50 percent) and that LV volume is normal. Echocardiography is also helpful in identifying causes of HF with an LVEF>50 percent other than HFpEF, including valvular and pericardial disease. For parameters defined in HFpEF that are measured by echocardiography, see Tables 9, 10, and 11. Echocardiography examination may also include assessment of right ventricle (RV) structure and function, including, but not limited to, RV and right atrial (RA) dimensions, and an estimation of RV systolic function and/or pulmonary arterial pressure. Among parameters reflecting RV systolic function, the following measures are of particular importance: tricuspid annular plane systolic excursion (TAPSE; abnormal TAPSE<17 mm indicates RV systolic dysfunction) and tissue Doppler-derived tricuspid lateral annular systolic velocity (s′) (s′ velocity <9.5 cm/s indicates RV systolic dysfunction). Systolic pulmonary artery pressure is derived from an optimal recording of maximal tricuspid regurgitant jet and the tricuspid systolic gradient, together with an estimate of RA pressure on the basis of inferior vena cava (IVC) size and its breathing-related collapse. Exercise or pharmacological stress echocardiography may be used for the assessment of inducible ischemia and/or myocardium viability and in some clinical scenarios of patients with valve disease (e.g. dynamic mitral regurgitation, low-flow-low-gradient aortic stenosis). There are also suggestions that stress echocardiography may allow the detection of diastolic dysfunction related to exercise exposure in patients with exertional dyspnea, preserved LVEF, and inconclusive diastolic parameters at rest.

Transthoracic echocardiography (TTE) is recommended for the assessment of myocardial structure and function in patients with suspected HF in order to establish a diagnosis of either HFrEF, HFmrEF or HFpEF. Furthermore, TTE is recommended to assess LVEF in order to identify patients with HF who would be suitable for evidence-based pharmacological and device (ICD, CRT) treatment recommended for HFrEF; for the assessment of valve disease, right ventricular function and pulmonary arterial pressure in patients with an already established diagnosis of either HFrEF, HFmrEF or HFpEF in order to identify those suitable for correction of valve disease; and/or for the assessment of myocardial structure and function in patients to be exposed to treatment which potentially can damage myocardium (e.g. chemotherapy). Other techniques (including systolic tissue Doppler velocities and deformation indices, i.e. strain and strain rate), should be considered in a TTE protocol in patients at risk of developing HF in order to identify myocardial dysfunction at the preclinical stage.

In HFpEF, EF is normal, and the principal hemodynamic derangement is an elevation in filling pressures. When pressures are high and congestion is present at rest, HFpEF is readily diagnosed based upon history, physical examination, radiography, NP levels, and echocardiography. However, many patients with early-stage HFpEF have significant symptoms of exertional intolerance in the absence of apparent volume overload. Invasive assessment in some patients may reveal pathologic elevation in filling pressures that had not been previously suspected, and a recent study found that even among patients with normal exam, echocardiography, NP, and normal resting hemodynamics, many patients may still develop pathologic elevations in filling pressures characteristic of HFpEF during the stress of exercise. Pulmonary artery pressures track very closely with left heart filling pressures in early-stage HFpEF, suggesting that if the former could be accurately estimated by echocardiography during exercise, this may serve as a useful non-invasive screen among patients with normal EF and exertional dyspnea. In some embodiments, a patient is examined for heart failure using echocardiography during exercise. In some embodiments, a patient is examined for HFpEF using echocardiography during exercise.

Cardiac Magnetic Resonance (CMR)

CMR is acknowledged as a gold standard for the measurements of volumes, mass and EF of both the left and right ventricles. It is the best alternative cardiac imaging modality for patients with nondiagnostic echocardiographic studies (particularly for imaging of the right heart) and is the method of choice in patients with complex congenital heart diseases. Cardiac magnetic resonance (CMR) measures both cardiac anatomical and functional quantification, with unique capabilities of non-invasive tissue characterization, complementing well with echocardiography. CMR imaging covering the LV in short axis from apex to base is used for measuring left ventricular (LV) volumes, ejection fraction (EF) and regional function. The 3D dataset is not affected by geometric assumptions and therefore less prone to error compared with two-dimensional (2D) echocardiography, particularly in remodeled ventricles. Novel CMR tissue characterization techniques are called CMR relaxometry (T1 and T2 mapping and extracellular volume fraction (ECV)) which allow a more detailed and quantitative approach to tissue characterization and 4D-Flow which provides quantitative information on intracavitary flows. Current applications appear particularly useful for diastolic dysfunction detection although they deserve a specific comparison with traditional Doppler and Tissue Doppler (e.g., echocardiography) analysis in order to confirm the applicability in clinical practice. Non-invasive stress imaging (CMR, stress echocardiography, SPECT, PET) may be considered for the assessment of myocardial ischemia and viability in patients with HF and CAD (considered suitable for coronary revascularization) before the decision on revascularization. In some embodiments, a patient is assessed for heart failure using CMR. In some embodiments, a patient is assessed for heart failure using CMR relaxometry (T1 and T2 mapping and extracellular volume fraction (ECV)). In some embodiments, a patient is assessed for heart failure using CMR and 4D-Flow.

CMR can provide information on LV structure and systolic function, including, but not limited to, LV wall thickness (LVWT), LV mass (LVM), LV end diastolic diameter (LVEDD), LV end systolic diameter (LVESD), fractional shortening (FS) (calculated using the equation FS=100%×[(EDD−ESD)/EDD]), LV end diastolic volume (LVEDV), LV end systolic volume (LVESV), ejection fraction (calculated using the equation EF=100%×[(EDV−ESV)/EDV]), Hypertrophy index (calculated as the ratio of LVM to LVESV), and relative wall thickness (calculated as the ratio of LVWT to LVESD). This information is crucial in establishing a diagnosis and in determining appropriate treatment. In some embodiments, a patient's LV wall thickness (LVWT) is measured using CMR. In some embodiments, a patient's LV mass (LVM) is measured using CMR. In some embodiments, a patient's LV end diastolic diameter (LVEDD) is measured using CMR. In some embodiments, a patient's LV end systolic diameter (LVESD) is measured using CMR. In some embodiments, a patient's fractional shortening (FS) is measured using CMR. In some embodiments, a patient's LV end diastolic volume (LVEDV) is measured using CMR. In some embodiments, a patient's LV end systolic volume (LVESV) is measured using CMR. In some embodiments, a patient's ejection fraction is measured using CMR. In some embodiments, a patient's hypertrophy index is measured using CMR. In some embodiments, a patient's relative wall thickness is measured using CMR.

CMR is a preferred imaging method to assess myocardial fibrosis using late gadolinium enhancement (LGE) along with T1 mapping and can be useful for establishing HF etiology. For example, CMR with LGE allows differentiation between ischemic and non-ischemic origins of HF and myocardial fibrosis/scars can be visualized. In addition, CMR allows the characterization of myocardial tissue of myocarditis, amyloidosis, sarcoidosis, Chagas disease, Fabry disease non-compaction cardiomyopathy and haemochromatosis. CMR may also be used for the assessment of myocardial ischemia and viability in patients with HF and coronary artery disease (CAD) (considered suitable for coronary revascularization). In some embodiments, a patient is assessed for heart failure using CMR with late gadolinium enhancement (LGE) and/or T1 mapping. In some embodiments, fibrosis and/or scars in a patient's heart is measured using CMR.

Clinical limitations of CMR include local expertise, lower availability and higher costs compared with echocardiography, uncertainty about safety in patients with metallic implants (including cardiac devices) and less reliable measurements in patients with tachyarrhythmias. Claustrophobia is an important limitation for CMR. Linear gadolinium-based contrast agents are contraindicated in individuals with a glomerular filtration rate (GFR)<30 mL/min/1.73 m², because they may trigger nephrogenic systemic fibrosis (this may be less of a concern with newer cyclic gadolinium-based contrast agents).

CMR is recommended for the assessment of myocardial structure and function (including right heart) in patients with poor acoustic window and patients with complex congenital heart diseases (taking account of cautions/contra-indications to CMR). CMR is recommended for the characterization of myocardial tissue in case of suspected myocarditis, amyloidosis, sarcoidosis, Chagas disease, Fabry disease non-compaction cardiomyopathy, and haemochromatosis (taking account of cautions/contraindications to CMR).

Multigated Acquisition (MUGA)

Radionuclide angiography is an area of nuclear medicine which specializes in imaging to show the functionality of the right and left ventricles of the heart, thus allowing informed diagnostic intervention in heart failure. It involves use of a radiopharmaceutical injected into a patient, and a gamma camera for acquisition. A MUGA scan (multigated acquisition) involves an acquisition triggered (gated) at different points of the cardiac cycle. MUGA scanning is also sometimes referred to as equilibrium radionuclide angiocardiography, radionuclide ventriculography (RNVG), or gated blood pool imaging, as well as SYMA scanning (synchronized multigated acquisition scanning). In some embodiments, a patient is assessed for heart failure using MUGA. In some embodiments, a patient is assessed for heart failure using equilibrium radionuclide angiocardiography. In some embodiments, a patient is assessed for heart failure using radionuclide ventriculography (RNVG). In some embodiments, a patient is assessed for heart failure using gated blood pool imaging.). In some embodiments, a patient is assessed for heart failure using SYMA scanning (synchronized multigated acquisition scanning).

MUGA uniquely provides a cine type of image (e.g., short movies that are able to show heart motion throughout the cardiac cycle) of the beating heart, and allows the interpreter to determine the efficiency of the individual heart valves and chambers. MUGA/Cine scanning represents a robust adjunct to an echocardiogram. Mathematics regarding acquisition of cardiac output (Q) is well served by both of these methods as well as other inexpensive models supporting ejection fraction as a product of the heart/myocardium in systole. One main advantage of a MUGA scan over an echocardiogram or an angiogram is its accuracy. An echocardiogram measures the shortening fraction of the ventricle and is limited by the user's ability. Furthermore, an angiogram is invasive and, often, more expensive. A MUGA scan provides a more accurate representation of cardiac ejection fraction.

Chest X-Ray

A chest X-ray is of limited use in the diagnostic work-up of patients with suspected HF. It is most useful in identifying an alternative, pulmonary explanation for a patient's symptoms and signs, (e.g., pulmonary malignancy and/or interstitial pulmonary disease), although computed tomography (CT) of the chest is currently the standard of care for these types of pulmonary diseases. For diagnosis of asthma or chronic obstructive pulmonary disease (COPD), pulmonary function testing with spirometry is needed. A chest X-ray may, however, show pulmonary venous congestion or edema in a patient with HF, and is more helpful in the acute setting than in the non-acute setting. In some embodiments, a patient is assessed for heart failure using chest X-ray.

A chest X-ray is commonly obtained in patients with HF to assess for signs of pulmonary edema and to identify other causes of dyspnea. A chest X-ray may show cardiomegaly and/or radiographic evidence of pulmonary edema. Most patients with HFpEF will have a normal chest X-ray. In some embodiments, a patient with HFpEF has a normal chest X-ray.

Single-Photon Emission Computed Tomography (SPECT) and Radionucleotide Ventriculography

Single-photon emission CT (SPECT) may be useful in assessing ischemia and myocardial viability. Gated SPECT can also yield information on ventricular volumes and function, but exposes the patient to ionizing radiation. 3,3-diphosphono-1,2-propanodicarboxylic acid (DPD) scintigraphy may be useful for the detection of transthyretin cardiac amyloidosis. In some embodiments, a patient is assessed for heart failure using SPECT.

Positron Emission Tomography (PET)

Positron emission tomography (PET) (alone or with CT) may be used to assess ischemia and viability, but flow tracers (N-13 ammonia or 0-15 water) require an on-site cyclotron. Rubidium is an alternative tracer for ischemia testing with PET, which can be produced locally at relatively low cost. Limited availability, radiation exposure and cost are the main limitations. In some embodiments, a patient is assessed for heart failure using PET.

Coronary Angiography

Coronary angiography is recommended in patients with HF who suffer from angina pectoris recalcitrant to medical therapy, provided the patient is otherwise suitable for coronary revascularization. Coronary angiography is also recommended in patients with a history of symptomatic ventricular arrhythmia or aborted cardiac arrest. Coronary angiography should be considered in patients with HF and intermediate to high pre-test probability of coronary artery disease (CAD) and the presence of ischemia in non-invasive stress tests in order to establish the ischemic etiology and CAD severity. In some embodiments, a patient is assessed for heart failure using coronary angiography.

Invasive coronary angiography is recommended in patients with HF and angina pectoris recalcitrant to pharmacological therapy or symptomatic ventricular arrhythmias or aborted cardiac arrest (who are considered suitable for potential coronary revascularization) in order to establish the diagnosis of CAD and its severity. Invasive coronary angiography should be considered in patients with HF and intermediate to high pre-test probability of CAD and the presence of ischemia in non-invasive stress tests (who are considered suitable for potential coronary revascularization) in order to establish the diagnosis of CAD and its severity.

Cardiac Computer Tomography (CT)

The main use of cardiac CT in patients with HF is as a non-invasive means to visualize the coronary anatomy in patients with HF with low intermediate pre-test probability of coronary artery disease (CAD) or those with equivocal non-invasive stress tests in order to exclude the diagnosis of CAD, in the absence of relative contraindications. However, the test is only required when its results might affect a therapeutic decision. Cardiac CT may be considered in patients with HF and low to intermediate pre-test probability of CAD or those with equivocal non-invasive stress tests in order to rule out coronary artery stenosis. In some embodiments, a patient is assessed for heart failure using cardiac computer tomography

Electrocardiogram (ECG or EKG)

An electrocardiogram (ECG or EKG) records the electrical signals in your heart. With each beat, an electrical impulse (or “wave”) travels through the heart. This wave causes the muscle to squeeze and pump blood from the heart. A normal heartbeat on ECG will show the timing of the top and lower chambers. The right and left atria or upper chambers make the first wave called a “P wave”, following a flat line when the electrical impulse goes to the bottom chambers. The right and left bottom chambers or ventricles make the next wave called a “QRS complex.” The final wave or “T wave” represents electrical recovery or return to a resting state for the ventricles. An ECG gives two major kinds of information. First, by measuring time intervals on the ECG, a doctor can determine how long the electrical wave takes to pass through the heart. Finding out how long a wave takes to travel from one part of the heart to the next shows if the electrical activity is normal or slow, fast or irregular. Second, by measuring the amount of electrical activity passing through the heart muscle, a cardiologist may be able to find out if parts of the heart are too large or are overworked. Table 11 shows typical trends in electrocardiography in HFpEF patients. In some embodiments, a patient is assessed for heart failure using an electrocardiogram.

Endomyocardial Biopsy

Endomyocardial biopsy (EMB) is a procedure that percutaneously obtains small amounts of myocardial tissue for diagnostic, therapeutic, and research purposes. It is primarily used to (1) follow the transplanted heart for myocardial rejection; (2) diagnose specific inflammatory, infiltrative, or familial myocardial disorders; and (3) sample unknown myocardial masses. EMB is the definitive procedure for examining the myocardium, but is limited by its invasiveness, sampling error and lack of generalized expertise in its performance. In some embodiments, a patient is assessed for heart failure using endomyocardial biopsy.

Measuring Hematologic Parameters in a Patient

In certain embodiments, the present disclosure provides methods for managing a patient that has been treated with, or is a candidate to be treated with, one or more one or more ActRII-ALK4 antagonists of the disclosure (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) by measuring one or more hematologic parameters in the patient. The hematologic parameters may be used to evaluate appropriate dosing for a patient who is a candidate to be treated with one or more ActRII-ALK4 antagonists of the present disclosure, to monitor the hematologic parameters during treatment, to evaluate whether to adjust the dosage during treatment with one or more ActRII-ALK4 antagonists of the disclosure, and/or to evaluate an appropriate maintenance dose of one or more ActRII-ALK4 antagonists of the disclosure. If one or more of the hematologic parameters are outside the normal level, dosing with one or more ActRII-ALK4 antagonists may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art recognized methods. Such parameters may be determined using a blood sample from a patient. Increases in red blood cell levels, hemoglobin levels, and/or hematocrit levels may cause increases in blood pressure.

In one embodiment, if one or more hematologic parameters are outside the normal range or on the high side of normal in a patient who is a candidate to be treated with one or more ActRII-ALK4 antagonists, then onset of administration of the one or more ActRII-ALK4 antagonists of the disclosure may be delayed until the hematologic parameters have returned to a normal or acceptable level either naturally or via therapeutic intervention. For example, if a candidate patient is hypertensive or pre-hypertensive, then the patient may be treated with a blood pressure lowering agent in order to reduce the patient's blood pressure. Any blood pressure lowering agent appropriate for the individual patient's condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate patient has iron stores that are lower than normal, or on the low side of normal, then the patient may be treated with an appropriate regimen of diet and/or iron supplements until the patient's iron stores have returned to a normal or acceptable level. For patients having higher than normal red blood cell levels and/or hemoglobin levels, then administration of the one or more ActRII-ALK4 antagonists of the disclosure may be delayed until the levels have returned to a normal or acceptable level.

In certain embodiments, if one or more hematologic parameters are outside the normal range or on the high side of normal in a patient who is a candidate to be treated with one or more ActRII-ALK4 antagonists, then the onset of administration may not be delayed. However, the dosage amount or frequency of dosing of the one or more ActRII-ALK4 antagonists of the disclosure may be set at an amount that would reduce the risk of an unacceptable increase in the hematologic parameters arising upon administration of the one or more ActRII-ALK4 antagonists of the disclosure. Alternatively, a therapeutic regimen may be developed for the patient that combines one or more ActRII-ALK4 antagonists with a therapeutic agent that addresses the undesirable level of the hematologic parameter. For example, if the patient has elevated blood pressure, then a therapeutic regimen may be designed involving administration of one or more ActRII-ALK4 antagonists and a blood pressure lowering agent. For a patient having lower than desired iron stores, a therapeutic regimen may be developed involving one or more ActRII-ALK4 antagonists of the disclosure and iron supplementation.

In one embodiment, baseline parameter(s) for one or more hematologic parameters may be established for a patient who is a candidate to be treated with one or more ActRII-ALK4 antagonists of the disclosure and an appropriate dosing regimen established for that patient based on the baseline value(s). Alternatively, established baseline parameters based on a patient's medical history could be used to inform an appropriate ActRII-ALK4 antagonist dosing regimen for a patient. For example, if a healthy patient has an established baseline blood pressure reading that is above the defined normal range it may not be necessary to bring the patient's blood pressure into the range that is considered normal for the general population prior to treatment with the one or more ActRII-ALK4 antagonists of the disclosure. A patient's baseline values for one or more hematologic parameters prior to treatment with one or more ActRII-ALK4 antagonists of the disclosure may also be used as the relevant comparative values for monitoring any changes to the hematologic parameters during treatment with the one or more ActRII-ALK4 antagonists of the disclosure.

In certain embodiments, one or more hematologic parameters are measured in patients who are being treated with one or more ActRII-ALK4 antagonists. The hematologic parameters may be used to monitor the patient during treatment and permit adjustment or termination of the dosing with the one or more ActRII-ALK4 antagonists of the disclosure or additional dosing with another therapeutic agent. For example, if administration of one or more ActRII-ALK4 antagonists results in an increase in blood pressure, red blood cell level, or hemoglobin level, or a reduction in iron stores, then the dose of the one or more ActRII-ALK4 antagonists of the disclosure may be reduced in amount or frequency in order to decrease the effects of the one or more ActRII-ALK4 antagonists of the disclosure on the one or more hematologic parameters. If administration of one or more ActRII-ALK4 antagonists results in a change in one or more hematologic parameters that is adverse to the patient, then the dosing of the one or more ActRII-ALK4 antagonists of the disclosure may be terminated either temporarily, until the hematologic parameter(s) return to an acceptable level, or permanently. Similarly, if one or more hematologic parameters are not brought within an acceptable range after reducing the dose or frequency of administration of the one or more ActRII-ALK4 antagonists of the disclosure, then the dosing may be terminated. As an alternative, or in addition to, reducing or terminating the dosing with the one or more ActRII-ALK4 antagonists of the disclosure, the patient may be dosed with an additional therapeutic agent that addresses the undesirable level in the hematologic parameter(s), such as, for example, a blood pressure lowering agent or an iron supplement. For example, if a patient being treated with one or more ActRII-ALK4 antagonists has elevated blood pressure, then dosing with the one or more ActRII-ALK4 antagonists of the disclosure may continue at the same level and a blood-pressure-lowering agent is added to the treatment regimen, dosing with the one or more antagonist of the disclosure may be reduced (e.g., in amount and/or frequency) and a blood-pressure-lowering agent is added to the treatment regimen, or dosing with the one or more antagonist of the disclosure may be terminated and the patient may be treated with a blood-pressure-lowering agent.

In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the method improves one or more hematologic parameters. In some embodiments, the method improves one or more hematologic parameters to a normal level compared to healthy people of similar age and sex.

### 7. Additional Treatments for Heart Failure and Co-Therapies

In certain aspects, the disclosure contemplates the use of an ActRII-ALK4 antagonist, in combination with one or more additional active agents or other supportive therapy for treating, preventing, or reducing the progression rate and/or severity of heart failure associated with aging. As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more ActRII-ALK4 antagonists of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the ActRII-ALK4 antagonist of the disclosure with the additional active agent or therapy and/or the desired effect.

Some goals of treatment in patients with HF is to improve their clinical status, functional capacity and quality of life, and/or prevent hospital admission and reduce mortality. Neuro-hormonal antagonists (e.g., ACEIs, MRAs and beta-blockers) have been shown to improve survival in patients with HFrEF and have been recommended for the treatment of patients with HFrEF, unless contraindicated or not tolerated. In certain aspects, the disclosure relates to methods of treating, preventing, or reducing the progression rate and/or severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist), wherein the patient is also administered one or more of an angiotensin-converting enzyme inhibitor (ACE inhibitor), beta-blocker, angiotensin II receptor blocker (ARB), Mineralocorticoid/aldosterone receptor antagonist (MRA) or implantable cardioverter defibrillator (ICD). In some embodiments, the method relates to administering an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) and an angiotensin-converting enzyme inhibitor (ACEI) to a patient in need thereof. In some embodiments, the method relates to administering an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) and a beta-blocker to a patient in need thereof. In some embodiments, the method relates to administering an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) and an angiotensin II receptor blocker (ARB) to a patient in need thereof. In some embodiments, the method relates to administering an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) and a mineralocorticoid/aldosterone receptor antagonist (MRA) to a patient in need thereof. In some embodiments, the method relates to administering an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) and an implantable cardioverter defibrillator (ICD) to a patient in need thereof. In some embodiments, the method relates to administering an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) and an angiotensin receptor neprilysin inhibitor (ARNI) to a patient in need thereof. In some embodiments, the method relates to administering an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) and a diuretic to a patient in need thereof. In some embodiments, the method relates to administering an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) and one or more of hydralazine and isosorbide dinitrate, digoxin, digitalis, N-3 polyunsaturated fatty acids (PUFA), and I_{f}-channel inhibitor (e.g., Ivabradine) to a patient in need thereof.

Optionally, methods disclosed herein for treating, preventing, or reducing the progression rate and/or severity of heart failure, particularly treating, preventing, or reducing the progression rate and/or severity of one or more comorbidities of heart failure, may further comprise administering to the patient one or more supportive therapies or additional active agents for treating heart failure. For example, the patient also may be administered one or more supportive therapies or active agents selected from the group consisting of: ACE inhibitors (e.g., benazepril, captopril, enalapril, lisinopril, perindopril, ramipril (e.g., ramipen), trandolapril, and zofenopril); beta blockers (e.g., acebutolol, atenolol, betaxolol, bisoprolol, carteolol, carvedilol, labetalol, metoprolol, nadolol, nebivolol, penbutolol, pindolol, propranolol, sotalol, and timolol); ARBs (e.g., losartan, irbesartan, olmesartan, candesartan, valsartan, fimasartan, azilsartan, salprisartan, and telmisartan); mineralocorticoid/aldosterone receptor antagonists (MRAs) (e.g., progesterone, eplerenone and spironolactone); glucocorticoids (e.g., beclomethasone, betamethasone, budesonide, cortisone, deflazacort, dexamethasone, hydrocortisone, methylprednisolone, prednisolone, methylprednisone, prednisone, triamcinolone, and finerenone); statins (e.g., atorvastatin (Lipitor), fluvastatin (Lescol), lovastatin (Mevacor, Altocor), pravastatin (Pravachol), pitavastatin (Livalo), simvastatin (Zocor), and rosuvastatin (Crestor)); Sodium-glucose co-transporter 2 (SGLT2) inhibitors (e.g., canagliflozin, dapagliflozin (e.g., Farxiga), and empagliflozin); an implantable cardioverter defibrillator (ICD); angiotensin receptor neprilysin inhibitors (ARNI) (e.g., valsartan and sacubitril (a neprilysin inhibitor)); diuretics (e.g., furosemide, bumetanide, torasemide, bendroflumethiazide, hydrochlorothiazide, metolazone, indapamidec, spironolactone/eplerenone, amiloride and triamterene); and other therapies including hydralazine and isosorbide dinitrate, digoxin, digitalis, N-3 polyunsaturated fatty acids (PUFA), and I_{f}-channel inhibitor (e.g., Ivabradine).

Angiotensin-Converting Enzyme (ACE) Inhibitors

An ACE inhibitor is recommended in patients with asymptomatic LV systolic dysfunction and a history of myocardial infarction in order to prevent or delay the onset of HF and prolong life, or in patients with asymptomatic LV systolic dysfunction without a history of myocardial infarction, in order to prevent or delay the onset of HF. ACE inhibitors should be considered in patients with stable CAD even if they do not have LV systolic dysfunction, in order to prevent or delay the onset of HF. ACE inhibitors have been shown to reduce mortality and morbidity in patients with HFrEF, and are recommended unless contraindicated or not tolerated in all symptomatic patients.

In some embodiments, the disclosure relates to a method of treating a patient with heart failure by administering an ACE inhibitor. In some embodiments, an ACE inhibitor is selected from the group consisting of benazepril, captopril, enalapril, lisinopril, perindopril, ramipril (e.g., ramipen), trandolapril, and zofenopril. In some embodiments, a patient is administered benazepril. In some embodiments, a patient is administered captopril. In some embodiments, a patient is administered enalapril. In some embodiments, a patient is administered lisinopril. In some embodiments, a patient is administered perindopril. In some embodiments, a patient is administered ramipril. In some embodiments, a patient is administered trandolapril. In some embodiments, a patient is administered zofenopril. In some embodiments, administration of an ACE inhibitor

In some embodiments, administration of an ACE inhibitor delays the onset of heart failure in a patient. In some embodiments, administration of an ACE inhibitor prevents the onset of heart failure in a patient. In some embodiments, administration of an ACE inhibitor increases length of life in a patient. In some embodiments, administration of an ACE inhibitor decreases length of a hospital stay in a patient. In some embodiments, administration of an ACE inhibitor prevents hospitalization of a patient.

Beta Blockers

A beta-blocker is recommended in patients with asymptomatic LV systolic dysfunction and a history of myocardial infarction, in order to prevent or delay the onset of HF or prolong life. Beta-blockers can reduce mortality and morbidity in symptomatic patients with HFrEF, despite treatment with an ACEI and, in most cases, a diuretic, but have not been tested in congested or decompensated patients. There is consensus that beta-blockers and ACEIs are complementary, and can be started together as soon as the diagnosis of HFrEF is made.

In some embodiments, the disclosure relates to a method of treating a patient having heart failure by administering one or more beta blockers. In some embodiments, the one or more beta blockers is selected from the group consisting of: acebutolol, atenolol, betaxolol, bisoprolol, carteolol, carvedilol, labetalol, metoprolol, nadolol, nebivolol, penbutolol, pindolol, propranolol, sotalol, and timolol. In some embodiments a patient is administered acebutolol. In some embodiments, a patient is administered atenolol. In some embodiments, a patient is administered betaxolol. In some embodiments, a patient is administered bisoprolol. In some embodiments, a patient is administered carteolol. In some embodiments, a patient is administered carvedilol. In some embodiments, a patient is administered labetalol. In some embodiments, a patient is administered metoprolol. In some embodiments, a patient is administered nadolol. In some embodiments, a patient is administered nebivolol. In some embodiments, a patient is administered penbutolol. In some embodiments, a patient is administered pindolol. In some embodiments, a patient is administered propranolol. In some embodiments, a patient is administered sotalol. In some embodiments, a patient is administered timolol.

In some embodiments, a patient is administered a beta blocker when the patient shows signs of heart failure. In some embodiments, a patient is administered a beta blocker when the patient is intolerant of ACE inhibitors. In some embodiments, a beta blocker delays onset of heart failure in a patient. In some embodiments, a beta blocker prevents onset of heart failure in a patient. In some embodiments, administration of a beta blocker increases length of life in a patient. In some embodiments, administration of a beta blocker decreases length of a hospital stay in a patient. In some embodiments, administration of a beta blocker prevents hospitalization of a patient.

Angiotensin II Receptor Blockers (ARBs)

Angiotensin II receptor blockers (ARBs) are an alternative in patients who may be intolerant of an ACE inhibitor. Candesartan has been shown to reduce cardiovascular mortality. Valsartan has showed an effect on hospitalization for HF (but not on all-cause hospitalizations) in patients with HFrEF receiving background ACEIs.

In some embodiments, the disclosure relates to a method of treating a patient having heart failure by administering one or more ARBs. In some embodiments the one or more ARBs is selected from the group consisting of: losartan, irbesartan, olmesartan, candesartan, valsartan, fimasartan, azilsartan, salprisartan, and telmisartan. In some embodiments a patient is administered losartan. In some embodiments, a patient is administered irbesartan. In some embodiments, a patient is administered olmesartan. In some embodiments, a patient is administered candesartan. In some embodiments, a patient is administered valsartan. In some embodiments, a patient is administered fimasartan. In some embodiments, a patient is administered azilsartan. In some embodiments, a patient is administered salprisartan. In some embodiments, a patient is administered telmisartan.

In some embodiments, a patient is administered an angiotensin antagonist (e.g., angiotensin receptor blocker, ARB), when the patient shows signs of heart failure. In some embodiments, a patient is administered an ARB when the patient is intolerant of ACE inhibitors. In some embodiments, an ARB delays onset of heart failure in a patient. In some embodiments, an ARB prevents onset of heart failure in a patient. In some embodiments, administration of an ARB increases length of life in a patient. In some embodiments, administration of an ARB decreases length of a hospital stay in a patient. In some embodiments, administration of an ARB prevents hospitalization of a patient.

Corticosteroids

Mineralocorticoid/aldosterone receptor antagonists (MRAs) block receptors that bind aldosterone and, with different degrees of affinity, other steroid hormone receptors (e.g. corticosteroids, androgens). Spironolactone or eplerenone are recommended in symptomatic heart failure patients (despite treatment with an ACE inhibitor and/or beta-blocker) with HFrEF and LVEF≤35%, to reduce mortality and HF hospitalization.

In some embodiments, the disclosure relates to a method of treating a patient with heart failure by administering a corticosteroid. In some embodiments, the patient is administered a Mineralocorticoid/aldosterone receptor antagonist (MRA). In some embodiments, the patient is administered a glucocorticoid. In some embodiments, a patient is administered one or more mineralocorticoid/aldosterone receptor antagonists (MRAs) selected from the group consisting of progesterone, eplerenone and spironolactone. In some embodiments a patient is administered eplerenone. In some embodiments, a patient is administered spironolactone.

In some embodiments, a patient is administered an MRA when the patient shows signs of heart failure. In some embodiments, an MRA delays onset of heart failure in a patient. In some embodiments, an MRA prevents onset of heart failure in a patient. In some embodiments, administration of an MRA increases length of life in a patient. In some embodiments, administration of an MRA decreases length of a hospital stay in a patient. In some embodiments, administration of an MRA prevents hospitalization of a patient.

In some embodiments, a patient with heart failure is administered one or more glucocorticoids. In some embodiments, administration of a glucocorticoid is an initial therapy. In some embodiments, a glucocorticoid is selected from the group consisting of beclomethasone, betamethasone, budesonide, cortisone, deflazacort, dexamethasone, hydrocortisone, methylprednisolone, prednisolone, methylprednisone, prednisone, triamcinolone, and finerenone. In some embodiments, a patient with heart failure is administered prednisone. In some embodiments, a patient with heart failure is administered prednisolone. In some embodiments, a patient with heart failure is administered finerenone. In some embodiments, a patient with heart failure is administered deflazacort.

In some embodiments, a patient is administered a glucocorticoid when the patient shows signs of heart failure. In some embodiments, a glucocorticoid delays onset of heart failure in a patient. In some embodiments, a glucocorticoid prevents onset of heart failure in a patient. In some embodiments, administration of a glucocorticoid increases length of life in a patient. In some embodiments, administration of a glucocorticoid decreases length of a hospital stay in a patient. In some embodiments, administration of a glucocorticoid prevents hospitalization of a patient.

Statins

Treatment with statins is recommended in patients with or at high-risk of CAD whether or not they have LV systolic dysfunction, in order to prevent or delay the onset of HF and prolong life.

In some embodiments, the disclosure relates to a method of treating a patient having heart failure by administering one or more statins. In some embodiments, the one or more statins is selected from the group consisting of: atorvastatin (Lipitor), fluvastatin (Lescol), lovastatin (Mevacor, Altocor), pravastatin (Pravachol), pitavastatin (Livalo), simvastatin (Zocor), and rosuvastatin (Crestor). In some embodiments a patient is administered atorvastatin. In some embodiments a patient is administered fluvastatin. In some embodiments a patient is administered lovastatin. In some embodiments a patient is administered pravastatin. In some embodiments a patient is administered pitavastatin. In some embodiments a patient is administered simvastatin. In some embodiments a patient is administered rosuvastatin.

In some embodiments, a patient is administered a statin when the patient shows signs of heart failure. In some embodiments, a patient is administered a statin when the patient is at high risk of coronary artery disease (CAD). In some embodiments, a patient is administered a statin when the patient has coronary artery disease (CAD). In some embodiments, a statin delays onset of heart failure in a patient. In some embodiments, a statin prevents onset of heart failure in a patient. In some embodiments, administration of a statin increases length of life in a patient. In some embodiments, administration of a statin decreases length of a hospital stay in a patient. In some embodiments, administration of a statin prevents hospitalization of a patient.

Sodium-Glucose Co-Transporter 2 (SGLT2) Inhibitors

Sodium-glucose co-transporter 2 (SGLT2) inhibitors are typically administered along with diet and exercise to lower blood sugar in adults with type 2 diabetes. SGLT2 inhibitors is lower blood sugar by causing the kidneys to remove sugar from the body through the urine. Treatment with SGCT2 inhibitors is recommended in patients with heart failure with reduced ejection fraction (HFrEF) to reduce the risk of cardiovascular death and hospitalization for heart failure.

In some embodiments, the disclosure relates to a method of treating a patient having heart failure by administering one or more SGLT2 inhibitor. In some embodiments, an SGLT2 inhibitor is a gliflozin. In some embodiments, a patient is administered one or more SGLT2 inhibitors selected from the group consisting of: canagliflozin, dapagliflozin (e.g., Farxiga), and empagliflozin. In some embodiments a patient is administered canagliflozin. In some embodiments a patient is administered dapagliflozin (e.g., Farxiga). In some embodiments a patient is administered empagliflozin.

In some embodiments, a patient is administered an SGLT2 inhibitor when the patient shows signs of heart failure. In some embodiments, a patient is administered an SGLT2 inhibitor when the patient does not have type 2 diabetes. In some embodiments, a patient is administered an SGLT2 inhibitor when the patient has type 2 diabetes. In some embodiments, an SGLT2 inhibitor delays onset of heart failure in a patient. In some embodiments, an SGLT2 inhibitor prevents onset of heart failure in a patient. In some embodiments, administration of an SGLT2 inhibitor increases length of life in a patient. In some embodiments, administration of an SGLT2 inhibitor decreases length of a hospital stay in a patient. In some embodiments, administration of an SGLT2 inhibitor prevents hospitalization of a patient. In some embodiments, an SGLT2 inhibitor reduces the risk of death of a patient.

Implantable Cardioverter Defibrillator (ICD)

Implantable cardioverter defibrillator (ICD) is recommended in patients with one or more of a) asymptomatic LV systolic dysfunction ((e.g., LVEF≤30%) of ischemic origin, who are at least 40 days after acute myocardial infarction, and b) asymptomatic non-ischemic dilated cardiomyopathy (e.g., LVEF≤30%), who receive osteopathic manipulative treatment (OMT), in order to prevent sudden death and prolong life. In some embodiments, the disclosure relates to a method of treating a patient having heart failure by administering an implantable cardioverter defibrillator (ICD).

In some embodiments, a patient is administered an ICD when the patient shows signs of heart failure. In some embodiments, a patient with asymptomatic LV systolic dysfunction (e.g., LVEF≤30%) of ischemic origin, who is at least 40 days after acute myocardial infarction, is administered an ICD. In some embodiments, a patient with asymptomatic LV systolic dysfunction (e.g., LVEF≤30%) of ischemic origin is administered an ICD. In some embodiments, a patient who is at least 40 days after acute myocardial infarction is administered an ICD. In some embodiments, a patient with asymptomatic non-ischemic dilated cardiomyopathy (e.g., LVEF≤30%), who receives optimal medical therapy (OMT) is administered an ICD. In some embodiments, a patient with asymptomatic non-ischemic dilated cardiomyopathy (e.g., LVEF≤30%) is administered an ICD. In some embodiments, a patient who receives optimal medical therapy is administered an ICD. In some embodiments, an ICD delays onset of heart failure in a patient. In some embodiments, an ICD prevents onset of heart failure in a patient. In some embodiments, administration of an ICD increases length of life in a patient. In some embodiments, administration of an ICD decreases length of a hospital stay in a patient. In some embodiments, administration of an ICD prevents hospitalization of a patient.

Angiotensin Receptor Neprilysin Inhibitor

A relatively new therapeutic class of agents acting on the renin-angiotensin-aldosterone system (RAAS) and the neutral endopeptidase system has been developed called angiotensin receptor neprilysin inhibitor (ARNI). The first in class is LCZ696, which is a molecule that combines the moieties of valsartan and sacubitril (a neprilysin inhibitor) in a single substance. By inhibiting neprilysin, the degradation of natriuretic peptides (NPs), bradykinin and other peptides is slowed.

High circulating A-type natriuretic peptide (ANP) and BNP exert physiologic effects through binding to NP receptors and the augmented generation of cGMP, thereby enhancing diuresis, natriuresis and myocardial relaxation and anti-remodeling. ANP and BNP also inhibit renin and aldosterone secretion. Selective AT1-receptor blockade reduces vasoconstriction, sodium and water retention and myocardial hypertrophy.

In some embodiments, the disclosure relates to a method of treating a patient having heart failure by administering an angiotensin-receptor neprilysin inhibitor. In some embodiments, a patient is administered sacubitril/valsartan (e.g. LCZ696, Entresto). In some embodiments, a patient with ambulatory, symptomatic HFrEF with LVEF≤35% is administered sacubitril/valsartan. In some embodiments, a patient with elevated plasma NP levels (BNP≥150 pg/mL and/or NT-proBNP 600 pg/mL (or, if they had been hospitalized for HF within the previous 12 months, BNP 100 pg/mL and/or NT-proBNP 400 pg/mL) is administered sacubitril/valsartan. In some embodiments, a patient with ambulatory, symptomatic HFrEF with LVEF≤35% is administered sacubitril/valsartan. In some embodiments, a patient with an estimated GFR (eGFR) 30 mL/min/1.73 m2 of body surface area is administered sacubitril/valsartan.

In some embodiments, a patient is administered sacubitril/valsartan when the patient shows signs of heart failure. In some embodiments, a patient is administered sacubitril/valsartan when the patient is intolerant of ACE inhibitors. In some embodiments, a patient is administered sacubitril/valsartan when the patient is intolerant of beta blockers. In some embodiments, a patient is administered sacubitril/valsartan when the patient is intolerant of MRAs. In some embodiments, a patient is administered sacubitril/valsartan when the patient has HFrEF and remains symptomatic despite treatment with one or more of an ACE inhibitor, a beta-blocker and an MRA. In some embodiments, sacubitril/valsartan delays onset of heart failure in a patient. In some embodiments, sacubitril/valsartan prevents onset of heart failure in a patient. In some embodiments, administration of sacubitril/valsartan increases length of life in a patient. In some embodiments, administration of sacubitril/valsartan decreases length of a hospital stay in a patient. In some embodiments, administration of sacubitril/valsartan prevents hospitalization of a patient.

In some embodiments, a patient is administered an ARNI when the patient shows signs of heart failure. In some embodiments, a patient is administered an ARNI when the patient is intolerant of ACE inhibitors. In some embodiments, a patient is administered an ARNI when the patient is intolerant of beta blockers. In some embodiments, a patient is administered an ARNI when the patient is intolerant of MRAs. In some embodiments, a patient is administered an ARNI when the patient has HFrEF and remains symptomatic despite treatment with one or more of an ACE inhibitor, a beta-blocker and an MRA. In some embodiments, an ARNI delays onset of heart failure in a patient. In some embodiments, an ARNI prevents onset of heart failure in a patient. In some embodiments, administration of an ARNI increases length of life in a patient. In some embodiments, administration of an ARNI decreases length of a hospital stay in a patient. In some embodiments, administration of an ARNI prevents hospitalization of a patient.

Diuretics

Diuretics are recommended to reduce signs and symptoms of congestion in patients with HFrEF. In patients with chronic HF, loop and thiazide diuretics may reduce the risk of death and worsening HF, and also possibly improve exercise capacity. Typically, loop diuretics produce a more intense and shorter diuresis than thiazides, although they act synergistically, and the combination may be used to treat resistant edema.

In some embodiments, the disclosure relates to a method of treating a patient having heart failure by administering one or more diuretics. In some embodiments, a patient is administered one or more diuretics selected from the group consisting of: furosemide, bumetanide, torasemide, bendroflumethiazide, hydrochlorothiazide, metolazone, indapamidec, spironolactone/eplerenone, amiloride and triamterene.

In some embodiments, a patient is administered one or more loop diuretics selected from the group consisting of furosemide, bumetanide and torasemide. In some embodiments a patient is administered furosemide. In some embodiments a patient is administered bumetanide. In some embodiments a patient is administered torasemide.

In some embodiments, a patient is administered one or more thiazide diuretics selected from the group consisting of bendroflumethiazide, hydrochlorothiazide, metolazone, and indapamidec. In some embodiments a patient is administered Bendroflumethiazide. In some embodiments a patient is administered hydrochlorothiazide. In some embodiments a patient is administered metolazone. In some embodiments a patient is administered indapamidec.

In some embodiments, a patient is administered one or more potassium-sparing diuretics selected from the group consisting of spironolactone/eplerenone, amiloride and triamterene. In some embodiments a patient is administered spironolactone/eplerenone. In some embodiments a patient is administered amiloride. In some embodiments a patient is administered triamterene.

In some embodiments, a patient is administered a diuretic when the patient shows signs of heart failure. In some embodiments, a patient is administered a diuretic when the patient shows signs congestion. In some embodiments, a patient is administered a diuretic when the patient is at high risk of coronary artery disease (CAD). In some embodiments, a patient is administered a diuretic when the patient has coronary artery disease (CAD). In some embodiments, a diuretic delays onset of heart failure in a patient. In some embodiments, a diuretic prevents onset of heart failure in a patient. In some embodiments, administration of a diuretic increases length of life in a patient. In some embodiments, administration of a diuretic decreases length of a hospital stay in a patient. In some embodiments, administration of a diuretic prevents hospitalization of a patient.. In some embodiments, administration of a diuretic improves a patient's six minute walk test.

Other

In some embodiments, a patient is administered one or more treatments selected from the group consisting of hydralazine and isosorbide dinitrate, digoxin, digitalis, N-3 polyunsaturated fatty acids (PUFA), and I_{f}-channel inhibitor (e.g., Ivabradine). In some embodiments a patient is administered hydralazine and isosorbide dinitrate. In some embodiments a patient is administered digoxin. In some embodiments a patient is administered digitalis. In some embodiments a patient is administered N-3 polyunsaturated fatty acids (PUFA). In some embodiments a patient is administered I_{f}-channel inhibitor (e.g., Ivabradine).

In some embodiments, a patient is administered one or more of hydralazine and isosorbide dinitrate, digoxin, digitalis, N-3 polyunsaturated fatty acids (PUFA), I_{f}-channel inhibitor (e.g., Ivabradine) when the patient shows signs of heart failure. In some embodiments, one or more of hydralazine and isosorbide dinitrate, digoxin, digitalis, N-3 polyunsaturated fatty acids (PUFA), If-channel inhibitor (e.g., Ivabradine) delays onset of heart failure in a patient. In some embodiments, one or more of hydralazine and isosorbide dinitrate, digoxin, digitalis, N-3 polyunsaturated fatty acids (PUFA), I_{f}-channel inhibitor (e.g., Ivabradine) prevents onset of heart failure in a patient. In some embodiments, administration of one or more of hydralazine and isosorbide dinitrate, digoxin, digitalis, N-3 polyunsaturated fatty acids (PUFA), If-channel inhibitor (e.g., Ivabradine) increases length of life in a patient. In some embodiments, administration of one or more of hydralazine and isosorbide dinitrate, digoxin, digitalis, N-3 polyunsaturated fatty acids (PUFA), If-channel inhibitor (e.g., Ivabradine) decreases length of a hospital stay in a patient. In some embodiments, administration of one or more of hydralazine and isosorbide dinitrate, digoxin, digitalis, N-3 polyunsaturated fatty acids (PUFA), If-channel inhibitor (e.g., Ivabradine) prevents hospitalization of a patient. In some embodiments, administration of one or more of hydralazine and isosorbide dinitrate, digoxin, digitalis, N-3 polyunsaturated fatty acids (PUFA), I_{f}-channel inhibitor (e.g., Ivabradine) improves a patient's six minute walk test.

### 8. Comorbidities

Comorbidities are important in HF and may affect the use of treatments for HF (e.g., it may not be possible to use renin-angiotensin system inhibitors in some patients with severe renal dysfunction). Furthermore, drugs used to treat comorbidities may cause worsening of HF (e.g., NSAIDs given for arthritis, some anti-cancer drugs, etc.). Therefore, management of comorbidities is a key component of the holistic care of patients with HF. In some embodiments, one or more comorbidities to consider in HF are selected from the group consisting of anemia, angina, arterial hypertension, arthritis, atrial fibrillation, cachexia, cancer, cognitive dysfunction, and coronary artery disease (CAD). diabetes, erectile dysfunction, gout, hypercholesterolemia, hyperkalemia, hyperkalemia, hyperlipidemia, hypertension, iron deficiency, kidney dysfunction, metabolic syndrome, obesity, physical deconditioning, potassium disorders, pulmonary disease (e.g., asthma, COPD), sarcopenia, sleep apnea, sleep disturbance, and valvular heart disease (e.g., aortic stenosis, aortic regurgitation, mitral regurgitation, tricuspid regurgitation). In some embodiments, one or more comorbidities to consider in HF are selected from the group consisting of anemia, atrial fibrillation, coronary artery disease (CAD), and sleep apnea.

In some embodiments, the disclosure contemplates methods of treating one or more comorbidities of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the disclosure contemplates methods of treating one or more comorbidities of heart failure (e.g., arterial hypertension, atrial fibrillation, cognitive dysfunction, diabetes, hypercholesterolemia, iron deficiency, kidney dysfunction, metabolic syndrome, obesity, physical deconditioning, potassium disorders, pulmonary disease (e.g., COPD), and sleep apnea) comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the one or more comorbidities of heart failure heart failure associated with aging are improved indirectly. In some embodiments, the disclosure contemplates methods of preventing one or more comorbidities of heart failure heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the disclosure contemplates methods of reducing the progression rate of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the disclosure contemplates methods of reducing the progression rate of one or more comorbidities of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the disclosure contemplates methods of reducing the severity of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist). In some embodiments, the disclosure contemplates methods of reducing the severity of one or more comorbidities of heart failure associated with aging, comprising administering to a patient in need thereof an effective amount of an ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist).

### 9. Screening Assays

In certain aspects, the present disclosure relates to the use of ActRII-ALK4 antagonist (e.g., an ActRII-ALK4 ligand trap antagonist, an ActRII-ALK4 antibody antagonist, an ActRII-ALK4 polynucleotide antagonist, and/or an ActRII-ALK4 small molecule antagonist) to identify compounds (agents) which may be used to treat, prevent, or reduce the progression rate and/or severity of heart failure associated with aging, particularly treating, preventing or reducing the progression rate and/or severity of one or more heart failure-associated comorbidities.

There are numerous approaches to screening for therapeutic agents for treating heart failure by targeting signaling (e.g., Smad signaling) of one or more ActRII-ALK4 ligands. In certain embodiments, high-throughput screening of compounds can be carried out to identify agents that perturb ActRII-ALK4 ligands-mediated effects on a selected cell line. In certain embodiments, the assay is carried out to screen and identify compounds that specifically inhibit or reduce binding of an ActRII-ALK4 ligand (e.g., activin A, activin B, activin AB, activin C, GDF3, BMP6, GDF8, GDF15, GDF11 or BMP10) to its binding partner, such as an a type II receptor (e.g., ActRIIA and/or ActRIIB). Alternatively, the assay can be used to identify compounds that enhance binding of an ActRII-ALK4 ligand to its binding partner such as a type II receptor. In a further embodiment, the compounds can be identified by their ability to interact with a type II receptor.

A variety of assay formats will suffice and, in light of the present disclosure, those not expressly described herein will nevertheless be comprehended by one of ordinary skill in the art. As described herein, the test compounds (agents) of the invention may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In certain embodiments, the test agent is a small organic molecule having a molecular weight of less than about 2,000 Daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S-transferase (GST), photoactivatable crosslinkers or any combinations thereof.

In many drug-screening programs which test libraries of compounds and natural extracts, high-throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between an ActRII-ALK4 ligand (e.g., activin A, activin B, activin AB, activin C, GDF8, GDF15, GDF11, GDF3, BMP6, or BMP10) to its binding partner, such as an a type II receptor (e.g., ActRIIA and/or ActRIIB).

Merely to illustrate, in an exemplary screening assay of the present disclosure, the compound of interest is contacted with an isolated and purified ActRIIB polypeptide which is ordinarily capable of binding to an ActRIIB ligand, as appropriate for the intention of the assay. To the mixture of the compound and ActRIIB polypeptide is then added to a composition containing an ActRIIB ligand (e.g., GDF11). Detection and quantification of ActRIIB/ActRIIB-ligand complexes provides a means for determining the compound's efficacy at inhibiting (or potentiating) complex formation between the ActRIIB polypeptide and its binding protein. The efficacy of the compound can be assessed by generating dose-response curves from data obtained using various concentrations of the test compound. Moreover, a control assay can also be performed to provide a baseline for comparison. For example, in a control assay, isolated and purified ActRIIB ligand is added to a composition containing the ActRIIB polypeptide, and the formation of ActRIIB/ActRIIB ligand complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between an ActRII-ALK4 ligand and its binding protein may be detected by a variety of techniques. For instance, modulation of the formation of complexes can be quantitated using, for example, detectably labeled proteins such as radiolabeled (e.g., ³²P, S, ¹⁴C or ³H), fluorescently labeled (e.g., FITC), or enzymatically labeled ActRIIB polypeptide and/or its binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between a GDF/BMP ligand and its binding protein. Further, other modes of detection, such as those based on optical waveguides (see, e.g., PCT Publication WO 96/26432 and U.S. Pat. No. 5,677,196), surface plasmon resonance (SPR), surface charge sensors, and surface force sensors, are compatible with many embodiments of the disclosure.

Moreover, the present disclosure contemplates the use of an interaction trap assay, also known as the “two-hybrid assay,” for identifying agents that disrupt or potentiate interaction between an ActRII-ALK4 ligand and its binding partner. See, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two-hybrid systems to identify compounds (e.g., small molecules or peptides) that dissociate interactions between an ActRII-ALK4 ligand and its binding protein [see, e.g., Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368].

In certain embodiments, the subject compounds are identified by their ability to interact with an ActRII-ALK4 ligand. The interaction between the compound and the ActRII-ALK4 ligand may be covalent or non-covalent. For example, such interaction can be identified at the protein level using in vitro biochemical methods, including photo-crosslinking, radiolabeled ligand binding, and affinity chromatography [see, e.g., Jakoby W B et al. (1974) Methods in Enzymology 46:1]. In certain cases, the compounds may be screened in a mechanism-based assay, such as an assay to detect compounds which bind to an ActRII-ALK4 ligand. This may include a solid-phase or fluid-phase binding event. Alternatively, the gene encoding ActRII-ALK4 ligand can be transfected with a reporter system (e.g., β-galactosidase, luciferase, or green fluorescent protein) into a cell and screened against the library preferably by high-throughput screening or with individual members of the library. Other mechanism-based binding assays may be used; for example, binding assays which detect changes in free energy. Binding assays can be performed with the target fixed to a well, bead or chip or captured by an immobilized antibody or resolved by capillary electrophoresis. The bound compounds may be detected usually using colorimetric endpoints or fluorescence or surface plasmon resonance.

### 10. Pharmaceutical Compositions

The therapeutic agents described herein (e.g., ActRII-ALK4 antagonists) may be formulated into pharmaceutical compositions. Pharmaceutical compositions for use in accordance with the present disclosure may be formulated in conventional manner using one or more physiologically acceptable carriers or excipients. Such formulations will generally be substantially pyrogen-free, in compliance with most regulatory requirements.

In certain embodiments, the therapeutic methods of the disclosure include administering the composition systemically, or locally as an implant or device. When administered, the therapeutic composition for use in this disclosure is in a substantially pyrogen-free, or pyrogen-free, physiologically acceptable form. Therapeutically useful agents other than the ActRII-ALK4 antagonists which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more ActRII-ALK4 antagonists in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In some embodiments, a parenteral route of administration is selected from the group consisting of intramuscular, intraperitoneal, intradermal, intravitreal, epidural, intracerebral, intra-arterial, intraarticular, intra-cavernous, intra-lesional, intraosseous, intraocular, intrathecal, intravenous, transdermal, trans-mucosal, extra-amniotic administration, subcutaneous, and combinations thereof. In some embodiments, a parenteral route of administration is subcutaneous. In some embodiments, a parenteral route of administration is a subcutaneous injection. In some embodiments, compositions of the present disclosure are administered by subcutaneous injection.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration

Further, the composition may be encapsulated or injected in a form for delivery to a target tissue site. In certain embodiments, compositions of the present invention may include a matrix capable of delivering one or more therapeutic compounds (e.g., ActRII-ALK4 antagonists) to a target tissue site, providing a structure for the developing tissue and optimally capable of being resorbed into the body. For example, the matrix may provide slow release of the ActRII-ALK4 antagonist. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In certain embodiments, methods of the invention can be administered for orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., ActRII-ALK4 antagonists). The various factors include, but are not limited to, the patient's age, sex, and diet, the severity disease, time of administration, and other clinical factors. Optionally, the dosage may vary with the type of matrix used in the reconstitution and the types of compounds in the composition. The addition of other known growth factors to the final composition, may also affect the dosage. Progress can be monitored by periodic assessment of bone growth and/or repair, for example, X-rays (including DEXA), histomorphometric determinations, and tetracycline labeling.

In certain embodiments, the present invention also provides gene therapy for the in vivo production of ActRII-ALK4 antagonists. Such therapy would achieve its therapeutic effect by introduction of the ActRII-ALK4 antagonist polynucleotide sequences into cells or tissues having the disorders as listed above. Delivery of ActRII-ALK4 antagonist polynucleotide sequences can be achieved using a recombinant expression vector such as a chimeric virus or a colloidal dispersion system. Preferred for therapeutic delivery of ActRII-ALK4 antagonist polynucleotide sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the ActRII-ALK4 antagonist. In one embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for ActRII-ALK4 antagonist polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

### EXEMPLIFICATION

The invention now being generally described, it will be more readily understood by reference to the following examples, which are included merely for purposes of illustration of certain embodiments of the present invention, and are not intended to limit the invention.

### Example 1: ActRIIA-Fc Fusion Proteins

A soluble ActRIIA fusion protein was constructed that has the extracellular domain of human ActRIIA fused to a human or mouse Fc domain with a minimal linker in between. The constructs are referred to as ActRIIA-hFc and ActRIIA-mFc, respectively.

ActRIIA-hFc is shown below as purified from CHO cell lines (SEQ ID NO: 380):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPPTGGGTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPVPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
K

An additional ActRIIA-hFc lacking the C-terminal lysine is shown below as purified from CHO cell lines (SEQ ID NO: 378):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPPTGGGTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPVPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG

The ActRIIA-hFc and ActRIIA-mFc proteins were expressed in CHO cell lines. Three different leader sequences were considered:

(i) Honey bee melittin (HBML):
(SEQ ID NO: 7)
MKFLVNVALVFMVVYISYIYA
(ii) Tissue plasminogen activator (TPA):
(SEQ ID NO: 8)
MDAMKRGLCCVLLLCGAVFVSP
(iii) Native:
(SEQ ID NO: 379)
MGAAAKLAFAVFLISCSSGA

The selected form employs the TPA leader and has the following unprocessed amino acid sequence:

(SEQ ID NO: 381)
MDAMKRGLCCVLLLCGAVFVSPGAAILGRSETQECLFFNANWEKDRTNQ
TGVEPCYGDKDKRRHCFATWKNISGSIEIVKQGCWLDDINCYDRTDCVE
KKDSPEVYFCCCEGNMCNEKFSYFPEMEVTQPTSNPVTPKPPTGGGTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPVPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGK

This polypeptide is encoded by the following nucleic acid sequence:

(SEQ ID NO: 382)
ATGGATGCAATGAAGAGAGGGCTCTGCTGTGTGCTGCTGCTGTGTGGAG
CAGTCTTCGTTTCGCCCGGCGCCGCTATACTTGGTAGATCAGAAACTCA
GGAGTGTCTTTTTTTAATGCTAATTGGGAAAAAGACAGAACCAATCAAA
CTGGTGTTGAACCGTGTTATGGTGACAAAGATAAACGGCGGCATTGTTT
TGCTACCTGGAAGAATATTTCTGGTTCCATTGAATAGTGAAACAAGGTT
GTTGGCTGGATGATATCAACTGCTATGACAGGACTGATTGTGTAGAAAA
AAAAGACAGCCCTGAAGTATATTTCTGTTGCTGTGAGGGCAATATGTGT
AATGAAAAGTTTTCTTATTTTCCGGAGATGGAAGTCACACAGCCCACTT
CAAATCCAGTTACACCTAAGCCACCCACCGGTGGTGGAACTCACACATG
CCCACCGTGCCCAGCACCTGAACTCCTGGGGGGACCGTCAGTCTTCCTC
TTCCCCCCAAAACCCAAGGACACCCTCATGATCTCCCGGACCCCTGAGG
TCACATGCGTGGTGGTGGACGTGAGCCACGAAGACCCTGAGGTCAAGTT
CAACTGGTACGTGGACGGCGTGGAGGTGCATAATGCCAAGACAAAGCCG
CGGGAGGAGCAGTACAACAGCACGTACCGTGTGGTCAGCGTCCTCACCG
TCCTGCACCAGGACTGGCTGAATGGCAAGGAGTACAAGTGCAAGGTCTC
CAACAAAGCCCTCCCAGTCCCCATCGAGAAAACCATCTCCAAAGCCAAA
GGGCAGCCCCGAGAACCACAGGTGTACACCCTGCCCCCATCCCGGGAGG
AGATGACCAAGAACCAGGTCAGCCTGACCTGCCTGGTCAAAGGCTTCTA
TCCCAGCGACATCGCCGTGGAGTGGGAGAGCAATGGGCAGCCGGAGAAC
AACTACAAGACCACGCCTCCCGTGCTGGACTCCGACGGCTCCTTCTTCC
TCTATAGCAAGCTCACCGTGGACAAGAGCAGGTGGCAGCAGGGGAACGT
CTTCTCATGCTCCGTGATGCATGAGGCTCTGCACAACCACTACACGCAG
AAGAGCCTCTCCCTGTCTCCGGGTAAATGAGAATTC

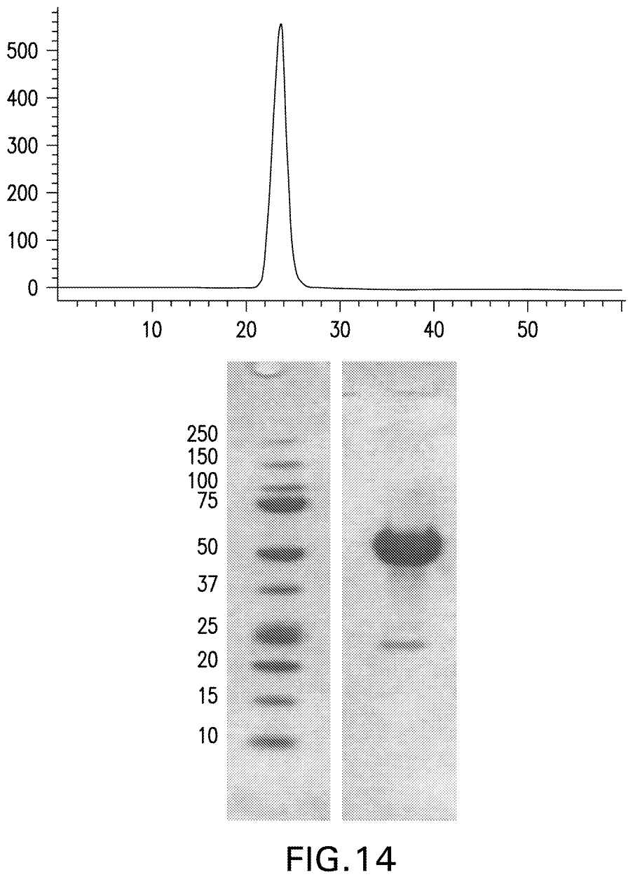

Both ActRIIA-hFc and ActRIIA-mFc were remarkably amenable to recombinant expression. As shown in FIG. 14, the protein was purified as a single, well-defined peak of protein. N-terminal sequencing revealed a single sequence of −ILGRSETQE (SEQ ID NO: 383). Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. The ActRIIA-hFc protein was purified to a purity of >98% as determined by size exclusion chromatography and >95% as determined by SDS PAGE.

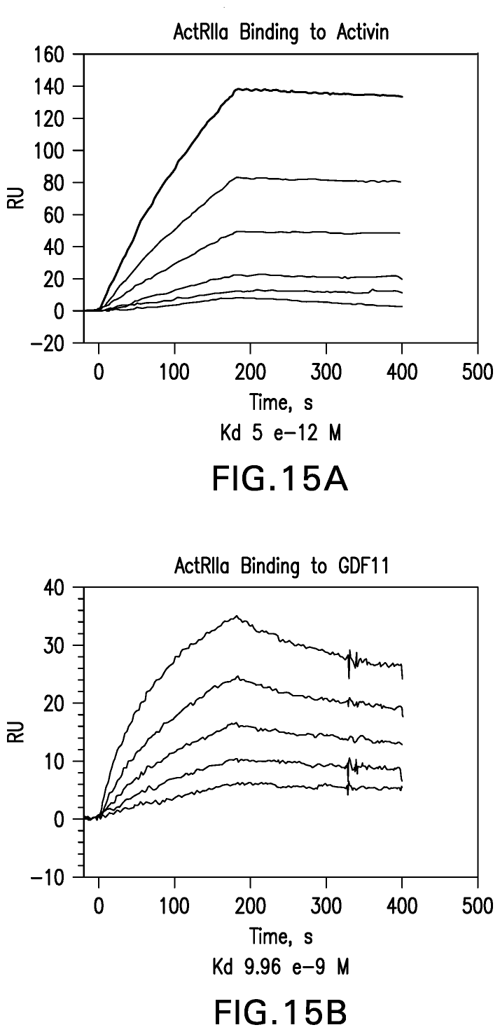

ActRIIA-hFc and ActRIIA-mFc showed a high affinity for ligands. GDF11 or activin A were immobilized on a Biacore™ CM5 chip using standard amine-coupling procedure. ActRIIA-hFc and ActRIIA-mFc proteins were loaded onto the system, and binding was measured. ActRIIA-hFc bound to activin with a dissociation constant (K_{D}) of 5×10⁻¹² and bound to GDF11 with a K_{D} of 9.96×10⁻⁹. See FIG. 15A-B. Using a similar binding assay, ActRIIA-hFc was determined to have high to moderate affinity for other TGF-beta superfamily ligands including, for example, activin B, GDF8, BMP6, and BMP10. ActRIIA-mFc behaved similarly.

The ActRIIA-hFc was very stable in pharmacokinetic studies. Rats were dosed with 1 mg/kg, 3 mg/kg, or 10 mg/kg of ActRIIA-hFc protein, and plasma levels of the protein were measured at 24, 48, 72, 144 and 168 hours. In a separate study, rats were dosed at 1 mg/kg, 10 mg/kg, or 30 mg/kg. In rats, ActRIIA-hFc had an 11-14 day serum half-life, and circulating levels of the drug were quite high after two weeks (11 μg/ml, 110 μg/ml, or 304 μg/ml for initial administrations of 1 mg/kg, 10 mg/kg, or 30 mg/kg, respectively.) In cynomolgus monkeys, the plasma half-life was substantially greater than 14 days, and circulating levels of the drug were 25 μg/ml, 304 μg/ml, or 1440 μg/ml for initial administrations of 1 mg/kg, 10 mg/kg, or 30 mg/kg, respectively.

### Example 2: Characterization of an ActRIIA-hFc Protein

ActRIIA-hFc fusion protein was expressed in stably transfected CHO-DUKX B11 cells from a pAID4 vector (SV40 on/enhancer, CMV promoter), using a tissue plasminogen leader sequence of SEQ ID NO: 8. The protein, purified as described above in Example 1, had a sequence of SEQ ID NO: 380. The Fc portion is a human IgG1 Fc sequence, as shown in SEQ ID NO: 380. Protein analysis reveals that the ActRIIA-hFc fusion protein is formed as a homodimer with disulfide bonding.

The CHO-cell-expressed material has a higher affinity for activin B ligand than that reported for an ActRIIA-hFc fusion protein expressed in human 293 cells [see, del Re et al. (2004) J Biol Chem. 279(51):53126-53135]. Additionally, the use of the TPA leader sequence provided greater production than other leader sequences and, unlike ActRIIA-Fc expressed with a native leader, provided a highly pure N-terminal sequence. Use of the native leader sequence resulted in two major species of ActRIIA-Fc, each having a different N-terminal sequence.

### Example 3: Alternative ActRIIA-Fc Proteins

A variety of ActRIIA variants that may be used according to the methods described herein are described in the International Patent Application published as WO2006/012627 (see e.g., pp. 55-58), incorporated herein by reference in its entirety. An alternative construct may have a deletion of the C-terminal tail (the final 15 amino acids of the extracellular domain of ActRIIA. The sequence for such a construct is presented below (Fc portion underlined) (SEQ ID NO: 384):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMTGGGTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPVPIEKTISKAKGQPREPQVYTLPPSREEMTKNQV
SLTCLVKGFYPSDIAVEWESNGQPENNYKTTPPVLDSDGSFFLYSKLTV
DKSRWQQGNVFSCSVMHEALHNHYTQKSLSLSPGK

### Example 4. Generation of an ActRIIB-Fc Fusion Polypeptide

Applicants constructed a soluble ActRIIB fusion polypeptide that has the extracellular domain of human ActRIIB fused to a human G1Fc domain with a linker (three glycine amino acids) in between. The construct is referred to as ActRIIB(20-134)-G1Fc.

ActRIIB(20-134)-G1Fc is shown below in SEQ ID NO: 5 (with the linker underlined) as purified from CHO cell lines:

(SEQ ID NO: 5)
GRGEAETRECIYYNANWELERTNQSGLERCEGEQDKRLHCYASWRNSSG
TIELVKKGCWLDDFNCYDRQECVATEENPQVYFCCCEGNFCNERFTHLP
EAGGPEVTYEPPPTAPTGGGTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK

An additional ActRIIB(20-134)-G1Fc lacking the C-terminal lysine is shown below as purified from CHO cell lines (SEQ ID NO: 385):

(SEQ ID NO: 385)
GRGEAETRECIYYNANWELERTNQSGLERCEGEQDKRLHCYASWRNSSG
TIELVKKGCWLDDFNCYDRQECVATEENPQVYFCCCEGNFCNERFTHLP
EAGGPEVTYEPPPTAPTGGGTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG

The ActRIIB(20-134)-G1Fc polypeptide was expressed in CHO cell lines. Three different leader sequences were considered:

(i) Honey bee melittin (HBML):
(SEQ ID NO: 7)
MKFLVNVALVFMVVYISYIYA
(ii) Tissue plasminogen activator (TPA):
(SEQ ID NO: 8)
MDAMKRGLCCVLLLCGAVFVSP
(iii) Native:
(SEQ ID NO: 9)
MTAPWVALALLWGSLCAG

The selected form employs the TPA leader and has the following unprocessed amino acid sequence:

(SEQ ID NO: 6)
MDAMKRGLCCVLLLCGAVFVSPGASGRGEAETRECIYYNANWELERTNQ
SGLERCEGEQDKRLHCYASWRNSSGTIELVKKGCWLDDFNCYDRQECVA
TEENPQVYFCCCEGNFCNERFTHLPEAGGPEVTYEPPPTAPTGGGTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNV
FSCSVMHEALHNHYTQKSLSLSPGK

This polypeptide is encoded by the following nucleic acid sequence (SEQ ID NO: 10):

(SEQ ID NO: 10)
ATGGATGCAAT GAAGAGAGGG CTCTGCTGTG TGCTGCTGCT
GTGTGGAGCA GTCTTCGTTT CGCCCGGCGC CTCTGGGCGT
GGGGAGGCTG AGACACGGGA GTGCATCTAC TACAACGCCA
ACTGGGAGCT GGAGCGCACC AACCAGAGCG GCCTGGAGCG
CTGCGAAGGC GAGCAGGACA AGCGGCTGCA CTGCTACGCC
TCCTGGCGCA ACAGCTCTGG CACCATCGAG CTCGTGAAGA
AGGGCTGCTG GCTAGATGAC TTCAACTGCT ACGATAGGCA
GGAGTGTGTG GCCACTGAGG AGAACCCCCA GGTGTACTTC
TGCTGCTGTG AAGGCAACTT CTGCAACGAG CGCTTCACTC
ATTTGCCAGA GGCTGGGGGC CCGGAAGTCA CGTACGAGCC
ACCCCCGACA GCCCCCACCG GTGGTGGAAC TCACACATGC
CCACCGTGCC CAGCACCTGA ACTCCTGGGG GGACCGTCAG
TCTTCCTCTT CCCCCCAAAA CCCAAGGACA CCCTCATGAT
CTCCCGGACC CCTGAGGTCA CATGCGTGGT GGTGGACGTG
AGCCACGAAG ACCCTGAGGT CAAGTTCAAC TGGTACGTGG
ACGGCGTGGA GGTGCATAAT GCCAAGACAA AGCCGCGGGA
GGAGCAGTAC AACAGCACGT ACCGTGTGGT CAGCGTCCTC
ACCGTCCTGC ACCAGGACTG GCTGAATGGC AAGGAGTACA
AGTGCAAGGT CTCCAACAAA GCCCTCCCAG CCCCCATCGA
GAAAACCATC TCCAAAGCCA AAGGGCAGCC CCGAGAACCA
CAGGTGTACA CCCTGCCCCC ATCCCGGGAG GAGATGACCA
AGAACCAGGT CAGCCTGACC TGCCTGGTCA AAGGCTTCTA
TCCCAGCGAC ATCGCCGTGG AGTGGGAGAG CAATGGGCAG
CCGGAGAACA ACTACAAGAC CACGCCTCCC GTGCTGGACT
CCGACGGCTC CTTCTTCCTC TATAGCAAGC TCACCGTGGA
CAAGAGCAGG TGGCAGCAGG GGAACGTCTT CTCATGCTCC
GTGATGCATG AGGCTCTGCA CAACCACTAC ACGCAGAAGA
GCCTCTCCCT GTCTCCGGGT AAATGA

N-terminal sequencing of the CHO-cell produced material revealed a major sequence of -GRGEAE (SEQ ID NO: 11). Notably, other constructs reported in the literature begin with an -SGR . . . sequence.

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

The ActRIIB(20-134)-Fc fusion polypeptide was also expressed in HEK293 cells and COS cells. Although material from all cell lines and reasonable culture conditions provided polypeptide with muscle-building activity in vivo, variability in potency was observed perhaps relating to cell line selection and/or culture conditions.

### Example 5. Computational Methods

The Activin IIB receptor (ActRIIB) binds multiple TGFβ superfamily ligands, including activin A, activin B, GDF8, and GDF11, that stimulate Smad2/3 activation, as well as bone morphogenic proteins (BMPs), such as BMP9 and BMP10, that stimulate Smad1/5/8 activation. ActRIIB-Fc fusion polypeptides can function as ligand traps that bind to soluble ligands and block Smad activation by preventing ligands from binding to cell surface receptors. ActRIIB-Fc antagonism of BMP9-mediated Smad1/5/8 activation has been known to result in undesired side effects, including epistaxis and telangiectasias (Campbell, C. et al. Muscle Nerve 55: 458-464, 2017). In order to design mutations in ActRIIB that diminish BMP9 binding, while retaining binding to ligands that stimulate Smad2/3 activation, we compared the crystal structures of three ActRIIB ligand complexes: (1) BMP9:ActRIIB:Alk1, PDB ID=4fao, (2) ActRIIB:Activin A, PDB ID:1s4y, and (3) GDF11:ActRIIB:Alk5, PDB ID: 6mac (available from the Protein Data Bank (PDB) https://www.rcsb.org/). Comparison of contacts between ActRIIB and the three ligands based on the crystal structures revealed residues for mutational focus based on charge, polarity, and hydrophobicity differences of the ligand residues contacted by the same corresponding ActRIIB residue. After identifying residues to target for mutation, the Schrödinger Bioluminate biologics modeling software platform (version 2017-4: Bioluminate, Schrödinger, LLC, New York, NY) was used to computationally predict mutations in ActRIIB that would diminish binding to BMP9, while maintaining other ligand-binding activities.

All residues identified from the comparison of the crystal structures were considered for mutation. Residue Scanning Calculations were performed considering both stability and affinity of the molecules in the structural complex, producing a specified list of potential mutations and energies for each molecule (ligand and receptor) and complex structure, as well as energy differences for both the wild type and the mutant form. After analyzing affinity/stability/prime energy, etc. parameters, the top 5%-10% of the single mutations were identified. This analysis was followed by potential combination of these mutations. Selected single mutations and mutation combinations were structurally analyzed in order to understand structural differences and formed/lost contacts. Ultimately, 817 single mutations were screened for each complex (ActRIIB:ligand), and top hits were selected based on Δaffinity, and also taking into selective consideration Δstability (solvated) and Δprime energy. Other properties were also considered when regarding striking of outliers.

### Example 6. Generation of Variant ActRIIB-Fc Polypeptides

Based on the findings described in Example 4, Applicants generated a series of mutations (sequence variations) in the extracellular domain of ActRIIB and produced these variant polypeptides as soluble homodimeric fusion polypeptides comprising a variant ActRIIB extracellular domain and an Fc domain joined by an optional linker. The background ActRIIB-Fc fusion used for the generation of variant ActRIIB-Fc polypeptides was ActRIIB-G1Fc, and is shown in Example 4 above as SEQ ID NO: 5.

Various substitution mutations were introduced into the background ActRIIB-G1Fc polypeptide. Based on the data presented in Example 4, it is expected that these constructs, if expressed with a TPA leader, will lack the N-terminal serine. Thus, the majority of mature sequences may begin with a glycine (lacking the N-terminal serine) but some species may be present with the N-terminal serine. Mutations were generated in the ActRIIB extracellular domain by PCR mutagenesis. After PCR, fragments were purified through a Qiagen column, digested with SfoI and AgeI and gel purified. These fragments were ligated into expression vector pAID4 (see WO2006/012627) such that upon ligation it created fusion chimera with human IgG1. Upon transformation into E. coli DH5 alpha, colonies were picked and DNA was isolated. For murine constructs (mFc), a murine IgG2a was substituted for the human IgG1. All mutants were sequence verified.

The amino acid sequence of unprocessed ActRIIB(F82I-N83R)-G1Fc is shown below (SEQ ID NO: 276). The signal sequence and linker sequence are indicated by solid underline, and the F82I and N83R substitutions are indicated by double underline. The amino acid sequence of SEQ ID NO: 276 may optionally be provided with the lysine removed from the C-terminus.

| (SEQ ID NO: 276) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDIRC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVESCSVMH |
| 351 | EALHNHYTQK SLSLSPGK |

This ActRIIB(F82I-N83R)-G1Fc fusion polypeptide is encoded by the following nucleic acid sequence (SEQ ID NO: 277):

| (SEQ ID NO: 277) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CATCCGTTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(F82I-N83R)-G1Fc fusion polypeptide (SEQ ID NO: 278) is as follows and may optionally be provided with the lysine removed from the C-terminus.

| (SEQ ID NO: 278) | |
|---|---|
| 1 | GRGEAETREC IYYNANWELE RTNQSGLERC EGEQDKRLHC YASWRNSSGT |
| 51 | IELVKKGCWL DDIRCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVES CSVMHEALHN HYTQKSLSLS PGK |

The amino acid sequence of unprocessed ActRIIB(F82K-N83R)-G1Fc is shown below (SEQ ID NO: 279). The signal sequence and linker sequence are indicated by solid underline, and the F82K and N83R substitutions are indicated by double underline. The amino acid sequence of SEQ ID NO: 279 may optionally be provided with the lysine removed from the C-terminus.

| (SEQ ID NO: 279) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDKRC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVESCSVMH |
| 351 | EALHNHYTQK SLSLSPGK |

This ActRIIB(F82K-N83R)-G1Fc fusion polypeptide is encoded by the following nucleic acid sequence (SEQ ID NO: 331):

| (SEQ ID NO: 331) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CAAGCGTTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(F82K-N83R)-G1Fc fusion polypeptide (SEQ ID NO: 332) is as follows and may optionally be provided with the lysine removed from the C-terminus.

| (SEQ ID NO: 332) | |
|---|---|
| 1 | GRGEAETREC IYYNANWELE RTNQSGLERC EGEQDKRLHC YASWRNSSGT |
| 51 | IELVKKGCWL DDKRCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVES CSVMHEALHN HYTQKSLSLS PGK |

The amino acid sequence of unprocessed ActRIIB(F82T-N83R)-G1Fc is shown below (SEQ ID NO: 333). The signal sequence and linker sequence are indicated by solid underline, and the F82T and N83R substitutions are indicated by double underline. The amino acid sequence of SEQ ID NO: 333 may optionally be provided with the lysine removed from the C-terminus.

| (SEQ ID NO: 333) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDTRC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVESCSVMH |
| 351 | EALHNHYTQK SLSLSPGK |

This ActRIIB(F82T-N83R)-G1Fc fusion polypeptide is encoded by the following nucleic acid sequence (SEQ ID NO: 334):

| (SEQ ID NO: 334) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CACCCGTTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(F82T-N83R)-G1Fc fusion polypeptide (SEQ ID NO: 335) is as follows and may optionally be provided with the lysine removed from the C-terminus.

| (SEQ ID NO: 335) | |
|---|---|
| 1 | GRGEAETREC IYYNANWELE RTNQSGLERC EGEQDKRLHC YASWRNSSGT |
| 51 | IELVKKGCWL DDTRCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSE |
| 301 | FLYSKLTVDK SRWQQGNVES CSVMHEALHN HYTQKSLSLS PGK |

The amino acid sequence of unprocessed ActRIIB(F82T)-G1Fc is shown below (SEQ ID NO: 336). The signal sequence and linker sequence are indicated by solid underline, and the F82T substitution is indicated by double underline. The amino acid sequence of SEQ ID NO: 336 may optionally be provided with the lysine removed from the C-terminus.

| (SEQ ID NO: 336) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDTNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVESCSVMH |
| 351 | EALHNHYTQK SLSLSPGK |

This ActRIIB(F82T)-G1Fc fusion polypeptide is encoded by the following nucleic acid sequence (SEQ ID NO: 337):

| (SEQ ID NO: 337) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CACCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(F82T)-G1Fc fusion polypeptide (SEQ ID NO: 338) is as follows and may optionally be provided with the lysine removed from the C-terminus.

| (SEQ ID NO: 338) | |
|---|---|
| 1 | GRGEAETREC IYYNANWELE RTNQSGLERC EGEQDKRLHC YASWRNSSGT |
| 51 | IELVKKGCWL DDTNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK |

The amino acid sequence of unprocessed ActRIIB(L79H-F82I)-G1Fc is shown below (SEQ ID NO: 339). The signal sequence and linker sequence are indicated by solid underline, and the L79H and F82I substitutions are indicated by double underline. The amino acid sequence of SEQ ID NO: 339 may optionally be provided with the lysine removed from the C-terminus.

| (SEQ ID NO: 339) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWHDDINC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVESCSVMH |
| 351 | EALHNHYTQK SLSLSPGK |

This ActRIIB(L79H-F82I)-G1Fc fusion polypeptide is encoded by the following nucleic acid sequence (SEQ ID NO: 340):

| (SEQ ID NO: 340) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCACGATGA CATCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(L79H-F82I)-G1Fc fusion polypeptide (SEQ ID NO: 341) is as follows and may optionally be provided with the lysine removed from the C-terminus.

| (SEQ ID NO: 341) | |
|---|---|
| 1 | GRGEAETREC IYYNANWELE RTNQSGLERC EGEQDKRLHC YASWRNSSGT |
| 51 | IELVKKGCWH DDINCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK |

The amino acid sequence of unprocessed ActRIIB(L79H)-G1Fc is shown below (SEQ ID NO: 342). The signal sequence and linker sequence are indicated by solid underline, and the L79H substitution is indicated by double underline. The amino acid sequence of SEQ ID NO: 342 may optionally be provided with the lysine removed from the C-terminus.

| (SEQ ID NO: 342) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWHDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK |

This ActRIIB(L79H)-G1Fc fusion polypeptide is encoded by the following nucleic acid sequence (SEQ ID NO: 343):

| (SEQ ID NO: 343) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCACGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(L79H)-G1Fc fusion polypeptide (SEQ ID NO: 344) is as follows and may optionally be provided with the lysine removed from the C-terminus.

| (SEQ ID NO: 344) | |
|---|---|
| 1 | GRGEAETREC IYYNANWELE RTNQSGLERC EGEQDKRLHC YASWRNSSGT |
| 51 | IELVKKGCWH DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK |

The amino acid sequence of unprocessed ActRIIB(L79H-F82K)-G1Fc is shown below (SEQ ID NO: 345). The signal sequence and linker sequence are indicated by solid underline, and the L79H and F82K substitutions are indicated by double underline. The amino acid sequence of SEQ ID NO: 345 may optionally be provided with the lysine removed from the C-terminus.

| (SEQ ID NO: 345) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWHDDKNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK |

This ActRIIB(L79H-F82K)-G1Fc fusion polypeptide is encoded by the following nucleic acid sequence (SEQ ID NO: 346):

| (SEQ ID NO: 346) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCACGATGA CAAGAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(L79H-F82K)-G1Fc fusion polypeptide (SEQ ID NO: 347) is as follows and may optionally be provided with the lysine removed from the C-terminus.

| (SEQ ID NO: 347) | |
|---|---|
| 1 | GRGEAETREC IYYNANWELE RTNQSGLERC EGEQDKRLHC YASWRNSSGT |
| 51 | IELVKKGCWH DDKNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK |

The amino acid sequence of unprocessed ActRIIB(E50L)-G1Fc is shown below (SEQ ID NO: 348). The signal sequence and linker sequence are indicated by solid underline, and the E50L substitution is indicated by double underline. The amino acid sequence of SEQ ID NO: 348 may optionally be provided with the lysine removed from the C-terminus.

| (SEQ ID NO: 348) | |
|---|---|
| 1 | MDAMKRGLCC VLLLCGAVFV SPGASGRGEA ETRECIYYNA NWELERTNQS |
| 51 | GLERCLGEQD KRLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK |

This ActRIIB(E50L)-G1Fc fusion polypeptide is encoded by the following nucleic acid sequence (codon optimized) (SEQ ID NO: 349):

| (SEQ ID NO: 349) | |
|---|---|
| 1 | ATGGATGCGA TGAAACGCGG CCTGTGCTGC GTGCTGCTGC TGTGCGGCGC |
| 51 | GGTGTTTGTG AGCCCGGGCG CCAGCGGCCG CGGCGAAGCG GAAACCCGCG |
| 101 | AATGCATTTA TTATAACGCG AACTGGGAAC TGGAACGCAC CAACCAGAGC |
| 151 | GGCCTGGAAC GCTGCCTGGG CGAACAGGAT AAACGCCTGC ATTGCTATGC |
| 201 | GAGCTGGCGC AACAGCAGCG GCACCATTGA ACTGGTGAAA AAAGGCTGCT |
| 251 | GGCTGGATGA TTTTAACTGC TATGATCGCC AGGAATGCGT GGCGACCGAA |
| 301 | GAAAACCCGC AGGTGTATTT TTGCTGCTGC GAAGGCAACT TTTGCAACGA |
| 351 | ACGCTTTACC CATCTGCCGG AAGCGGGCGG CCCGGAAGTG ACCTATGAAC |
| 401 | CGCCGCCGAC CGCGCCGACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(E50L)-G1Fc fusion polypeptide (SEQ ID NO: 350) is as follows and may optionally be provided with the lysine removed from the C-terminus.

| (SEQ ID NO: 350) | |
|---|---|
| 1 | GRGEAETREC IYYNANWELE RTNQSGLERC LGEQDKRLHC YASWRNSSGT |
| 51 | IELVKKGCWL DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK |

The amino acid sequence of unprocessed ActRIIB(L38N-L79R)-G1Fc is shown below (SEQ ID NO: 351). The signal sequence and linker sequence are indicated by solid underline, and the L38N and L79R substitutions are indicated by double underline. The amino acid sequence of SEQ ID NO: 351 may optionally be provided with the lysine removed from the C-terminus.

| (SEQ ID NO: 351) | |
|---|---|
| 1 | MDAMKRGLCC VLLLCGAVFV SPGASGRGEA ETRECIYYNA NWENERTNQS |
| 51 | GLERCEGEQD KRLHCYASWR NSSGTIELVK KGCWRDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK |

This ActRIIB(L38N-L79R)-G1Fc fusion polypeptide is encoded by the following nucleic acid sequence (SEQ ID NO: 352):

| (SEQ ID NO: 352) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGA ACGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCGCGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(L38N-L79R)-G1Fc fusion polypeptide (SEQ ID NO: 353) is as follows and may optionally be provided with the lysine removed from the C-terminus.

| (SEQ ID NO: 353) | |
|---|---|
| 1 | GRGEAETREC IYYNANWENE RTNQSGLERC EGEQDKRLHC YASWRNSSGT |
| 51 | IELVKKGCWR DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK |

The amino acid sequence of unprocessed ActRIIB(V99G)-G1Fc is shown below (SEQ ID NO: 354). The signal sequence and linker sequence are indicated by solid underline, and the V99G substitution is indicated by double underline. The amino acid sequence of SEQ ID NO: 354 may optionally be provided with the lysine removed from the C-terminus.

| (SEQ ID NO: 354) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDFNC YDRQECVATE |
| 101 | ENPQGYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK |

This ActRIIB(V99G)-G1Fc fusion polypeptide is encoded by the following nucleic acid sequence (codon optimized) (SEQ ID NO: 355):

| (SEQ ID NO: 355) | |
|---|---|
| 1 | ATGGATGCGA TGAAACGCGG CCTGTGCTGC GTGCTGCTGC TGTGCGGCGC |
| 51 | GGTGTTTGTG AGCCCGGGCG CCAGCGGCCG CGGCGAAGCG GAAACCCGCG |
| 101 | AATGCATTTA TTATAACGCG AACTGGGAAC TGGAACGCAC CAACCAGAGC |
| 151 | GGCCTGGAAC GCTGCGAAGG CGAACAGGAT AAACGCCTGC ATTGCTATGC |
| 201 | GAGCTGGCGC AACAGCAGCG GCACCATTGA ACTGGTGAAA AAAGGCTGCT |
| 251 | GGCTGGATGA TTTTAACTGC TATGATCGCC AGGAATGCGT GGCGACCGAA |
| 301 | GAAAACCCGC AGGGCTATTT TTGCTGCTGC GAAGGCAACT TTTGCAACGA |
| 351 | ACGCTTTACC CATCTGCCGG AAGCGGGGG CCCGGAAGTG ACCTATGAAC |
| 401 | CGCCGCCGAC CGCGCCGACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(V99G)-G1Fc fusion polypeptide (SEQ ID NO: 356) is as follows and may optionally be provided with the lysine removed from the C-terminus.

| (SEQ ID NO: 356) | |
|---|---|
| 1 | GRGEAETREC IYYNANWELE RTNQSGLERC EGEQDKRLHC YASWRNSSGT |
| 51 | IELVKKGCWL DDENCYDRQE CVATEENPQG YFCCCEGNFC NERFTHLPEA |
| 101 | GGPEVTYEPP PTAPTGGGTH TCPPCPAPEL LGGPSVELFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSE |
| 301 | FLYSKLTVDK SRWQQGNVES CSVMHEALHN HYTQKSLSLS PGK |

Constructs were expressed in COS or CHO cells by transient infection and purified by filtration and protein A chromatography. In some instances, assays were performed with conditioned medium rather than purified polypeptides. Purity of samples for reporter gene assays was evaluated by SDS-PAGE and analytical size exclusion chromatography.

Mutants were tested in binding assays and/or bioassays described below.

Alternatively, similar mutations could be introduced into an ActRIIB extracellular domain possessing an N-terminal truncation of five amino acids and a C-terminal truncation of three amino acids as shown below (SEQ ID NO: 357). This truncated ActRIIB extracellular domain is denoted ActRIIB(25-131) based on numbering in SEQ ID NO: 2.

| (SEQ ID NO: 357) | |
|---|---|
| 25 | ETRECIYYNA NWELERTNQS GLERCEGEQD KRLHCYASWR NSSGTIELVK |
| 75 | KGCWLDDFNC YDRQECVATE ENPQVYFCCC EGNFCNERFT HLPEAGGPEV |
| 125 | TYEPPPT |

The corresponding background fusion polypeptide, ActRIIB(25-131)-G1Fc, is shown below (SEQ ID NO: 12).

| (SEQ ID NO: 12) | |
|---|---|
| 1 | ETRECIYYNA NWELERTNQS GLERCEGEQD KRLHCYASWR NSSGTIELVK |
| 51 | KGCWLDDFNC YDRQECVATE ENPQVYFCCC EGNECNERFT HLPEAGGPEV |
| 101 | TYEPPPTGGG THTCPPCPAP ELLGGPSVFL FPPKPKDTLM ISRTPEVTCV |
| 151 | VVDVSHEDPE VKFNWYVDGV EVHNAKTKPR EEQYNSTYRV VSVLTVLHQD |
| 201 | WLNGKEYKCK VSNKALPAPI EKTISKAKGQ PREPQVYTLP PSREEMTKNQ |
| 251 | VSLTCLVKGF YPSDIAVEWE SNGQPENNYK TTPPVLDSDG SFFLYSKLTV |
| 301 | DKSRWQQGNV FSCSVMHEAL HNHYTQKSLS LSPGK |

### Example 7. Activity and Ligand Binding Profiles of Variant ActRIIB-Fc Polypeptides

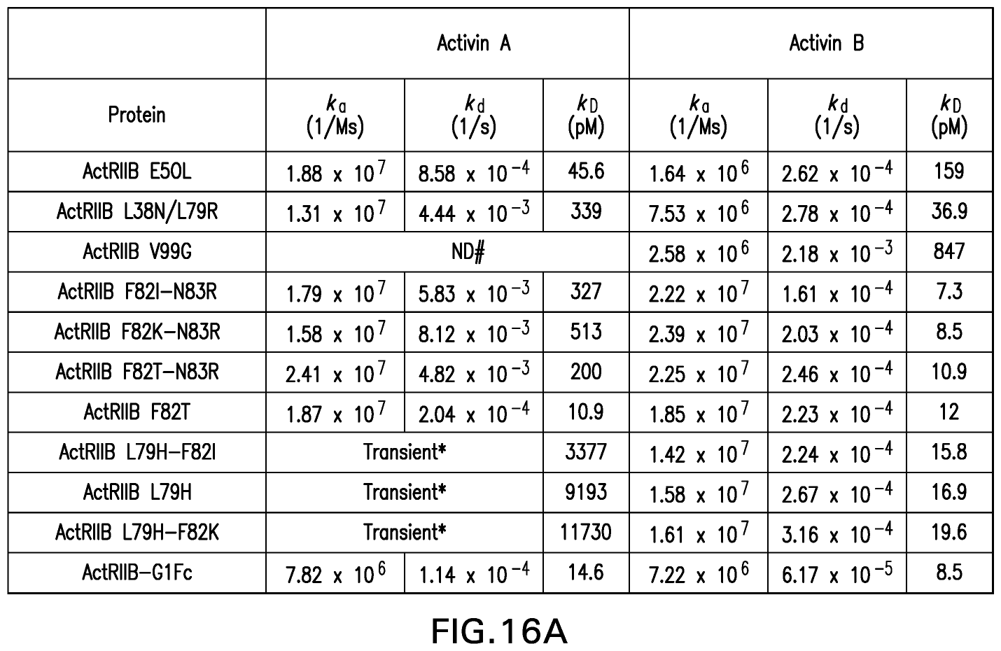

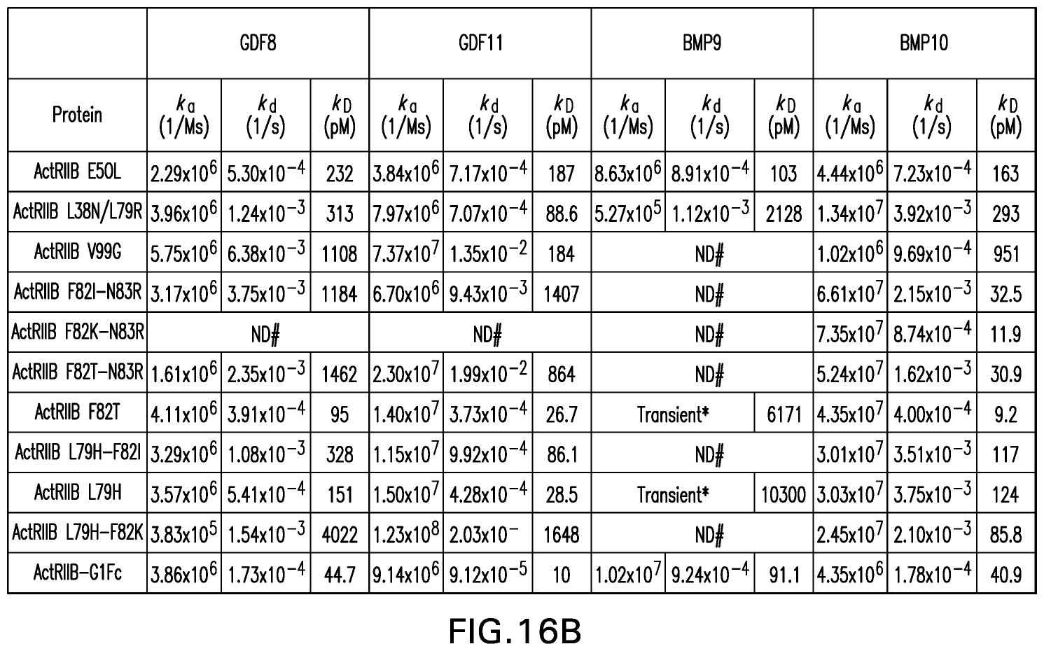

To determine ligand binding profiles of variant ActRIIB-Fc homodimers, a Biacore™_based binding assay was used to compare ligand binding kinetics of certain variant ActRIIB-Fc polypeptides. ActRIIB-Fc polypeptides to be tested were independently captured onto the system using an anti-Fc antibody. Ligands were then injected and allowed to flow over the captured receptor protein. Results of variant ActRIIB-Fc polypeptides analyzed at 37° C. are shown in FIGS. 16A and 16B. ActRIIB-G1Fc was used as the control polypeptide.

To determine activity of variant ActRIIB-Fc polypeptides, an A204 cell-based assay was used to compare effects among variant ActRIIB-Fc polypeptides on signaling by activin A, activin B, GDF8, GDF11, BMP9, and BMP10, in comparison to ActRIIB-G1Fc. In brief, this assay uses a human A204 rhabdomyosarcoma cell line (ATCC®: HTB-82™) derived from muscle and the reporter vector pGL3(CAGA)12 (Dennler et al., 1998, EMBO 17: 3091-3100) as well as a Renilla reporter plasmid (pRLCMV) to control for transfection efficiency. The CAGA12 motif is present in TGF-β responsive genes (e.g., PAI-1 gene), so this vector is of general use for ligands that can signal through Smad2/3, including activin A, GDF11, and BMP9.

On day 1, A204 cells were transferred into one or more 48-well plates. On day 2, these cells were transfected with 10 pg pGL3(CAGA)12 or pGL3(CAGA)12(10 pg)+pRLCMV (1 pg) and Fugene. On day 3, ligands diluted in medium containing 0.1% BSA were preincubated with ActRIIB-Fc polypeptides for 1 hr before addition to cells. Approximately six hour later, the cells were rinsed with PBS and lysed. Cell lysates were analyzed in a luciferase assay to determine the extent of Smad activation.

This assay was used to screen variant ActRIIB-Fc polypeptides for inhibitory effects on cell signaling by activin A, activin B, GDF8, GDF11, BMP9, and BMP10. Potencies of homodimeric Fc fusion polypeptides incorporating amino acid substitutions in the human ActRIIB extracellular domain were compared with that of an Fc fusion polypeptide comprising unmodified human ActRIIB extracellular domain, ActRIIB-G1Fc. For some variants tested, it was not possible to calculate an accurate IC₅₀, but signs of inhibition in the slope of the curves were detectable. For these variants, an estimate was included of the order of magnitude of the relative IC50, i.e. >10 nM or >100 nM instead of a definite number. Such data points are indicated by a (*) in Table 13 below. For some variants tested, there was no detectable inhibition in the slope of the curves over the concentration range tested, which is indicated by “ND” in Table 13.

TABLE 13
Inhibitory Potency of Homodimeric ActRIIB-Fc Constructs.
Inhibitory Potency of Homodimeric ActRIIB-Fc Constructs

| ActRIIB | IC₅₀ (nM) | | | | | |
|---|---|---|---|---|---|---|
| polypeptide | GDF8 | GDF11 | Activin A | Activin B | BMP9 | BMP10 |
| ActRIIB- | 0.95 | 0.12 | 0.05 | 0.067 | 1.82 | 0.036 |
| G1Fc | | | | | | |
| F82I-N83R | ND | 9.95 | 1.67 | 0.08 | ND | 13.25 |
| F82K-N83R | ND | ND | 1.32 | 0.09 | ND | 0.53 |
| F82T-N83R | ND | 17.94 | 1.52 | 0.11 | ND | 12.57 |
| F82T | 2.17 | 0.27 | 0.10 | 0.09 | ND | 0.07 |
| L79H-F82I | >10* | 0.36 | >100* | 0.15 | ND | >100* |
| L79H | 5.76 | 0.24 | >10* | 0.07 | ND | >100* |
| L79H-F82K | ND | >100* | ND | 0.10 | ND | >100* |

ND: not detectable over concentration range tested
*estimate of the order of magnitude of the IC₅₀

As shown in Table 13 above as well as in FIGS. 16A and 16B, amino acid substitutions in the ActRIIB extracellular domain can alter the balance between ActRIIB:ligand binding and downstream signaling activities in various in vitro assays. In general, applicant achieved the goal of generating variants in the ActRIIB extracellular domain that exhibited decreased or non-detectable binding to BMP9, compared to a fusion polypeptide containing unmodified ActRIIB extracellular domain (ActRIIB-G1Fc), while retaining other ligand binding properties.

Additionally, variants ActRIIB (L79H-F82I), ActRIIB (L79H), and ActRIIB (L79H-F82K), while demonstrating a decrease in binding to BMP9, also exhibited a significant decrease in activin A binding while retaining relatively high affinity for activin B, as compared to ActRIIB-G1Fc. IC₅₀ values showing inhibitory potency in Table 13 are consistent with this ligand binding trend. Similarly, variants ActRIIB (F82K-N83R), ActRIIB (F82I-N83R), and ActRIIB (F82T-N83R) demonstrate a similar trend.

Furthermore, variants ActRIIB (F82K-N83R), ActRIIB (F82I-N83R), ActRIIB (F82T-N83R), and ActRIIB (L79H-F82K), while demonstrating a decrease in binding to BMP9 and retaining relatively high affinity for activin B, also exhibited a significant decrease in GDF8 and GDF11 binding, as compared to ActRIIB-G1Fc. IC₅₀ values showing inhibitory potency in Table 13 are consistent with this ligand binding trend.

It was further noted that, variants ActRIIB (L79H-F82I), ActRIIB (L79H), and ActRIIB (L79H-F82K), while demonstrating a decrease in binding to BMP9 and retaining relatively high affinity for activin B, also exhibited a decrease in BMP10 binding as compared to ActRIIB-G1Fc. IC₅₀ values showing inhibitory potency in Table 13 are consistent with this ligand binding trend.

Therefore, in addition to achieving the goal of producing ActRIIB variants that exhibit reduced to non-detectable binding to BMP9, Applicant has generated a diverse array of novel variant polypeptides, many of which are characterized in part by unique ligand binding/inhibition profiles. Accordingly, these variants may be more useful than ActRIIB-G1Fc in certain applications where such selective antagonism is advantageous. Examples include therapeutic applications where it is desirable to retain antagonism of activin B, while reducing antagonism of BMP9 and optionally one or more of activin A, GDF8, GDF11 and BMP10.

### Example 8. Generation of Variant ActRIIB-Fc Polypeptides

Applicants generated a series of mutations (sequence variations) in the extracellular domain of ActRIIB and produced these variant polypeptides as soluble homodimeric fusion polypeptides comprising a variant ActRIIB extracellular domain and an Fc domain joined by an optional linker. The background ActRIIB-Fc fusion was ActRIIB-G1Fc as shown in SEQ ID NO: 5.

Various substitution mutations were introduced into the background ActRIIB-Fc polypeptide. Based on the data presented in Example 4, it is expected that these constructs, if expressed with a TPA leader, will lack the N-terminal serine. Mutations were generated in the ActRIIB extracellular domain by PCR mutagenesis. After PCR, fragments were purified through a Qiagen column, digested with SfoI and AgeI and gel purified. These fragments were ligated into expression vector pAID4 (see WO2006/012627) such that upon ligation it created fusion chimera with human IgG1. Upon transformation into E. coli DH5 alpha, colonies were picked and DNA was isolated. For murine constructs (mFc), a murine IgG2a was substituted for the human IgG1. All mutants were sequence verified.

The amino acid sequence of unprocessed ActRIIB(K55A)-G1Fc is shown below (SEQ ID NO: 31). The signal sequence and linker sequence are indicated by solid underline, and the K55A substitution is indicated by double underline. The amino acid sequence of SEQ ID NO:31 may optionally be provided with the lysine removed from the C-terminus.

| (SEQ ID NO: 31) | |
|---|---|
| 1 | MDAMKRGLCC VLLLCGAVFV SPGASGRGEA ETRECIYYNA NWELERTNQS |
| 51 | GLERCEGEQD ARLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVESCSVMH |
| 351 | EALHNHYTQK SLSLSPGK |

This ActRIIB(K55A)-G1Fc fusion polypeptide is encoded by the following nucleic acid sequence (SEQ ID NO: 32):

| (SEQ ID NO: 32) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC GCCCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCCCCGGG |
| 1101 | TAAA |

The mature ActRIIB(K55A)-G1Fc fusion polypeptide (SEQ ID NO: 33) is as follows and may optionally be provided with the lysine removed from the C-terminus.

| (SEQ ID NO: 33) | |
|---|---|
| 1 | GRGEAETREC IYYNANWELE RTNQSGLERC EGEQDARLHC YASWRNSSGT |
| 51 | IELVKKGCWL DDENCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSE |
| 301 | FLYSKLTVDK SRWQQGNVFS CSVMHEALHN HYTQKSLSLS PGK |

The amino acid sequence of unprocessed ActRIIB(K55E)-G1Fc is shown below (SEQ ID NO: 34). The signal sequence and linker sequence are indicated by solid underline, and the K55E substitution is indicated by double underline. The amino acid sequence of SEQ ID NO:34 may optionally be provided with the lysine removed from the C-terminus.

| (SEQ ID NO: 34) | |
|---|---|
| 1 | MDAMKRGLCC VLLLCGAVFV SPGASGRGEA ETRECIYYNA NWELERTNQS |
| 51 | GLERCEGEQD ERLHCYASWR NSSGTIELVK KGCWLDDFNC YDRQECVATE |
| 101 | ENPQVYFCCC EGNECNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVESCSVMH |
| 351 | EALHNHYTQK SLSLSPGK |

This ActRIIB(K55E)-G1Fc fusion polypeptide is encoded by the following nucleic acid sequence (SEQ ID NO: 35):

| (SEQ ID NO: 35) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC GAGCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCCCCGGG |
| 1101 | TAAA |

The mature ActRIIB(K55E)-G1Fc fusion polypeptide (SEQ ID NO: 36) is as follows and may optionally be provided with the lysine removed from the C-terminus.

| (SEQ ID NO: 36) | |
|---|---|
| 1 | GRGEAETREC IYYNANWELE RTNQSGLERC EGEQDERLHC YASWRNSSGT |
| 51 | IELVKKGCWL DDENCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSE |
| 301 | FLYSKLTVDK SRWQQGNVES CSVMHEALHN HYTQKSLSLS PGK |

The amino acid sequence of unprocessed ActRIIB(F82I)-G1Fc is shown below (SEQ ID NO: 37). The signal sequence and linker sequence are indicated by solid underline, and the F82I substitution is indicated by double underline. The amino acid sequence of SEQ ID NO: 37 may optionally be provided with the lysine removed from the C-terminus.

| (SEQ ID NO: 37) | |
|---|---|
| 1 | MDAMKRGLCC VLLLCGAVEV SPGASGRGEA ETRECIYYNA NWELERTNQS |
| 51 | GLERCEGEQD KRLHCYASWR NSSGTIELVK KGCWLDDINC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVESCSVMH |
| 351 | EALHNHYTQK SLSLSPGK |

This ActRIIB(F82I)-G1Fc fusion polypeptide is encoded by the following nucleic acid sequence (SEQ ID NO: 38):

| (SEQ ID NO: 38) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CATCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAA |

The mature ActRIIB(F82I)-G1Fc fusion polypeptide (SEQ ID NO: 39) is as follows and may optionally be provided with the lysine removed from the C-terminus.

| (SEQ ID NO: 39) | |
|---|---|
| 1 | GRGEAETREC IYYNANWELE RTNQSGLERC EGEQDKRLHC YASWRNSSGT |
| 51 | IELVKKGCWL DDINCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVES CSVMHEALHN HYTQKSLSLS PGK |

The amino acid sequence of unprocessed ActRIIB(F82K)-G1Fc is shown below (SEQ ID NO: 40). The signal sequence and linker sequence are indicated by solid underline, and the F82K substitution is indicated by double underline. The amino acid sequence of SEQ ID NO: 40 may optionally be provided with the lysine removed from the C-terminus.

| (SEQ ID NO: 40) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDKNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVESCSVMH |
| 351 | EALHNHYTQK SLSLSPGK |

This ActRIIB(F82K)-G1Fc fusion polypeptide is encoded by the following nucleic acid sequence (SEQ ID NO: 41):

| (SEQ ID NO: 41) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CAAGAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAA |

The mature ActRIIB(F82K)-G1Fc fusion polypeptide (SEQ ID NO: 42) is as follows and may optionally be provided with the lysine removed from the C-terminus.

```
(SEQ ID NO: 42)
    1 GRGEAETREC IYYNANWELE RTNQSGLERC
        EGEQDKRLHC YASWRNSSGT
  51 IELVKKGCWL DDKNCYDRQE CVATEENPQV
        YFCCCEGNFC NERFTHLPEA
101 GGPEVTYEPP PTAPTGGGTH TCPPCPAPEL
        LGGPSVELFP PKPKDTLMIS
151 RTPEVTCVVV DVSHEDPEVK FNWYVDGVEV
        HNAKTKPREE QYNSTYRVVS
201 VLTVLHQDWL NGKEYKCKVS NKALPAPIEK
        TISKAKGQPR EPQVYTLPPS
251 REEMTKNQVS LTCLVKGFYP SDIAVEWESN
        GQPENNYKTT PPVLDSDGSF
301 FLYSKLTVDK SRWQQGNVES CSVMHEALHN
        HYTQKSLSLS PGK
```

Constructs were expressed in COS or CHO cells and purified by filtration and protein A chromatography. In some instances, assays were performed with conditioned medium rather than purified proteins. Purity of samples for reporter gene assays was evaluated by SDS-PAGE and Western blot analysis.

Mutants were tested in binding assays and/or bioassays described below.

Alternatively, similar mutations could be introduced into an ActRIIB extracellular domain possessing an N-terminal truncation of five amino acids and a C-terminal truncation of three amino acids as shown below (SEQ ID NO: 53). This truncated ActRIIB extracellular domain is denoted ActRIIB(25-131) based on numbering in SEQ ID NO: 2.

```
(SEQ ID NO: 53)
  25 ETRECIYYNA NWELERTNQS GLERCEGEQD
      KRLHCYASWR NSSGTIELVK
  75 KGCWLDDFNC YDRQECVATE ENPQVYFCCC
        EGNFCNERFT HLPEAGGPEV
125 TYEPPPT
```

The corresponding background fusion polypeptide, ActRIIB(25-131)-G1Fc, is shown below (SEQ ID NO: 12).

```
(SEQ ID NO: 12)
    1 ETRECIYYNA NWELERINQS GLERCEGEQD
        KRLHCYASWR NSSGTIELVK
  51 KGCWLDDENC YDRQECVATE ENPQVYFCCC
        EGNECNERFT HLPEAGGPEV
101 TYEPPPTGGG THTCPPCPAP ELLGGPSVEL
        FPPKPKDTLM ISRTPEVTCV
151 VVDVSHEDPE VKENWYVDGV EVHNAKTKPR
        EEQYNSTYRV VSVLTVLHQD
201 WLNGKEYKCK VSNKALPAPI EKTISKAKGQ
        PREPQVYTLP PSREEMTKNQ
251 VSLTCLVKGF YPSDIAVEWE SNGQPENNYK
        TTPPVLDSDG SFFLYSKLTV
301 DKSRWQQGNV FSCSVMHEAL HNHYTQKSLS
        LSPGK
```

### Example 9. Ligand Binding Profiles of Variant ActRIIB-Fc Homodimers and Activity of Variant ActRIIB-Fc Polypeptides in a Cell-Based Assay

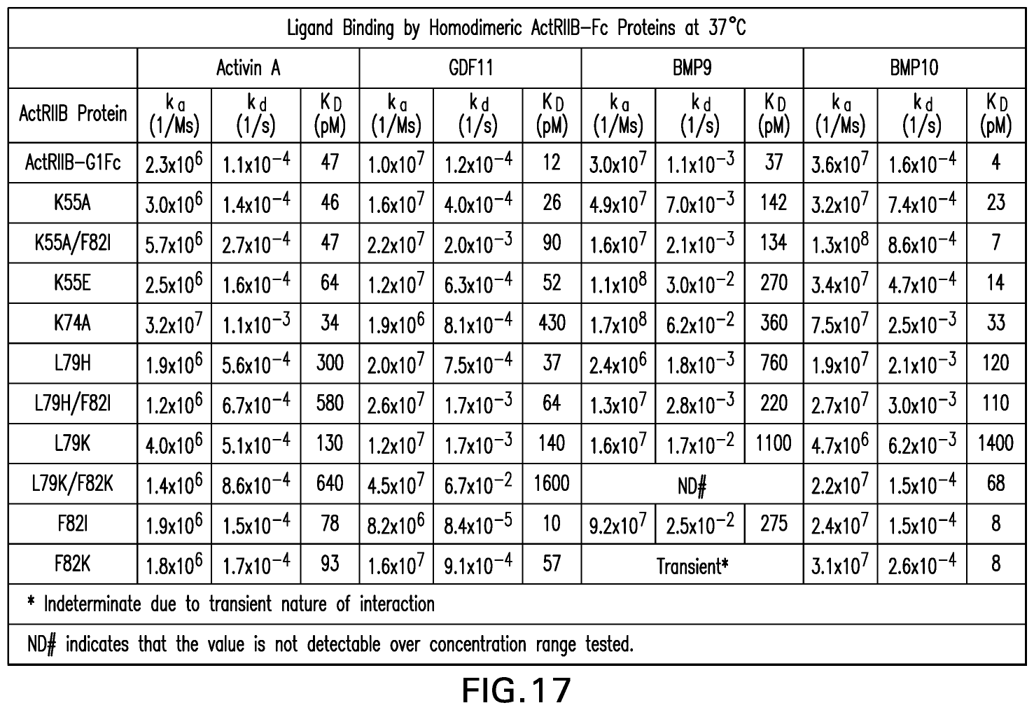

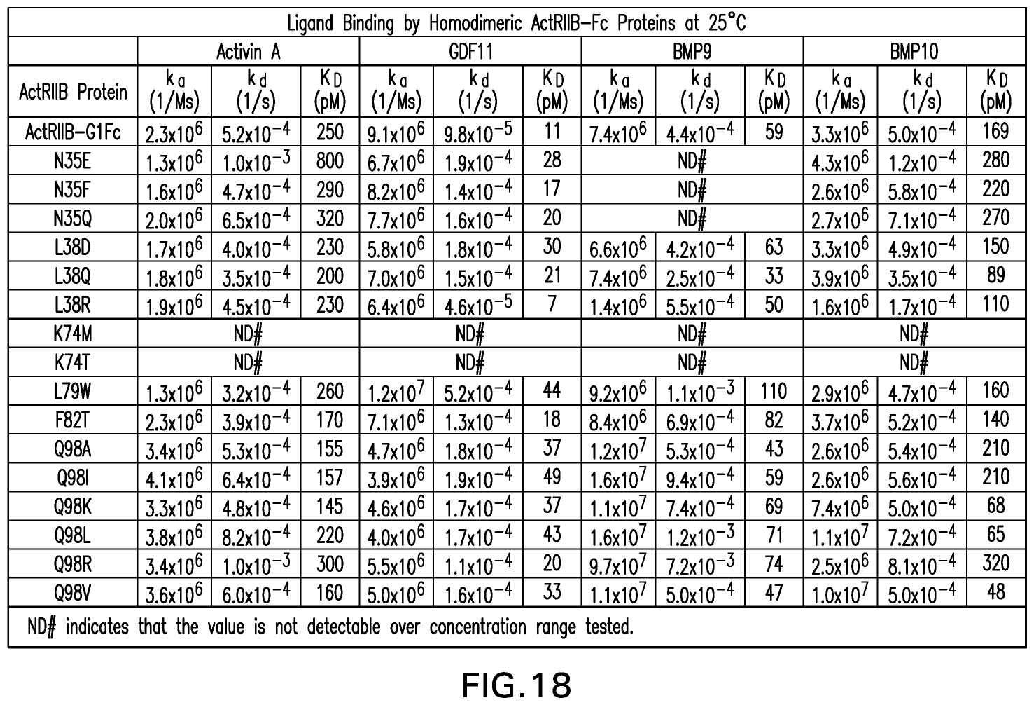

To determine ligand binding profiles of variant ActRIIB-Fc homodimers, a Biacore™_based binding assay was used to compare ligand binding kinetics of certain variant ActRIIB-Fc polypeptides. ActRIIB-Fc polypeptides to be tested were independently captured onto the system using an anti-Fc antibody. Ligands were then injected and allowed to flow over the captured receptor protein. Results of variant ActRIIB-Fc polypeptides analyzed at 37° C. are shown in FIG. 17. Compared to Fc-fusion polypeptide comprising unmodified ActRIIB extracellular domain, the variant polypeptides ActRIIB(K55A)-Fc, ActRIIB(K55E)-Fc, ActRIIB(F82I)-Fc, and ActRIIB(F82K)-Fc exhibited greater reduction in their affinity for BMP9 than for GDF11. Results of additional variant ActRIIB-Fc polypeptides analyzed at 25° C. are shown in FIG. 18.

These results confirm K55A, K55E, F82I, and F82K as substitutions that reduce ActRIIB binding affinity for BMP9 more than they reduce ActRIIB affinity for activin A or GDF11. Accordingly, these variant ActRIIB-Fc polypeptides may be more useful than unmodified ActRIIB-Fc polypeptide in certain applications where such selective antagonism is advantageous. Examples include therapeutic applications where it is desirable to retain antagonism of one or more of activin A, activin B, GDF8, and GDF11 while reducing antagonism of BMP9.

To determine activity of variant ActRIIB-Fc polypeptides, an A204 cell-based assay was used to compare effects among variant ActRIIB-Fc polypeptides on signaling by activin A, GDF11, and BMP9. In brief, this assay uses a human A204 rhabdomyosarcoma cell line (ATCC®: HTB-82™) derived from muscle and the reporter vector pGL3(CAGA)12 (Dennler et al., 1998, EMBO 17: 3091-3100) as well as a Renilla reporter plasmid (pRLCMV) to control for transfection efficiency. The CAGA12 motif is present in TGF-β responsive genes (e.g., PAI-1 gene), so this vector is of general use for ligands that can signal through Smad2/3, including activin A, GDF11, and BMP9.

On day 1, A-204 cells were transferred into one or more 48-well plates. On day 2, these cells were transfected with 10 pg pGL3(CAGA)12 or pGL3(CAGA)12(10 pg)+pRLCMV (1 pg) and Fugene. On day 3, ligands diluted in medium containing 0.1% BSA were preincubated with ActRIIB-Fc polypeptides for 1 hr before addition to cells. Approximately six hour later, the cells were rinsed with PBS and lysed. Cell lysates were analyzed in a luciferase assay to determine the extent of Smad activation.

This assay was used to screen variant ActRIIB-Fc polypeptides for inhibitory effects on cell signaling by activin A, GDF11, and BMP9. Potencies of homodimeric Fc fusion polypeptides incorporating amino acid substitutions in the human ActRIIB extracellular domain were compared with that of an Fc fusion polypeptide comprising unmodified human ActRIIB extracellular domain.

TABLE 14
Inhibitory Potency of Homodimeric ActRIIB-Fc Constructs

| ActRIIB | IC₅₀ (ng/mL) | | |
|---|---|---|---|
| polypeptide | Activin A | GDF11 | BMP9 |
| Wild-type | 8 | 9 | 31 |
| A24N | 128 | 99 | 409 |
| R40A | — | 591 | 1210 |
| E50K | 132 | 180 | 721 |
| E50P | 756 | 638 | ~3000 |
| E52A | 198 | 71 | 359 |
| E52K | 762 | 296 | ~10000 |
| K55A | 15 | 11 | 122 |
| K55D | 396 | 365 | 5500 |
| K55E | 19 | 14 | 290 |
| K55R | 206 | 318 | 777 |
| Y60K | — | 414 | ND |
| Y60P | — | 544 | ND |
| K74R | — | 45 | 165 |
| K74Y | — | ND | ND |
| K74A/L79P | — | ND | ND |
| L79K | — | 477 | ND |
| L79P | — | ND | ND |
| L79R | — | 234 | ND |
| D80A | — | ND | ND |
| F82I | 11 | 9 | 277 |
| F82K | 10 | 15 | ~5000 |
| F82W | — | 276 | ND |
| F82W/N83A | — | 389 | ~40000 |
| V99E | — | ND | ND |
| V99K | — | ND | — |

ND: not detectable over concentration range tested
— Not tested

As shown in the table above, single amino acid substitutions in the ActRIIB extracellular domain can alter the balance between activin A or GDF11 inhibition and BMP9 inhibition in a cell-based reporter gene assay. Compared to a fusion polypeptide containing unmodified ActRIIB extracellular domain, the variants ActRIIB(K55A)-Fc, ActRIIB(K55E)-Fc, ActRIIB(F82I)-Fc, and ActRIIB(F82K)-Fc showed less potent inhibition of BMP9 (increased IC₅₀ values) while maintaining essentially undiminished inhibition of activin A and GDF11.

These results indicate that variant ActRIIB-Fc polypeptides such as ActRIIB(K55A)-Fc, ActRIIB(K55E)-Fc, ActRIIB(F82I)-Fc, and ActRIIB(F82K)-Fc are more selective antagonists of activin A and GDF11 compared to an Fc fusion polypeptide comprising unmodified ActRIIB extracellular domain. Accordingly, these variants may be more useful than ActRIIB-Fc in certain applications where such selective antagonism is advantageous. Examples include therapeutic applications where it is desirable to retain antagonism of one or more of activin A, GDF8, and GDF11 while reducing antagonism of BMP9 and potentially BMP10.

### Example 10. Generation of an ActRIIB-Fc:ActRIIB(L79E)-Fc Heterodimer

Applicants envision generation of a soluble ActRIIB-Fc:ActRIIB(L79E)-Fc heteromeric complex comprising the extracellular domains of unmodified human ActRIIB and human ActRIIB with a leucine-to-glutamate substitution at position 79, which are each separately fused to an G1Fc domain with a linker positioned between the extracellular domain and the G1Fc domain. The individual constructs are referred to as ActRIIB-Fc fusion polypeptide and ActRIIB(L79E)-Fc fusion polypeptide, respectively, and the sequences for each are provided below.

A methodology for promoting formation of ActRIIB-Fc:ActRIIB(L79E)-Fc heteromeric complexes, as opposed to the ActRIIB-Fc or ActRIIB(L79E)-Fc homodimeric complexes, is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the ActRIIB(L79E)-Fc and ActRIIB-Fc polypeptide sequences of SEQ ID NOs: 43-45 and 46-48, respectively, one Fc domain can be altered to introduce cationic amino acids at the interaction face, while the other Fc domain can be altered to introduce anionic amino acids at the interaction face. The ActRIIB(L79E)-Fc fusion polypeptide and ActRIIB-Fc fusion polypeptide can each employ the TPA leader (SEQ ID NO: 8).

The ActRIIB(L79E)-Fc polypeptide sequence (SEQ ID NO: 43) is shown below:

```
(SEQ ID NO: 43)
    1 MDAMKRGLCC VLLLCGAVEV SPGASGRGEA
        ETRECIYYNA NWELERTNQS
  51 GLERCEGEQD KRLHCYASWR NSSGTIELVK
        KGCWEDDENC YDRQECVATE
101 ENPQVYFCCC EGNFCNERFT HLPEAGGPEV
        TYEPPPTAPT GGGTHTCPPC
151 PAPELLGGPS VFLFPPKPKD TLMISRTPEV
        TCVVVDVSHE DPEVKENWYV
201 DGVEVHNAKT KPREEQYNST YRVVSVLTVL
        HQDWINGKEY KCKVSNKALP
251 APIEKTISKA KGQPREPQVY TLPPSREEMT
        KNQVSLTCLV KGFYPSDIAV
301 EWESNGQPEN NYDTTPPVLD SDGSFFLYSD
        LTVDKSRWQQ GNVESCSVMH
351 EALHNHYTQK SLSLSPG
```

The leader (signal) sequence and linker are underlined, and the L79E substitution is indicated by double underline. To promote formation of the ActRIIB-Fc:ActRIIB(L79E)-Fc heterodimer rather than either of the possible homodimeric complexes, two amino acid substitutions (replacing lysines with acidic amino acids) can be introduced into the Fc domain of the ActRIIB fusion polypeptide as indicated by double underline above. The amino acid sequence of SEQ ID NO: 43 may optionally be provided with lysine added to the C-terminus.

This ActRIIB(L79E)-Fc fusion polypeptide can be encoded by the following nucleic acid sequence (SEQ ID NO: 44):

(SEQ ID NO: 44)

| | |
|---|---|
| 1 | ATGGATGCAA TGAAGAGAGG GCTCTGCTGT |
| | GTGCTGCTGC TGTGTGGAGC |
| 51 | AGTCTTCGTT TCGCCCGGCG CCTCTGGGCG |
| | TGGGGAGGCT GAGACACGGG |
| 101 | AGTGCATCTA CTACAACGCC AACTGGGAGC |
| | TGGAGCGCAC CAACCAGAGC |
| 151 | GGCCTGGAGC GCTGCGAAGG CGAGCAGGAC |
| | AAGCGGCTGC ACTGCTACGC |
| 201 | CTCCTGGCGC AACAGCTCTG GCACCATCGA |
| | GCTCGTGAAG AAGGGCTGCT |
| 251 | GGGAAGATGA CTTCAACTGC TACGATAGGC |
| | AGGAGTGTGT GGCCACTGAG |
| 301 | GAGAACCCCC AGGTGTACTT CTGCTGCTGT |
| | GAAGGCAACT TCTGCAACGA |
| 351 | GCGCTTCACT CATTTGCCAG AGGCTGGGGG |
| | CCCGGAAGTC ACGTACGAGC |
| 401 | CACCCCCGAC AGCCCCCACC GGTGGTGGAA |
| | CTCACACATG CCCACCGTGC |
| 451 | CCAGCACCTG AACTCCTGGG GGGACCGTCA |
| | GTCTTCCTCT TCCCCCCAAA |
| 501 | ACCCAAGGAC ACCCTCATGA TCTCCCGGAC |
| | CCCTGAGGTC ACATGCGTGG |
| 551 | TGGTGGACGT GAGCCACGAA GACCCTGAGG |
| | TCAAGTTCAA CTGGTACGTG |
| 601 | GACGGCGTGG AGGTGCATAA TGCCAAGACA |
| | AAGCCGCGGG AGGAGCAGTA |
| 651 | CAACAGCACG TACCGTGTGG TCAGCGTCCT |
| | CACCGTCCTG CACCAGGACT |
| 701 | GGCTGAATGG CAAGGAGTAC AAGTGCAAGG |
| | TCTCCAACAA AGCCCTCCCA |
| 751 | GCCCCCATCG AGAAAACCAT CTCCAAAGCC |
| | AAAGGGCAGC CCCGAGAACC |
| 801 | ACAGGTGTAC ACCCTGCCCC CATCCCGGGA |
| | GGAGATGACC AAGAACCAGG |
| 851 | TCAGCCTGAC CTGCCTGGTC AAAGGCTTCT |
| | ATCCCAGCGA CATCGCCGTG |
| 901 | GAGTGGGAGA GCAATGGGCA GCCGGAGAAC |
| | AACTACGACA CCACGCCTCC |
| 951 | CGTGCTGGAC TCCGACGGCT CCTTCTTCCT |
| | CTATAGCGAC CTCACCGTGG |
| 1001 | ACAAGAGCAG GTGGCAGCAG GGGAACGTCT |
| | TCTCATGCTC CGTGATGCAT |
| 1051 | GAGGCTCTGC ACAACCACTA CACGCAGAAG |
| | AGCCTCTCCC TGTCTCCGGG |
| 1101 | T |

The mature ActRIIB(L79E)-Fc fusion polypeptide (SEQ ID NO: 45) is as follows, and may optionally be provided with lysine added to the C-terminus.

```
(SEQ ID NO: 45)
    1 GRGEAETREC IYYNANWELE RTNQSGLERC
        EGEQDKRLHC YASWRNSSGT
  51 IELVKKGCWE DDENCYDRQE CVATEENPQV
        YFCCCEGNFC NERFTHLPEA
101 GGPEVTYEPP PTAPTGGGTH TCPPCPAPEL
        LGGPSVELFP PKPKDTLMIS
151 RTPEVTCVVV DVSHEDPEVK FNWYVDGVEV
        HNAKTKPREE QYNSTYRVVS
201 VLTVLHQDWL NGKEYKCKVS NKALPAPIEK
        TISKAKGQPR EPQVYTLPPS
251 REEMTKNQVS LTCLVKGFYP SDIAVEWESN
        GQPENNYDTT PPVLDSDGSE
301 FLYSDLTVDK SRWQQGNVES CSVMHEALHN
        HYTQKSLSLS PG
```

The complementary form of ActRIIB-Fc fusion polypeptide (SEQ ID NO: 46) is as follows:

```
(SEQ ID NO: 46)
    1 MDAMKRGLCC VLLLCGAVEV SPGASGRGEA
        ETRECIYYNA NWELERTNQS
  51 GLERCEGEQD KRLHCYASWR NSSGTIELVK
        KGCWLDDENC YDRQECVATE
101 ENPQVYFCCC EGNFCNERFT HLPEAGGPEV
        TYEPPPTAPT GGGTHTCPPC
151 PAPELLGGPS VFLFPPKPKD TLMISRTPEV
        TCVVVDVSHE DPEVKENWYV
201 DGVEVHNAKT KPREEQYNST YRVVSVLTVL
        HQDWLNGKEY KCKVSNKALP
251 APIEKTISKA KGQPREPQVY TLPPSRKEMT
        KNQVSLTCLV KGFYPSDIAV
301 EWESNGQPEN NYKTTPPVLK SDGSFFLYSK
        LTVDKSRWQQ GNVFSCSVMH
351 EALHNHYTQK SLSLSPGK
```

The leader sequence and linker sequence are underlined. To guide heterodimer formation with the ActRIIB(L79E)-Fc fusion polypeptide of SEQ ID NOs: 43 and 45 above, two amino acid substitutions (replacing a glutamate and an aspartate with lysines) can be introduced into the Fc domain of the ActRIIB-Fc fusion polypeptide as indicated by double underline above. The amino acid sequence of SEQ ID NO: 46 may optionally be provided with lysine removed from the C-terminus.

This ActRIIB-Fc fusion polypeptide can be encoded by the following nucleic acid (SEQ ID NO: 47):

(SEQ ID NO: 47)

| | |
|---|---|
| 1 | ATGGATGCAA TGAAGAGAGG GCTCTGCTGT |
| | GTGCTGCTGC TGTGTGGAGC |
| 51 | AGTCTTCGTT TCGCCCGGCG CCTCTGGGCG |
| | TGGGGAGGCT GAGACACGGG |
| 101 | AGTGCATCTA CTACAACGCC AACTGGGAGC |
| | TGGAGCGCAC CAACCAGAGC |
| 151 | GGCCTGGAGC GCTGCGAAGG CGAGCAGGAC |
| | AAGCGGCTGC ACTGCTACGC |
| 201 | CTCCTGGCGC AACAGCTCTG GCACCATCGA |
| | GCTCGTGAAG AAGGGCTGCT |
| 251 | GGCTAGATGA CTTCAACTGC TACGATAGGC |
| | AGGAGTGTGT GGCCACTGAG |
| 301 | GAGAACCCCC AGGTGTACTT CTGCTGCTGT |
| | GAAGGCAACT TCTGCAACGA |
| 351 | GCGCTTCACT CATTTGCCAG AGGCTGGGGG |
| | CCCGGAAGTC ACGTACGAGC |
| 401 | CACCCCCGAC AGCCCCCACC GGTGGTGGAA |
| | CTCACACATG CCCACCGTGC |
| 451 | CCAGCACCTG AACTCCTGGG GGGACCGTCA |
| | GTCTTCCTCT TCCCCCCAAA |
| 501 | ACCCAAGGAC ACCCTCATGA TCTCCCGGAC |
| | CCCTGAGGTC ACATGCGTGG |
| 551 | TGGTGGACGT GAGCCACGAA GACCCTGAGG |
| | TCAAGTTCAA CTGGTACGTG |
| 601 | GACGGCGTGG AGGTGCATAA TGCCAAGACA |
| | AAGCCGCGGG AGGAGCAGTA |
| 651 | CAACAGCACG TACCGTGTGG TCAGCGTCCT |
| | CACCGTCCTG CACCAGGACT |
| 701 | GGCTGAATGG CAAGGAGTAC AAGTGCAAGG |
| | TCTCCAACAA AGCCCTCCCA |
| 751 | GCCCCCATCG AGAAAACCAT CTCCAAAGCC |
| | AAAGGGCAGC CCCGAGAACC |
| 801 | ACAGGTGTAC ACCCTGCCCC CATCCCGGAA |
| | GGAGATGACC AAGAACCAGG |
| 851 | TCAGCCTGAC CTGCCTGGTC AAAGGCTTCT |
| | ATCCCAGCGA CATCGCCGTG |
| 901 | GAGTGGGAGA GCAATGGGCA GCCGGAGAAC |
| | AACTACAAGA CCACGCCTCC |
| 951 | CGTGCTGAAG TCCGACGGCT CCTTCTTCCT |
| | CTATAGCAAG CTCACCGTGG |
| 1001 | ACAAGAGCAG GTGGCAGCAG GGGAACGTCT |
| | TCTCATGCTC CGTGATGCAT |
| 1051 | GAGGCTCTGC ACAACCACTA CACGCAGAAG |
| | AGCCTCTCCC TGTCTCCGGG |
| 1101 | TAAA |

The mature ActRIIB-Fc fusion polypeptide sequence (SEQ ID NO: 48) is as follows and may optionally be provided with lysine removed from the C-terminus:

| (SEQ ID NO: 48) | |
|---|---|
| 1 | GRGEAETREC IYYNANWELE RTNQSGLERC |
| | EGEQDKRLHC YASWRNSSGT |
| 51 | IELVKKGCWL DDENCYDRQE CVATEENPQV |
| | YFCCCEGNEC NERFTHLPEA |
| 101 | GGPEVTYEPP PTAPTGGGTH TCPPCPAPEL |
| | LGGPSVELEP PKPKDTLMIS |
| 151 | RTPEVTCVVV DVSHEDPEVK FNWYVDGVEV |
| | HNAKTKPREE QYNSTYRVVS |
| 201 | VLTVLHQDWL NGKEYKCKVS NKALPAPIEK |
| | TISKAKGQPR EPQVYTLPPS |
| 251 | RKEMTKNQVS LTCLVKGFYP SDIAVEWESN |
| | GQPENNYKTT PPVLKSDGSF |
| 301 | FLYSKLTVDK SRWQQGNVES CSVMHEALHN |
| | HYTQKSLSLS PGK |

The ActRIIB(L79E)-Fc and ActRIIB-Fc polypeptides of SEQ ID NO: 45 and SEQ ID NO: 48, respectively, may be co-expressed and purified from a CHO cell line, to give rise to a heteromeric polypeptide complex comprising ActRIIB-Fc:ActRIIB(L79E)-Fc.

In another approach to promote the formation of heteromultimer complexes using asymmetric Fc fusion polypeptides, the Fc domains can be altered to introduce complementary hydrophobic interactions and an additional intermolecular disulfide bond as illustrated in the ActRIIB(L79E)-Fc and ActRIIB-Fc polypeptide sequences of SEQ ID NOs: 49-50 and 51-52, respectively. The ActRIIB(L79E)-Fc fusion polypeptide and ActRIIB-Fc fusion polypeptide can each employ the TPA leader (SEQ ID NO: 8). ActRIIB(L79E)-Fc polypeptide sequence (SEQ ID NO: 49) is shown below:

```
(SEQ ID NO: 49)
    1 MDAMKRGLCC VLLLCGAVEV SPGASGRGEA
        ETRECIYYNA NWELERTNQS
  51 GLERCEGEQD KRLHCYASWR NSSGTIELVK
        KGCWEDDENC YDRQECVATE
101 ENPQVYFCCC EGNFCNERFT HLPEAGGPEV
        TYEPPPTAPT GGGTHTCPPC
151 PAPELLGGPS VFLFPPKPKD TLMISRTPEV
        TCVVVDVSHE DPEVKENWYV
201 DGVEVHNAKT KPREEQYNST YRVVSVLTVL
        HQDWLNGKEY KCKVSNKALP
251 APIEKTISKA KGQPREPQVY TLPPCREEMT
        KNQVSLWCLV KGFYPSDIAV
301 EWESNGQPEN NYKTTPPVLD SDGSFFLYSK
        LTVDKSRWQQ GNVESCSVMH
351 EALHNHYTQK SLSLSPG
```

The signal sequence and linker sequence are underlined, and the L79E substitution is indicated by double underline. To promote formation of the ActRIIB-Fc:ActRIIB(L79E)-Fc heterodimer rather than either of the possible homodimeric complexes, two amino acid substitutions (replacing a serine with a cysteine and a threonine with a tryptophan) can be introduced into the Fc domain of the fusion polypeptide as indicated by double underline above. The amino acid sequence of SEQ ID NO: 49 may optionally be provided with lysine added to the C-terminus. Mature ActRIIB(L79E)-Fc fusion polypeptide (SEQ ID NO: 50) is as follows:

| (SEQ ID NO: 50) | |
|---|---|
| 1 | GRGEAETREC IYYNANWELE RTNQSGLERC |
| | EGEQDKRLHC YASWRNSSGT |
| 51 | IELVKKGCWE DDENCYDRQE CVATEENPQV |
| | YFCCCEGNFC NERFTHLPEA |
| 101 | GGPEVTYEPP PTAPTGGGTH TCPPCPAPEL |
| | LGGPSVELFP PKPKDTLMIS |
| 151 | RTPEVTCVVV DVSHEDPEVK FNWYVDGVEV |
| | HNAKTKPREE QYNSTYRVVS |
| 201 | VLTVLHQDWL NGKEYKCKVS NKALPAPIEK |
| | TISKAKGQPR EPQVYTLPPC |
| 251 | REEMTKNQVS LWCLVKGFYP SDIAVEWESN |
| | GQPENNYKTT PPVLDSDGSE |
| 301 | FLYSKLTVDK SRWQQGNVES CSVMHEALHN |
| | HYTQKSLSLS PG |

The complementary form of ActRIIB-Fc fusion polypeptide (SEQ ID NO: 51) is as follows and may optionally be provided with lysine removed from the C-terminus.

```
(SEQ ID NO: 51)
    1 MDAMKRGLCC VLLLCGAVEV SPGASGRGEA
        ETRECIYYNA NWELERTNQS
  51 GLERCEGEQD KRLHCYASWR NSSGTIELVK
        KGCWLDDENC YDRQECVATE
101 ENPQVYFCCC EGNFCNERFT HLPEAGGPEV
        TYEPPPTAPT GGGTHTCPPC
151 PAPELLGGPS VFLFPPKPKD TLMISRTPEV
        TCVVVDVSHE DPEVKENWYV
201 DGVEVHNAKT KPREEQYNST YRVVSVLTVL
        HQDWLNGKEY KCKVSNKALP
251 APIEKTISKA KGQPREPQVC TLPPSREEMT
        KNQVSLSCAV KGFYPSDIAV
301 EWESNGQPEN NYKTTPPVLD SDGSFFLVSK
        LTVDKSRWQQ GNVESCSVMH
351 EALHNHYTQK SLSLSPGK
```

The leader sequence and linker are underlined. To guide heterodimer formation with the ActRIIB(L79E)-Fc fusion polypeptide of SEQ ID NOs: 49-50 above, four amino acid substitutions (replacement of tyrosine with cysteine, threonine with serine, leucine with alanine, and tyrosine with valine) can be introduced into the Fc domain of the ActRIIB-Fc fusion polypeptide as indicated by double underline above. The amino acid sequence of SEQ ID NO: 51 may optionally be provided with lysine removed from the C-terminus.

The mature ActRIIB-Fc fusion polypeptide sequence is as follows and may optionally be provided with lysine removed from the C-terminus.

```
(SEQ ID NO: 52)
    1 GRGEAETREC IYYNANWELE RTNQSGLERC
        EGEQDKRLHC YASWRNSSGT
  51 IELVKKGCWL DDENCYDRQE CVATEENPQVY
        FCCCEGNFC NERFTHLPEA
101 GGPEVTYEPP PTAPTGGGTH TCPPCPAPEL
        LGGPSVELFP PKPKDTLMIS
151 RTPEVTCVVV DVSHEDPEVK FNWYVDGVEV
        HNAKTKPREE QYNSTYRVVS
201 VLTVLHQDWL NGKEYKCKVS NKALPAPIEK
        TISKAKGQPR EPQVCTLPPS
251 REEMTKNQVS LSCAVKGFYP SDIAVEWESN
        GQPENNYKTT PPVLDSDGSF
301 FLVSKLTVDK SRWQQGNVES
        CSVMHEALHN HYTQKSLSLS PGK
```

The ActRIIB(L79E)-Fc and ActRIIB-Fc polypeptides of SEQ ID NO: 50 and SEQ ID NO: 52, respectively, may be co-expressed and purified from a CHO cell line, to give rise to a heteromeric polypeptide complex comprising ActRIIB-Fc:ActRIIB(L79E)-Fc.

Purification of various ActRIIB-Fc:ActRIIB(L79E)-Fc complexes can be achieved by a series of column chromatography steps, including, for example, three or more of the following, in any order: protein A chromatography, Q sepharose chromatography, phenylsepharose chromatography, size exclusion chromatography, cation exchange chromatography, multimodal chromatography (e.g., with resin containing both electrostatic and hydrophobic ligands), and epitope-based affinity chromatography (e.g., with an antibody or functionally equivalent ligand directed against an epitope of ActRIIB). The purification can be completed with viral filtration and buffer exchange.

### Example 11. Ligand Binding Profile of ActRIIB-Fc:ActRIIB(L79E)-Fc Heteromer

A Biacore™_based binding assay was used to compare the ligand binding kinetics of an ActRIIB-Fc:ActRIIB(L79E)-Fc heterodimer with those of unmodified ActRIIB-Fc homodimer. Fusion proteins were captured onto the system using an anti-Fc antibody. Ligands were then injected and allowed to flow over the captured receptor protein at 37° C. Results are summarized in the table below, in which ligand off-rates (k_{d}) most indicative of effective ligand traps are denoted in bold.

TABLE 15
Ligand binding of ActRIIB-Fc:ActRIIB(L79E)-Fc heterodimer
compared to ActRII-Fc homodimer at 37° C.

| | ActRIIB-Fc | | | ActRIIB-Fc:ActRIIB(L79E)-Fc | | |
|---|---|---|---|---|---|---|
| | homodimer | | | heterodimer | | |
| | kₐ | k_{d} | K_{D} | kₐ | k_{d} | K_{D} |
| Ligand | (1/Ms) | (1/s) | (pM) | (1/Ms) | (1/s) | (pM) |
| Activin | 7.4 × 10⁶ | 1.9 × 10⁻⁴ | 25 | 8.8 × 10⁶ | 1.5 × 10⁻³ | 170 |
| A | | | | | | |
| Activin | 8.1 × 10⁶ | 6.6 × 10⁻⁵ | 8 | 8.3 × 10⁶ | 2.1 × 10⁻⁴ | 25 |
| B | | | | | | |
| GDF3 | 1.4 × 10⁶ | 2.2 × 10⁻³ | 1500 | 5.8 × 10⁵ | 5.9 × 10⁻³ | 10000 |
| GDF8 | 3.8 × 10⁶ | 2.6 × 10⁻⁴ | 70 | 3.4 × 10⁶ | 5.0 × 10⁻⁴ | 150 |
| GDF11 | 4.1 × 10⁷ | 1.7 × 10⁻⁴ | 4 | 4.0 × 10⁷ | 3.6 × 10⁻⁴ | 9 |
| BMP6 | 1.3 × 10⁸ | 7.4 × 10⁻³ | 56 | 3.3 × 10⁸ | 1.8 × 10⁻² | 56 |
| BMP9 | 5.0 × 10⁶ | 1.3 × 10⁻³ | 250 | Transient* | | >2800 |
| BMP10 | 5.1 × 10⁷ | 2.0 × 10⁻⁴ | 4 | 4.8 × 10⁷ | 2.0 × 10⁻³ | 42 |

*Indeterminate due to transient nature of interaction

In this example, a single amino acid substitution in one of two ActRIIB polypeptide chains altered ligand binding selectivity of the Fc-fusion polypeptide relative to unmodified ActRIIB-Fc homodimer. Compared to ActRIIB-Fc homodimer, the ActRIIB(L79E)-Fc heterodimer largely retained high-affinity binding to activin B, GDF8, GDF11, and BMP6 but exhibited approximately ten-fold faster off-rates for activin A and BMP10 and an even greater reduction in the strength of binding to BMP9. Accordingly, a variant ActRIIB-Fc heteromer may be more useful than unmodified ActRIIB-Fc homodimer in certain applications where such selective antagonism is advantageous. Examples include therapeutic applications where it is desirable to retain antagonism of one or more of activin B, GDF8, GDF11, and BMP6, while reducing antagonism of activin A, BMP9, or BMP10.9.

### Generation of ActRIIB Mutants:

A series of mutations in the extracellular domain of ActRIIB were generated and these mutant polypeptides were produced as soluble fusion polypeptides between extracellular ActRIIB and an Fc domain. A co-crystal structure of Activin and extracellular ActRIIB did not show any role for the final (C-terminal) 15 amino acids (referred to as the “tail” herein) of the extracellular domain in ligand binding. This sequence failed to resolve on the crystal structure, suggesting that these residues are present in a flexible loop that did not pack uniformly in the crystal. Thompson EMBO J. 2003 Apr. 1; 22(7):1555-66. This sequence is also poorly conserved between ActRIIB and ActRIIA. Accordingly, these residues were omitted in the basic, or background, ActRIIB-Fc fusion construct. Additionally, in this example position 64 in the background form is occupied by an alanine. Thus, the background ActRIIB-Fc fusion in this example has the sequence (Fc portion underlined)(SEQ ID NO: 54):

SGRGEAETRECIYYNANWELERTNQSGLERCEGEQDKRLHCYASW
ANSSGTIELVKKGCWLDDFNCYDRQECVATEENPQVYFCCCEGNF
CNERFTHLPEAGGGTHTCPPCPAPELLGGPSVFLFPPKPKDTLMI
SRTPEVTCVVVDVSHEDPEVKFNWYVDGVEVHNAKTKPREEQYNS
TYRVVSVLTVLHQDWLNGKEYKCKVSNKALPVPIEKTISKAKGQP
REPQVYTLPPSREEMTKNQVSLTCLVKGFYPSDIAVEWESNGQPE
NNYKTTPPVLDSDGSFFLYSKLTVDKSRWQQGNVFSCSVMHEALH
NHYTQKSLSLSPGK

Surprisingly, as discussed below, the C-terminal tail was found to enhance activin and GDF-11 binding, thus a preferred version of ActRIIB-Fc has a sequence (Fc portion underlined)(SEQ ID NO: 55):

SGRGEAETRECIYYNANWELERTNQSGLERCEGEQDKRLHCYASW
ANSSGTIELVKKGCWLDDFNCYDRQECVATEENPQVYFCCCEGNF
CNERFTHLPEAGGPEVTYEPPPTAPTGGGTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PVPIEKTISKAKGQPREPQVYTLPPSREEMTKNQVSLTCLVKGFY
PSDIAVEWESNGQPENNYKTTPPVLDSDGSFFLYSKLTVDKSRWQ
QGNVFSCSVMHEALHNHYTQKSLSLSPGK

Various mutations were introduced into the background ActRIIB-Fc polypeptide. Mutations were generated in ActRIIB extracellular domain by PCR mutagenesis. After PCR, fragments were purified thru Qiagen column, digested with SfoI and AgeI and gel purified. These fragments were ligated into expression vector pAID4 such that upon ligation it created fusion chimera with human IgG1. DNAs were isolated. All of the mutants were produced in HEK293T cells by transient transfection. In summary, in a 500 ml spinner, HEK293T cells were set up at 6×10⁵ cells/ml in Freestyle (Invitrogen) media in 250 ml volume and grown overnight. Next day, these cells were treated with DNA:PEI (1:1) complex at 0.5 ug/ml final DNA concentration. After 4 hrs, 250 ml media was added and cells were grown for 7 days. Conditioned media was harvested by spinning down the cells and concentrated.

All the mutants were purified over protein A column and eluted with low pH (3.0) glycine buffer. After neutralization, these were dialyzed against PBS.

Mutants were also produced in CHO cells by similar methodology.

Mutants were tested in binding assays and bioassays described below. Proteins expressed in CHO cells and HEK293 cells were indistinguishable in the binding assays and bioassays.

### Example 12: Generation of an ActRIIB-ALK4 heterodimer

An ActRIIB-Fc:ALK4-Fc heteromeric complex was constructed comprising the extracellular domains of human ActRIIB and human ALK4, which are each separately fused to an Fc domain with a linker positioned between the extracellular domain and the Fc domain. The individual constructs are referred to as ActRIIB-Fc fusion polypeptide and ALK4-Fc fusion polypeptide, respectively, and the sequences for each are provided below.

A methodology for promoting formation of ActRIIB-Fc:ALK4-Fc heteromeric complexes, as opposed to ActRIIB-Fc or ALK4-Fc homodimeric complexes, is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the ActRIIB-Fc and ALK4-Fc polypeptide sequences of SEQ ID NOs: 396 and 398 and SEQ ID Nos: 88 and 89, respectively, one Fc domain is altered to introduce cationic amino acids at the interaction face, while the other Fc domain is altered to introduce anionic amino acids at the interaction face. ActRIIB-Fc fusion polypeptide and ALK4-Fc fusion polypeptide each employ the tissue plasminogen activator (TPA) leader.

The ActRIIB-Fc polypeptide sequence (SEQ ID NO: 396) is shown below:

| (SEQ ID NO: 396) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDENC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSRKEMT KNQVSLTCLV KGFYPSDIAV |
| 301 | EWESNGQPEN NYKTTPPVLK SDGSFFLYSK LTVDKSRWQQ GNVESCSVMH |
| 351 | EALHNHYTQK SLSLSPGK |

The leader (signal) sequence and linker are underlined. To promote formation of ActRIIB-Fc:ALK4-Fc heterodimer rather than either of the possible homodimeric complexes, two amino acid substitutions (replacing acidic amino acids with lysine) can be introduced into the Fc domain of the ActRIIB fusion protein as indicated by double underline above. The amino acid sequence of SEQ ID NO: 396 may optionally be provided with lysine (K) removed from the C-terminus.

This ActRIIB-Fc fusion protein is encoded by the following nucleic acid sequence (SEQ ID NO: 397):

| (SEQ ID NO: 397) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGAA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGAAG TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAA |

A mature ActRIIB-Fc fusion polypeptide (SEQ ID NO: 398) is as follows, and may optionally be provided with lysine (K) removed from the C-terminus.

| (SEQ ID NO: 398) | |
|---|---|
| 1 | GRGEAETREC IYYNANWELE RTNQSGLERC EGEQDKRLHC YASWRNSSGT |
| 51 | IELVKKGCWL DDENCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | RKEMTKNQVS LTCLVKGFYP SDIAVEWESN GQPENNYKTT PPVLKSDGSE |
| 301 | FLYSKLTVDK SRWQQGNVES CSVMHEALHN HYTQKSLSLS PGK |

A complementary form of ALK4-Fc fusion polypeptide (SEQ ID NO: 88) is as follows:

| (SEQ ID NO: 88) | |
|---|---|
| 1 | MDAMKRGLCC VLLLCGAVFV SPGASGPRGV QALLCACTSC LQANYTCETD |
| 51 | GACMVSIFNL DGMEHHVRTC IPKVELVPAG KPFYCLSSED LRNTHCCYTD |
| 101 | YCNRIDLRVP SGHLKEPEHP SMWGPVETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVYTL PPSREEMTKN QVSLTCLVKG FYPSDIAVEW ESNGQPENNY |
| 301 | DTTPPVLDSD GSFFLYSDLT VDKSRWQQGN VESCSVMHEA LHNHYTQKSL |
| 351 | SLSPG |

The leader sequence and linker are underlined. To guide heterodimer formation with the ActRIIB-Fc fusion polypeptide of SEQ ID NOs: 396 and 398 above, two amino acid substitutions (replacing lysines with aspartic acids) can be introduced into the Fc domain of the ALK4-Fc fusion polypeptide as indicated by double underline above. The amino acid sequence of SEQ ID NO: 88 may optionally be provided with lysine (K) added at the C-terminus.

This ALK4-Fc fusion protein is encoded by the following nucleic acid (SEQ ID NO: 243):

| (SEQ ID NO: 243) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCCGGGCC CCGGGGGGTC CAGGCTCTGC |
| 101 | TGTGTGCGTG CACCAGCTGC CTCCAGGCCA ACTACACGTG TGAGACAGAT |
| 151 | GGGGCCTGCA TGGTTTCCAT TTTCAATCTG GATGGGATGG AGCACCATGT |
| 201 | GCGCACCTGC ATCCCCAAAG TGGAGCTGGT CCCTGCCGGG AAGCCCTTCT |
| 251 | ACTGCCTGAG CTCGGAGGAC CTGCGCAACA CCCACTGCTG CTACACTGAC |
| 301 | TACTGCAACA GGATCGACTT GAGGGTGCCC AGTGGTCACC TCAAGGAGCC |
| 351 | TGAGCACCCG TCCATGTGGG GCCCGGTGGA GACCGGTGGT GGAACTCACA |
| 401 | CATGCCCACC GTGCCCAGCA CCTGAACTCC TGGGGGGACC GTCAGTCTTC |
| 451 | CTCTTCCCCC CAAAACCCAA GGACACCCTC ATGATCTCCC GGACCCCTGA |
| 501 | GGTCACATGC GTGGTGGTGG ACGTGAGCCA CGAAGACCCT GAGGTCAAGT |
| 551 | TCAACTGGTA CGTGGACGGC GTGGAGGTGC ATAATGCCAA GACAAAGCCG |
| 601 | CGGGAGGAGC AGTACAACAG CACGTACCGT GTGGTCAGCG TCCTCACCGT |
| 651 | CCTGCACCAG GACTGGCTGA ATGGCAAGGA GTACAAGTGC AAGGTCTCCA |
| 701 | ACAAAGCCCT CCCAGCCCCC ATCGAGAAAA CCATCTCCAA AGCCAAAGGG |
| 751 | CAGCCCCGAG AACCACAGGT GTACACCCTG CCCCCATCCC GGGAGGAGAT |
| 801 | GACCAAGAAC CAGGTCAGCC TGACCTGCCT GGTCAAAGGC TTCTATCCCA |
| 851 | GCGACATCGC CGTGGAGTGG GAGAGCAATG GGCAGCCGGA GAACAACTAC |
| 901 | GACACCACGC CTCCCGTGCT GGACTCCGAC GGCTCCTTCT TCCTCTATAG |
| 951 | CGACCTCACC GTGGACAAGA GCAGGTGGCA GCAGGGGAAC GTCTTCTCAT |
| 1001 | GCTCCGTGAT GCATGAGGCT CTGCACAACC ACTACACGCA GAAGAGCCTC |
| 1051 | TCCCTGTCTC CGGGT |

A mature ALK4-Fc fusion protein sequence (SEQ ID NO: 89) is as follows and may optionally be provided with lysine (K) added at the C-terminus.

| (SEQ ID NO: 89) | |
|---|---|
| 1 | SGPRGVQALL CACTSCLQAN YTCETDGACM VSIFNLDGME HHVRTCIPKV |
| 51 | ELVPAGKPFY CLSSEDLRNT HCCYTDYCNR IDLRVPSGHL KEPEHPSMWG |
| 101 | PVETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVYTLPPSR EEMTKNQVSL |
| 251 | TCLVKGFYPS DIAVEWESNG QPENNYDTTP PVLDSDGSFF LYSDLTVDKS |
| 301 | RWQQGNVESC SVMHEALHNH YTQKSLSLSP G |

The ActRIIB-Fc and ALK4-Fc proteins of SEQ ID NO: 398 and SEQ ID NO: 89, respectively, may be co-expressed and purified from a CHO cell line, to give rise to a heteromeric complex comprising ActRIIB-Fc:ALK4-Fc.

In another approach to promote the formation of heteromultimer complexes using asymmetric Fc fusion proteins the Fc domains are altered to introduce complementary hydrophobic interactions and an additional intermolecular disulfide bond as illustrated in the ActRIIB-Fc and ALK4-Fc polypeptide sequences of SEQ ID NOs: 402 and 403 and SEQ ID Nos: 92 and 93, respectively. The ActRIIB-Fc fusion polypeptide and ALK4-Fc fusion polypeptide each employ the tissue plasminogen activator (TPA) leader:

(SEQ ID NO: 8)
MDAMKRGLCCVLLLCGAVFVSP.

The ActRIIB-Fc polypeptide sequence (SEQ ID NO: 402) is shown below:

| (SEQ ID NO: 402) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDENC YDRQECVATE |
| 101 | ENPQVYFCCC EGNECNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPCREEMT KNQVSLWCLV KGFYPSDIAV |
| 301 | EWESNGQPEN NYKTTPPVLD SDGSFFLYSK LTVDKSRWQQ GNVESCSVMH |
| 351 | EALHNHYTQK SLSLSPGK |

The leader (signal) sequence and linker are underlined. To promote formation of the ActRIIB-Fc:ALK4-Fc heterodimer rather than either of the possible homodimeric complexes, two amino acid substitutions (replacing a serine with a cysteine and a threonine with a tryptophan) can be introduced into the Fc domain of the fusion protein as indicated by double underline above. The amino acid sequence of SEQ ID NO: 402 may optionally be provided with lysine (K) removed from the C-terminus.

A mature ActRIIB-Fc fusion polypeptide is as follows:

| (SEQ ID NO: 403) | |
|---|---|
| 1 | GRGEAETREC IYYNANWELE RTNQSGLERC EGEQDKRLHC YASWRNSSGT |
| 51 | IELVKKGCWL DDENCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPC |
| 251 | REEMTKNQVS LWCLVKGFYP SDIAVEWESN GQPENNYKTT PPVLDSDGSF |
| 301 | FLYSKLTVDK SRWQQGNVES CSVMHEALHN HYTQKSLSLS PGK |

A complementary form of ALK4-Fc fusion polypeptide (SEQ ID NO: 92) is as follows and may optionally be provided with lysine (K) removed from the C-terminus.

| (SEQ ID NO: 92) | |
|---|---|
| 1 | MDAMKRGLCC VLLLCGAVFV SPGASGPRGV QALLCACTSC LQANYTCETD |
| 51 | GACMVSIFNL DGMEHHVRTC IPKVELVPAG KPFYCLSSED LRNTHCCYTD |
| 101 | YCNRIDLRVP SGHLKEPEHP SMWGPVETGG GTHTCPPCPA PELLGGPSVF |
| 151 | LFPPKPKDTL MISRTPEVTC VVVDVSHEDP EVKFNWYVDG VEVHNAKTKP |
| 201 | REEQYNSTYR VVSVLTVLHQ DWLNGKEYKC KVSNKALPAP IEKTISKAKG |
| 251 | QPREPQVCTL PPSREEMTKN QVSLSCAVKG FYPSDIAVEW ESNGQPENNY |
| 301 | KTTPPVLDSD GSFFLVSKLT VDKSRWQQGN VESCSVMHEA LHNHYTQKSL |
| 351 | SLSPGK |

The leader sequence and the linker are underlined. To guide heterodimer formation with the ActRIIB-Fc fusion polypeptide of SEQ ID NOs: 402 and 403 above, four amino acid substitutions can be introduced into the Fc domain of the ALK4 fusion polypeptide as indicated by double underline above. The amino acid sequence of SEQ ID NO: 92 may optionally be provided with lysine (K) removed from the C-terminus.

A mature ALK4-Fc fusion protein sequence is as follows and may optionally be provided with lysine (K) removed from the C-terminus.

| (SEQ ID NO: 93) | |
|---|---|
| 1 | SGPRGVQALL CACTSCLQAN YTCETDGACM VSIFNLDGME HHVRTCIPKV |
| 51 | ELVPAGKPFY CLSSEDLRNT HCCYTDYCNR IDLRVPSGHL KEPEHPSMWG |
| 101 | PVETGGGTHT CPPCPAPELL GGPSVFLFPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVCTLPPSR EEMTKNQVSL |
| 251 | SCAVKGFYPS DIAVEWESNG QPENNYKTTP PVLDSDGSFF LVSKLTVDKS |
| 301 | RWQQGNVFSC SVMHEALHNH YTQKSLSLSP GK |

ActRIIB-Fc and ALK4-Fc proteins of SEQ ID NO: 403 and SEQ ID NO: 93 respectively, may be co-expressed and purified from a CHO cell line, to give rise to a heteromeric complex comprising ActRIIB-Fc:ALK4-Fc.

Purification of various ActRIIB-Fc:ALK4-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

In another approach to promote the formation of heteromultimer complexes using asymmetric Fc fusion proteins, the Fc domains are altered to introduce complementary hydrophobic interactions, an additional intermolecular disulfide bond, and electrostatic differences between the two Fc domains for facilitating purification based on net molecular charge, as illustrated in the ActRIIB-Fc and ALK4-Fc polypeptide sequences of SEQ ID NOs: 118-121 and 122-125, respectively. The ActRIIB-Fc fusion polypeptide and ALK4-Fc fusion polypeptide each employ the tissue plasminogen activator (TPA) leader).

The ActRIIB-Fc polypeptide sequence (SEQ ID NO: 406) is shown below:

| (SEQ ID NO: 406) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDENC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPCREEMT ENQVSLWCLV KGFYPSDIAV |
| 301 | EWESNGQPEN NYKTTPPVLD SDGSFFLYSK LTVDKSRWQQ GNVESCSVMH |
| 351 | EALHNHYTQD SLSLSPG |

The leader sequence and linker are underlined. To promote formation of the ActRIIB-Fc:ALK4-Fc heterodimer rather than either of the possible homodimeric complexes, two amino acid substitutions (replacing a serine with a cysteine and a threonine with a tryptophan) can be introduced into the Fc domain of the fusion protein as indicated by double underline above. To facilitate purification of the ActRIIB-Fc:ALK4-Fc heterodimer, two amino acid substitutions (replacing lysines with acidic amino acids) can also be introduced into the Fc domain of the fusion protein as indicated by double underline above. The amino acid sequence of SEQ ID NO: 118 may optionally be provided with a lysine added at the C-terminus.

This ActRIIB-Fc fusion protein is encoded by the following nucleic acid (SEQ ID NO: 407):

| (SEQ ID NO: 407) | |
|---|---|
| 1 | ATGGATGCAA TGAAGAGAGG GCTCTGCTGT GTGCTGCTGC TGTGTGGAGC |
| 51 | AGTCTTCGTT CTACAACGCC AACTGGGAGC TGGAGCGCAC CAACCAGAGC |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATGCCGGGA GGAGATGACC GAGAACCAGG |
| 851 | TCAGCCTGTG GTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGGAC AGCCTCTCCC TGTCTCCGGG |
| 1101 | T |

The mature ActRIIB-Fc fusion polypeptide is as follows (SEQ ID NO: 408) and may optionally be provided with a lysine added to the C-terminus.

| (SEQ ID NO: 408) | |
|---|---|
| 1 | GRGEAETREC IYYNANWELE RTNQSGLERC EGEQDKRLHC YASWRNSSGT |
| 51 | IELVKKGCWL DDENCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPC |
| 251 | REEMTENQVS LWCLVKGFYP SDIAVEWESN GQPENNYKTT PPVLDSDGSE |
| 301 | FLYSKLTVDK SRWQQGNVES CSVMHEALHN HYTQDSLSLS PG |

This ActRIIB-Fc fusion polypeptide is encoded by the following nucleic acid (SEQ ID NO: 409):

| (SEQ ID NO: 409) | |
|---|---|
| 1 | GGGCGTGGGG AGGCTGAGAC ACGGGAGTGC ATCTACTACA ACGCCAACTG |
| 51 | GGAGCTGGAG CGCACCAACC AGAGCGGCCT GGAGCGCTGC GAAGGCGAGC |
| 101 | AGGACAAGCG GCTGCACTGC TACGCCTCCT GGCGCAACAG CTCTGGCACC |
| 151 | ATCGAGCTCG TGAAGAAGGG CTGCTGGCTA GATGACTTCA ACTGCTACGA |
| 201 | TAGGCAGGAG TGTGTGGCCA CTGAGGAGAA CCCCCAGGTG TACTTCTGCT |
| 251 | GCTGTGAAGG CAACTTCTGC AACGAGCGCT TCACTCATTT GCCAGAGGCT |
| 301 | GGGGGCCCGG AAGTCACGTA CGAGCCACCC CCGACAGCCC CCACCGGTGG |
| 351 | TGGAACTCAC ACATGCCCAC CGTGCCCAGC ACCTGAACTC CTGGGGGGAC |
| 401 | CGTCAGTCTT CCTCTTCCCC CCAAAACCCA AGGACACCCT CATGATCTCC |
| 451 | CGGACCCCTG AGGTCACATG CGTGGTGGTG GACGTGAGCC ACGAAGACCC |
| 501 | TGAGGTCAAG TTCAACTGGT ACGTGGACGG CGTGGAGGTG CATAATGCCA |
| 551 | AGACAAAGCC GCGGGAGGAG CAGTACAACA GCACGTACCG TGTGGTCAGC |
| 601 | GTCCTCACCG TCCTGCACCA GGACTGGCTG AATGGCAAGG AGTACAAGTG |
| 651 | CAAGGTCTCC AACAAAGCCC TCCCAGCCCC CATCGAGAAA ACCATCTCCA |
| 701 | AAGCCAAAGG GCAGCCCCGA GAACCACAGG TGTACACCCT GCCCCCATGC |
| 751 | CGGGAGGAGA TGACCGAGAA CCAGGTCAGC CTGTGGTGCC TGGTCAAAGG |
| 801 | CTTCTATCCC AGCGACATCG CCGTGGAGTG GGAGAGCAAT GGGCAGCCGG |
| 851 | AGAACAACTA CAAGACCACG CCTCCCGTGC TGGACTCCGA CGGCTCCTTC |
| 901 | TTCCTCTATA GCAAGCTCAC CGTGGACAAG AGCAGGTGGC AGCAGGGGAA |
| 951 | CGTCTTCTCA TGCTCCGTGA TGCATGAGGC TCTGCACAAC CACTACACGC |
| 1001 | AGGACAGCCT CTCCCTGTCT CCGGGT |

The complementary form of ALK4-Fc fusion polypeptide (SEQ ID NO: 247) is as follows and may optionally be provided with lysine removed from the C-terminus.

| (SEQ ID NO: 247) | |
|---|---|
| 1 | MDAMKRGLCC VLLLCGAVEV SPGASGPRGV QALLCACTSC LQANYTCETD |
| 51 | GACMVSIFNL DGMEHHVRTC IPKVELVPAG KPFYCLSSED LRNTHCCYTD |
| 101 | YCNRIDLRVP SGHLKEPEHP SMWGPVETGG GTHTCPPCPA PELLGGPSVE |
| 151 | LFPPKPKDTL MISRTPEVTC VVVDVSHEDP EVKENWYVDG VEVHNAKTKP |
| 201 | REEQYNSTYR VVSVLIVLHQ DWLNGKEYKC KVSNKALPAP IEKTISKAKG |
| 251 | QPREPQVCTL PPSREEMTKN QVSLSCAVKG FYPSDIAVEW ESRGQPENNY |
| 301 | KTTPPVLDSR GSFFLVSKLT VDKSRWQQGN VESCSVMHEA LHNHYTQKSL |
| 351 | SLSPGK |

The leader sequence and the linker are underlined. To guide heterodimer formation with the ActRIIB-Fc fusion polypeptide of SEQ ID NOs: 406 and 408 above, four amino acid substitutions (replacing a tyrosine with a cysteine, a threonine with a serine, a leucine with an alanine, and a tyrosine with a valine) can be introduced into the Fc domain of the ALK4 fusion polypeptide as indicated by double underline above. To facilitate purification of the ActRIIB-Fc:ALK4-Fc heterodimer, two amino acid substitutions (replacing an asparagine with an arginine and an aspartate with an arginine) can also be introduced into the Fc domain of the ALK4-Fc fusion polypeptide as indicated by double underline above. The amino acid sequence of SEQ ID NO: 247 may optionally be provided with lysine removed from the C-terminus.

This ALK4-Fc fusion polypeptide is encoded by the following nucleic acid (SEQ ID NO: 248):

| (SEQ ID NO: 248) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCCGGGCC CCGGGGGGTC CAGGCTCTGC |
| 101 | TGTGTGCGTG CACCAGCTGC CTCCAGGCCA ACTACACGTG TGAGACAGAT |
| 151 | GGGGCCTGCA TGGTTTCCAT TTTCAATCTG GATGGGATGG AGCACCATGT |
| 201 | GCGCACCTGC ATCCCCAAAG TGGAGCTGGT CCCTGCCGGG AAGCCCTTCT |
| 251 | ACTGCCTGAG CTCGGAGGAC CTGCGCAACA CCCACTGCTG CTACACTGAC |
| 301 | TACTGCAACA GGATCGACTT GAGGGTGCCC AGTGGTCACC TCAAGGAGCC |
| 351 | TGAGCACCCG TCCATGTGGG GCCCGGTGGA GACCGGTGGT GGAACTCACA |
| 401 | CATGCCCACC GTGCCCAGCA CCTGAACTCC TGGGGGGACC GTCAGTCTTC |
| 451 | CTCTTCCCCC CAAAACCCAA GGACACCCTC ATGATCTCCC GGACCCCTGA |
| 501 | GGTCACATGC GTGGTGGTGG ACGTGAGCCA CGAAGACCCT GAGGTCAAGT |
| 551 | TCAACTGGTA CGTGGACGGC GTGGAGGTGC ATAATGCCAA GACAAAGCCG |
| 601 | CGGGAGGAGC AGTACAACAG CACGTACCGT GTGGTCAGCG TCCTCACCGT |
| 651 | CCTGCACCAG GACTGGCTGA ATGGCAAGGA GTACAAGTGC AAGGTCTCCA |
| 701 | ACAAAGCCCT CCCAGCCCCC ATCGAGAAAA CCATCTCCAA AGCCAAAGGG |
| 751 | CAGCCCCGAG AACCACAGGT GTGCACCCTG CCCCCATCCC GGGAGGAGAT |
| 801 | GACCAAGAAC CAGGTCAGCC TGTCCTGCGC CGTCAAAGGC TTCTATCCCA |
| 851 | GCGACATCGC CGTGGAGTGG GAGAGCCGCG GGCAGCCGGA GAACAACTAC |
| 901 | AAGACCACGC CTCCCGTGCT GGACTCCCGC GGCTCCTTCT TCCTCGTGAG |
| 951 | CAAGCTCACC GTGGACAAGA GCAGGTGGCA GCAGGGGAAC GTCTTCTCAT |
| 1001 | GCTCCGTGAT GCATGAGGCT CTGCACAACC ACTACACGCA GAAGAGCCTC |
| 1051 | TCCCTGTCTC CGGGTAAA |

The mature ALK4-Fc fusion polypeptide sequence is as follows (SEQ ID NO: 249) and may optionally be provided with lysine removed from the C-terminus.

| (SEQ ID NO: 249) | |
|---|---|
| 1 | SGPRGVQALL CACTSCLQAN YTCETDGACM VSIENLDGME HHVRTCIPKV |
| 51 | ELVPAGKPFY CLSSEDLRNT HCCYTDYCNR IDLRVPSGHL KEPEHPSMWG |
| 101 | PVETGGGTHT CPPCPAPELL GGPSVELEPP KPKDTLMISR TPEVTCVVVD |
| 151 | VSHEDPEVKF NWYVDGVEVH NAKTKPREEQ YNSTYRVVSV LTVLHQDWLN |
| 201 | GKEYKCKVSN KALPAPIEKT ISKAKGQPRE PQVCTLPPSR EEMTKNQVSL |
| 251 | SCAVKGFYPS DIAVEWESRG QPENNYKTTP PVLDSRGSFF LVSKLTVDKS |
| 301 | RWQQGNVESC SVMHEALHNH YTQKSLSLSP GK |

This ALK4-Fc fusion polypeptide is encoded by the following nucleic acid (SEQ ID NO: 250):

| (SEQ ID NO: 250) | |
|---|---|
| 1 | TCCGGGCCCC GGGGGGTCCA GGCTCTGCTG TGTGCGTGCA CCAGCTGCCT |
| 51 | CCAGGCCAAC TACACGTGTG AGACAGATGG GGCCTGCATG GTTTCCATTT |
| 101 | TCAATCTGGA TGGGATGGAG CACCATGTGC GCACCTGCAT CCCCAAAGTG |
| 151 | GAGCTGGTCC CTGCCGGGAA GCCCTTCTAC TGCCTGAGCT CGGAGGACCT |
| 201 | GCGCAACACC CACTGCTGCT ACACTGACTA CTGCAACAGG ATCGACTTGA |
| 251 | GGGTGCCCAG TGGTCACCTC AAGGAGCCTG AGCACCCGTC CATGTGGGGC |
| 301 | CCGGTGGAGA CCGGTGGTGG AACTCACACA TGCCCACCGT GCCCAGCACC |
| 351 | TGAACTCCTG GGGGGACCGT CAGTCTTCCT CTTCCCCCCA AAACCCAAGG |
| 401 | ACACCCTCAT GATCTCCCGG ACCCCTGAGG TCACATGCGT GGTGGTGGAC |
| 451 | GTGAGCCACG AAGACCCTGA GGTCAAGTTC AACTGGTACG TGGACGGCGT |
| 501 | GGAGGTGCAT AATGCCAAGA CAAAGCCGCG GGAGGAGCAG TACAACAGCA |
| 551 | CGTACCGTGT GGTCAGCGTC CTCACCGTCC TGCACCAGGA CTGGCTGAAT |
| 601 | GGCAAGGAGT ACAAGTGCAA GGTCTCCAAC AAAGCCCTCC CAGCCCCCAT |
| 651 | CGAGAAAACC ATCTCCAAAG CCAAAGGGCA GCCCCGAGAA CCACAGGTGT |
| 701 | GCACCCTGCC CCCATCCCGG GAGGAGATGA CCAAGAACCA GGTCAGCCTG |
| 751 | TCCTGCGCCG TCAAAGGCTT CTATCCCAGC GACATCGCCG TGGAGTGGGA |
| 801 | GAGCCGCGGG CAGCCGGAGA ACAACTACAA GACCACGCCT CCCGTGCTGG |
| 851 | ACTCCCGCGG CTCCTTCTTC CTCGTGAGCA AGCTCACCGT GGACAAGAGC |
| 901 | AGGTGGCAGC AGGGGAACGT CTTCTCATGC TCCGTGATGC ATGAGGCTCT |
| 951 | GCACAACCAC TACACGCAGA AGAGCCTCTC CCTGTCTCCG GGTAAA |

ActRIIB-Fc and ALK4-Fc proteins of SEQ ID NO: 120 and SEQ ID NO: 249, respectively, may be co-expressed and purified from a CHO cell line, to give rise to a heteromeric complex comprising ALK4-Fc:ActRIIB-Fc.

In certain embodiments, the ALK4-Fc fusion polypeptide is SEQ ID NO: 92 (shown above), which contains four amino acid substitutions to guide heterodimer formation certain Fc fusion polypeptides disclosed herein, and may optionally be provided with lysine removed from the C-terminus.

This ALK4-Fc fusion polypeptide is encoded by the following nucleic acid (SEQ ID NO: 251):

| (SEQ ID NO: 251) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCCGGGCC CCGGGGGGTC CAGGCTCTGC |
| 101 | TGTGTGCGTG CACCAGCTGC CTCCAGGCCA ACTACACGTG TGAGACAGAT |
| 151 | GGGGCCTGCA TGGTTTCCAT TTTCAATCTG GATGGGATGG AGCACCATGT |
| 201 | GCGCACCTGC ATCCCCAAAG TGGAGCTGGT CCCTGCCGGG AAGCCCTTCT |
| 251 | ACTGCCTGAG CTCGGAGGAC CTGCGCAACA CCCACTGCTG CTACACTGAC |
| 301 | TACTGCAACA GGATCGACTT GAGGGTGCCC AGTGGTCACC TCAAGGAGCC |
| 351 | TGAGCACCCG TCCATGTGGG GCCCGGTGGA GACCGGTGGT GGAACTCACA |
| 401 | CATGCCCACC GTGCCCAGCA CCTGAACTCC TGGGGGGACC GTCAGTCTTC |
| 451 | CTCTTCCCCC CAAAACCCAA GGACACCCTC ATGATCTCCC GGACCCCTGA |
| 501 | GGTCACATGC GTGGTGGTGG ACGTGAGCCA CGAAGACCCT GAGGTCAAGT |
| 551 | TCAACTGGTA CGTGGACGGC GTGGAGGTGC ATAATGCCAA GACAAAGCCG |
| 601 | CGGGAGGAGC AGTACAACAG CACGTACCGT GTGGTCAGCG TCCTCACCGT |
| 651 | CCTGCACCAG GACTGGCTGA ATGGCAAGGA GTACAAGTGC AAGGTCTCCA |
| 701 | ACAAAGCCCT CCCAGCCCCC ATCGAGAAAA CCATCTCCAA AGCCAAAGGG |
| 751 | CAGCCCCGAG AACCACAGGT GTGCACCCTG CCCCCATCCC GGGAGGAGAT |
| 801 | GACCAAGAAC CAGGTCAGCC TGTCCTGCGC CGTCAAAGGC TTCTATCCCA |
| 851 | GCGACATCGC CGTGGAGTGG GAGAGCAATG GGCAGCCGGA GAACAACTAC |
| 901 | AAGACCACGC CTCCCGTGCT GGACTCCGAC GGCTCCTTCT TCCTCGTGAG |
| 951 | CAAGCTCACC GTGGACAAGA GCAGGTGGCA GCAGGGGAAC GTCTTCTCAT |
| 1001 | GCTCCGTGAT GCATGAGGCT CTGCACAACC ACTACACGCA GAAGAGCCTC |
| 1051 | TCCCTGTCTC CGGGTAAA |

The mature ALK4-Fc fusion polypeptide sequence is SEQ ID NO: 93 (shown above) and may optionally be provided with lysine removed from the C-terminus.

This ALK4-Fc fusion polypeptide is encoded by the following nucleic acid (SEQ ID NO: 252):

| (SEQ ID NO: 252) | |
|---|---|
| 1 | TCCGGGCCCC GGGGGGTCCA GGCTCTGCTG TGTGCGTGCA CCAGCTGCCT |
| 51 | CCAGGCCAAC TACACGTGTG AGACAGATGG GGCCTGCATG GTTTCCATTT |
| 101 | TCAATCTGGA TGGGATGGAG CACCATGTGC GCACCTGCAT CCCCAAAGTG |
| 151 | GAGCTGGTCC CTGCCGGGAA GCCCTTCTAC TGCCTGAGCT CGGAGGACCT |
| 201 | GCGCAACACC CACTGCTGCT ACACTGACTA CTGCAACAGG ATCGACTTGA |
| 251 | GGGTGCCCAG TGGTCACCTC AAGGAGCCTG AGCACCCGTC CATGTGGGGC |
| 301 | CCGGTGGAGA CCGGTGGTGG AACTCACACA TGCCCACCGT GCCCAGCACC |
| 351 | TGAACTCCTG GGGGGACCGT CAGTCTTCCT CTTCCCCCCA AAACCCAAGG |
| 401 | ACACCCTCAT GATCTCCCGG ACCCCTGAGG TCACATGCGT GGTGGTGGAC |
| 451 | GTGAGCCACG AAGACCCTGA GGTCAAGTTC AACTGGTACG TGGACGGCGT |
| 501 | GGAGGTGCAT AATGCCAAGA CAAAGCCGCG GGAGGAGCAG TACAACAGCA |
| 551 | CGTACCGTGT GGTCAGCGTC CTCACCGTCC TGCACCAGGA CTGGCTGAAT |
| 601 | GGCAAGGAGT ACAAGTGCAA GGTCTCCAAC AAAGCCCTCC CAGCCCCCAT |
| 651 | CGAGAAAACC ATCTCCAAAG CCAAAGGGCA GCCCCGAGAA CCACAGGTGT |
| 701 | GCACCCTGCC CCCATCCCGG GAGGAGATGA CCAAGAACCA GGTCAGCCTG |
| 751 | TCCTGCGCCG TCAAAGGCTT CTATCCCAGC GACATCGCCG TGGAGTGGGA |
| 801 | GAGCAATGGG CAGCCGGAGA ACAACTACAA GACCACGCCT CCCGTGCTGG |
| 851 | ACTCCGACGG CTCCTTCTTC CTCGTGAGCA AGCTCACCGT GGACAAGAGC |
| 901 | AGGTGGCAGC AGGGGAACGT CTTCTCATGC TCCGTGATGC ATGAGGCTCT |
| 951 | GCACAACCAC TACACGCAGA AGAGCCTCTC CCTGTCTCCG GGTAAA |

Purification of various ActRIIB-Fc:ALK4-Fc complexes could be achieved by a series of column chromatography steps, including, for example, three or more of the following, in any order: protein A chromatography, Q sepharose chromatography, phenylsepharose chromatography, size exclusion chromatography, cation exchange chromatography, epitope-based affinity chromatography (e.g., with an antibody or functionally equivalent ligand directed against an epitope on ALK4 or ActRIIB), and multimodal chromatography (e.g., with resin containing both electrostatic and hydrophobic ligands). The purification could be completed with viral filtration and buffer exchange.

### Example 13. Ligand Binding Profile of ActRIIB-Fc:ALK4-Fc Heterodimer Compared to ActRIIB-Fc Homodimer and ALK4-Fc Homodimer

A Biacore™-based binding assay was used to compare ligand binding selectivity of the ActRIIB-Fc:ALK4-Fc heterodimeric complex described above with that of ActRIIB-Fc and ALK4-Fc homodimer complexes. The ActRIIB-Fc:ALK4-Fc heterodimer, ActRIIB-Fc homodimer, and ALK4-Fc homodimer were independently captured onto the system using an anti-Fc antibody. Ligands were injected and allowed to flow over the captured receptor protein. Results are summarized in the table below, in which ligand off-rates (k_{d}) most indicative of effective ligand traps are denoted by bold font gray shading.

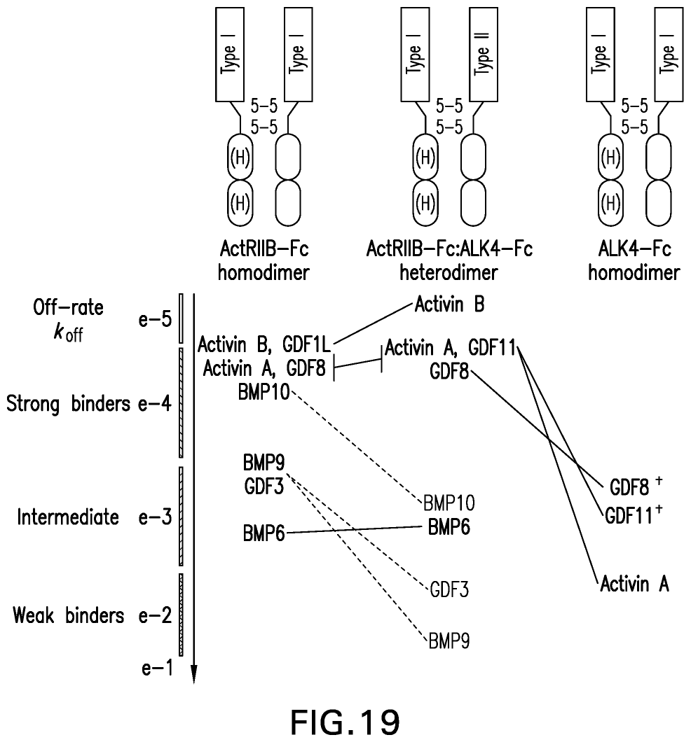

These comparative binding data demonstrate that ActRIIB-Fc:ALK4-Fc heterodimer has an altered binding profile/selectivity relative to either ActRIIB-Fc or ALK4-Fc homodimers. ActRIIB-Fc:ALK4-Fc heterodimer displays enhanced binding to activin B compared with either homodimer, retains strong binding to activin A, GDF8, and GDF11 as observed with ActRIIB-Fc homodimer, and exhibits substantially reduced binding to BMP9, BMP10, and GDF3. In particular, BMP9 displays low or no observable affinity for ActRIIB-Fc:ALK4-Fc heterodimer, whereas this ligand binds strongly to ActRIIB-Fc homodimer. Like the ActRIIB-Fc homodimer, the heterodimer retains intermediate-level binding to BMP6. See FIG. 19.

In addition, an A-204 Reporter Gene Assay was used to evaluate the effects of ActRIIB-Fc:ALK4-Fc heterodimer and ActRIIB-Fc:ActRIIB-Fc homodimer on signaling by activin A, activin B, GDF11, GDF8, BMP10, and BMP9. Cell line: Human Rhabdomyosarcoma (derived from muscle). Reporter vector: pGL3(CAGA)12 (as described in Dennler et al, 1998, EMBO 17: 3091-3100). The CAGA12 motif is present in TGFβ responsive genes (PAI-1 gene), so this vector is of general use for factors signaling through Smad2 and 3. An exemplary A-204 Reporter Gene Assay is outlined below.
Day 1: Split A-204 cells into 48-well plate.
Day 2: A-204 cells transfected with 10 ug pGL3(CAGA)12 or pGL3(CAGA)12(10 ug)+pRLCMV (1 ug) and Fugene.
Day 3: Add factors (diluted into medium+0.1% BSA). Inhibitors need to be preincubated with Factors for about one hr before adding to cells. About six hrs later, cells are rinsed with PBS and then lysed.

Following the above steps, a Luciferase assay was performed.

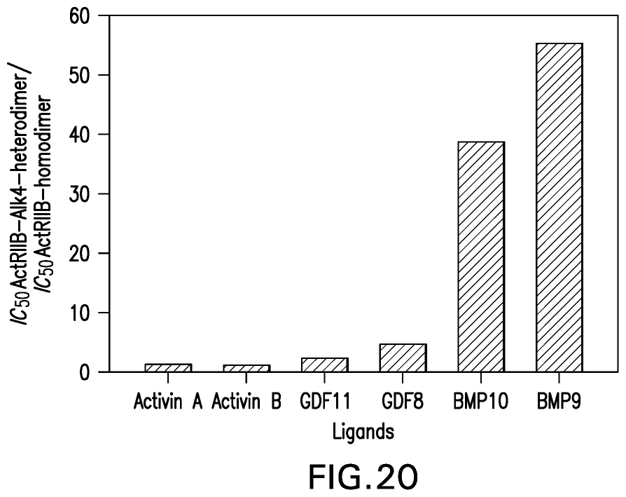

Both the ActRIIB-Fc:ALK4-Fc heterodimer and ActRIIB-Fc:ActRIIB-Fc homodimer were determined to be potent inhibitors of activin A, activin B, GDF11, and GDF8 in this assay. In particular, as can be seen in the comparative homodimer/heterodimer IC₅₀ data illustrated in FIG. 20, ActRIIB-Fc:ALK4-Fc heterodimer inhibits activin A, activin B, GDF8, and GDF11 signaling pathways similarly to the ActRIIB-Fc:ActRIIB-Fc homodimer. However, ActRIIB-Fc:ALK4-Fc heterodimer inhibition of BMP9 and BMP10 signaling pathways is significantly reduced compared to the ActRIIB-Fc:ActRIIB-Fc homodimer. This data is consistent with the above-discussed binding data in which it was observed that both the ActRIIB-Fc:ALK4-Fc heterodimer and ActRIIB-Fc:ActRIIB-Fc homodimer display strong binding to activin A, activin B, GDF8, and GDF11, but BMP10 and BMP9 have significantly reduced affinity for the ALK4-Fc:ActRIIB-Fc heterodimer compared to the ActRIIB-Fc:ActRIIB-Fc homodimer.

Together, these data therefore demonstrate that ActRIIB-Fc:ALK4-Fc heterodimer is a more selective antagonist of activin A, activin B, GDF8, and GDF11 compared to ActRIIB-Fc homodimer. Accordingly, an ActRIIB-Fc:ALK4-Fc heterodimer will be more useful than an ActRIIB-Fc homodimer in certain applications where such selective antagonism is advantageous. Examples include therapeutic applications where it is desirable to retain antagonism of one or more of activin A, activin B, activin AC, GDF8, and GDF11 but minimize antagonism of one or more of BMP9, BMP10, GDF3, and BMP6.

### Example 14. Generation of an ActRIIB-Fc:ALK7-Fc Heterodimer

Applicants constructed a soluble ActRIIB-Fc:ALK7-Fc heteromeric complex comprising the extracellular domains of human ActRIIB and human ALK7, which are each fused to an Fc domain with a linker positioned between the extracellular domain and the Fc domain. The individual constructs are referred to as ActRIIB-Fc and ALK7-Fc, respectively.

A methodology for promoting formation of ActRIIB-Fc:ALK7-Fc heteromeric complexes, as opposed to the ActRIIB-Fc or ALK7-Fc homodimeric complexes, is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the ActRIIB-Fc and ALK7-Fc polypeptide sequences disclosed below, respectively, one Fc domain is altered to introduce cationic amino acids at the interaction face, while the other Fc domain is altered to introduce anionic amino acids at the interaction face. The ActRIIB-Fc fusion polypeptide and ALK7-Fc fusion polypeptide each employ the tissue plasminogen activator (TPA) leader: MDAMKRGLCCVLLLCGAVFVSP (SEQ ID NO: 8).

The ActRIIB-Fc polypeptide sequence (SEQ ID NO: 396) is shown below:

```
(SEQ ID NO: 396)
    1 MDAMKRGLCC VLLLCGAVEV SPGASGRGEA
        ETRECIYYNA NWELERTNQS
  51 GLERCEGEQD KRLHCYASWR NSSGTIELVK
        KGCWLDDENC YDRQECVATE
101 ENPQVYFCCC EGNFCNERFT HLPEAGGPEV
        TYEPPPTAPT GGGTHTCPPC
151 PAPELLGGPS VELFPPKPKD TLMISRTPEV
        TCVVVDVSHE DPEVKENWYV
201 DGVEVHNAKT KPREEQYNST YRVVSVLTVL
        HQDWLNGKEY KCKVSNKALP
251 APIEKTISKA KGQPREPQVY TLPPSRKEMT
        KNQVSLTCLV KGFYPSDIAV
301 EWESNGQPEN NYKTTPPVLK SDGSFFLYSK
        LTVDKSRWQQ GNVESCSVMH
351 EALHNHYTQK SLSLSPGK
```

The leader (signal) sequence and linker are underlined. To promote formation of the ActRIIB-Fc:ALK7-Fc heterodimer rather than either of the possible homodimeric complexes, two amino acid substitutions (replacing acidic amino acids with lysine) can be introduced into the Fc domain of the ActRIIB fusion protein as indicated by double underline above. The amino acid sequence of SEQ ID NO: 396 may optionally be provided with lysine (K) removed from the C-terminus.

This ActRIIB-Fc fusion protein is encoded by the following nucleic acid sequence (SEQ ID NO: 397):

| (SEQ ID NO: 397) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGAA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGAAG TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAA |

The mature ActRIIB-Fc fusion polypeptide (SEQ ID NO: 398) is as follows, and may optionally be provided with lysine removed from the C-terminus.

| (SEQ ID NO: 398) | |
|---|---|
| 1 | GRGEAETREC IYYNANWELE RTNQSGLERC EGEQDKRLHC YASWRNSSGT |
| 51 | IELVKKGCWL DDENCYDRQE CVATEENPQV YFCCCEGNFC NERFTHLPEA |
| 101 | GGPEVTYEPP PTAPTGGGTH TCPPCPAPEL LGGPSVELFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | RKEMTKNQVS LTCLVKGFYP SDIAVEWESN GQPENNYKTT PPVLKSDGSF |
| 301 | FLYSKLTVDK SRWQQGNVES CSVMHEALHN HYTQKSLSLS PGK |

The complementary form of ALK7-Fc fusion protein (SEQ ID NO: 129) is as follows:

| (SEQ ID NO: 129) | |
|---|---|
| 1 | MDAMKRGLCC VLLLCGAVFV SPGAGLKCVC LLCDSSNFTC QTEGACWASV |
| 51 | MLTNGKEQVI KSCVSLPELN AQVFCHSSNN VTKTECCFTD FCNNITLHLP |
| 101 | TASPNAPKLG PMETGGGTHT CPPCPAPELL GGPSVFLFPP KPKDTLMISR |
| 151 | TPEVTCVVVD VSHEDPEVKF NWYVDGVEVH NAKTKPREEQ YNSTYRVVSV |
| 201 | LTVLHQDWLN GKEYKCKVSN KALPAPIEKT ISKAKGQPRE PQVYTLPPSR |
| 251 | EEMTKNQVSL TCLVKGFYPS DIAVEWESNG QPENNYDTTP PVLDSDGSFF |
| 301 | LYSDLTVDKS RWQQGNVFSC SVMHEALHNH YTQKSLSLSP G |

The signal sequence and linker sequence are underlined. To promote formation of the ActRIIB-Fc:ALK7-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 129 may optionally be provided with a lysine added at the C-terminus.

This ALK7-Fc fusion protein is encoded by the following nucleic acid (SEQ ID NO: 255):

| (SEQ ID NO: 255) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCGGACTGAA GTGTGTATGT CTTTTGTGTG |
| 101 | ATTCTTCAAA CTTTACCTGC CAAACAGAAG GAGCATGTTG GGCATCAGTC |
| 151 | ATGCTAACCA ATGGAAAAGA GCAGGTGATC AAATCCTGTG TCTCCCTTCC |
| 201 | AGAACTGAAT GCTCAAGTCT TCTGTCATAG TTCCAACAAT GTTACCAAAA |
| 251 | CCGAATGCTG CTTCACAGAT TTTTGCAACA ACATAACACT GCACCTTCCA |
| 301 | ACAGCATCAC CAAATGCCCC AAAACTTGGA CCCATGGAGA CCGGTGGTGG |
| 351 | AACTCACACA TGCCCACCGT GCCCAGCACC TGAACTCCTG GGGGGACCGT |
| 401 | CAGTCTTCCT CTTCCCCCCA AAACCCAAGG ACACCCTCAT GATCTCCCGG |
| 451 | ACCCCTGAGG TCACATGCGT GGTGGTGGAC GTGAGCCACG AAGACCCTGA |
| 501 | GGTCAAGTTC AACTGGTACG TGGACGGCGT GGAGGTGCAT AATGCCAAGA |
| 551 | CAAAGCCGCG GGAGGAGCAG TACAACAGCA CGTACCGTGT GGTCAGCGTC |
| 601 | CTCACCGTCC TGCACCAGGA CTGGCTGAAT GGCAAGGAGT ACAAGTGCAA |
| 651 | GGTCTCCAAC AAAGCCCTCC CAGCCCCCAT CGAGAAAACC ATCTCCAAAG |
| 701 | CCAAAGGGCA GCCCCGAGAA CCACAGGTGT ACACCCTGCC CCCATCCCGG |
| 751 | GAGGAGATGA CCAAGAACCA GGTCAGCCTG ACCTGCCTGG TCAAAGGCTT |
| 801 | CTATCCCAGC GACATCGCCG TGGAGTGGGA GAGCAATGGG CAGCCGGAGA |
| 851 | ACAACTACGA CACCACGCCT CCCGTGCTGG ACTCCGACGG CTCCTTCTTC |
| 901 | CTCTATAGCG ACCTCACCGT GGACAAGAGC AGGTGGCAGC AGGGGAACGT |
| 951 | CTTCTCATGC TCCGTGATGC ATGAGGCTCT GCACAACCAC TACACGCAGA |
| 1001 | AGAGCCTCTC CCTGTCTCCG GGT |

The mature ALK7-Fc fusion protein sequence (SEQ ID NO: 130) is expected to be as follows and may optionally be provided with a lysine added at the C-terminus.
1 GLKCVCLLCD SSNFTCQTEG ACWASVMLTN GKEQVIKSCV SLPELNAQVF

| (SEQ ID NO: 130) | |
|---|---|
| 51 | CHSSNNVIKT ECCFTDECNN ITLHLPTASP NAPKLGPMET GGGTHTCPPC |
| 101 | PAPELLGGPS VELFPPKPKD TLMISRTPEV TCVVVDVSHE DPEVKENWYV |
| 151 | DGVEVHNAKT KPREEQYNST YRVVSVLTVL HQDWLNGKEY KCKVSNKALP |
| 201 | APIEKTISKA KGQPREPQVY TLPPSREEMT KNQVSLTCLV KGFYPSDIAV |
| 251 | EWESNGQPEN NYDTTPPVLD SDGSFFLYSD LTVDKSRWQQ GNVESCSVMH |
| 301 | EALHNHYTQK SLSLSPG |

The ActRIIB-Fc and ALK7-Fc fusion proteins of SEQ ID NO: 396 and SEQ ID NO: 129, respectively, may be co-expressed and purified from a CHO cell line to give rise to a heteromeric complex comprising ActRIIB-Fc:ALK7-Fc.

In another approach to promote the formation of heteromultimer complexes using asymmetric Fc fusion proteins, the Fc domains are altered to introduce complementary hydrophobic interactions and an additional intermolecular disulfide bond as illustrated in the ActRIIB-Fc and ALK7-Fc polypeptide sequences of disclosed below.

The ActRIIB-Fc polypeptide sequence (SEQ ID NO: 402) is shown below:

| (SEQ ID NO: 402) | |
|---|---|
| 1 | MDAMKRGLCC VLLLCGAVEV SPGASGRGEA ETRECIYYNA NWELERTNQS |
| 51 | GLERCEGEQD KRLHCYASWR NSSGTIELVK KGCWLDDENC YDRQECVATE |
| 101 | ENPQVYFCCC EGNFCNERFT HLPEAGGPEV TYEPPPTAPT GGGTHTCPPC |
| 151 | PAPELLGGPS VELFPPKPKD TLMISRTPEV TCVVVDVSHE DPEVKENWYV |
| 201 | DGVEVHNAKT KPREEQYNST YRVVSVLTVL HQDWLNGKEY KCKVSNKALP |
| 251 | APIEKTISKA KGQPREPQVY TLPPCREEMT KNQVSLWCLV KGFYPSDIAV |
| 301 | EWESNGQPEN NYKTTPPVLD SDGSFFLYSK LTVDKSRWQQ GNVESCSVMH |
| 351 | EALHNHYTQK SLSLSPGK |

The leader sequence and linker are underlined. To promote formation of the ActRIIB-Fc:ALK7-Fc heterodimer rather than either of the possible homodimeric complexes, two amino acid substitutions (replacing a serine with a cysteine and a threonine with a tryptophan) can be introduced into the Fc domain of the fusion protein as indicated by double underline above. The amino acid sequence of SEQ ID NO: 402 may optionally be provided with lysine removed from the C-terminus.

The mature ActRIIB-Fc fusion polypeptide (SEQ ID NO: 403) is as follows and may optionally be provided with lysine removed from the C-terminus.

| (SEQ ID NO: 403) | |
|---|---|
| 1 | GRGEAETREC IYYNANWELE RINQSGLERC EGEQDKRLHC YASWRNSSGT |
| 51 | IELVKKGCWL DDENCYDRQE CVATEENPQV YFCCCEGNEC NERFTHLPEA |
| 101 | GGPEVTYEPP PTAPTGGGTH TCPPCPAPEL LGGPSVELFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPC |
| 251 | REEMTKNQVS LWCLVKGFYP SDIAVEWESN GQPENNYKTT PPVLDSDGSE |
| 301 | FLYSKLTVDK SRWQQGNVES CSVMHEALHN HYTQKSLSLS PGK |

The complementary form of ALK7-Fc fusion polypeptide (SEQ ID NO: 133) is as follows:

| (SEQ ID NO: 133) | |
|---|---|
| 1 | MDAMKRGLCC VLLLCGAVEV SPGAGLKCVC LLCDSSNFTC QTEGACWASV |
| 51 | MLTNGKEQVI KSCVSLPELN AQVECHSSNN VIKTECCFTD FCNNITLHLP |
| 101 | TASPNAPKLG PMETGGGTHT CPPCPAPELL GGPSVELFPP KPKDTLMISR |
| 151 | TPEVTCVVVD VSHEDPEVKE NWYVDGVEVH NAKTKPREEQ YNSTYRVVSV |
| 201 | LTVLHQDWLN GKEYKCKVSN KALPAPIEKT ISKAKGQPRE PQVCTLPPSR |
| 251 | EEMTKNQVSL SCAVKGFYPS DIAVEWESNG QPENNYKTTP PVLDSDGSFF |
| 301 | LVSKLTVDKS RWQQGNVESC SVMHEALHNH YTQKSLSLSP GK |

The leader sequence and linker sequence are underlined. To guide heterodimer formation with the ActRIIB-Fc fusion polypeptide of SEQ ID NOs 130 and 403 above, four amino acid substitutions can be introduced into the Fc domain of the ALK7 fusion polypeptide as indicated by double underline above. The amino acid sequence of SEQ ID NO: 133 may optionally be provided with the lysine removed from the C-terminus.

The mature ALK7-Fc fusion protein sequence (SEQ ID NO: 134) is expected to be as follows and may optionally be provided with the lysine removed from the C-terminus.

| (SEQ ID NO: 134) | |
|---|---|
| 1 | GLKCVCLLCD SSNFTCQTEG ACWASVMLIN GKEQVIKSCV SLPELNAQVE |
| 51 | CHSSNNVIKT ECCFTDECNN ITLHLPTASP NAPKLGPMET GGGTHTCPPC |
| 101 | PAPELLGGPS VELFPPKPKD TLMISRTPEV TCVVVDVSHE DPEVKENWYV |
| 151 | DGVEVHNAKT KPREEQYNST YRVVSVLTVL HQDWLNGKEY KCKVSNKALP |
| 201 | APIEKTISKA KGQPREPQVC TLPPSREEMT KNQVSLSCAV KGFYPSDIAV |
| 251 | EWESNGQPEN NYKTTPPVLD SDGSFFLVSK LTVDKSRWQQ GNVESCSVMH |
| 301 | EALHNHYTQK SLSLSPGK |

The ActRIIB-Fc and ALK7-Fc proteins of SEQ ID NO: 402 and SEQ ID NO: 133, respectively, may be co-expressed and purified from a CHO cell line, to give rise to a heteromeric complex comprising ActRIIB-Fc:ALK7-Fc.

Purification of various ActRIIB-Fc:ALK7-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 15. Ligand Binding Profile of ActRIIB-Fc:ALK7-Fc Heterodimer Compared to ActRIIB-Fc Homodimer and ALK7-Fc Homodimer

A Biacore™-based binding assay was used to compare ligand binding selectivity of the ActRIIB-Fc:ALK7-Fc heterodimeric complex described above with that of ActRIIB-Fc and ALK7-Fc homodimeric complexes. The ActRIIB-Fc:ALK7-Fc heterodimer, ActRIIB-Fc homodimer, and ALK7-Fc homodimer were independently captured onto the system using an anti-Fc antibody. Ligands were injected and allowed to flow over the captured receptor protein. Results are summarized in the table below, in which ligand off-rates (k_{d}) most indicative of effective ligand traps are denoted by bold font.

Ligand binding profile of ActRIIB-Fc:ALK7-Fc heterodimer
compared to ActRIIB-Fc homodimer and ALK7-Fc homodimer

| | ActRIIB-Fc | | | ALK7-Fc | | | ActRIIB-Fc:ALK7-Fc | | |
|---|---|---|---|---|---|---|---|---|---|
| | homodimer | | | homodimer | | | heterodimer | | |
| | kₐ | k_{d} | K_{D} | kₐ | k_{d} | K_{D} | kₐ | k_{d} | K_{D} |
| Ligand | (1/Ms) | (1/s) | (pM) | (1/Ms) | (1/s) | (pM) | (1/Ms) | (1/s) | (pM) |

| activin A | 1.3 × 10⁷ | 1.4 × 10⁻⁴ | 11 | No binding | 4.4 × 10⁷ | 1.9 × 10⁻³ | 43 |
|---|---|---|---|---|---|---|---|
| activin B | 1.5 × 10⁷ | 1.6 × 10⁻⁴ | 8 | No binding | 1.2 × 10⁷ | 2.0 × 10⁻⁴ | 17 |
| activin C | No binding | | | No binding | 3.5 × 10⁵ | 2.4 × 10⁻³ | 6900 |
| activin AC | 2.0 × 10⁷ | 3.1 × 10⁻³ | 160 | No binding | 2.6 × 10⁶ | 5.7 × 10⁻⁴ | 220 |
| BMP5 | 2.6 × 10⁷ | 7.5 × 10⁻² | 2900 | No binding | 1.5 × 10⁵ | 8.5 × 10⁻³ | 57000 |
| BMP6 | 2.4 × 10⁷ | 3.9 × 10⁻³ | 160 | No binding | 1.2 × 10⁶ | 6.3 × 10⁻³ | 5300 |
| BMP9 | 1.2 × 10⁸ | 1.2 × 10⁻³ | 10 | No binding | Transient* | | >1400 |
| BMP10 | 5.9 × 10⁶ | 1.5 × 10⁻⁴ | 25 | No binding | 1.5 × 10⁷ | 2.8 × 10⁻³ | 190 |
| GDF3 | 1.4 × 10⁶ | 2.2 × 10⁻³ | 1500 | No binding | 2.3 × 10⁶ | 1.0 × 10⁻² | 4500 |
| GDF8 | 3.5 × 10⁶ | 2.4 × 10⁻⁴ | 69 | No binding | 3.7 × 10⁶ | 1.0 × 10⁻³ | 270 |
| GDF11 | 9.6 × 10⁷ | 1.5 × 10⁻⁴ | 2 | No binding | 9.5 × 10⁷ | 7.5 × 10⁻⁴ | 8 |

*Indeterminate due to transient nature of interaction
— Not tested

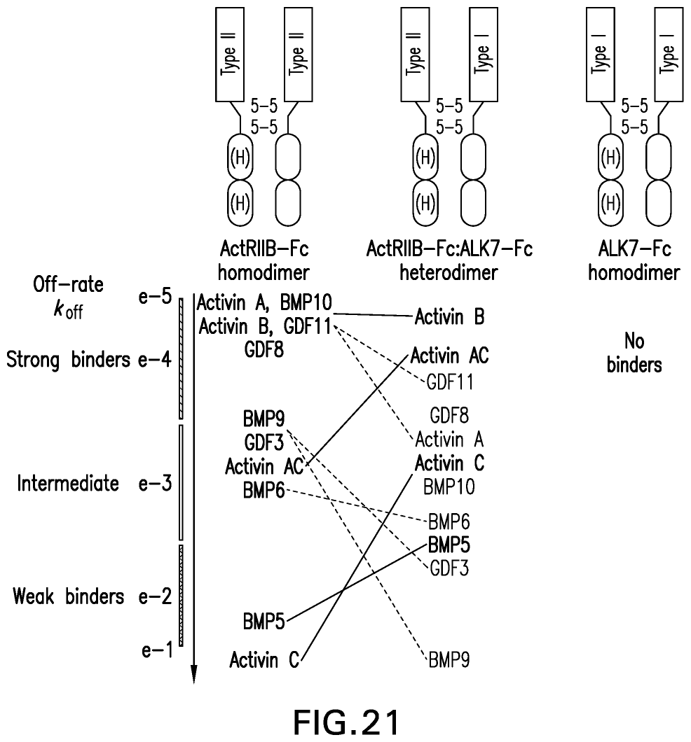

These comparative binding data demonstrate that the ActRIIB-Fc:ALK7-Fc heterodimer has an altered binding profile/selectivity relative to either the ActRIIB-Fc homodimer or ALK7-Fc homodimer. Interestingly, four of the five ligands with the strongest binding to ActRIIB-Fc homodimer (activin A, BMP10, GDF8, and GDF11) exhibit reduced binding to the ActRIIB-Fc:ALK7-Fc heterodimer, the exception being activin B which retains tight binding to the heterodimer. Similarly, three of the four ligands with intermediate binding to ActRIIB-Fc homodimer (GDF3, BMP6, and particularly BMP9) exhibit reduced binding to the ActRIIB-Fc:ALK7-Fc heterodimer, whereas binding to activin AC is increased to become the second strongest ligand interaction with the heterodimer overall. Finally, activin C and BMP5 unexpectedly bind the ActRIIB-Fc:ALK7 heterodimer with intermediate strength despite no binding (activin C) or weak binding (BMP5) to ActRIIB-Fc homodimer. The net result is that the ActRIIB-Fc:ALK7-Fc heterodimer possesses a ligand-binding profile distinctly different from that of either ActRIIB-Fc homodimer or ALK7-Fc homodimer, which binds none of the foregoing ligands. See FIG. 21.

These results therefore demonstrate that the ActRIIB-Fc:ALK7-Fc heterodimer is a more selective antagonist of activin B and activin AC compared to ActRIIB-Fc homodimer. Moreover, ActRIIB-Fc:ALK7-Fc heterodimer exhibits the unusual property of robust binding to activin C. Accordingly, an ActRIIB-Fc:ALK7-Fc heterodimer will be more useful than an ActRIIB-Fc homodimer in certain applications where such selective antagonism is advantageous. Examples include therapeutic applications where it is desirable to retain antagonism of activin B or activin AC but decrease antagonism of one or more of activin A, GDF3, GDF8, GDF11, BMP9, or BMP10. Also included are therapeutic, diagnostic, or analytic applications in which it is desirable to antagonize activin C or, based on the similarity between activin C and activin E, activin E.

### Example 16. The Role of ActRIIB-Fc:ALK4-Fc on Cardio-Protection in Heart Failure with Preserved Ejection Fraction (HFpEF) in Aging Mice

Effects of ActRIIB-Fc:ALK4-Fc on cardio-protection were examined in a murine model of physiological cardiac aging using aged C57BL6 mice (“Old”). “Old” mice show structural and functional changes that are similar to those observed in the senescent human heart (e.g., phenotypes of HFpEF), including LV diastolic dysfunction, and no reduction in ejection fraction (See, Merentie et al., 2015; Lucia et al., 2018; Roh et al., 2019; Mesquita et al., 2020). Studies using aged C57BL6 mice were conducted to assess if ActRIIB-Fc:ALK4-Fc was able to restore cardiac functional alterations under remodeling.

Thirteen male mice at 24-months of age (“Old”) and 10 mice at 4-months of age (“Young”) were studied. Groups of “Old” and “Young” mice received phosphate-buffered saline (PBS) twice per week subcutaneously for 8 weeks (“Young-Vehicle” or “Old-Vehicle”, respectively). Another group of “Old” mice received ActRIIB-Fc:ALK4-Fc (10 mg/kg) twice per week subcutaneously for 8 weeks (“Old-ActRIIB-Fc:ALK4-Fc”). The volume of vehicle and volume of ActRIIB-Fc:ALK4-Fc administered was the same.

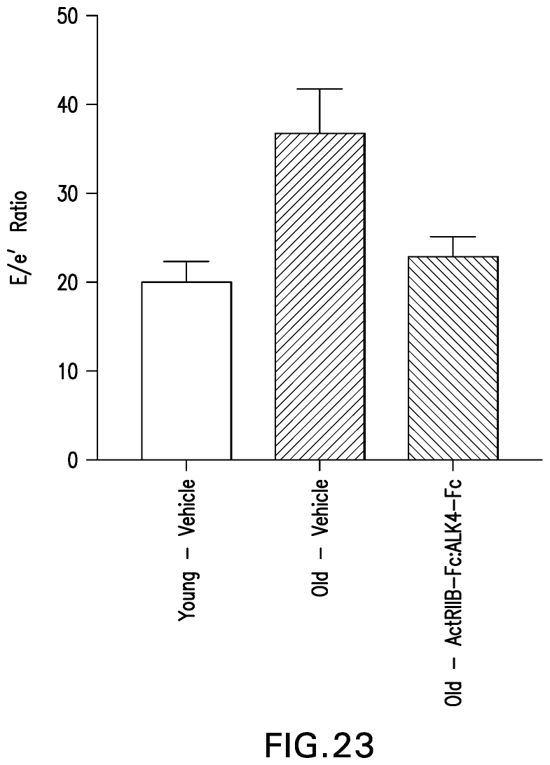

At the end of the study, before animals were euthanized, in vivo cardiac structure and function were assessed by transthoracic echocardiography (VisualSonics Vevo3100, 30 MHz transducer; Fujifilm) while mice were under anesthesia. Diastolic function was assessed by pulsed wave Doppler recordings of the maximal early (E) diastolic transmitral flow velocity and Doppler tissue imaging recordings of peak early (e′) transmitral valve annulus velocity in apical 4-chamber view. Changes in the ratio of peak transmitral flow velocity to peak transmitral valve annulus velocity (E/e′) was used to estimate diastolic function. (FIG. 23). Data are presented as mean±standard error of the mean. Statistical tests (one-way ANOVA with post-hoc analysis using Tukey's test for multiple comparisons) were performed, with a significance level set as p<0.05. In particular, *p<0.05, **p<0.01, ***p<0.001.

By the end of the study, “Old-Vehicle” mice displayed characteristic features of HFpEF, such as no reduction in ejection fraction (EF), and an increase in BNP levels compared to “Young-Vehicle” mice. ActRIIB-Fc:ALK4-Fc treatment presented a trend in no reduction of EF, and also a trend in reduction of BNP expression (“Old-ActRIIB-Fc:ALK4-Fc”). “Old-Vehicle” mice presented a trend of increased lung weight compared to “Young-Vehicle” mice, an indication of congestive lung in aged mice. Lung weight in “Old-ActRIIB-Fc:ALK4-Fc” mice presented a trend of reduction in lung weight compared to “Old-Vehicle” mice.

Cardiac remodeling (i.e., LV hypertrophy) in aged mice altered cardiac function, specifically diastolic function as measured by E/e′ (FIG. 23). “Old-Vehicle” mice presented an increased E/e′ compared to “Young-Vehicle” mice, which is an indicator of filling pressure in clinical practice and diastolic dysfunction (FIG. 23). Strikingly, the E/e′ ratio, a hallmark diastolic function measurement, was significantly decreased in “Old-ActRIIB-Fc:ALK4-Fc” mice compared to “Old-Vehicle” mice.

These data demonstrate that ActRIIB-Fc:ALK4-Fc is effective to reverse trends of diastolic dysfunction while not also reducing ejection fraction in a physiological cardiac aging model. In particular, E/e′ was significantly reduced in ActRIIB-Fc:ALK4-Fc treated mice compared to untreated aged mice, an indication that ActRIIB-Fc:ALK4-Fc helped to improve LV relaxation, a sign of diastolic dysfunction. The data further suggest that, in addition to ActRIIB:ALK4 heteromultimers, other ActRII-ALK4 antagonists may be useful in treating heart failure.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1. A method of treating heart failure associated with aging, comprising administering to a patient in need thereof an effective amount of an ActRII-ALK4 antagonist.

2. The method of claim 1, wherein the heart failure is heart failure associated with preserved ejection fraction (HFpEF).

3. The method of claim 1, wherein the patient has left ventricular (LV) hypertrophy, a diastolic dysfunction, or an elevated brain natriuretic peptide (BNP) level as compared to a healthy patient.

4. The method of claim 1, wherein the method decreases LV hypertrophy in the patient; increases ventricular relaxation and decreases filling pressures in the patient; improves the patient's diastolic dysfunction; or decreases BNP levels in the patient.

5. (canceled)

6. The method of claim 3, wherein the patient's ratio of early diastolic transmitral flow to early diastolic mitral annular tissue velocity (E/e′ ratio) is increased in comparison to healthy people of similar age and sex.

7-10. (canceled)

11. The method of claim 1, wherein the ActRII-ALK4 antagonist comprises an ActRIIA polypeptide.

12. The method of claim 11, wherein the ActRIIA polypeptide comprises an amino acid sequence that is at least 90% identical to an amino acid sequence that begins at any one of amino acids 21, 22, 23, 24, 25, 26, 27, 28, 29, or 30 of SEQ ID NO: 366 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 366.

13. The method of claim 11, wherein the ActRIIA polypeptide comprises an amino acid sequence that is at least 90% identical to an amino acid sequence of SEQ ID NO: 367 or SEQ ID NO: 368.

14. (canceled)

15. The method of claim 11, wherein the ActRIIA polypeptide is a fusion polypeptide comprising an ActRIIA polypeptide domain and one or more heterologous domains.

16. The method of claim 15, wherein the fusion polypeptide is an ActRIIA-Fc fusion polypeptide.

17. The method of claim 15, wherein the fusion polypeptide further comprises a linker domain positioned between the ActRIIA polypeptide domain and i) the one or more heterologous domains.

18. (canceled)

19. The method of claim 16, wherein the polypeptide comprises an amino acid sequence that is at least 90% identical to the amino acid sequence of SEQ ID NO: 380 or SEQ ID NO:378.

20-22. (canceled)

23. The method of claim 1, wherein the ActRII-ALK4 antagonist is a heteromultimer polypeptide.

24. The method of claim 23, wherein the heteromultimer polypeptide comprises an ActRIIB polypeptide, and an ALK4 polypeptide or an ALK7 polypeptide.

25. (canceled)

26. The method of claim 24, wherein the heteromultimer polypeptide comprises an ALK4 polypeptide comprising an amino acid sequence that is at least 90% identical to an amino acid sequence selected from the group consisting of SEQ ID NOs: 84, 85, 86, 87, 88, 89, 92, 93, 247, 249, 421, and 422.

27. The method of claim 24, wherein the heteromultimer polypeptide comprises an ALK7 polypeptide comprising an amino acid sequence that is at least 90% identical to an amino acid sequence selected from the group consisting of SEQ ID NOs: 120, 121, 122, 123, 124, 125, 126, 127, 128, 129, 130, 133, and 134.

28. The method of claim 24, wherein the heteromultimer polypeptide comprises an ALK4 polypeptide that is a fusion polypeptide comprising an ALK4 polypeptide domain and one or more heterologous domains.

29. The method of claim 24, wherein the heteromultimer polypeptide comprises an ALK7 polypeptide that is a fusion polypeptide comprising an ALK7 polypeptide domain and one or more heterologous domains.

30. The method of claim 28, wherein the fusion polypeptide is an ALK4-Fc fusion polypeptide, and wherein the ALK4-Fc fusion polypeptide optionally further comprises a linker domain positioned between the ALK4 polypeptide domain and the Fc domain.

31. The method of claim 29, wherein the fusion polypeptide is an ALK7-Fc fusion polypeptide, and wherein the ALK7-Fc fusion polypeptide further comprises a linker domain positioned between the ALK7 polypeptide domain and the Fc domain.

32-35. (canceled)

36. The method of claim 23, wherein the ActRII-ALK4 heteromultimer comprises an ActRIIB polypeptide.

37. The method of claim 36, wherein the ActRIIB polypeptide comprises an amino acid sequence that is at least 90% identical to an amino acid sequence that begins at any one of amino acid residues 20, 21, 22, 23, 24, 25, 26, 27, 28, or 29 of SEQ ID NO: 2 and ends at any one of amino acid residues 109, 110, 111, 112, 113, 114, 115, 116, 117, 118, 119, 120, 121, 122, 123, 124, 125, 126, 127, 128, 129, 130, 131, 132, 133, or 134 of SEQ ID NO: 2.

38. The method of claim 36, wherein the ActRIIB polypeptide comprises an amino acid sequence that is at least 90% identical to amino acids 29-109, 25-131, or 20-134 of SEQ ID NO: 2: or at least 90% identical to the amino acid sequence of SEQ ID NO: 53, SEQ ID NO: 388, or SEQ ID NO: 380.

39-43. (canceled)

44. The method of claim 36, wherein the ActRIIB polypeptide is a fusion polypeptide comprising an ActRIIB polypeptide domain and one or more heterologous domains.

45. The method of claim 44, wherein the fusion polypeptide is an ActRIIB-Fc fusion polypeptide, and wherein the fusion polypeptide optionally further comprises a linker domain positioned between the ActRIIB polypeptide domain and the Fc domain.

46-47. (canceled)

48. The method of claim 45, wherein the fusion polypeptide comprises an amino acid sequence that is at least 90% identical to the amino acid sequence of SEQ ID NO: 5 or SEQ ID NO: 12.

49. (canceled)

50. The method of claim 36, wherein the ActRIIB polypeptide comprises one or more amino acid substitutions with respect to the amino acid sequence of SEQ ID NO: 2 selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N.

51. The method of claim 36, wherein the ActRIIB polypeptide comprises one or more amino acid substitutions with respect to the amino acid sequence of SEQ ID NO: 2 selected from the group consisting of: L38N, E50L, E52D, E52N, E52Y, L57E, L57I, L57R, L57T, L57V, Y60D, G68R, K74E, W78Y, L79E, L79F, L79H, L79R, L79S, L79T, L79W, F82D, F82E, F82I, F82K, F82L, F82S, F82T, F82Y, N83R, E94K, and V99G.
